(19) United States
(12) Patent Application Publication
(10) Pub. No.: US 2024/0352016 A1
(43) Pub. Date: Oct. 24, 2024

(54) A CYCLIN-DEPENDENT KINASE INHIBITOR

(71) Applicant: TAIZHOU EOC PHARMA CO., LTD., Taizhou, Jiangsu (CN)

(72) Inventors: Lei Mo, Taizhou (CN); Junkai Huang, Taizhou (CN); Kai Li, Taizhou (CN); Li Zhou, Taizhou (CN); Xiaohui Hu, Taizhou (CN)

(21) Appl. No.: 18/682,476

(22) PCT Filed: Aug. 10, 2022
(86) PCT No.: PCT/CN2022/111324

§ 371 Date: Feb. 9, 2024

(30) Foreign Application Priority Data
Aug. 11, 2021 (WO) PCT/CN2021/111977

(51) Int. Cl. C07D 473/16; A61K 31/52; C07D 519/00

(52) U.S. Cl. CPC C07D 473/16; A61K 31/52; C07D 519/00

(57) ABSTRACT

Provided are compounds as potent inhibitors of cyclin-dependent kinase (CDK), or pharmaceutically acceptable salts thereof. Corresponding compositions are also provided.

### BACKGROUND OF THE INVENTION

Cyclin-dependent kinase (CDK) family members that trigger passage through the cell cycle are being considered as attractive therapeutic targets, especially for cancer. There is a continuing need for CDK inhibitors.

### SUMMARY OF THE INVENTION

The present disclosure provides a compound having the structure of formula (I)

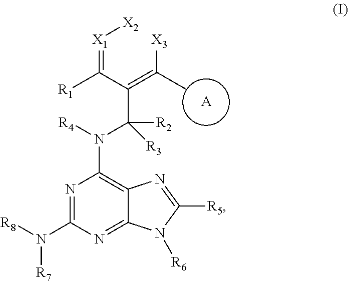

or a pharmaceutically acceptable salt, or prodrug thereof, or a solvate or hydrate of any of the forgoing,
wherein, each X₁, X₂, and X₃ is independently selected from optionally substituted —CH═, and —N═, each R₁, R₂, R₃, R₄, R₅, R₆, R₇, and R₈, is independently absent or is independently selected from optional substituents, or R₇ and R₈ combined with the atoms to which they are attached form an optionally substituted ring,
wherein, ring A is optionally substituted 5-ring-membered heteroaryl containing one or more N(nitrogen).

In some embodiment the compound having the structure of formula (I), said X₁ is optionally substituted —CH═.

In some embodiment the compound having the structure of formula (I), said X₂ is optionally substituted —CH═.

In some embodiment the compound having the structure of formula (I), said X₃ is optionally substituted —CH═ or —N═.

In some embodiment the compound having the structure of formula (I), said R₁ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₁ is selected from the group consisting of hydrogen, protium, deuterium, tritium, and halogen.

In some embodiment the compound having the structure of formula (I), said R₁ is H(hydrogen), F(fluorine) or Cl (chlorine).

In some embodiment the compound having the structure of formula (I), said R₂ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₂ is H.

In some embodiment the compound having the structure of formula (I), said R₃ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₃ is H.

In some embodiment the compound having the structure of formula (I), said R₄ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₄ is H.

In some embodiment the compound having the structure of formula (I), said R₅ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₅ is H.

In some embodiment the compound having the structure of formula (I), said R₆ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₆ is selected from the group consisting of hydrogen, protium, deuterium, tritium, optionally substituted (C₁-C₆)alkyl, and 3 to 12-ring-membered carbocycle.

In some embodiment the compound having the structure of formula (I), said R₆ is optionally substituted (C₁-C₆)alkyl.

In some embodiment the compound having the structure of formula (I), said R₆ is optionally substituted ethyl or optionally substituted propyl.

In some embodiment the compound having the structure of formula (I), said R₆ is optionally substituted isopropyl.

In some embodiment the compound having the structure of formula (I), said R₆ is optionally substituted 3 to 12-ring-membered carbocycle.

In some embodiment the compound having the structure of formula (I), said R₆ is optionally substituted 5-ring-membered carbocycle.

In some embodiment the compound having the structure of formula (I), said R₆ is optionally substituted cyclopentanyl.

In some embodiment the compound having the structure of formula (I), said R₇ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₇ is selected from the group consisting of hydrogen, protium, deuterium, tritium, optionally substituted (C₁-C₆)acyl, optionally substituted (C₁-C₆)alkyl, optionally substituted 3 to 12-ring-membered carbocycle, and optionally substituted 3 to 12-ring-membered heterocycle.

In some embodiment the compound having the structure of formula (I), said R₇ is optionally substituted (C₁-C₆)acyl.

In some embodiment the compound having the structure of formula (I), said R₇ is optionally substituted formyl.

In some embodiment the compound having the structure of formula (I), said R₇ is optionally substituted (C₁-C₆)alkyl.

In some embodiment the compound having the structure of formula (I), said R₇ is optionally substituted methyl or optionally substituted propyl.

In some embodiment the compound having the structure of formula (I), said R₇ is optionally substituted 3 to 12-ring-membered carbocycle.

In some embodiment the compound having the structure of formula (I), said R₇ is optionally substituted cyclohexyl.

In some embodiment the compound having the structure of formula (I), said R₇ is optionally substituted 3 to 12-ring-membered heterocycle.

In some embodiment the compound having the structure of formula (I), said R₇ is optionally substituted 6-ring-membered heterocycle.

In some embodiment the compound having the structure of formula (I), said R₇ is optionally substituted piperidinyl.

In some embodiment the compound having the structure of formula (I), said R₇ is optionally substituted 1-azabicyclo [2.2.2] octane.

In some embodiment the compound having the structure of formula (I), said R₇ is substituted with one or more R₇₋₁, each said R₇₋₁ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₇₋₁ is selected from the group consisting of hydrogen, protium, deuterium, tritium, optionally substituted hydroxy, optionally substituted amino, optionally substituted 3 to 12-ring-membered carbocycle, and optionally substituted 3 to 12-ring-membered heterocycle.

In some embodiment the compound having the structure of formula (I), said R₇₋₁ is optionally substituted hydroxy.

In some embodiment the compound having the structure of formula (I), said R₇₋₁ is optionally substituted amino.

In some embodiment the compound having the structure of formula (I), said R₇₋₁ is optionally substituted 3 to 12-ring-membered carbocycle.

In some embodiment the compound having the structure of formula (I), said R₇₋₁ is optionally substituted cyclohexyl.

In some embodiment the compound having the structure of formula (I), said R₇₋₁ is optionally substituted 3 to 12-ring-membered heterocycle.

In some embodiment the compound having the structure of formula (I), said R₇₋₁ is optionally substituted 6-ring-membered heterocycle.

In some embodiment the compound having the structure of formula (I), said R₇₋₁ is optionally substituted piperidinyl.

In some embodiment the compound having the structure of formula (I), said R₇₋₁ is substituted with one or more R₇₋₂, each said R₇₋₂ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R₇₋₂ is selected from the group consisting of hydrogen, protium, deuterium, tritium, optionally substituted hydroxy, and optionally substituted amino.

In some embodiment the compound having the structure of formula (I), said R₈ is H.

In some embodiment the compound having the structure of formula (I), said R₇ and said R₈ combined with the atoms to which they are attached form an optionally substituted ring B, said ring B is selected from the group consisting of optionally substituted 3 to 12-ring-membered heterocycle, and optionally substituted 5 to 12-ring-membered heteroaryl.

In some embodiment the compound having the structure of formula (I), said ring B is optionally substituted 3 to 12-ring-membered heterocycle.

In some embodiment the compound having the structure of formula (I), said ring B is optionally substituted 5 to 6-ring-membered heterocycle.

In some embodiment the compound having the structure of formula (I), said ring B is optionally substituted 5 to 6-ring-membered heterocycle containing one or two N.

In some embodiment the compound having the structure of formula (I), said ring B is optionally substituted piperidinyl, optionally substituted piperazinyl, or optionally substituted tetrahydropyrrolyl.

In some embodiment the compound having the structure of formula (I), said ring B is substituted with one or more R_{B-1}, each said R_{B-1} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{B-1} is selected from the group consisting of hydrogen, protium, deuterium, tritium, optionally substituted hydroxy, optionally substituted amino, optionally substituted (C₁-C₆)acyl, optionally substituted (C₁-C₆)alkyl, and optionally substituted 3 to 12-ring-membered heterocycle.

In some embodiment the compound having the structure of formula (I), said R_{B-1} is optionally substituted (C₁-C₆)acyl.

In some embodiment the compound having the structure of formula (I), said R_{B-1} is optionally substituted formyl.

In some embodiment the compound having the structure of formula (I), said R_{B-1} is optionally substituted (C₁-C₆)alkyl.

In some embodiment the compound having the structure of formula (I), said R_{B-1} is optionally substituted methyl.

In some embodiment the compound having the structure of formula (I), said R_{B-1} is optionally substituted 3 to 12-ring-membered heterocycle.

In some embodiment the compound having the structure of formula (I), said R_{B-1} is optionally substituted 6-ring-membered heterocycle.

In some embodiment the compound having the structure of formula (I), said R_{B-1} is optionally substituted piperidinyl.

In some embodiment the compound having the structure of formula (I), said R_{B-1} is substituted with one or more R_{B-2}, each said R_{B-2} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{B-2} is selected from the group consisting of hydrogen, protium, deuterium, tritium, optionally substituted hydroxy, optionally substituted amino, optionally substituted (C₁-C₆)alkyl, and optionally substituted 5 to 12-ring-membered heteroaryl.

In some embodiment the compound having the structure of formula (I), said R_{B-2} is optionally substituted (C₁-C₆)alkyl.

In some embodiment the compound having the structure of formula (I), said R_{B-2} is optionally substituted methyl, or optionally substituted ethyl.

In some embodiment the compound having the structure of formula (I), said R_{B-2} is optionally substituted 5-ring-membered heteroaryl.

In some embodiment the compound having the structure of formula (I), said R_{B-2} is optionally substituted 5-ring-membered heteroaryl containing two N.

In some embodiment the compound having the structure of formula (I), said R_{B-2} is optionally substituted pyrazolyl.

In some embodiment the compound having the structure of formula (I), said R_{B-2} is substituted with one or more R_{B-3}, each said R_{B-3} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{B-3} is selected from the group consisting of hydrogen, protium, deuterium, tritium, optionally substituted amino, and optionally substituted (C₁-C₆)alkyl.

In some embodiment the compound having the structure of formula (I), said R_{B-3} is optionally substituted (C₁-C₆)alkyl.

In some embodiment the compound having the structure of formula (I), said R_{B-3} is optionally substituted methyl.

In some embodiment the compound having the structure of formula (I), said R_{B-3} is substituted with one or more R_{B-4}, each said R_{B-4} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{B-4} is selected from the group consisting of hydrogen, protium, deuterium, tritium, and halogen.

In some embodiment the compound having the structure of formula (I), said R_{B-4} is F.

In some embodiment the compound having the structure of formula (I), said ring A is optionally substituted 5-ring-membered heteroaryl containing two N.

In some embodiment the compound having the structure of formula (I), said ring A is optionally substituted pyrazolyl.

In some embodiment the compound having the structure of formula (I), said ring A is substituted with one or more R_{A-1}, each said R_{A-1} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{A-1} is selected from the group consisting of hydrogen, protium, deuterium, tritium, optionally substituted (C₁-C₆)acyl, optionally substituted (C₁-C₆)alkyl, and optionally substituted 3 to 12-ring-membered heterocycle.

In some embodiment the compound having the structure of formula (I), said R_{A-1} is optionally substituted (C₁-C₆)acyl.

In some embodiment the compound having the structure of formula (I), said R_{A-1} is optionally substituted formyl.

In some embodiment the compound having the structure of formula (I), said R_{A-1} is optionally substituted (C₁-C₆)alkyl.

In some embodiment the compound having the structure of formula (I), said R_{A-1} is optionally substituted methyl, or optionally substituted propyl.

In some embodiment the compound having the structure of formula (I), said R_{A-1} is optionally substituted isopropyl.

In some embodiment the compound having the structure of formula (I), said R_{A-1} is optionally substituted 6-ring-membered heterocycle.

In some embodiment the compound having the structure of formula (I), said R_{A-1} is optionally substituted 6-ring-membered heterocycle containing two N.

In some embodiment the compound having the structure of formula (I), said R_{A-1} is optionally substituted piperazinyl.

In some embodiment the compound having the structure of formula (I), said R_{A-1} is substituted with one or more R_{A-2}, each said R_{A-2} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{A-2} is selected from the group consisting of hydrogen, protium, deuterium, tritium, optionally substituted hydroxy, optionally substituted (C₁-C₆)alkyl, and optionally substituted 3 to 12-ring-membered heterocycle.

In some embodiment the compound having the structure of formula (I), said R_{A-2} is optionally substituted (C₁-C₆)alkyl.

In some embodiment the compound having the structure of formula (I), said R_{A-2} is optionally substituted propyl.

In some embodiment the compound having the structure of formula (I), said R_{A-2} is optionally substituted isopropyl.

In some embodiment the compound having the structure of formula (I), said R_{A-2} is optionally substituted 6-ring-membered heterocycle.

In some embodiment the compound having the structure of formula (I), said R_{A-1} is optionally substituted 6-ring-membered heterocycle containing two N.

In some embodiment the compound having the structure of formula (I), said R_{A-2} is optionally substituted piperazinyl.

In some embodiment the compound having the structure of formula (I), said R_{A-2} is substituted with one or more R_{A-3}, each said R_{A-3} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{A-3} is selected from the group consisting of hydrogen, protium, deuterium, tritium, and optionally substituted (C₁-C₆)alkyl.

In some embodiment the compound having the structure of formula (I), said R_{A-3} is optionally substituted (C₁-C₆)alkyl.

In some embodiment the compound having the structure of formula (I), said R_{A-3} is optionally substituted methyl or optionally substituted ethyl.

In some embodiment the compound having the structure of formula (I), said R_{A-3} is substituted with one or more R_{A-4}, each said R_{A-4} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I), said R_{A-4} is selected from the group consisting of hydrogen, protium, deuterium, tritium, and optionally substituted hydroxy.

The present disclosure provides a compound or a pharmaceutically acceptable salt, prodrug, or metabolite thereof, or a solvate or hydrate of any of the foregoing, wherein, said compound is selected from the group consisting of

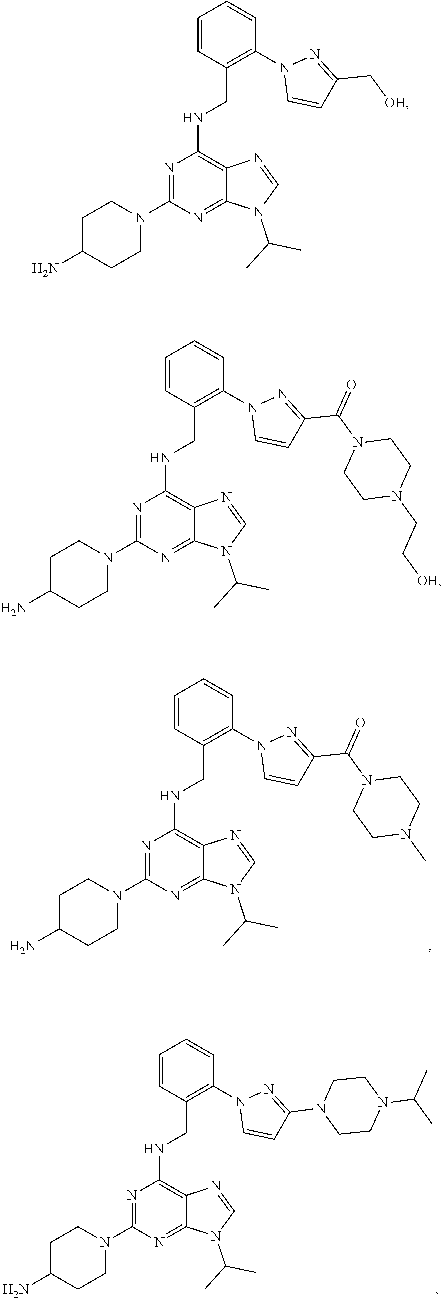 [SMILES: CC(C)n1cnc2c(NCc3ccccc3-n3ccc(CO)n3)nc(N3CCC(N)CC3)nc21 ; CC(C)n1cnc2c(NCc3ccccc3-n3ccc(C(=O)N4CCN(CCO)CC4)n3)nc(N3CCC(N)CC3)nc21 ; CC(C)n1cnc2c(NCc3ccccc3-n3ccc(C(=O)N4CCN(C)CC4)n3)nc(N3CCC(N)CC3)nc21 ; CC(C)N1CCN(c2ccn(-c3ccccc3CNc3nc(N4CCC(N)CC4)nc4c3ncn4C(C)C)n2)CC1]

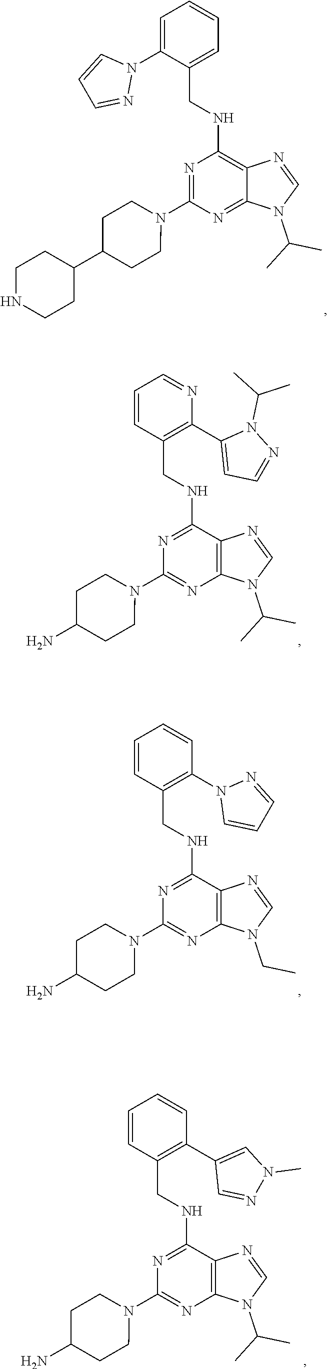 [SMILES: CC(C)n1cnc2c(NCc3ccccc3-n3cccn3)nc(N3CCC(C4CCNCC4)CC3)nc21 ; CC(C)n1nccc1-c1ncccc1CNc1nc(N2CCC(N)CC2)nc2c1ncn2C(C)C ; CCn1cnc2c(NCc3ccccc3-n3cccn3)nc(N3CCC(N)CC3)nc21 ; CC(C)n1cnc2c(NCc3ccccc3-c3cnn(C)c3)nc(N3CCC(N)CC3)nc21]

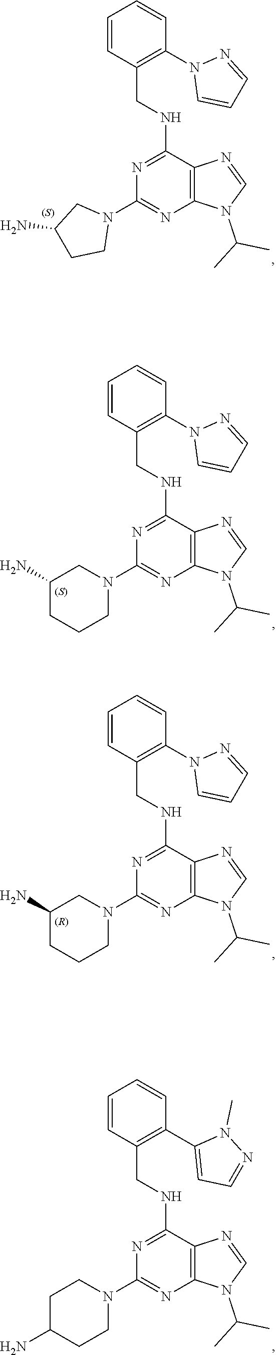

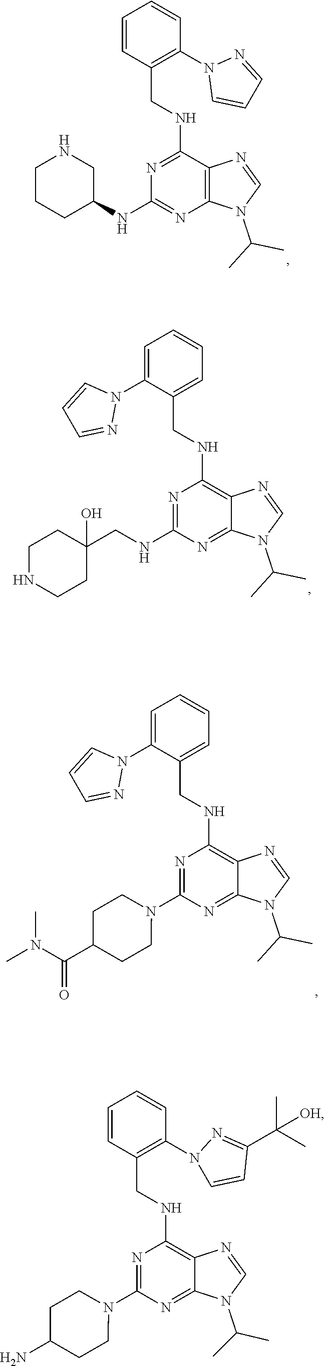

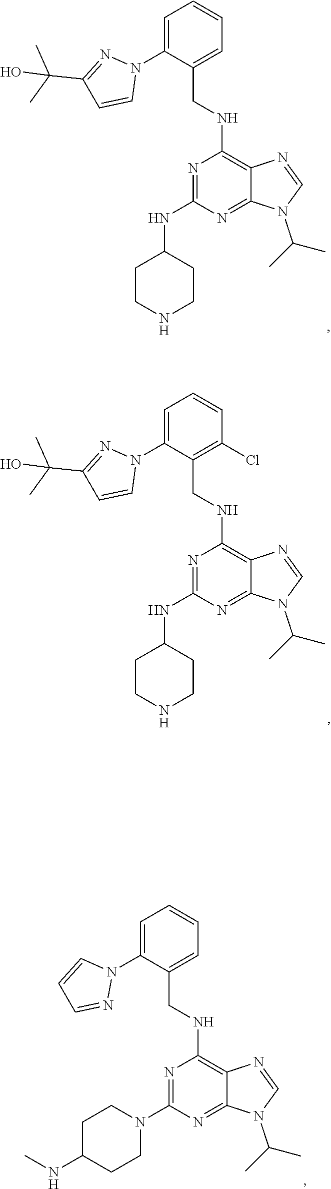

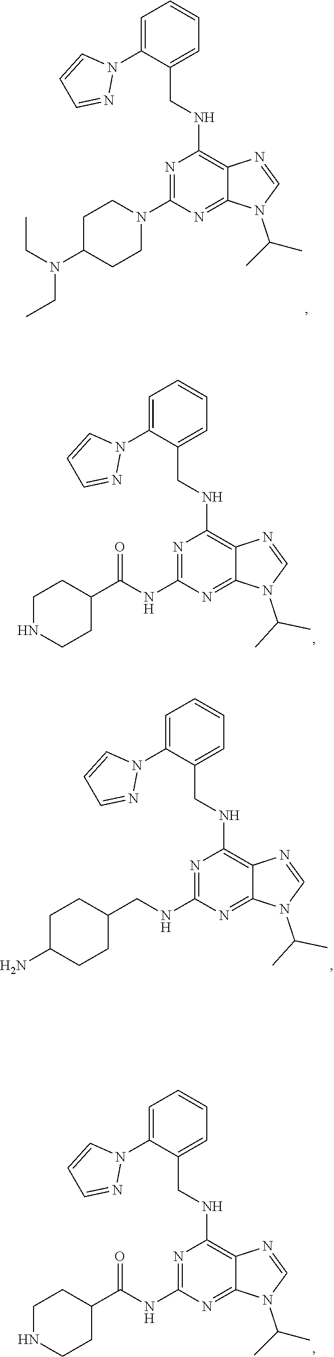 [SMILES: CCN(CC)C1CCN(c2nc(NCc3ccccc3-n3cccn3)c3ncn(C(C)C)c3n2)CC1 ; CC(C)n1cnc2c(NCc3ccccc3-n3cccn3)nc(NC(=O)C3CCNCC3)nc21 ; CC(C)n1cnc2c(NCc3ccccc3-n3cccn3)nc(NCC3CCC(N)CC3)nc21 ; CC(C)n1cnc2c(NCc3ccccc3-n3cccn3)nc(NC(=O)C3CCNCC3)nc21]

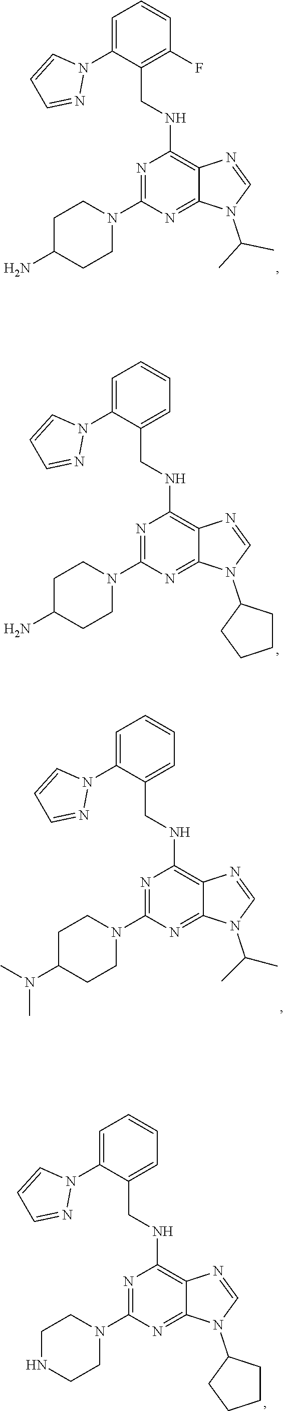

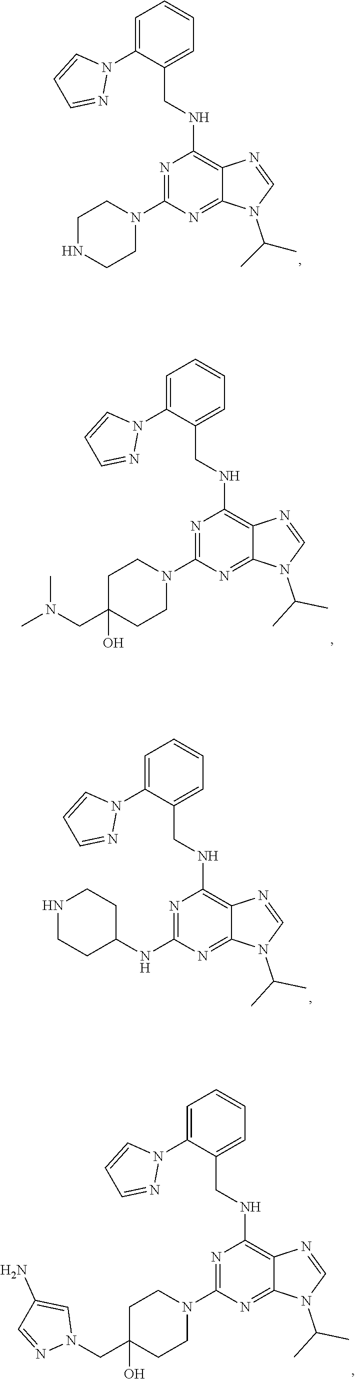

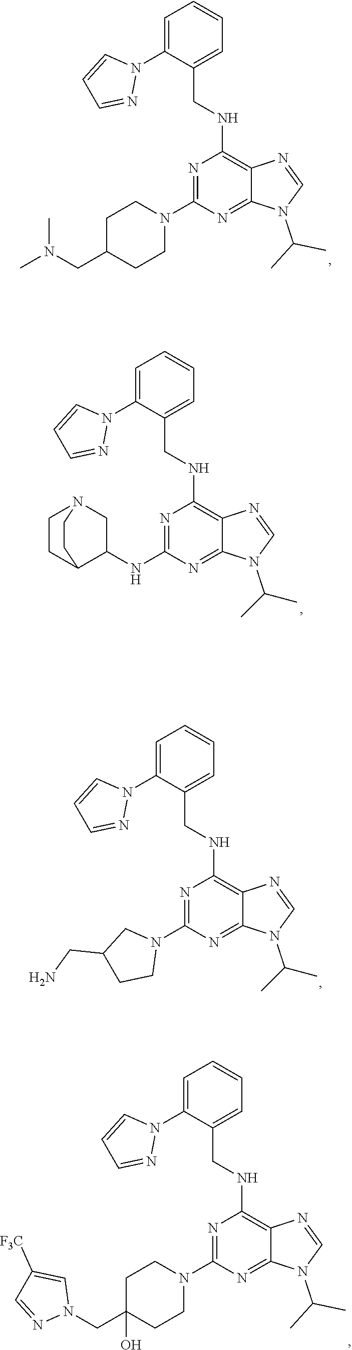 [SMILES: CC(C)n1cnc2c(NCc3ccccc3-n3cccn3)nc(N3CCC(CN(C)C)CC3)nc21 ; CC(C)n1cnc2c(NCc3ccccc3-n3cccn3)nc(NC3CN4CCC3CC4)nc21 ; CC(C)n1cnc2c(NCc3ccccc3-n3cccn3)nc(N3CCC(CN)C3)nc21 ; CC(C)n1cnc2c(NCc3ccccc3-n3cccn3)nc(N3CCC(O)(Cn4cc(C(F)(F)F)cn4)CC3)nc21]

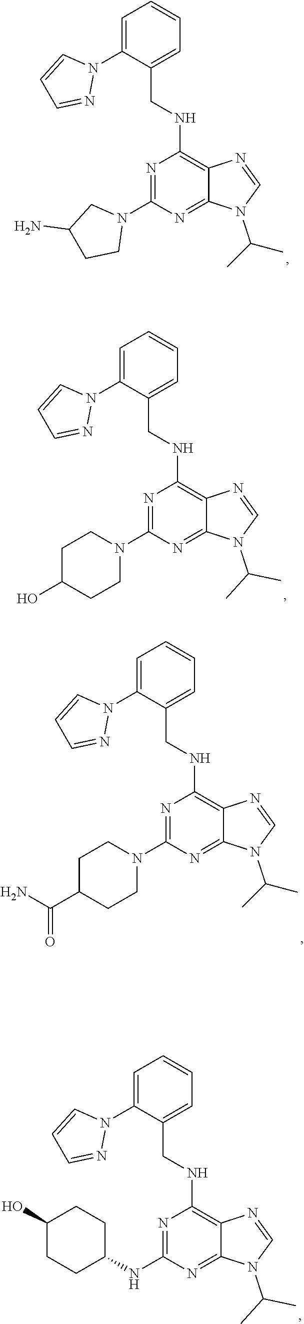 [SMILES: CC(C)n1cnc2c(NCc3ccccc3-n3cccn3)nc(N3CCC(N)C3)nc21 ; CC(C)n1cnc2c(NCc3ccccc3-n3cccn3)nc(N3CCC(O)CC3)nc21 ; CC(C)n1cnc2c(NCc3ccccc3-n3cccn3)nc(N3CCC(C(N)=O)CC3)nc21 ; CC(C)n1cnc2c(NCc3ccccc3-n3cccn3)nc(N[C@H]3CC[C@H](O)CC3)nc21]

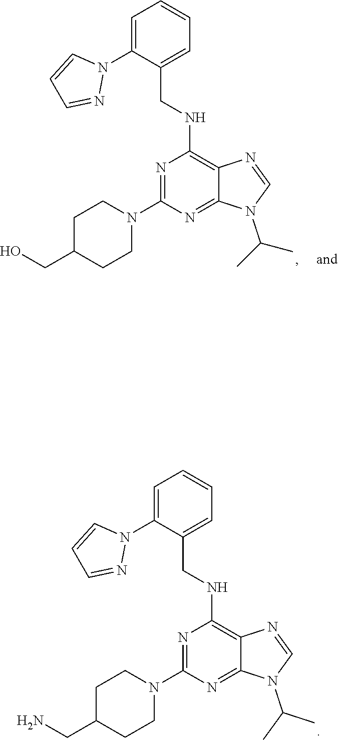 [SMILES: CC(C)n1cnc2c(NCc3ccccc3-n3cccn3)nc(N3CCC(CO)CC3)nc21 ; CC(C)n1cnc2c(NCc3ccccc3-n3cccn3)nc(N3CCC(CN)CC3)nc21]

The present disclosure provides a compound having the structure of formula (I-A)

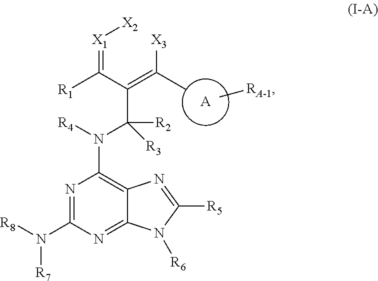

or a pharmaceutically acceptable salt, or prodrug thereof, or a solvate or hydrate of any of the forgoing,
wherein, each X₁, X₂, and X₃ is independently selected from optionally substituted —CH═, and —N═, each R₁, R₂, R₃, R₄, R₅, R₆, R₇, and R₈, is independently absent or is independently selected from optional substituents, or R₇ and R₈ combined with the atoms to which they are attached form an optionally substituted ring,
wherein, ring A is 5-ring-membered heteroaryl containing one or more N(nitrogen), said ring A is substituted with R_{A-1}, said R_{A-1} is optionally substituted alkyl, said ring A is further optionally substituted with optional substituents.

In some embodiment the compound having the structure of formula (I-A), said X₁ is optionally substituted —CH═.

In some embodiment the compound having the structure of formula (I-A), said X₂ is optionally substituted —CH═.

In some embodiment the compound having the structure of formula (I-A), said X₃ is optionally substituted —CH═ or —N═.

In some embodiment the compound having the structure of formula (I-A), said R₁ is H(hydrogen), or Cl (chlorine).

In some embodiment the compound having the structure of formula (I-A), said R₂ is H.

In some embodiment the compound having the structure of formula (I-A), said R₃ is H.

In some embodiment the compound having the structure of formula (I-A), said R₄ is H.

In some embodiment the compound having the structure of formula (I-A), said R₅ is H.

In some embodiment the compound having the structure of formula (I-A), said R₆ is optionally substituted (C₁-C₆)alkyl.

In some embodiment the compound having the structure of formula (I-A), said R₆ is optionally substituted propyl.

In some embodiment the compound having the structure of formula (I-A), said R₆ is optionally substituted isopropyl.

In some embodiment the compound having the structure of formula (I-A), said R₇ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (I-A), said R₇ is selected from the group consisting of hydrogen, protium, deuterium, tritium, and optionally substituted 3 to 12-ring-membered heterocycle.

In some embodiment the compound having the structure of formula (I-A), said R₇ is optionally substituted 6-ring-membered heterocycle.

In some embodiment the compound having the structure of formula (I-A), said R₇ is optionally substituted piperidinyl.

In some embodiment the compound having the structure of formula (I-A), said R₈ is H.

In some embodiment the compound having the structure of formula (I-A), said R₇ and said R₈ combined with the atoms to which they are attached form an optionally substituted ring B, said ring B is optionally substituted 3 to 12-ring-membered heterocycle.

In some embodiment the compound having the structure of formula (I-A), said ring B is optionally substituted 6-ring-membered heterocycle.

In some embodiment the compound having the structure of formula (I-A), said ring B is optionally substituted piperidinyl.

In some embodiment the compound having the structure of formula (I-A), said ring B is substituted with one or more R_{B-1}, each said R_{B-1} is independently optional substituent.

In some embodiment the compound having the structure of formula (I-A), said R_{B-1} is selected from the group consisting of hydrogen, protium, deuterium, tritium, optionally substituted hydroxy, and optionally substituted amino.

In some embodiment the compound having the structure of formula (I-A), said ring A is optionally substituted 5-ring-membered heteroaryl containing two N.

In some embodiment the compound having the structure of formula (I-A), said ring A is optionally substituted pyrazolyl.

In some embodiment the compound having the structure of formula (I-A), said R_{A-1} is optionally substituted (C₁-C₆)alkyl.

In some embodiment the compound having the structure of formula (I-A), said R_{A-1} is optionally substituted methyl, or optionally substituted propyl.

In some embodiment the compound having the structure of formula (I-A), said R_{A-1} is optionally substituted isopropyl.

In some embodiment the compound having the structure of formula (I-A), said R_{A-1} is substituted with one or more R_{A-2}, each said R_{A-2} is independently optional substituent.

In some embodiment the compound having the structure of formula (I-A), said R_{A-2} is selected from the group consisting of hydrogen, protium, deuterium, tritium, ═O, optionally substituted hydroxy, and optionally substituted 3 to 12-ring-membered heterocycle.

In some embodiment the compound having the structure of formula (I-A), said R_{A-2} is optionally substituted 6-ring-membered heterocycle.

In some embodiment the compound having the structure of formula (I-A), said R_{A-2} is optionally substituted 6-ring-membered heterocycle containing two N.

In some embodiment the compound having the structure of formula (I-A), said R_{A-2} is optionally substituted piperazinyl.

In some embodiment the compound having the structure of formula (I-A), said R_{A-2} is substituted with one or more R_{A-3}, each said R_{A-3} is independently optional substituent.

In some embodiment the compound having the structure of formula (I-A), said R_{A-3} is selected from the group consisting of hydrogen, protium, deuterium, tritium, and optionally substituted (C₁-C₆)alkyl.

In some embodiment the compound having the structure of formula (I-A), said R_{A-3} is optionally substituted (C₁-C₆)alkyl.

In some embodiment the compound having the structure of formula (I-A), said R_{A-3} is optionally substituted methyl or optionally substituted ethyl.

In some embodiment the compound having the structure of formula (I-A), said R_{A-3} is substituted with one or more R_{A-4}, each said R_{A-4} is independently optional substituent.

In some embodiment the compound having the structure of formula (I-A), said R_{A-4} is selected from the group consisting of hydrogen, protium, deuterium, tritium, and optionally substituted hydroxy.

The present disclosure provides a compound or a pharmaceutically acceptable salt, prodrug, or metabolite thereof, or a solvate or hydrate of any of the foregoing, wherein, said compound is selected from the group consisting of:

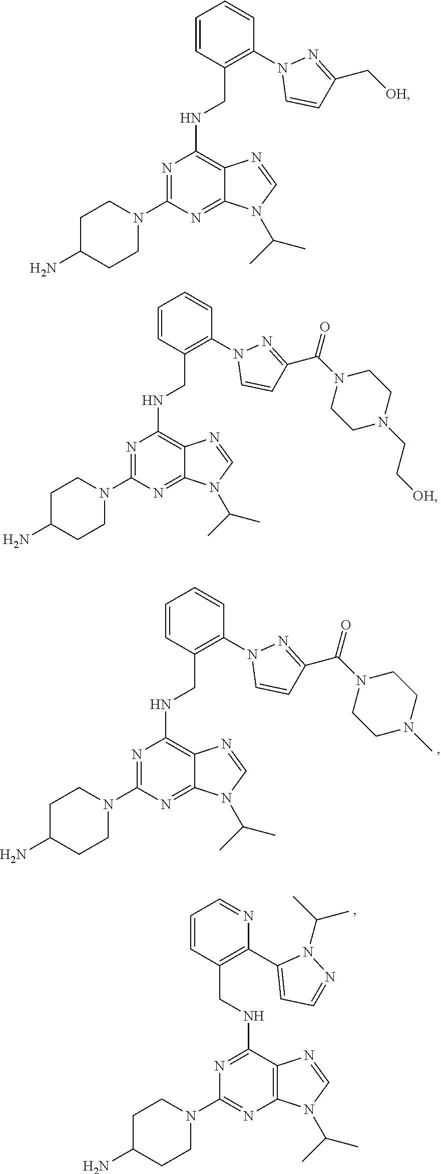 [SMILES: CC(C)n1cnc2c(NCc3ccccc3-n3ccc(CO)n3)nc(N3CCC(N)CC3)nc21 ; CC(C)n1cnc2c(NCc3ccccc3-n3ccc(C(=O)N4CCN(CCO)CC4)n3)nc(N3CCC(N)CC3)nc21 ; CC(C)n1cnc2c(NCc3ccccc3-n3ccc(C(=O)N4CCN(C)CC4)n3)nc(N3CCC(N)CC3)nc21 ; CC(C)n1nccc1-c1ncccc1CNc1nc(N2CCC(N)CC2)nc2c1ncn2C(C)C]

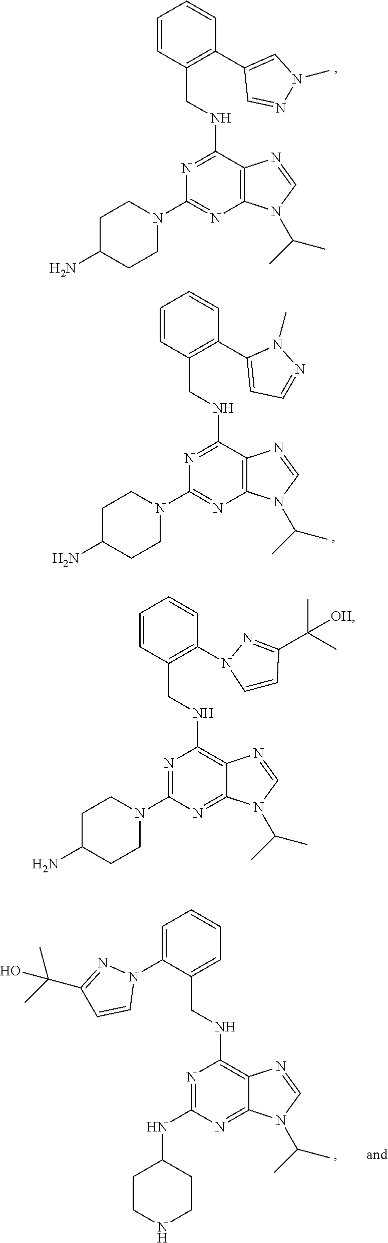 [SMILES: CC(C)n1cnc2c(NCc3ccccc3-c3cnn(C)c3)nc(N3CCC(N)CC3)nc21 ; CC(C)n1cnc2c(NCc3ccccc3-c3ccnn3C)nc(N3CCC(N)CC3)nc21 ; CC(C)n1cnc2c(NCc3ccccc3-n3ccc(C(C)(C)O)n3)nc(N3CCC(N)CC3)nc21 ; CC(C)n1cnc2c(NCc3ccccc3-n3ccc(C(C)(C)O)n3)nc(NC3CCNCC3)nc21]

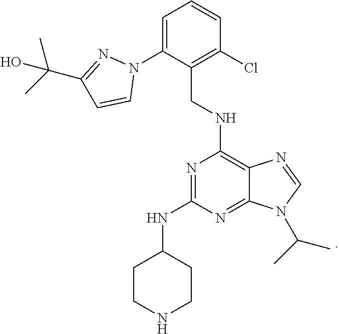 [SMILES: CC(C)n1cnc2c(NCc3c(Cl)cccc3-n3ccc(C(C)(C)O)n3)nc(NC3CCNCC3)nc21]

The present disclosure provides a compound having the structure of formula (I-B)

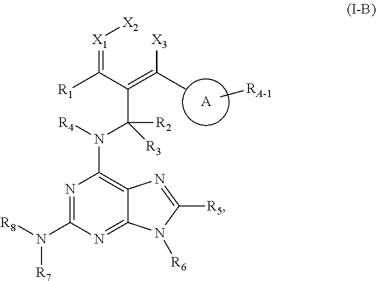

or a pharmaceutically acceptable salt, or prodrug thereof, or a solvate or hydrate of any of the forgoing,
wherein, each X₁, X₂, and X₃ is independently selected from optionally substituted —CH═, and —N═, each R₁, R₂, R₃, R₄, R₅, R₆, R₇, and R₈, is independently absent or is independently selected from optional substituents, or R₇ and R₈ combined with the atoms to which they are attached form an optionally substituted ring,
wherein, ring A is 5-ring-membered heteroaryl containing one or more N(nitrogen), said ring A is substituted with R_{A-1}, said R_{A-1} is optionally substituted ring, said ring A is further optionally substituted with optional substituents.

In some embodiment the compound having the structure of formula (I-B), said X₁ is optionally substituted —CH═.

In some embodiment the compound having the structure of formula (I-B), said X₂ is optionally substituted —CH═.

In some embodiment the compound having the structure of formula (I-B), said X₃ is optionally substituted —CH═.

In some embodiment the compound having the structure of formula (I-B), said R₁ is H(hydrogen).

In some embodiment the compound having the structure of formula (I-B), said R₂ is H.

In some embodiment the compound having the structure of formula (I-B), said R₃ is H.

In some embodiment the compound having the structure of formula (I-B), said R₄ is H.

In some embodiment the compound having the structure of formula (I-B), said R₅ is H.

In some embodiment the compound having the structure of formula (I-B), said R₆ is optionally substituted (C₁-C₆)alkyl.

In some embodiment the compound having the structure of formula (I-B), said R₆ is optionally substituted propyl.

In some embodiment the compound having the structure of formula (I-B), said R₆ is optionally substituted isopropyl.

In some embodiment the compound having the structure of formula (I-B), said R₇ and said R₈ combined with the atoms to which they are attached form an optionally substituted ring B, said ring B is optionally substituted 3 to 12-ring-membered heterocycle.

In some embodiment the compound having the structure of formula (I-B), said ring B is optionally substituted 6-ring-membered heterocycle.

In some embodiment the compound having the structure of formula (I-B), said ring B is optionally substituted piperidinyl.

In some embodiment the compound having the structure of formula (I-B), said ring B is substituted with one or more R_{B-1}, each said R_{B-1} is optional substituent.

In some embodiment the compound having the structure of formula (I-B), said R_{B-1} is selected from the group consisting of hydrogen, protium, deuterium, tritium, and optionally substituted amino.

In some embodiment the compound having the structure of formula (I-B), said ring A is optionally substituted 5-ring-membered heteroaryl containing two N.

In some embodiment the compound having the structure of formula (I-B), said ring A is optionally substituted pyrazolyl.

In some embodiment the compound having the structure of formula (I-B), said R_{A-1} is optionally substituted 3 to 12-ring-membered heterocycle.

In some embodiment the compound having the structure of formula (I-B), said R_{A-1} is optionally substituted 6-ring-membered heterocycle.

In some embodiment the compound having the structure of formula (I-B), said R_{A-1} is optionally substituted 6-ring-membered heterocycle containing two N.

In some embodiment the compound having the structure of formula (I-B), said R_{A-1} is optionally substituted piperazinyl.

In some embodiment the compound having the structure of formula (I-B), said R_{A-1} is substituted with one or more R_{A-2}, each said R_{A-2} is independently optional substituent.

In some embodiment the compound having the structure of formula (I-B), said R_{A-2} is optionally substituted (C₁-C₆)alkyl.

In some embodiment the compound having the structure of formula (I-B), said R_{A-2} is optionally substituted propyl.

In some embodiment the compound having the structure of formula (I-B), said R_{A-2} is optionally substituted isopropyl.

The present disclosure provides a compound or a pharmaceutically acceptable salt, prodrug, or metabolite thereof, or a solvate or hydrate of any of the foregoing, wherein, said compound is

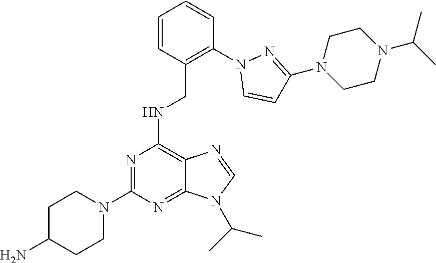 [SMILES: CC(C)N1CCN(c2ccn(-c3ccccc3CNc3nc(N4CCC(N)CC4)nc4c3ncn4C(C)C)n2)CC1]

The present disclosure provides a compound having the structure of formula (I-C)

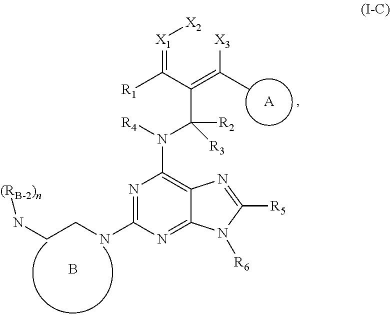

or a pharmaceutically acceptable salt, or prodrug thereof, or a solvate or hydrate of any of the forgoing,
wherein, each X₁, X₂, and X₃ is independently selected from optionally substituted —CH═, and —N═, each R₁, R₂, R₃, R₄, R₅, R₆, and R_{B-2}, is independently absent or is independently selected from optional substituents, n is 0 or more,
wherein, ring A is 5-ring-membered heteroaryl containing one or more N(nitrogen), said ring B is 5 to 6-ring-membered heterocycle.

In some embodiment the compound having the structure of formula (I-C), said X₁ is optionally substituted —CH═.

In some embodiment the compound having the structure of formula (I-C), said X₂ is optionally substituted —CH═.

In some embodiment the compound having the structure of formula (I-C), said X₃ is optionally substituted —CH═.

In some embodiment the compound having the structure of formula (I-C), said R₁ is H(hydrogen).

In some embodiment the compound having the structure of formula (I-C), said R₂ is H.

In some embodiment the compound having the structure of formula (I-C), said R₃ is H.

In some embodiment the compound having the structure of formula (I-C), said R₄ is H.

In some embodiment the compound having the structure of formula (I-C), said R₅ is H.

In some embodiment the compound having the structure of formula (I-C), said R₆ is optionally substituted (C₁-C₆)alkyl.

In some embodiment the compound having the structure of formula (I-C), said R₆ is optionally substituted propyl.

In some embodiment the compound having the structure of formula (I-C), said R₆ is optionally substituted isopropyl.

In some embodiment the compound having the structure of formula (I-C), said ring B is optionally substituted piperidinyl or optionally substituted tetrahydropyrrolyl.

In some embodiment the compound having the structure of formula (I-C), said R_{B-2} is H.

In some embodiment the compound having the structure of formula (I-C), said n is 2.

In some embodiment the compound having the structure of formula (I-C), said ring A is optionally substituted 5-ring-membered heteroaryl containing two N.

In some embodiment the compound having the structure of formula (I-C), said ring A is optionally substituted pyrazolyl.

The present disclosure provides a compound or a pharmaceutically acceptable salt, prodrug, or metabolite thereof, or a solvate or hydrate of any of the foregoing, wherein, said compound is

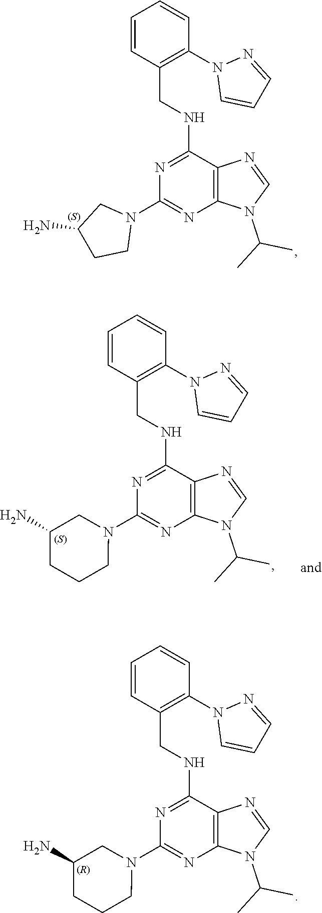

The present disclosure provides a compound having the structure of formula (I-D)

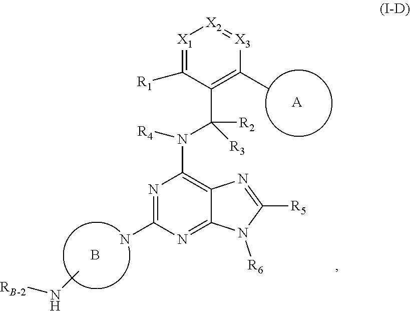

or a pharmaceutically acceptable salt, or prodrug thereof, or a solvate or hydrate of any of the forgoing,
wherein, each X₁, X₂, and X₃ is independently selected from optionally substituted —CH═, and —N═, each R₁, R₂, R₃, R₄, R₅, and R₆, is independently absent or is independently selected from optional substituents,
wherein, ring A is 5-ring-membered heteroaryl containing one or more N(nitrogen),
said R_{B-2} is optionally substituted (C₁-C₆)alkyl.

In some embodiment the compound having the structure of formula (I-D), said X₁ is optionally substituted —CH═.

In some embodiment the compound having the structure of formula (I-D), said X₂ is optionally substituted —CH═.

In some embodiment the compound having the structure of formula (I-D), said X₃ is optionally substituted —CH═.

In some embodiment the compound having the structure of formula (I-D), said R₁ is H(hydrogen).

In some embodiment the compound having the structure of formula (I-D), said R₂ is H.

In some embodiment the compound having the structure of formula (I-D), said R₃ is H.

In some embodiment the compound having the structure of formula (I-D), said R₄ is H.

In some embodiment the compound having the structure of formula (I-D), said R₅ is H.

In some embodiment the compound having the structure of formula (I-D), said R₆ is optionally substituted (C₁-C₆)alkyl.

In some embodiment the compound having the structure of formula (I-D), said R₆ is optionally substituted propyl.

In some embodiment the compound having the structure of formula (I-D), said R₆ is optionally substituted isopropyl.

In some embodiment the compound having the structure of formula (I-D), said ring B is optionally substituted piperidinyl.

In some embodiment the compound having the structure of formula (I-D), said R_{B-2} is optionally substituted methyl.

In some embodiment the compound having the structure of formula (I-D), said ring A is optionally substituted 5-ring-membered heteroaryl containing two N.

In some embodiment the compound having the structure of formula (I-D), said ring A is optionally substituted pyrazolyl.

The present disclosure provides a compound or a pharmaceutically acceptable salt, prodrug, or metabolite thereof, or a solvate or hydrate of any of the foregoing, wherein, said compound is:

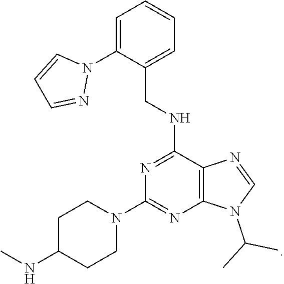 [SMILES: CNC1CCN(c2nc(NCc3ccccc3-n3cccn3)c3ncn(C(C)C)c3n2)CC1]

The present disclosure provides a compound having the structure of formula (I-E)

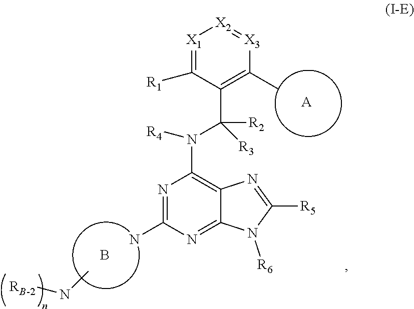

or a pharmaceutically acceptable salt, or prodrug thereof, or a solvate or hydrate of any of the forgoing,
wherein, each X₁, X₂, and X₃ is independently selected from optionally substituted —CH═, and —N═, each R₁, R₂, R₃, R₄, R₅, R₆, and R_{B-2} is independently absent or is independently selected from optional substituents, n is 0 or more,
wherein, ring A is 5-ring-membered heteroaryl containing one or more N(nitrogen), one of said R_{B-2} is optionally substituted (C₂-C₆)alkyl.

In some embodiment the compound having the structure of formula (I-E), said X₁ is optionally substituted —CH═.

In some embodiment the compound having the structure of formula (I-E), said X₂ is optionally substituted —CH═.

In some embodiment the compound having the structure of formula (I-E), said X₃ is optionally substituted —CH═.

In some embodiment the compound having the structure of formula (I-E), said R₁ is H(hydrogen).

In some embodiment the compound having the structure of formula (I-E), said R₂ is H.

In some embodiment the compound having the structure of formula (I-E), said R₃ is H.

In some embodiment the compound having the structure of formula (I-E), said R₄ is H.

In some embodiment the compound having the structure of formula (I-E), said R₅ is H.

In some embodiment the compound having the structure of formula (I-E), said R₆ is optionally substituted (C₁-C₆)alkyl.

In some embodiment the compound having the structure of formula (I-E), said R₆ is optionally substituted propyl.

In some embodiment the compound having the structure of formula (I-E), said R₆ is optionally substituted isopropyl.

In some embodiment the compound having the structure of formula (I-E), said ring B is optionally substituted piperidinyl.

In some embodiment the compound having the structure of formula (I-E), said R_{B-2} is optionally substituted ethyl.

In some embodiment the compound having the structure of formula (I-E), said n is 2.

In some embodiment the compound having the structure of formula (I-E), said ring A is optionally substituted 5-ring-membered heteroaryl containing two N.

In some embodiment the compound having the structure of formula (I-E), said ring A is optionally substituted pyrazolyl.

The present disclosure provides a compound or a pharmaceutically acceptable salt, prodrug, or metabolite thereof, or a solvate or hydrate of any of the foregoing, wherein, said compound is:

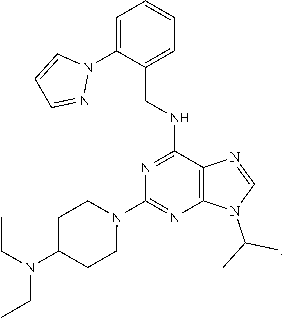 [SMILES: CCN(CC)C1CCN(c2nc(NCc3ccccc3-n3cccn3)c3ncn(C(C)C)c3n2)CC1]

The present disclosure provides a compound or a pharmaceutically acceptable salt, prodrug, or metabolite thereof, or a solvate or hydrate of any of the foregoing, wherein, said compound is selected from the group consisting of

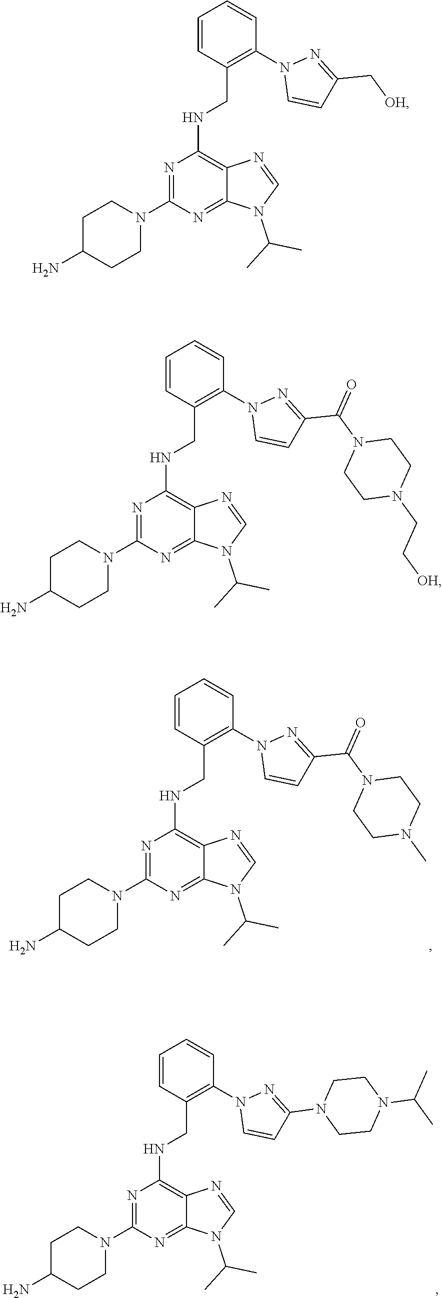 [SMILES: CC(C)n1cnc2c(NCc3ccccc3-n3ccc(CO)n3)nc(N3CCC(N)CC3)nc21 ; CC(C)n1cnc2c(NCc3ccccc3-n3ccc(C(=O)N4CCN(CCO)CC4)n3)nc(N3CCC(N)CC3)nc21 ; CC(C)n1cnc2c(NCc3ccccc3-n3ccc(C(=O)N4CCN(C)CC4)n3)nc(N3CCC(N)CC3)nc21 ; CC(C)N1CCN(c2ccn(-c3ccccc3CNc3nc(N4CCC(N)CC4)nc4c3ncn4C(C)C)n2)CC1]

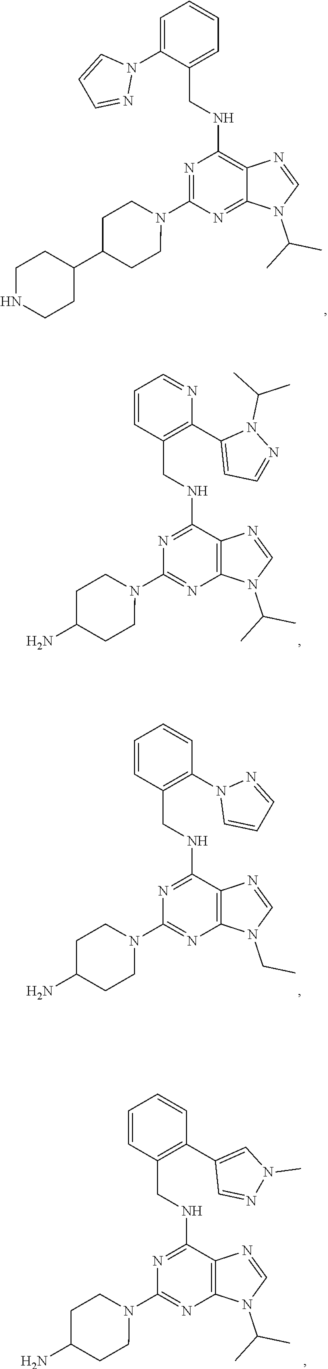 [SMILES: CC(C)n1cnc2c(NCc3ccccc3-n3cccn3)nc(N3CCC(C4CCNCC4)CC3)nc21 ; CC(C)n1nccc1-c1ncccc1CNc1nc(N2CCC(N)CC2)nc2c1ncn2C(C)C ; CCn1cnc2c(NCc3ccccc3-n3cccn3)nc(N3CCC(N)CC3)nc21 ; CC(C)n1cnc2c(NCc3ccccc3-c3cnn(C)c3)nc(N3CCC(N)CC3)nc21]

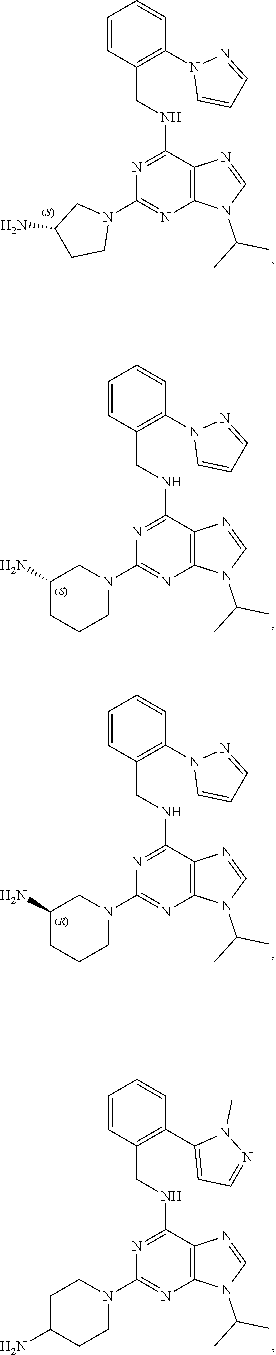

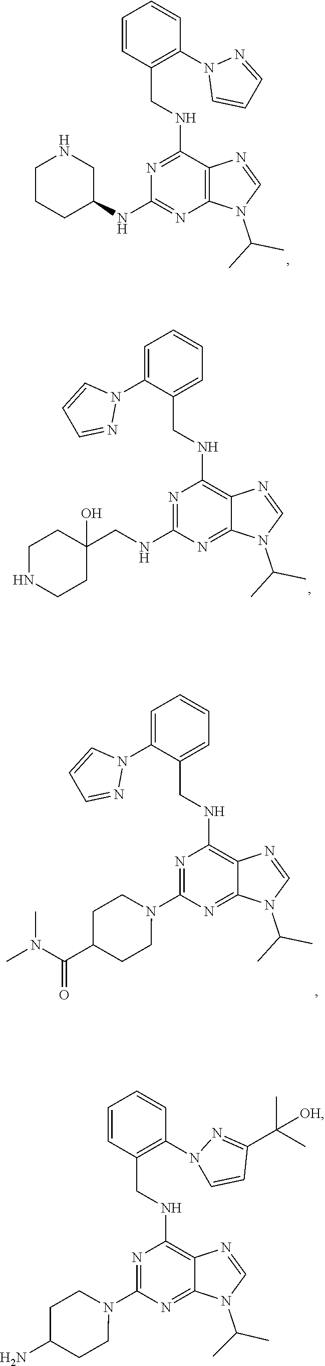

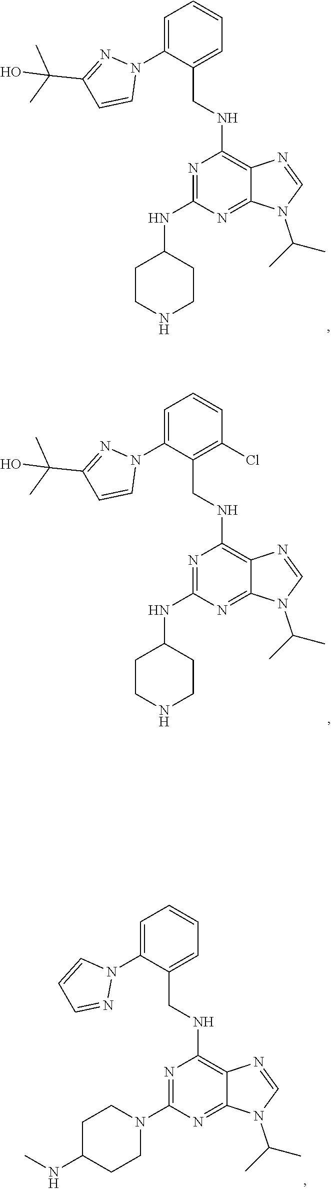

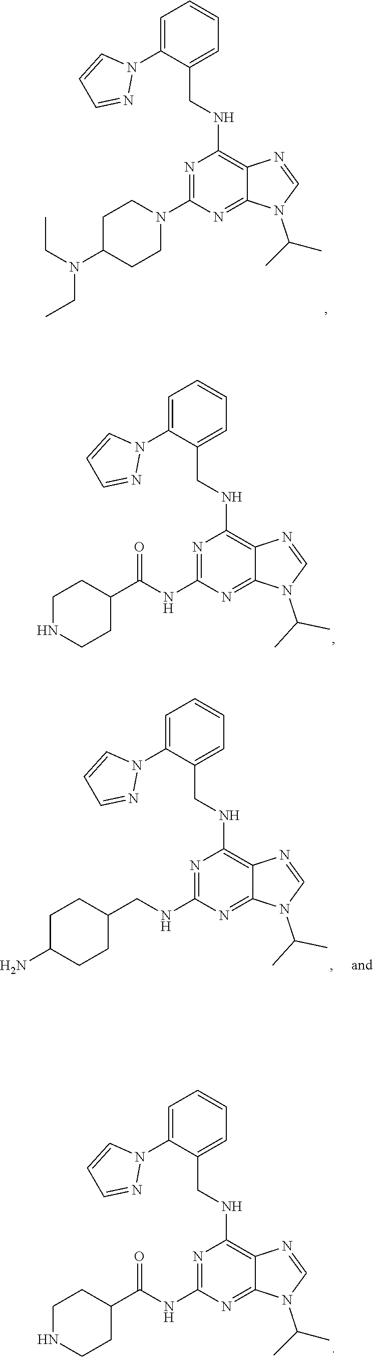 [SMILES: CCN(CC)C1CCN(c2nc(NCc3ccccc3-n3cccn3)c3ncn(C(C)C)c3n2)CC1 ; CC(C)n1cnc2c(NCc3ccccc3-n3cccn3)nc(NC(=O)C3CCNCC3)nc21 ; CC(C)n1cnc2c(NCc3ccccc3-n3cccn3)nc(NCC3CCC(N)CC3)nc21 ; CC(C)n1cnc2c(NCc3ccccc3-n3cccn3)nc(NC(=O)C3CCNCC3)nc21]

The present disclosure provides a compound having the structure of formula (II),

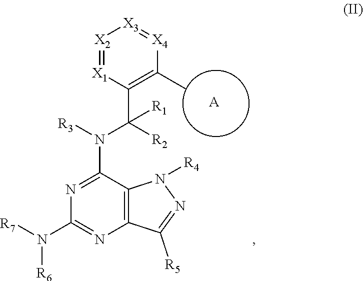

or a pharmaceutically acceptable salt, or prodrug thereof, or a solvate or hydrate of any of the forgoing,
wherein, each X₁, X₂, X₃, and X₄ is independently selected from optionally substituted —CH═, and —N═,
each R₁, R₂, R₃, R₄, R₅, R₆, and R₇, is independently absent or is independently selected from optional substituents, or R₆ and R₇ combined with the atoms to which they are attached form an optionally substituted ring,
wherein, ring A is optionally substituted ring.

In some embodiment the compound having the structure of formula (II), said X₁ is optionally substituted —CH═.

In some embodiment the compound having the structure of formula (II), said X₂ is optionally substituted —CH═.

In some embodiment the compound having the structure of formula (II), said X₃ is optionally substituted —CH═.

In some embodiment the compound having the structure of formula (II), said X₄ is optionally substituted —CH═.

In some embodiment the compound having the structure of formula (II), said R₁ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (II), said R₁ is selected from the group consisting of hydrogen, protium, deuterium, tritium, and halogen.

In some embodiment the compound having the structure of formula (II), said R₁ is H(hydrogen).

In some embodiment the compound having the structure of formula (II), said R₂ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (II), said R₂ is H.

In some embodiment the compound having the structure of formula (II), said R₃ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (II), said R₃ is H.

In some embodiment the compound having the structure of formula (II), said R₄ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (II), said R₄ is H.

In some embodiment the compound having the structure of formula (II), said R₅ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (II), said R₅ is selected from the group consisting of hydrogen, protium, deuterium, tritium, and optionally substituted (C₁-C₆)alkyl.

In some embodiment the compound having the structure of formula (II), said R₅ is optionally substituted (C₁-C₆)alkyl.

In some embodiment the compound having the structure of formula (II), said R₅ is optionally substituted propyl.

In some embodiment the compound having the structure of formula (II), said R₅ is optionally substituted isopropyl.

In some embodiment the compound having the structure of formula (II), said R₆ is selected from the group consisting of hydrogen, protium, deuterium, tritium, and optionally substituted 3 to 12-ring-membered heterocycle.

In some embodiment the compound having the structure of formula (II), said R₆ is optionally substituted 3 to 12-ring-membered heterocycle.

In some embodiment the compound having the structure of formula (II), said R₆ is optionally substituted 6-ring-membered heterocycle.

In some embodiment the compound having the structure of formula (II), said R₆ is optionally substituted piperidinyl.

In some embodiment the compound having the structure of formula (II), said R₇ is H.

In some embodiment the compound having the structure of formula (II), said R₆ and said R₇ combined with the atoms to which they are attached form an optionally substituted ring B, said ring B is optionally substituted 3 to 12-ring-membered heterocycle.

In some embodiment the compound having the structure of formula (II), said ring B is optionally substituted 6-ring-membered heterocycle.

In some embodiment the compound having the structure of formula (II), said ring B is optionally substituted piperidinyl.

In some embodiment the compound having the structure of formula (II), said ring B is substituted with one or more R_{B-1}, each said R_{B-1} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

In some embodiment the compound having the structure of formula (II), said R_{B-1} is selected from the group consisting of hydrogen, protium, deuterium, tritium, and optionally substituted amino.

In some embodiment the compound having the structure of formula (II), said ring A is optionally substituted 5 to 12-ring-membered heteroaryl.

In some embodiment the compound having the structure of formula (II), said ring A is optionally substituted 5-ring-membered heteroaryl.

In some embodiment the compound having the structure of formula (II), said ring A is optionally substituted 5-ring-membered heteroaryl containing two N.

In some embodiment the compound having the structure of formula (II), said ring A is optionally substituted pyrazolyl.

The present disclosure provides a compound or a pharmaceutically acceptable salt, prodrug, or metabolite thereof, or a solvate or hydrate of any of the foregoing, wherein, said compound is selected from the group consisting of

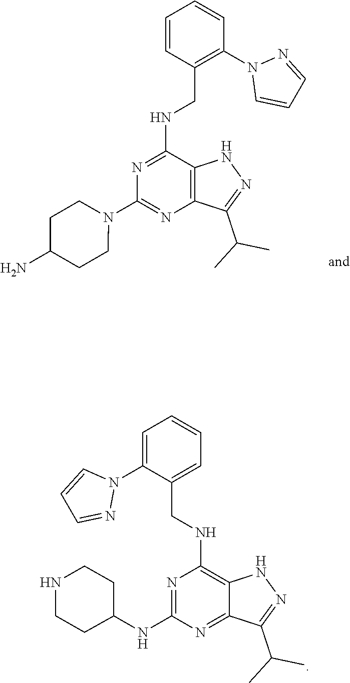 [SMILES: CC(C)c1n[nH]c2c(NCc3ccccc3-n3cccn3)nc(N3CCC(N)CC3)nc12 ; CC(C)c1n[nH]c2c(NCc3ccccc3-n3cccn3)nc(NC3CCNCC3)nc12]

The present disclosure provides a composition comprising a compound of present disclosure, or a pharmaceutically acceptable salt, prodrug, or metabolite thereof, or a solvate or hydrate of any of the foregoing, and optionally a pharmaceutically acceptable carrier.

The present disclosure provides a method for inhibiting cyclin-dependent kinase (CDK) activity, said method comprising administering to a subject in need thereof an effective amount of the compound of present disclosure, or a pharmaceutically acceptable salt, prodrug, or metabolite thereof, or a solvate or hydrate of any of the foregoing.

In some embodiment, wherein said cyclin-dependent kinase (CDK) is CDK 7.

In some embodiment, wherein said method is selected from the group consisting of an in vitro method, an ex vivo method, and an in vivo method.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCES

All publications, patents, and patent applications mentioned in this specification are herein incorporated by reference to the same extent as if each individual publication, patent, or patent application was specifically and individually indicated to be incorporated by reference.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the term “alkyl”, either alone or within other terms, generally refers to a linear or branched-chain saturated hydrocarbyl substituent (i.e., a substituent obtained from a hydrocarbon by removal of a hydrogen) containing from one to twenty carbon atoms; for example, from one to twelve carbon atoms; in another example, from one to ten carbon atoms; in another embodiment, from one to six carbon atoms; and in another embodiment, from one to four carbon atoms (such as 1, 2, 3 or more carbon atoms). Examples of such substituents may include e.g., methyl, ethyl, propyl (including n-propyl and isopropyl), butyl (including n-butyl, isobutyl, sec-butyl and tert-butyl), pentyl, isoamyl, hexyl and the like. In some instances, the number of carbon atoms in a hydrocarbyl substituent (i.e., alkyl, alkenyl, cycloalkyl, aryl, etc.) may be indicated by the prefix “Cₐ-C_{b}” wherein a is the minimum and b is the maximum number of carbon atoms in the substituent. Thus, for example, “C₁-C₆ alkyl” may refer to an alkyl substituent containing from 1 to 6 carbon atoms. The “alkyl” groups may be optionally substituted with one or more substitutions.

As used herein, the term “alkenyl”, either alone or within other terms, generally refers to a linear or branched-carbon radicals having at least one carbon-carbon double bond. The term “alkenyl” may contain conjugated and non-conjugated carbon-carbon double bonds or combinations thereof. An alkenyl group, for example and without being limited thereto, may contain two to about twenty carbon atoms or, in a particular embodiment, two to about twelve carbon atoms. In embodiments, alkenyl groups may contain two to about four carbon atoms (such as 2, 3 or more carbon atoms). Examples of alkenyl groups include, but are not limited thereto, ethenyl, propenyl, allyl, propenyl, butenyl and 4-methylbutenyl. The terms “alkenyl” contain groups having “cis” and “trans” orientations, or alternatively, “E” and “Z” orientations. In some instances, the number of carbon atoms may be indicated by the prefix “Cₐ-C_{b}” wherein a is the minimum and b is the maximum number of carbon atoms in the substituent. The “alkenyl” groups may be optionally substituted with one or more substitutions.

As used herein, the term “alkynyl”, either alone or within other terms, generally refers to linear or branched carbon radicals having at least one carbon-carbon triple bond. The term “alkynyl” may contain conjugated and non-conjugated carbon-carbon triple bonds or combinations thereof. Alkynyl group, for example and without being limited thereto, may contain two to about twenty carbon atoms or, in a particular embodiment, two to about twelve carbon atoms. In embodiments, alkynyl groups may contain two to about ten carbon atoms. Some examples may be alkynyl having two to about four carbon atoms (such as 2, 3 or more carbon atoms). In some instances, the number of carbon atoms may be indicated by the prefix “Cₐ-C_{b}” wherein a is the minimum and b is the maximum number of carbon atoms in the substituent. Examples of such groups include propargyl, butynyl, and the like. The “alkynyl” groups may be optionally substituted with one or more substitutions.

As used herein, the term “amino”, either alone or within other terms, generally refers to formula —NH₂ group. The “amino” groups may be optionally substituted with one or more substitutions.

As used herein, the term “ring” generally refers to a ring system or a ring structure. For example, ring may comprise carbocycle, heterocycle, aryl, or heteroaryl.

As used herein, the term “ring membered”, or “membered ring” generally refers to a ring system having ring atoms. For example, “n ring membered” or “n-membered ring” generally refers to a ring system having n ring atoms. For example, “5 ring membered” or “5-membered ring” generally refers to a ring system having 5 ring atoms which optionally contains one or more further heteroatom(s) selected from O, S or N.

As used herein, the term “carbocycle”, either alone or within other terms, generally refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles may have 3 to 6 ring atoms, or 5 to 6 ring atoms. Bicyclic carbocycles may have 7 to 12 ring atoms, e.g., arranged as a bicyclo[4,5], [5,5], [5,6] or [6,6] system, or 9 or 10 ring atoms arranged as a bicyclo[5,6] or [6,6] system. The term “carbocycle” may contain, for example, a monocyclic carbocycle ring fused to an aryl ring (e.g., a monocyclic carbocycle ring fused to a benzene ring). Carbocyles may have 3 to 8 carbon ring atoms. In some instances, the number of carbon atoms may be indicated by the prefix “Cₐ-C_{b}” wherein a is the minimum and b is the maximum number of carbon atoms in the substituent. The “carbocycle” groups may be optionally substituted with one or more substitutions.

As used herein, the term “heterocycle”, either alone or within other terms, generally refers to a monocyclic, bicyclic, or polycyclic ring system having from 3 to 14 ring atoms (also referred to as ring members) wherein at least one ring atom in at least one ring may be a heteroatom selected from N, O, P, or S (and all combinations and subcombinations of ranges and specific numbers of carbon atoms and heteroatoms therein). The heterocycle may have from 1 to 4 ring heteroatoms independently selected from N, O, P, or S. One or more N, C, or S atoms in a heterocycle may be oxidized. A monocylic heterocycle may have 3 to 7 ring members (e.g., 2 to 6 carbon atoms and 1 to 3 heteroatoms independently selected from N, O, P, or S), and a bicyclic heterocycle may have 5 to 10 ring members (e.g., 4 to 9 carbon atoms and 1 to 3 heteroatoms independently selected from N, O, P, or S). The heterocycle that contains the heteroatom may be non-aromatic. Unless otherwise noted, the heterocycle is attached to its pendant group at any heteroatom or carbon atom that results in a stable structure. In some instances, the number of carbon atoms may be indicated by the prefix “Cₐ-C_{b}” wherein a is the minimum and b is the maximum number of carbon atoms in the substituent. The “heterocycle” groups may be optionally substituted with one or more substitutions.

As used herein, the term “aryl”, either alone or within other terms, generally refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” may also contain substituents such as phenyl, naphthyl and anthracenyl that are fused to a C₄-C₁₀ carbocyclic ring, such as a C₅ or a C₆ carbocyclic ring, or to a 4- to 10-membered heterocyclic ring, wherein a group having such a fused aryl group as a substituent is bound to an aromatic carbon of the aryl group. When such a fused aryl group is substituted with one more substituent, the one or more substituents, unless otherwise specified, may be each bound to an aromatic carbon of the fused aryl group. The fused C₄-C₁₀ carbocyclic or 4- to 10-membered heterocyclic ring may optionally be optionally substituted. Examples of aryl groups may include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl. In some instances, the number of carbon atoms may be indicated by the prefix “Cₐ-C_{b}” wherein a is the minimum and b is the maximum number of carbon atoms in the substituent. The “aryl” groups may be optionally substituted with one or more substitutions.

As used herein, the term “heteroaryl”, either alone or within other terms, generally refers to an aromatic ring structure containing from 5 to 14 ring atoms in which at least one of the ring atoms is a heteroatom (for example, oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents may include but not limited to: 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1, 2, 4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused ring substituents such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. In some instances, the number of carbon atoms may be indicated by the prefix “Cₐ-C_{b}” wherein a is the minimum and b is the maximum number of carbon atoms in the substituent. The “heteroaryl” groups may be optionally substituted with one or more substitutions.

As used herein, the term “halogen”, either alone or within other terms, generally refers to fluorine (which may be depicted as —F), chlorine (which may be depicted as —Cl), bromine (which may be depicted as —Br), or iodine (which may be depicted as —I). In one embodiment, the halogen may be chlorine. In another embodiment, the halogen may be fluorine. In another embodiment, the halogen may be bromine.

As used herein, the term “cyano”, either alone or within other terms, generally refers to formula —CN group.

As used herein, the term “nitro”, either alone or within other terms, generally refers to formula —NO₂ group.

As used herein, the term “hydroxy”, either alone or within other terms, generally refers to formula —OH group. The “hydroxy” groups may be optionally substituted with one or more substitutions.

As used herein, the term “phosphorous-containing group”, either alone or within other terms, generally refers to functional group containing on or more phosphorous atoms. The phosphorous-containing group may refer to —O—P—(OH)₂, —O—PH—(OH), —O—PH₂, —P—(OH)₂, —PH—(OH), —PH₄, —PH₂═CH₂, —CH═PH₃, —O—P(═O)₂, —O—P(═O)—(OH)₂, —O—PH(═O)—OH, —P(═O)—(OH)₂, —O—PH₂(═O), —PH(═O)—OH, —PH₂(═O), —O—P(═O)(OH)—P(═O)(OH)₂, —O—P(═O)(OH)—O—P(═O)(OH)₂, —PH—PH₂, or —P═PH. The “phosphorous-containing group” may be optionally substituted with one or more substitutions.

As used herein, the term “silicon-containing group”, either alone or within other terms, generally refers to functional group containing on or more silicon atoms. The silicon-containing group may refer to —SiH₃. The “silicon-containing group” may be optionally substituted with one or more substitutions.

As used herein, the term “thio”, either alone or within other terms, generally refers to formula —SH group. The “thio” groups may be optionally substituted with one or more substitutions.

As used herein, the term “carboxyl”, either alone or within other terms, generally refers to formula —C(═O)OH group. The “carboxyl” groups may be optionally substituted with one or more substitutions.

As used herein, the term “sulfonyl”, either alone or within other terms, generally refers to formula —S(═O)₂—H, group. The “sulfonyl” groups may be optionally substituted with one or more substitutions.

As used herein, the term “sulfinyl”, either alone or within other terms, generally refers to formula —S(═O)—H group. The “sulfinyl” groups may be optionally substituted with one or more substitutions.

As used herein, the term “acyl”, either alone or within other terms, generally refers to a carboxylic acid ester of the formula —C(O)R in which the non-carbonyl moiety of the ester group (i.e., R) may be selected from straight, branched, or cyclic alkyl. The term acyl may include but not limited to acetyl, propionyl, butyryl and pentanoyl. In some instances, the number of carbon atoms may be indicated by the prefix “Cₐ-C_{b}” wherein a is the minimum and b is the maximum number of carbon atoms in the substituent. The “acyl” groups may be optionally substituted with one or more substitutions.

As used herein, the term “thioacyl”, either alone or within other terms, generally refers to the formula —C(S)R in which the moiety of the ester group (i.e., R) may be selected from straight, branched, or cyclic alkyl. In some instances, the number of carbon atoms may be indicated by the prefix “Cₐ-C_{b}” wherein a is the minimum and b is the maximum number of carbon atoms in the substituent. The “thioacyl” groups may be optionally substituted with one or more substitutions.

As used herein, the term “ring”, either alone or within other terms, generally refers to any covalently closed structure. Rings may include, for example, carbocycles, heterocycles, aryls and heteroaryls. Rings may be monocyclic or polycyclic. The “ring” groups may be optionally substituted with one or more substitutions.

As used herein, the term “pharmaceutically acceptable salt” generally refers to a salt that may be pharmaceutically acceptable and that may possess the desired pharmacological activity of the parent compound. Such salts may include: acid addition salts, formed with inorganic acids or formed with organic acids or basic addition salts formed with the conjugate bases of any of the inorganic acids wherein the conjugate bases comprise a cationic component.

As used herein, the term “pharmaceutically acceptable carrier” generally refers to aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles may include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms may be made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release may be controlled. Depot injectable formulations may be also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers may include sugars such as lactose. Desirably, at least 95% by weight of the particles of the active ingredient may have an effective particle size in the range of 0.01 to 10 micrometers.

As used herein, the term “prodrug” generally refers to a compound that is metabolized, for example hydrolyzed or oxidized, in the host to form the compound of the present invention. Typical examples of prodrugs may include compounds that have biologically labile protecting groups on a functional moiety of the active compound. Prodrugs may include compounds that can be oxidized, reduced, aminated, deaminated, hydroxylated, dehydroxylated, hydrolyzed, dehydrolyzed, alkylated, dealkylated, acylated, dedcylated, phosphorylated, dephosphorylated to produce the active compound.

As used herein, the term “cyclin-dependent kinase” or “CDK” generally refers to a protein having an activity of regulating the cell cycle. For example, inhibiting cyclin-dependent kinase (CDK) activity may comprise interacting with a cyclin-CDK complex to block kinase activity. For example, CDK may comprise CDK7. The UniPort ID for CDK7 may be P50613.

As used herein, a substituent is “substitutable” or can be “substituted” if it comprises at least one atom that is bonded to one or more hydrogen atoms. If a substituent is described as being “substituted,” hydrogen or a non-hydrogen substituent is in the place of a hydrogen substituent on a atom of the substituent. Thus, for example, a substituted alkyl substituent is an alkyl substituent wherein at least one hydrogen or a non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each substituent may be identical or different (unless otherwise stated).

If substituents are described as being “independently selected” from a group, each substituent may be selected independent of the other(s). Each substituent therefore may be identical to or different from the other substituent(s).

As used herein, the term “optionally substituted” or “optional substituent(s)” generally refers to a given moiety may consist of only hydrogen substituents through available valencies (unsubstituted) or may further comprise one or more non-hydrogen substituents through available valencies (substituted) that are not otherwise specified by the name of the given moiety. For example, “R^{x} is optionally substituted” or R^{x} is optionally substituted with R^{y}” may mean that R^{x} may be substituted with 0, 1, 2, 3, 4, 5, 6, 7, 8, or 9 R^{y}, for example, R^{x} may be substituted with 0, 1, 2, 3, 4, or 5 R^{y}, for example, R^{x} may be substituted with 1, 2, or 3 R^{y}, for example, R^{x} may be substituted with one R^{y}, for example, R^{x} may be substituted with 2 R^{y}, for example, R^{x} may be substituted with 3 R^{y}, for example, R^{x} may be substituted with 4 R^{y}, for example, R^{x} may be substituted with 5 R^{y}, for example, R^{x} may be substituted with 6 R^{y}, for example, R^{x} may be substituted with 7 R^{y}, for example, R^{x} may be substituted with 8 R^{y}, for example, R^{x} may be substituted with 9 R^{y}. In general, a non-hydrogen substituent may be any substituent that may be bound to an atom of the given moiety that is specified to be substituted. Examples of substituents include, but are not limited to, hydrogen, protium, deuterium, tritium, halogen, cyano, nitro, ═S, ═O, N₃, trifluoromethyl, hydroxy, phosphorous-containing group, silicon-containing group, thio, amino, carboxyl, sulfonyl, sulfinyl, (C₁-C₆)acyl, (C₁-C₆)thioacyl, (C₁-C₆)alkyl, (C₂-C₆)alkenyl, (C₂-C₆)alkynyl, (C₃-C₁₀)carbocycle, (C₂-C₉)heterocycle, (C₆-C₁₀)aryl, (C₁-C₉)heteroaryl, trifluoromethyl(C₁-C₆)alkyl, cyano(C₁-C₆)alkyl, halo(C₁-C₆)alkyl, nitro(C₁-C₆)alkyl, hydroxy(C₁-C₆)alkyl, (C₁-C₆)alkoxy, (C₁-C₆)alkylthio, thio(C₁-C₆)alkyl, amino(C₁-C₆)alkyl, (C₁-C₆)alkylamino, ((C₁-C₆)alkyl)₂amino, (C₁-C₆)acyl(C₁-C₆)alkyl, (C₁-C₆)alkylsulfonyl, (C₁-C₆)alkylsulfinyl, hydroxysulfonyl, hydroxysulfinyl, (C₃-C₁₀)carbocycle(C₁-C₆)alkyl, (C₂-C₉)heterocycle(C₁-C₆)alkyl, (C₆-C₁₀)aryl(C₁-C₆)alkyl, and (C₁-C₉)heteroaryl(C₁-C₆)alkyl. In addition, the substituent is itself optionally substituted by a further substituent. In one particular embodiment, examples of the further substituent include, but are not limited to, hydrogen, protium, deuterium, tritium, halogen, cyano, nitro, ═S, ═O, N₃, trifluoromethyl, hydroxy, phosphorous-containing group, silicon-containing group, thio, amino, carboxyl, sulfonyl, sulfinyl, (C₁-C₆)acyl, (C₁-C₆)thioacyl, (C₁-C₆)alkyl, (C₂-C₆)alkenyl, (C₂-C₆)alkynyl, (C₃-C₁₀)carbocycle, (C₂-C₉)heterocycle, (C₆-C₁₀)aryl, (C₁-C₉)heteroaryl, trifluoromethyl(C₁-C₆)alkyl, cyano(C₁-C₆)alkyl, halo(C₁-C₆)alkyl, nitro(C₁-C₆)alkyl, hydroxy(C₁-C₆)alkyl, (C₁-C₆)alkoxy, (C₁-C₆)alkylthio, thio(C₁-C₆)alkyl, amino(C₁-C₆)alkyl, (C₁-C₆)alkylamino, ((C₁-C₆)alkyl)₂amino, (C₁-C₆)acyl(C₁-C₆)alkyl, (C₁-C₆)alkylsulfonyl, (C₁-C₆)alkylsulfinyl, hydroxysulfonyl, hydroxysulfinyl, (C₃-C₁₀)carbocycle(C₁-C₆)alkyl, (C₂-C₉)heterocycle(C₁-C₆)alkyl, (C₆-C₁₀)aryl(C₁-C₆)alkyl, and (C₁-C₉)heteroaryl(C₁-C₆)alkyl.

As used herein, the term “formula” may be hereinafter referred to as a “compound(s) of the invention”. Such terms are also defined to include all forms of the compound of formula, including hydrates, solvates, isomers, crystalline and non-crystalline forms, isomorphs, polymorphs, and metabolites thereof. For example, the compounds of formula, or pharmaceutically acceptable salts thereof, may exist in unsolvated and solvated forms. When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

The compounds of “formula” may have asymmetric carbon atoms. The carbon-carbon bonds of the compounds of formula may be depicted herein using a solid line, a solid wedge, or a dotted wedge. The use of a solid line to depict bonds to asymmetric carbon atoms may be meant to indicate that all possible stereoisomers (e.g. specific enantiomers, racemic mixtures, etc.) at that carbon atom are included. The use of either a solid or dotted wedge to depict bonds to asymmetric carbon atoms may be meant to indicate that only the stereoisomer shown is meant to be included. It is possible that compounds of the present application may contain more than one asymmetric carbon atom. In those compounds, the use of a solid line to depict bonds to asymmetric carbon atoms may be meant to indicate that all possible stereoisomers are meant to be included. For example, unless stated otherwise, it may be intended that the compounds of formula can exist as enantiomers and diastereomers or as racemates and mixtures thereof. The use of a solid line to depict bonds to one or more asymmetric carbon atoms in a compound of formula and the use of a solid or dotted wedge to depict bonds to other asymmetric carbon atoms in the same compound may be meant to indicate that a mixture of diastereomers is present.

The compounds of the present application (e.g., the compounds of formula) may exist as clathrates or other complexes. Included within the scope of the invention are complexes such as clathrates, drug-host inclusion complexes wherein, in contrast to the aforementioned solvates, the drug and host may be present in stoichiometric or non-stoichiometric amounts. Also included may be complexes of formula containing two or more organic and/or inorganic components which may be in stoichiometric or non-stoichiometric amounts. The resulting complexes may be ionized, partially ionized, or non-ionized.

Stereoisomers of formula may include cis and trans isomers, optical isomers such as R and S enantiomers, diastereomers, geometric isomers, rotational isomers, conformational isomers, and tautomers of the compounds of formula, including compounds exhibiting more than one type of isomerism; and mixtures thereof (such as racemates and diastereomeric pairs). Also included may be acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of formula may exhibit the phenomena of tautomerism and structural isomerism. For example, the compounds of formula may exist in several tautomeric forms, including the enol and imine forms, and the keto and enamine forms, and geometric isomers and mixtures thereof. All such tautomeric forms may be included within the scope of compounds of formula. Tautomers may exist as mixtures of a tautomeric set in solution. In solid form, usually one tautomer predominates. Even though one tautomer may be described, the present invention includes all tautomers of the compounds of formula.

The present invention also includes isotopically-labeled compounds, which are identical to those recited in formula above, but for the fact that one or more atoms may be replaced by an atom having an atomic mass or mass number different from the atomic mass or mass number usually found in nature. Examples of isotopes that may be incorporated into compounds of formula include isotopes of hydrogen, carbon, nitrogen, oxygen, phosphorus, fluorine and chlorine, such as, but not limited to, ²H, ³H, ¹³C, ¹⁴C, ¹⁵N, ¹⁸O, ¹⁷O, ³¹P, ³²P, ³⁵S, ¹⁸F, and ³⁶Cl. Certain isotopically-labeled compounds of formula, for example those into which radioactive isotopes such as ³H and ¹⁴C are incorporated, may be useful in drug and/or substrate tissue distribution assays. Tritiated, i.e., ³H, and carbon-14, i.e., ¹⁴C, isotopes may be particularly used for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be used in some circumstances. Isotopically-labeled compounds of formula may generally be prepared by carrying out the procedures disclosed in the Schemes and/or in the Examples below, by substituting an isotopically-labeled reagent for a non-isotopically-labeled reagent.

The compounds of the present application may be used in the form of salts derived from inorganic or organic acids. Depending on the particular compound, a salt of the compound may be advantageous due to one or more of the salt's physical properties, such as enhanced pharmaceutical stability in differing temperatures and humidity, or a desirable solubility in water or oil. In some instances, a salt of a compound also may be used as an aid in the isolation, purification, and/or resolution of the compound.

### Compounds

In one aspect, the present application provides a compound having the structure of formula (I),

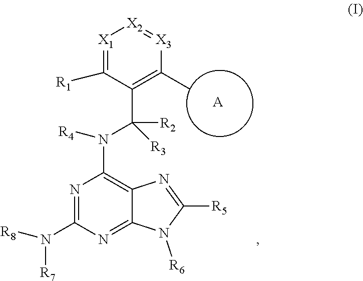

or a pharmaceutically acceptable salt, or prodrug thereof, or a solvate or hydrate of any of the forgoing, wherein, each X₁, X₂, and X₃ may be independently selected from optionally substituted —CH═, and —N═, each R₁, R₂, R₃, R₄, R₅, R₆, R₇, and R₈, may be independently absent or may be independently selected from optional substituents, or R₇ and R₈ combined with the atoms to which they are attached may form an optionally substituted ring, wherein, ring A may be optionally substituted 5-ring-membered heteroaryl containing one or more N(nitrogen).

For example, as a compound having the structure of formula (I), said X₁ may be optionally substituted —CH═.

For example, as a compound having the structure of formula (I), said X₂ may be optionally substituted —CH═

For example, as a compound having the structure of formula (I), said X₃ may be optionally substituted —CH═ or —N═. For example, said X₃ may be optionally substituted —CH═ or said X₃ may be optionally substituted —N═.

For example, as a compound having the structure of formula (I), said R₁ may be selected from the group consisting of hydrogen, protium, deuterium, tritium, and halogen. For example, said R₁ may be H(hydrogen), F(fluorine) or Cl (chlorine). For example, said R₁ may be H(hydrogen), said R₁ may be F(fluorine) or said R₁ may be Cl (chlorine).

For example, as a compound having the structure of formula (I), said R₂ may be H.

For example, as a compound having the structure of formula (I), said R₃ may be H.

For example, as a compound having the structure of formula (I), said R₄ may be H.

For example, as a compound having the structure of formula (I), said R₅ may be H.

For example, as a compound having the structure of formula (I), said R₆ may be selected from the group consisting of hydrogen, protium, deuterium, tritium, optionally substituted (C₁-C₆)alkyl, and 3 to 12-ring-membered carbocycle. For example, R₆ may be optionally substituted (C₁-C₆)alkyl or 3 to 12-ring-membered carbocycle. For example, R₆ may be optionally substituted ethyl, optionally substituted propyl or optionally substituted 5-ring-membered carbocycle. For example, R₆ may be optionally substituted ethyl, R₆ may be optionally substituted isopropyl or R₆ may be optionally substituted cyclopentanyl.

For example, as a compound having the structure of formula (I), said R₇ may be selected from the group consisting of hydrogen, protium, deuterium, tritium, optionally substituted (C₁-C₆)acyl, optionally substituted (C₁-C₆)alkyl, optionally substituted 3 to 12-ring-membered carbocycle, and optionally substituted 3 to 12-ring-membered heterocycle. For example, said R₇ may be optionally substituted (C₁-C₆)acyl, optionally substituted (C₁-C₆)alkyl, optionally substituted 3 to 12-ring-membered carbocycle, or optionally substituted 3 to 12-ring-membered heterocycle. For example, said R₇ may be optionally substituted formyl, optionally substituted methyl, optionally substituted propyl, optionally substituted cyclohexyl, optionally substituted piperidinyl or optionally substituted 1-azabicyclo [2.2.2] octane.

For example, as a compound having the structure of formula (I), said R₇ may be substituted with one or more R₇₋₁, each said R₇₋₁ may be independently selected from optional substituents. For example, said R₇₋₁ may be selected from the group consisting of hydrogen, protium, deuterium, tritium, optionally substituted hydroxy, optionally substituted amino, optionally substituted 3 to 12-ring-membered carbocycle, and optionally substituted 3 to 12-ring-membered heterocycle. For example, said R₇₋₁ may be optionally substituted hydroxy, optionally substituted amino, optionally substituted 3 to 12-ring-membered carbocycle, or optionally substituted 3 to 12-ring-membered heterocycle. For example, said R₇₋₁ may be optionally substituted hydroxy, optionally substituted amino, optionally substituted cyclohexyl, or optionally substituted 6-ring-membered heterocycle. For example, said R₇₋₁ may be optionally substituted hydroxy, optionally substituted amino, optionally substituted cyclohexyl, or optionally substituted piperidinyl.

For example, as a compound having the structure of formula (I), said R₇ may be optionally substituted (C₁-C₆)acyl, and said R₇₋₁ may be optionally substituted 3 to 12-ring-membered heterocycle. For example, said R₇ may be optionally substituted formyl, and said R₇₋₁ may be optionally substituted 3 to 12-ring-membered heterocycle. For example, said R₇ may be optionally substituted formyl, and said R₇₋₁ may be optionally substituted 6-ring-membered heterocycle. For example, said R₇ may be optionally substituted formyl, and said R₇₋₁ may be optionally substituted piperidinyl.

For example, as a compound having the structure of formula (I), said R₇ may be optionally substituted (C₁-C₆)alkyl, and said R₇₋₁ may be hydrogen, protium, deuterium, tritium, optionally substituted hydroxy, optionally substituted amino, optionally substituted 3 to 12-ring-membered carbocycle, or optionally substituted 3 to 12-ring-membered heterocycle. For example, said R₇ may be optionally substituted methyl or optionally substituted propyl, and said R₇₋₁ may be hydrogen, protium, deuterium, tritium, optionally substituted hydroxy, optionally substituted amino, optionally substituted 3 to 12-ring-membered carbocycle, or optionally substituted 3 to 12-ring-membered heterocycle. For example, said R₇ may be optionally substituted methyl, and said R₇₋₁ may be hydrogen, protium, deuterium, tritium, optionally substituted 3 to 12-ring-membered carbocycle, or optionally substituted 3 to 12-ring-membered heterocycle. For example, said R₇ may be optionally substituted methyl, and said R₇₋₁ may be hydrogen, protium, deuterium, tritium, optionally substituted cyclohexyl, or optionally substituted 6-ring-membered heterocycle. For example, said R₇ may be optionally substituted methyl, and said R₇₋₁ may be hydrogen, protium, deuterium, tritium, optionally substituted cyclohexyl, or optionally substituted piperidinyl. For example, said R₇ may be optionally substituted propyl, and said R₇₋₁ may be hydrogen, protium, deuterium, tritium, optionally substituted hydroxy, or optionally substituted amino.

For example, as a compound having the structure of formula (I), said R₇ may be optionally substituted 3 to 12-ring-membered carbocycle, and said R₇₋₁ may be hydrogen, protium, deuterium, tritium, or optionally substituted hydroxy. For example, said R₇ may be optionally substituted cyclohexyl, and said R₇₋₁ may be hydrogen, protium, deuterium, tritium, or optionally substituted hydroxy.

For example, as a compound having the structure of formula (I), said R₇ may be substituted with one or more R₇₋₁, said R₇₋₁ may be substituted with one or more R₇₋₂, each said R₇₋₂ may be independently selected from optional substituents. For example, said R₇₋₂ may be selected from the group consisting of hydrogen, protium, deuterium, tritium, optionally substituted hydroxy, and optionally substituted amino. For example, said R₇₋₂ may be hydrogen, protium, deuterium, tritium, optionally substituted hydroxy, or optionally substituted amino.

For example, as a compound having the structure of formula (I), said R₇ may be optionally substituted (C₁-C₆)alkyl, said R₇₋₁ may be optionally substituted 3 to 12-ring-membered carbocycle, or optionally substituted 3 to 12-ring-membered heterocycle, and said R₇₋₂ may be hydrogen, protium, deuterium, tritium, optionally substituted hydroxy, or optionally substituted amino. For example, said R₇ may be optionally substituted methyl, said R₇₋₁ may be optionally substituted cyclohexyl, or optionally substituted 6-ring-membered heterocycle, and said R₇₋₂ may be hydrogen, protium, deuterium, tritium, optionally substituted hydroxy, or optionally substituted amino. For example, said R₇ may be optionally substituted methyl, said R₇₋₁ may be optionally substituted cyclohexyl, and said R₇₋₂ may be hydrogen, protium, deuterium, tritium, or optionally substituted amino. For example, said R₇ may be optionally substituted methyl, said R₇₋₁ may be optionally substituted piperidinyl, and said R₇₋₂ may be hydrogen, protium, deuterium, tritium, or optionally substituted hydroxy.

For example, as a compound having the structure of formula (I), said R₈ may be hydrogen.

For example, as a compound having the structure of formula (I), said R₇ and said R₈ combined with the atoms to which they are attached may form an optionally substituted ring B, said ring B may be optionally substituted 3 to 12-ring-membered heterocycle. For example, said ring B may be optionally substituted 5 to 6-ring-membered heterocycle. For example, said ring B may be optionally substituted piperidinyl, optionally substituted piperazinyl, or optionally substituted tetrahydropyrrolyl.

For example, as a compound having the structure of formula (I), said ring B may be substituted with one or more R_{B-1}, each said R_{B-1} may be independently selected from optional substituents. For example, said R_{B-1} may be selected from the group consisting of hydrogen, protium, deuterium, tritium, optionally substituted hydroxy, optionally substituted amino, optionally substituted (C₁-C₆)acyl, optionally substituted (C₁-C₆)alkyl, and optionally substituted 3 to 12-ring-membered heterocycle. For example, said R_{B-1} may be hydroxy, optionally substituted amino, optionally substituted formyl, optionally substituted methyl, or optionally substituted 6-ring-membered heterocycle. For example, said R_{B-1} may be hydroxy, optionally substituted amino, optionally substituted formyl, optionally substituted methyl, or optionally substituted piperidinyl.

For example, as a compound having the structure of formula (I), said ring B may be optionally substituted piperidinyl, and said R_{B-1} may be selected from the group consisting of hydrogen, protium, deuterium, tritium, optionally substituted hydroxy, optionally substituted amino, optionally substituted (C₁-C₆)acyl, optionally substituted (C₁-C₆)alkyl, and optionally substituted 3 to 12-ring-membered heterocycle. For example, said ring B may be optionally substituted piperidinyl, and said R_{B-1} may be selected from the group consisting of hydrogen, protium, deuterium, tritium, optionally substituted hydroxy, optionally substituted amino, optionally substituted formyl, optionally substituted methyl, and optionally substituted piperidinyl.

For example, as a compound having the structure of formula (I), said ring B may be optionally substituted piperazinyl, and said R_{B-1} may be selected from the group consisting of hydrogen, protium, and deuterium.

For example, as a compound having the structure of formula (I), said ring B may be optionally substituted tetrahydropyrrolyl, and said R_{B-1} may be selected from the group consisting of hydrogen, protium, deuterium, tritium, optionally substituted amino, and optionally substituted (C₁-C₆)alkyl. For example, said ring B may be optionally substituted tetrahydropyrrolyl, and said R_{B-1} may be hydrogen, protium, deuterium, tritium, optionally substituted amino, or optionally substituted methyl.

For example, as a compound having the structure of formula (I), said ring B may be substituted with one or more R_{B-1}, said R_{B-1} may be substituted with one or more R_{B-2}, each said R_{B-2} may be independently selected from optional substituents. For example, said R_{B-2} may be selected from the group consisting of hydrogen, protium, deuterium, tritium, optionally substituted hydroxy, optionally substituted amino, optionally substituted (C₁-C₆)alkyl, and optionally substituted 5 to 12-ring-membered heteroaryl. For example, said R_{B-2} may be hydrogen, protium, deuterium, tritium, optionally substituted hydroxy, optionally substituted amino, optionally substituted methyl, optionally substituted ethyl, or optionally substituted 5-ring-membered heteroaryl. For example, said R_{B-2} may be hydrogen, protium, deuterium, tritium, optionally substituted hydroxy, optionally substituted amino, optionally substituted methyl, optionally substituted ethyl, or optionally substituted pyrazolyl.

For example, as a compound having the structure of formula (I), said ring B may be optionally substituted piperidinyl, said R_{B-1} may be optionally substituted amino, and said R_{B-2} may be selected from the group consisting of hydrogen, protium, deuterium, tritium, and optionally substituted (C₁-C₆)alkyl. For example, said ring B may be optionally substituted piperidinyl, said R_{B-1} may be optionally substituted amino, and said R_{B-2} may be hydrogen, protium, deuterium, tritium, optionally substituted methyl, or optionally substituted ethyl.

For example, as a compound having the structure of formula (I), said ring B may be optionally substituted piperidinyl, said R_{B-1} may be optionally substituted (C₁-C₆)acyl, and said R_{B-2} may be selected from the group consisting of hydrogen, protium, deuterium, tritium, and optionally substituted amino. For example, said ring B may be optionally substituted piperidinyl, said R_{B-1} may be optionally substituted formyl, and said R_{B-2} may be hydrogen, protium, deuterium, tritium, or optionally substituted amino.

For example, as a compound having the structure of formula (I), said ring B may be optionally substituted piperidinyl, said R_{B-1} may be optionally substituted (C₁-C₆)alkyl, and said R_{B-2} may be selected from the group consisting of hydrogen, protium, deuterium, tritium, optionally substituted hydroxy, optionally substituted amino, and optionally substituted 5 to 12-ring-membered heteroaryl. For example, said ring B may be optionally substituted piperidinyl, said R_{B-1} may be optionally substituted methyl, and said R_{B-2} may be hydrogen, protium, deuterium, tritium, optionally substituted hydroxy, optionally substituted amino, or optionally substituted 5 to 12-ring-membered heteroaryl. For example, said ring B may be optionally substituted piperidinyl, said R_{B-1} may be optionally substituted methyl, and said R_{B-2} may be hydrogen, protium, deuterium, tritium, optionally substituted hydroxy, optionally substituted amino, or optionally substituted pyrazolyl.

For example, as a compound having the structure of formula (I), said ring B may be optionally substituted tetrahydropyrrolyl, said R_{B-1} may be optionally substituted (C₁-C₆)alkyl, and said R_{B-2} may be selected from the group consisting of hydrogen, protium, deuterium, tritium, and optionally substituted amino. For example, said ring B may be optionally substituted tetrahydropyrrolyl, said R_{B}-1 may be optionally substituted methyl, and said R_{B-2} may be hydrogen, protium, deuterium, tritium, or optionally substituted amino.

For example, as a compound having the structure of formula (I), said ring B may be substituted with one or more R_{B-1}, said R_{B-1} may be substituted with one or more R_{B-2}, said R_{B-2} may be substituted with one or more R_{B-3}, each said R_{B-3} may be independently selected from optional substituents. For example, said R_{B-3} may be selected from the group consisting of hydrogen, protium, deuterium, tritium, optionally substituted amino, and optionally substituted (C₁-C₆)alkyl. For example, said R_{B-3} may be hydrogen, protium, deuterium, tritium, optionally substituted amino, or optionally substituted methyl.

For example, as a compound having the structure of formula (I), said ring B may be optionally substituted piperidinyl, said R_{B-1} may be optionally substituted (C₁-C₆)acyl, said R_{B-2} may be optionally substituted amino, and said R_{B-3} may be hydrogen, protium, deuterium, tritium, or optionally substituted (C₁-C₆)alkyl. For example, said ring B may be optionally substituted piperidinyl, said R_{B-1} may be optionally substituted formyl, said R_{B-2} may be optionally substituted amino, and said R_{B-3} may be hydrogen, protium, deuterium, tritium, or optionally substituted (C₁-C₆)alkyl. For example, said ring B may be optionally substituted piperidinyl, said R_{B-1} may be optionally substituted formyl, said R_{B-2} may be optionally substituted amino, and said R_{B-3} may be hydrogen, protium, deuterium, tritium, or optionally substituted methyl.

For example, as a compound having the structure of formula (I), said ring B may be optionally substituted piperidinyl, said R_{B-1} may be optionally substituted (C₁-C₆)alkyl, said R_{B-2} may be optionally substituted amino or optionally substituted 3 to 12-ring-membered heterocycle, and said R_{B-3} may be hydrogen, protium, deuterium, tritium, optionally substituted amino, and optionally substituted (C₁-C₆)alkyl. For example, said ring B may be optionally substituted piperidinyl, said R_{B-1} may be optionally substituted methyl, said R_{B-2} may be optionally substituted amino or optionally substituted pyrazolyl, and said R_{B-3} may be hydrogen, protium, deuterium, tritium, optionally substituted amino, and optionally substituted (C₁-C₆)alkyl. For example, said ring B may be optionally substituted piperidinyl, said R_{B-1} may be optionally substituted methyl, said R_{B-2} may be optionally substituted amino, and said R_{B-3} may be hydrogen, protium, deuterium, tritium, and optionally substituted (C₁-C₆)alkyl. For example, said ring B may be optionally substituted piperidinyl, said R_{B}-1 may be optionally substituted methyl, said R_{B-2} may be optionally substituted amino, and said R_{B-3} may be hydrogen, protium, deuterium, tritium, and optionally substituted methyl. For example, said ring B may be optionally substituted piperidinyl, said R_{B-1} may be optionally substituted methyl, said R_{B-2} may be optionally substituted pyrazolyl, and said R_{B-3} may be hydrogen, protium, deuterium, tritium, optionally substituted amino, and optionally substituted (C₁-C₆)alkyl. For example, said ring B may be optionally substituted piperidinyl, said R_{B-1} may be optionally substituted methyl, said R_{B-2} may be optionally substituted pyrazolyl, and said R_{B-3} may be hydrogen, protium, deuterium, tritium, optionally substituted amino, and optionally substituted methyl.

For example, as a compound having the structure of formula (I), said ring B may be substituted with one or more R_{B-1}, said R_{B-1} may be substituted with one or more R_{B-2}, said R_{B-2} may be substituted with one or more R_{B-3}, said R_{B-3} may be substituted with one or more R_{B-4}, each said R_{B-4} may be independently selected from optional substituents. For example, said R_{B-4} may be selected from the group consisting of hydrogen, protium, deuterium, tritium, and halogen. For example, said R_{B-4} may be F.

For example, as a compound having the structure of formula (I), said ring B may be optionally substituted piperidinyl, said R_{B-1} may be optionally substituted methyl, said R_{B-2} may be optionally substituted pyrazolyl, said R_{B-3} may be methyl, and said R_{B-4} may be selected from the group consisting of hydrogen, protium, deuterium, tritium, and halogen. For example, said ring B may be optionally substituted piperidinyl, said R_{B-1} may be optionally substituted methyl, said R_{B-2} may be optionally substituted pyrazolyl, said R_{B-3} may be methyl, and said R_{B-4} may be selected from the group consisting of hydrogen, protium, deuterium, tritium, and F.

For example, as a compound having the structure of formula (I), said ring A may be optionally substituted 5-ring-membered heteroaryl containing two N. For example, said ring A may be optionally substituted pyrazolyl.

For example, as a compound having the structure of formula (I), said ring A may be substituted with one or more R_{A-1}, each said R_{A-1} may be independently selected from optional substituents. For example, said R_{A-1} may be selected from the group consisting of hydrogen, protium, deuterium, tritium, optionally substituted (C₁-C₆)acyl, optionally substituted (C₁-C₆)alkyl, and optionally substituted 3 to 12-ring-membered heterocycle. For example, said R_{A-1} may be hydrogen, protium, deuterium, tritium, optionally substituted formyl, optionally substituted methyl, optionally substituted propyl, or optionally substituted 6-ring-membered heterocycle. For example, said R_{A-1} may be hydrogen, protium, deuterium, tritium, optionally substituted formyl, optionally substituted methyl, optionally substituted isopropyl, or optionally substituted piperazinyl.

For example, as a compound having the structure of formula (I), said ring A may be optionally substituted pyrazolyl, and said R_{A-1} may be selected from the group consisting of hydrogen, protium, deuterium, tritium, optionally substituted (C₁-C₆)acyl, optionally substituted (C₁-C₆)alkyl, and optionally substituted 3 to 12-ring-membered heterocycle. For example, said ring A may be optionally substituted pyrazolyl, and said R_{A-1} may be hydrogen, protium, deuterium, tritium, optionally substituted formyl, optionally substituted methyl, optionally substituted isopropyl, or optionally substituted piperazinyl.

For example, as a compound having the structure of formula (I), said ring A may be substituted with one or more R_{A-1}, said R_{A-1} may be substituted with one or more R_{A-2}, each said R_{A-2} may be independently selected from optional substituents. For example, said R_{A-2} may be selected from the group consisting of hydrogen, protium, deuterium, tritium, optionally substituted hydroxy, optionally substituted (C₁-C₆)alkyl, and optionally substituted 3 to 12-ring-membered heterocycle. For example, said R_{A-2} may be hydrogen, protium, deuterium, tritium, optionally substituted hydroxy, optionally substituted propyl, or optionally substituted 6-ring-membered heterocycle. For example, said R_{A-2} may be hydrogen, protium, deuterium, tritium, optionally substituted hydroxy, optionally substituted isopropyl, or optionally substituted piperazinyl.

For example, as a compound having the structure of formula (I), said ring A may be optionally substituted pyrazolyl, said R_{A-1} may be optionally substituted (C₁-C₆)acyl, and said R_{A-2} may be selected from the group consisting of hydrogen, protium, deuterium, tritium, and optionally substituted 3 to 12-ring-membered heterocycle. For example, said ring A may be optionally substituted pyrazolyl, said R_{A-1} may be optionally substituted formyl, and said R_{A-2} may be selected from the group consisting of hydrogen, protium, deuterium, tritium, and optionally substituted 3 to 12-ring-membered heterocycle. For example, said ring A may be optionally substituted pyrazolyl, said R_{A-1} may be optionally substituted formyl, and said R_{A-2} may be hydrogen, protium, deuterium, tritium, or optionally substituted 6-ring-membered heterocycle. For example, said ring A may be optionally substituted pyrazolyl, said R_{A-1} may be optionally substituted formyl, and said R_{A-2} may be hydrogen, protium, deuterium, tritium, or optionally substituted piperazinyl.

For example, as a compound having the structure of formula (I), said ring A may be optionally substituted pyrazolyl, said R_{A-1} may be optionally substituted (C₁-C₆)alkyl, and said R_{A-2} may be selected from the group consisting of hydrogen, protium, deuterium, tritium, and optionally substituted hydroxy. For example, said ring A may be optionally substituted pyrazolyl, said R_{A-1} may be optionally substituted methyl, or optionally substituted isopropyl, and said R_{A-2} may be selected from the group consisting of hydrogen, protium, deuterium, tritium, and optionally substituted hydroxy.

For example, as a compound having the structure of formula (I), said ring A may be optionally substituted pyrazolyl, said R_{A-1} may be optionally substituted 3 to 12-ring-membered heterocycle, and said R_{A-2} may be selected from the group consisting of hydrogen, protium, deuterium, tritium, and optionally substituted (C₁-C₆)alkyl. For example, said ring A may be optionally substituted pyrazolyl, said R_{A-1} may be optionally substituted piperazinyl, and said R_{A-2} may be selected from the group consisting of hydrogen, protium, deuterium, tritium, and optionally substituted (C₁-C₆)alkyl. For example, said ring A may be optionally substituted pyrazolyl, said R_{A-1} may be optionally substituted piperazinyl, and said R_{A-2} may be selected from the group consisting of hydrogen, protium, deuterium, tritium, and optionally substituted isopropyl.

For example, as a compound having the structure of formula (I), said ring A may be substituted with one or more R_{A-1}, said R_{A-1} may be substituted with one or more R_{A-2}, said R_{A-2} may be substituted with one or more R_{A-3}, each said R_{A-3} may be independently selected from optional substituents. For example, said R_{A-3} may be selected from the group consisting of hydrogen, protium, deuterium, tritium, and optionally substituted (C₁-C₆)alkyl. For example, said R_{A-3} may be hydrogen, protium, deuterium, tritium, optionally substituted methyl or optionally substituted ethyl.

For example, as a compound having the structure of formula (I), said ring A may be optionally substituted pyrazolyl, said R_{A-1} may be optionally substituted (C₁-C₆)acyl, said R_{A-2} may be optionally substituted 3 to 12-ring-membered heterocycle, and said R_{A-3} may be selected from the group consisting of hydrogen, protium, deuterium, tritium, and optionally substituted (C₁-C₆)alkyl. For example, said ring A may be optionally substituted pyrazolyl, said R_{A-1} may be optionally substituted formyl, said R_{A-2} may be optionally substituted piperazinyl, and said R_{A-3} may be selected from the group consisting of hydrogen, protium, deuterium, tritium, and optionally substituted (C₁-C₆)alkyl. For example, said ring A may be optionally substituted pyrazolyl, said R_{A-1} may be optionally substituted formyl, said R_{A-2} may be optionally substituted piperazinyl, and said R_{A-3} may be hydrogen, protium, deuterium, tritium, optionally substituted methyl or optionally substituted ethyl.

For example, as a compound having the structure of formula (I), said ring A may be substituted with one or more R_{A-1}, said R_{A-1} may be substituted with one or more R_{A-2}, said R_{A-2} may be substituted with one or more R_{A-3}, said R_{A-3} may be substituted with one or more R_{A-4}, each said R_{A-4} may be independently selected from optional substituents. For example, said R_{A-4} may be selected from the group consisting of hydrogen, protium, deuterium, tritium, and optionally substituted hydroxy.

For example, as a compound having the structure of formula (I), said ring A may be optionally substituted pyrazolyl, said R_{A-1} may be optionally substituted (C₁-C₆)acyl, said R_{A-2} may be optionally substituted 3 to 12-ring-membered heterocycle, said R_{A-3} may be optionally substituted (C₁-C₆)alkyl, and R_{A-4} may be selected from the group consisting of hydrogen, protium, deuterium, tritium, and optionally substituted hydroxy. For example, said ring A may be optionally substituted pyrazolyl, said R_{A-1} may be optionally substituted formyl, said R_{A-2} may be optionally substituted piperazinyl, said R_{A-3} may be optionally substituted ethyl, and R_{A-4} may be selected from the group consisting of hydrogen, protium, deuterium, tritium, and optionally substituted hydroxy.

In another aspect, the present application provides a compound having the structure of formula (I-A),

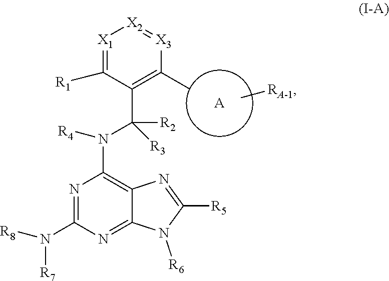

or a pharmaceutically acceptable salt, or prodrug thereof, or a solvate or hydrate of any of the forgoing, wherein, each X₁, X₂, and X₃ may be independently selected from optionally substituted —CH═, and —N═, each R₁, R₂, R₃, R₄, R₅, R₆, R₇, and R₈, may be independently absent or may be independently selected from optional substituents, or R₇ and R₈ combined with the atoms to which they are attached may form an optionally substituted ring, wherein, ring A may be 5-ring-membered heteroaryl containing one or more N(nitrogen), said ring A may be substituted with R_{A-1}, said R_{A-1} may be optionally substituted alkyl, said ring A may be further optionally substituted with optional substituents.

For example, as a compound having the structure of formula (I-A), said X₁ may be optionally substituted —CH═.

For example, as a compound having the structure of formula (I-A), said X₂ may be optionally substituted —CH═.

For example, as a compound having the structure of formula (I-A), said X₃ may be optionally substituted —CH═ or —N═. For example, said X₃ may be optionally substituted —CH═ or said X₃ may be optionally substituted —N═.

For example, as a compound having the structure of formula (I-A), said R₁ may be selected from the group consisting of hydrogen, protium, deuterium, tritium, and halogen. For example, said R₁ may be H(hydrogen), F(fluorine) or Cl (chlorine). For example, said R₁ may be H(hydrogen), said R₁ may be F(fluorine) or said R₁ may be Cl (chlorine).

For example, as a compound having the structure of formula (I-A), said R₂ may be H.

For example, as a compound having the structure of formula (I-A), said R₃ may be H.

For example, as a compound having the structure of formula (I-A), said R₄ may be H.

For example, as a compound having the structure of formula (I-A), said R₅ may be H.

For example, as a compound having the structure of formula (I-A), said R₆ may be selected from the group consisting of hydrogen, protium, deuterium, tritium, and optionally substituted (C₁-C₆)alkyl. For example, R₆ may be optionally substituted propyl. For example, R₆ may be optionally substituted isopropyl.

For example, as a compound having the structure of formula (I-A), said R₇ may be selected from the group consisting of hydrogen, protium, deuterium, tritium, and optionally substituted 3 to 12-ring-membered heterocycle. For example, said R₇ may be optionally substituted 6-ring-membered heterocycle. For example, said R₇ may be optionally substituted piperidinyl. For example, said R₈ may be H.

For example, as a compound having the structure of formula (I-A), said R₇ and said R₈ combined with the atoms to which they are attached may form an optionally substituted ring B. For example, said ring B is optionally substituted 3 to 12-ring-membered heterocycle. For example, said ring B is optionally substituted 6-ring-membered heterocycle. For example, said ring B is optionally substituted piperidinyl.

For example, as a compound having the structure of formula (I-A), said ring B may be substituted with one or more R_{B-1}, each said R_{B-1} may be independently optional substituent. For example, said R_{B-1} may be selected from the group consisting of hydrogen, protium, deuterium, tritium, optionally substituted hydroxy, and optionally substituted amino.

For example, as a compound having the structure of formula (I-A), said ring B may be optionally substituted 3 to 12-ring-membered heterocycle, and said R_{B-1} may be selected from the group consisting of hydrogen, protium, deuterium, tritium, optionally substituted hydroxy, and optionally substituted amino. For example, said ring B may be optionally substituted piperidinyl, and said R_{B-1} may be selected from the group consisting of hydrogen, protium, deuterium, tritium, optionally substituted hydroxy, and optionally substituted amino.

For example, as a compound having the structure of formula (I-A), said ring A may be optionally substituted 5-ring-membered heteroaryl containing two N. For example, said ring A may be optionally substituted pyrazolyl.

For example, as a compound having the structure of formula (I-A), said ring A may be substituted with one or more R_{A-1}, each said R_{A-1} may be independently selected from optional substituents. For example, said R_{A-1} may be optionally substituted (C₁-C₆)alkyl. For example, said R_{A-1} may be optionally substituted methyl or optionally substituted propyl. For example, said R_{A-1} may be optionally substituted methyl or optionally substituted isopropyl.

For example, as a compound having the structure of formula (I-A), said ring A may be optionally substituted pyrazolyl, and said R_{A-1} may be optionally substituted (C₁-C₆)alkyl. For example, said ring A may be optionally substituted pyrazolyl, and said R_{A-1} may be optionally substituted methyl or optionally substituted isopropyl.

For example, as a compound having the structure of formula (I-A), said ring A may be substituted with one or more R_{A-1}, said R_{A-1} may be substituted with one or more R_{A-2}, each said R_{A-2} may be independently selected from optional substituents. For example, said R_{A-2} may be selected from the group consisting of hydrogen, protium, deuterium, tritium, ═O, optionally substituted hydroxy, and optionally substituted 3 to 12-ring-membered heterocycle. For example, said R_{A-2} may be hydrogen, protium, deuterium, tritium, ═O, optionally substituted hydroxy, or optionally substituted 6-ring-membered heterocycle. For example, said R_{A-2} may be hydrogen, protium, deuterium, tritium, ═O, optionally substituted hydroxy, or optionally substituted piperazinyl.

For example, as a compound having the structure of formula (I-A), said ring A may be optionally substituted pyrazolyl, said R_{A-1} may be optionally substituted (C₁-C₆)alkyl, and said R_{A-2} may be selected from the group consisting of hydrogen, protium, deuterium, tritium, ═O, optionally substituted hydroxy, and optionally substituted 3 to 12-ring-membered heterocycle. For example, said ring A may be optionally substituted pyrazolyl, said R_{A-1} may be optionally substituted methyl, and said R_{A-2} may be selected from the group consisting of hydrogen, protium, deuterium, tritium, ═O, optionally substituted hydroxy, and optionally substituted piperazinyl. For example, said ring A may be optionally substituted pyrazolyl, said R_{A-1} may be optionally substituted isopropyl, and said R_{A-2} may be selected from the group consisting of hydrogen, protium, deuterium, tritium, and optionally substituted hydroxy.

For example, as a compound having the structure of formula (I-A), said ring A may be substituted with one or more R_{A-1}, said R_{A-1} may be substituted with one or more R_{A-2}, said R_{A-2} may be substituted with one or more R_{A-3}, each said R_{A-3} may be independently selected from optional substituents. For example, said R_{A-3} may be selected from the group consisting of hydrogen, protium, deuterium, tritium, and optionally substituted (C₁-C₆)alkyl. For example, said R_{A-3} may be hydrogen, protium, deuterium, tritium, optionally substituted methyl or optionally substituted ethyl.

For example, as a compound having the structure of formula (I-A), said ring A may be optionally substituted pyrazolyl, said R_{A-1} may be optionally substituted (C₁-C₆)alkyl, said R_{A-2} may be optionally substituted piperazinyl, and said R_{A-3} may be selected from the group consisting of hydrogen, protium, deuterium, tritium, and optionally substituted (C₁-C₆)alkyl. For example, said ring A may be optionally substituted pyrazolyl, said R_{A-1} may be optionally substituted methyl, said R_{A-2} may be optionally substituted piperazinyl, and said R_{A-3} may be hydrogen, protium, deuterium, tritium, optionally substituted methyl or optionally substituted ethyl.

For example, as a compound having the structure of formula (I-A), said ring A may be substituted with one or more R_{A-1}, said R_{A-1} may be substituted with one or more R_{A-2}, said R_{A-2} may be substituted with one or more R_{A-3}, said R_{A-3} may be substituted with one or more R_{A-4}, each said R_{A-4} may be independently selected from optional substituents. For example, said R_{A-4} may be selected from the group consisting of hydrogen, protium, deuterium, tritium, and optionally substituted hydroxy.

For example, as a compound having the structure of formula (I-A), said ring A may be optionally substituted pyrazolyl, said R_{A-1} may be optionally substituted (C₁-C₆)alkyl, said R_{A-2} may be optionally substituted piperazinyl, said R_{A-3} may be optionally substituted (C₁-C₆)alkyl, and said R_{A-4} may be selected from the group consisting of hydrogen, protium, deuterium, tritium, and optionally substituted hydroxy. For example, said ring A may be optionally substituted pyrazolyl, said R_{A-1} may be optionally substituted methyl, said R_{A-2} may be optionally substituted piperazinyl, said R_{A-3} may be optionally substituted ethyl, and said R_{A-4} may be selected from the group consisting of hydrogen, protium, deuterium, tritium, and optionally substituted hydroxy.

In another aspect, the present application provides a compound having the structure of formula (I-B),

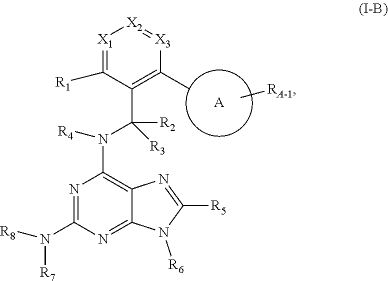

or a pharmaceutically acceptable salt, or prodrug thereof, or a solvate or hydrate of any of the forgoing, wherein, each X₁, X₂, and X₃ may be independently selected from optionally substituted —CH═, and —N═, each R₁, R₂, R₃, R₄, R₅, R₆, R₇, and R₈, may be independently absent or may be independently selected from optional substituents, or R₇ and R₈ combined with the atoms to which they are attached may form an optionally substituted ring, wherein, ring A may be 5-ring-membered heteroaryl containing one or more N(nitrogen), said ring A may be substituted with R_{A-1}, said R_{A-1} may be optionally substituted ring, said ring A may be further optionally substituted with optional substituents.

For example, as a compound having the structure of formula (I-B), said X₁ may be optionally substituted —CH═.

For example, as a compound having the structure of formula (I-B), said X₂ may be optionally substituted —CH═.

For example, as a compound having the structure of formula (I-B), said X₃ may be optionally substituted —CH═ or —N═. For example, said X₃ may be optionally substituted —CH═ or said X₃ may be optionally substituted —N═.

For example, as a compound having the structure of formula (I-B), said R₁ may be H(hydrogen).

For example, as a compound having the structure of formula (I-B), said R₂ may be H.

For example, as a compound having the structure of formula (I-B), said R₃ may be H.

For example, as a compound having the structure of formula (I-B), said R₄ may be H.

For example, as a compound having the structure of formula (I-B), said R₅ may be H.

For example, as a compound having the structure of formula (I-B), said R₆ may be optionally substituted (C₁-C₆)alkyl. For example, said R₆ may be optionally substituted propyl. For example, said R₆ may be optionally substituted isopropyl.

For example, as a compound having the structure of formula (I-B), said R₇ and said R₈ combined with the atoms to which they are attached may form an optionally substituted ring B. For example, said ring B may be optionally substituted 3 to 12-ring-membered heterocycle. For example, said ring B may be optionally substituted 6-ring-membered heterocycle. For example, said ring B may be optionally substituted piperidinyl.

For example, as a compound having the structure of formula (I-B), said ring B may be substituted with one or more R_{B-1}, each said R_{B-1} may be independently optional substituent. For example, said R_{B-1} may be selected from the group consisting of hydrogen, protium, deuterium, tritium, and optionally substituted amino.

For example, as a compound having the structure of formula (I-B), said ring B may be optionally substituted piperidinyl, said R_{B-1} may be selected from the group consisting of hydrogen, protium, deuterium, tritium, and optionally substituted amino.

For example, as a compound having the structure of formula (I-B), said ring A may be optionally substituted 5-ring-membered heteroaryl containing two N. For example, said ring A may be optionally substituted pyrazolyl.

For example, as a compound having the structure of formula (I-B), said R_{A-1} may be optionally substituted 3 to 12-ring-membered heterocycle. For example, said R_{A-1} may be optionally substituted 6-ring-membered heterocycle. For example, said R_{A-1} may be optionally substituted 6-ring-membered heterocycle containing two N. For example, said R_{A-1} may be optionally substituted piperazinyl.

For example, as a compound having the structure of formula (I-B), said R_{A-1} may be substituted with one or more R_{A-2}, each said R_{A-2} may be independently optional substituent. For example, said R_{A-2} may be optionally substituted (C₁-C₆)alkyl. For example, said R_{A-2} may be optionally substituted propyl. For example, said R_{A-2} may be optionally substituted isopropyl.

For example, as a compound having the structure of formula (I-B), said R_{A-1} may be optionally substituted piperazinyl, and said R_{A-2} may be optionally substituted isopropyl.

In another aspect, the present application provides a compound having the structure of formula (I-C),

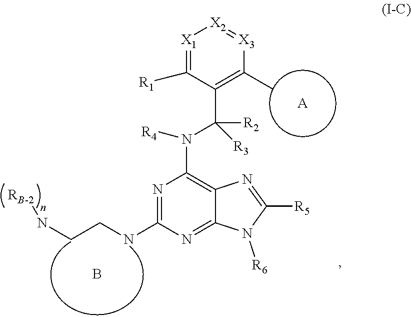

or a pharmaceutically acceptable salt, or prodrug thereof, or a solvate or hydrate of any of the forgoing, wherein, each X₁, X₂, and X₃ may be independently selected from optionally substituted —CH═, and —N═, each R₁, R₂, R₃, R₄, R₅, R₆, and R_{B-2}, may be independently absent or may be independently selected from optional substituents, n may be 0 or more, wherein, ring A may be 5-ring-membered heteroaryl containing one or more N(nitrogen), said ring B may be 5 to 6-ring-membered heterocycle.

For example, as a compound having the structure of formula (I-C), said X₁ may be optionally substituted —CH═.

For example, as a compound having the structure of formula (I-C), said X₂ may be optionally substituted —CH═.

For example, as a compound having the structure of formula (I-C), said X₃ may be optionally substituted —CH═.

For example, as a compound having the structure of formula (I-C), said R₁ may be H(hydrogen).

For example, as a compound having the structure of formula (I-C), said R₂ may be H.

For example, as a compound having the structure of formula (I-C), said R₃ may be H.

For example, as a compound having the structure of formula (I-C), said R₄ may be H.

For example, as a compound having the structure of formula (I-C), said R₅ may be H.

For example, as a compound having the structure of formula (I-C), said R₆ may be optionally substituted (C₁-C₆)alkyl. For example, said R₆ may be optionally substituted propyl. For example, said R₆ may be optionally substituted isopropyl.

For example, as a compound having the structure of formula (I-C), said ring B may be optionally substituted piperidinyl or optionally substituted tetrahydropyrrolyl.

For example, as a compound having the structure of formula (I-C), R_{B-2} may be H. For example, said n may be 2.

For example, as a compound having the structure of formula (I-C), said ring A may be optionally substituted 5-ring-membered heteroaryl containing two N. For example, said ring A may be optionally substituted pyrazolyl.

In another aspect, the present application provides a compound having the structure of formula (I-D),

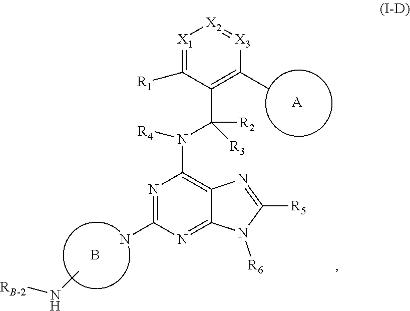

or a pharmaceutically acceptable salt, or prodrug thereof, or a solvate or hydrate of any of the forgoing, wherein, each X₁, X₂, and X₃ may be independently selected from optionally substituted —CH═, and —N═, each R₁, R₂, R₃, R₄, R₅, and R₆, may be independently absent or may be independently selected from optional substituents, wherein, ring A may be 5-ring-membered heteroaryl containing one or more N(nitrogen), said R_{B-2} may be optionally substituted (C₁-C₆)alkyl.

For example, as a compound having the structure of formula (I-D), said X₁ may be optionally substituted —CH═.

For example, as a compound having the structure of formula (I-D), said X₂ may be optionally substituted —CH═.

For example, as a compound having the structure of formula (I-D), said X₃ may be optionally substituted —CH═.

For example, as a compound having the structure of formula (I-D), said R₁ may be H(hydrogen).

For example, as a compound having the structure of formula (I-D), said R₂ may be H.

For example, as a compound having the structure of formula (I-D), said R₃ may be H.

For example, as a compound having the structure of formula (I-D), said R₄ may be H.

For example, as a compound having the structure of formula (I-D), said R₅ may be H.

For example, as a compound having the structure of formula (I-D), said R₆ may be optionally substituted (C₁-C₆)alkyl. For example, said R₆ may be optionally substituted propyl. For example, said R₆ may be optionally substituted isopropyl.

For example, as a compound having the structure of formula (I-D), said ring B may be optionally substituted piperidinyl.

For example, as a compound having the structure of formula (I-D), R_{B-2} may be optionally substituted methyl.

For example, as a compound having the structure of formula (I-D), said ring A may be optionally substituted 5-ring-membered heteroaryl containing two N. For example, said ring A may be optionally substituted pyrazolyl.

In another aspect, the present application provides a compound having the structure of formula (I-E),

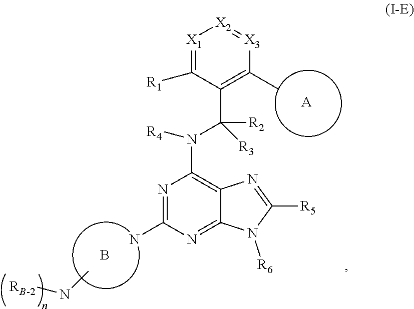

or a pharmaceutically acceptable salt, or prodrug thereof, or a solvate or hydrate of any of the forgoing, wherein, each X₁, X₂, and X₃ may be independently selected from optionally substituted —CH═, and —N═, each R₁, R₂, R₃, R₄, R₅, R₆, and R_{B-2} may be independently absent or may be independently selected from optional substituents, n may be 0 or more, wherein, ring A may be 5-ring-membered heteroaryl containing one or more N(nitrogen), one of said R_{B-2} may be optionally substituted (C₂-C₆)alkyl.

For example, as a compound having the structure of formula (I-E), said X₁ may be optionally substituted —CH═.

For example, as a compound having the structure of formula (I-E), said X₂ may be optionally substituted —CH═.

For example, as a compound having the structure of formula (I-E), said X₃ may be optionally substituted —CH═.

For example, as a compound having the structure of formula (I-E), said R₁ may be H(hydrogen).

For example, as a compound having the structure of formula (I-E), said R₂ may be H.

For example, as a compound having the structure of formula (I-E), said R₃ may be H.

For example, as a compound having the structure of formula (I-E), said R₄ may be H.

For example, as a compound having the structure of formula (I-E), said R₅ may be H.

For example, as a compound having the structure of formula (I-E), said R₆ may be optionally substituted (C₁-C₆)alkyl. For example, said R₆ may be optionally substituted propyl. For example, said R₆ may be optionally substituted isopropyl.

For example, as a compound having the structure of formula (I-E), said ring B may be optionally substituted piperidinyl.

For example, as a compound having the structure of formula (I-E), R_{B-2} may be optionally substituted ethyl. For example, said n may be 2.

For example, as a compound having the structure of formula (I-E), said ring A may be optionally substituted 5-ring-membered heteroaryl containing two N. For example, said ring A may be optionally substituted pyrazolyl.

In some cases, the present application provides a compound or a pharmaceutically acceptable salt, prodrug, or metabolite thereof, or a solvate or hydrate of any of the foregoing, wherein, said compound may be selected from the group consisting of

| Compound | Structure |
|---|---|
| I-1 | 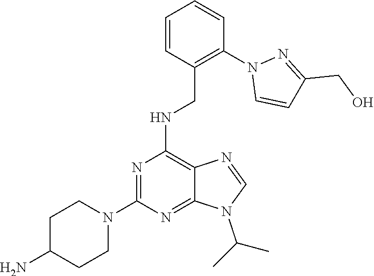 [SMILES: CC(C)n1cnc2c(NCc3ccccc3-n3ccc(CO)n3)nc(N3CCC(N)CC3)nc21] |
| I-2 | 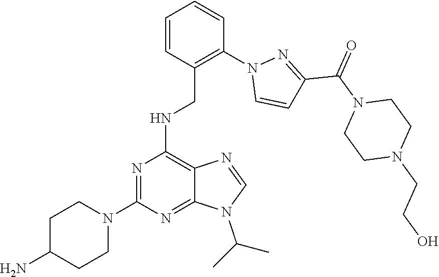 [SMILES: CC(C)n1cnc2c(NCc3ccccc3-n3ccc(C(=O)N4CCN(CCO)CC4)n3)nc(N3CCC(N)CC3)nc21] |
| I-3 | 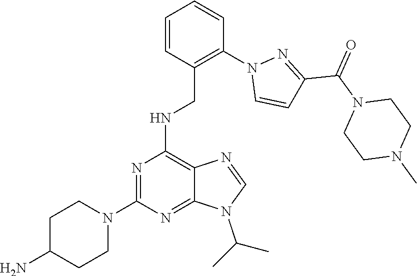 [SMILES: CC(C)n1cnc2c(NCc3ccccc3-n3ccc(C(=O)N4CCN(C)CC4)n3)nc(N3CCC(N)CC3)nc21] |
| I-4 | 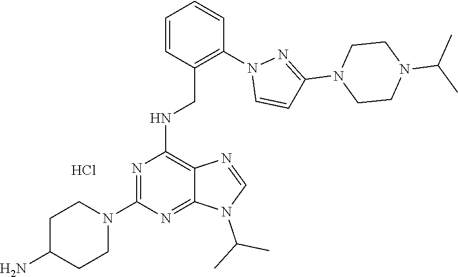 [SMILES: CC(C)N1CCN(c2ccn(-c3ccccc3CNc3nc(N4CCC(N)CC4)nc4c3ncn4C(C)C)n2)CC1 ; Cl] |
| I-5 | 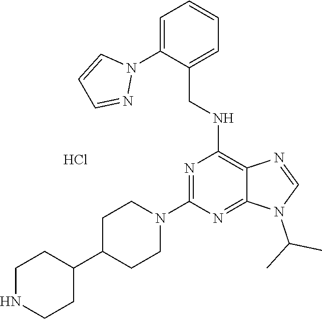 [SMILES: CC(C)n1cnc2c(NCc3ccccc3-n3cccn3)nc(N3CCC(C4CCNCC4)CC3)nc21 ; Cl] |
| I-6 | 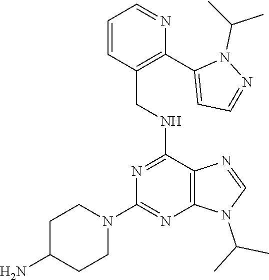 [SMILES: CC(C)n1nccc1-c1ncccc1CNc1nc(N2CCC(N)CC2)nc2c1ncn2C(C)C] |
| I-7 | 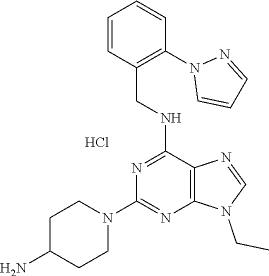 [SMILES: CCn1cnc2c(NCc3ccccc3-n3cccn3)nc(N3CCC(N)CC3)nc21 ; Cl] |
| I-8 | 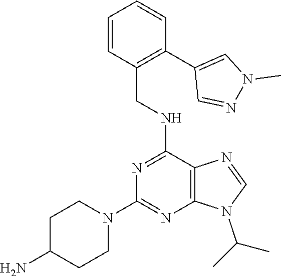 [SMILES: CC(C)n1cnc2c(NCc3ccccc3-c3cnn(C)c3)nc(N3CCC(N)CC3)nc21] |
| I-9 | 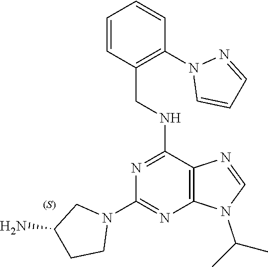 |
| I-10 | 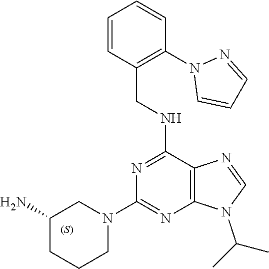 |
| I-11 | 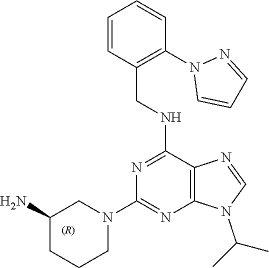 |
| I-12 | 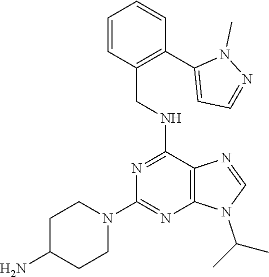 [SMILES: CC(C)n1cnc2c(NCc3ccccc3-c3ccnn3C)nc(N3CCC(N)CC3)nc21] |
| I-13 | 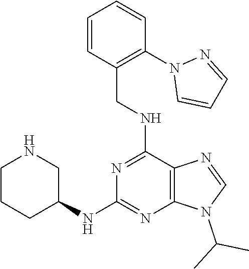 [SMILES: CC(C)n1cnc2c(NCc3ccccc3-n3cccn3)nc(N[C@H]3CCCNC3)nc21] |
| I-14 | 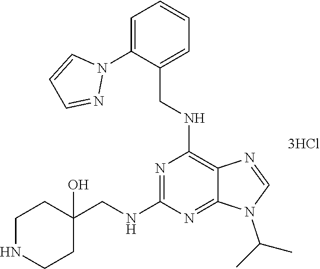 [SMILES: CC(C)n1cnc2c(NCc3ccccc3-n3cccn3)nc(NCC3(O)CCNCC3)nc21] |
| I-15 | 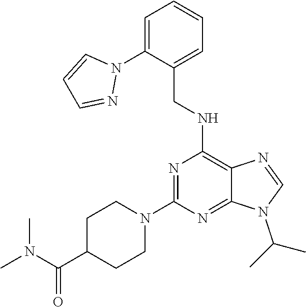 [SMILES: CC(C)n1cnc2c(NCc3ccccc3-n3cccn3)nc(N3CCC(C(=O)N(C)C)CC3)nc21] |
| I-16 | 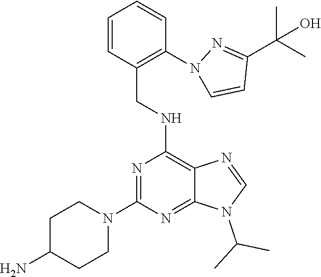 [SMILES: CC(C)n1cnc2c(NCc3ccccc3-n3ccc(C(C)(C)O)n3)nc(N3CCC(N)CC3)nc21] |
| I-17 | 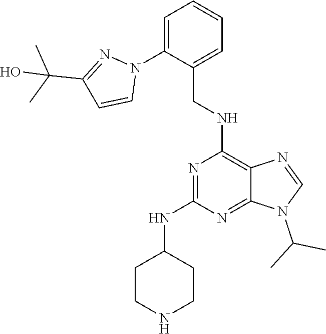 [SMILES: CC(C)n1cnc2c(NCc3ccccc3-n3ccc(C(C)(C)O)n3)nc(NC3CCNCC3)nc21] |
| I-18 | 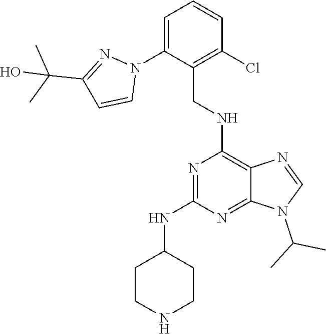 [SMILES: CC(C)n1cnc2c(NCc3c(Cl)cccc3-n3ccc(C(C)(C)O)n3)nc(NC3CCNCC3)nc21] |
| I-19 | 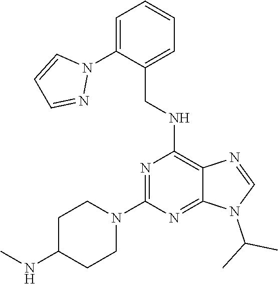 [SMILES: CNC1CCN(c2nc(NCc3ccccc3-n3cccn3)c3ncn(C(C)C)c3n2)CC1] |
| I-20 | 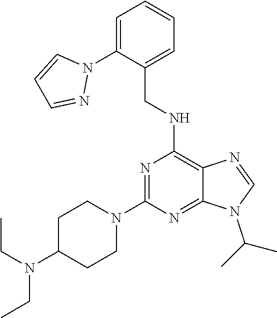 [SMILES: CCN(CC)C1CCN(c2nc(NCc3ccccc3-n3cccn3)c3ncn(C(C)C)c3n2)CC1] |
| I-21 | 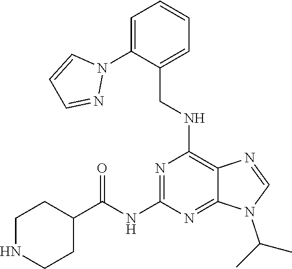 [SMILES: CC(C)n1cnc2c(NCc3ccccc3-n3cccn3)nc(NC(=O)C3CCNCC3)nc21] |
| I-22 | 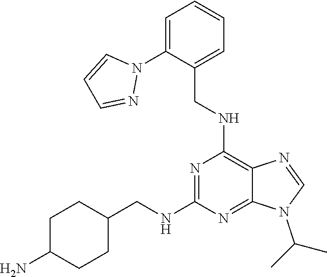 [SMILES: CC(C)n1cnc2c(NCc3ccccc3-n3cccn3)nc(NCC3CCC(N)CC3)nc21] |
| I-23 | 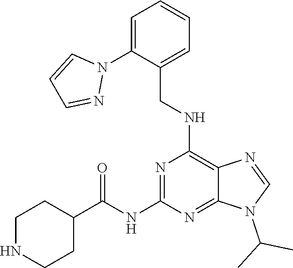 [SMILES: CC(C)n1cnc2c(NCc3ccccc3-n3cccn3)nc(NC(=O)C3CCNCC3)nc21] |
| I-24 | 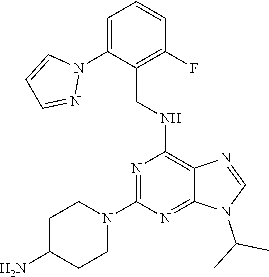 [SMILES: CC(C)n1cnc2c(NCc3c(F)cccc3-n3cccn3)nc(N3CCC(N)CC3)nc21] |
| I-25 | 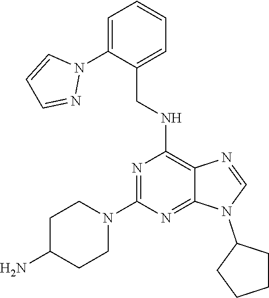 [SMILES: NC1CCN(c2nc(NCc3ccccc3-n3cccn3)c3ncn(C4CCCC4)c3n2)CC1] |
| I-26 | 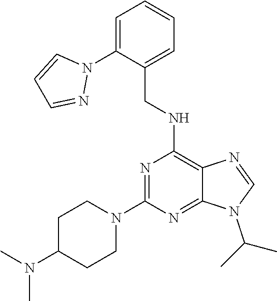 [SMILES: CC(C)n1cnc2c(NCc3ccccc3-n3cccn3)nc(N3CCC(N(C)C)CC3)nc21] |
| I-27 | 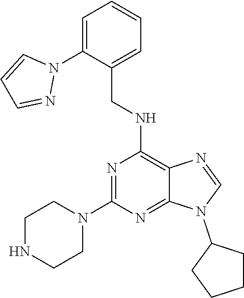 [SMILES: c1ccc(-n2cccn2)c(CNc2nc(N3CCNCC3)nc3c2ncn3C2CCCC2)c1] |
| I-28 | 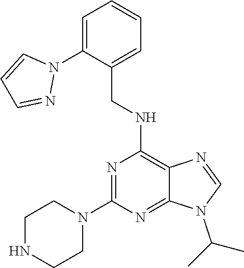 [SMILES: CC(C)n1cnc2c(NCc3ccccc3-n3cccn3)nc(N3CCNCC3)nc21] |
| I-29 | 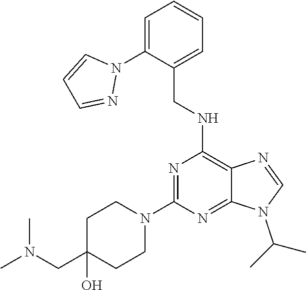 [SMILES: CC(C)n1cnc2c(NCc3ccccc3-n3cccn3)nc(N3CCC(O)(CN(C)C)CC3)nc21] |
| I-30 | 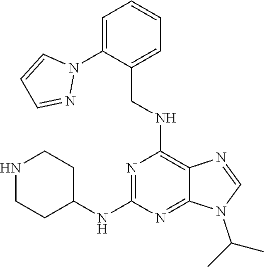 [SMILES: CC(C)n1cnc2c(NCc3ccccc3-n3cccn3)nc(NC3CCNCC3)nc21] |
| I-31 | 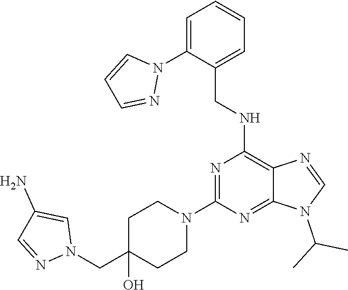 [SMILES: CC(C)n1cnc2c(NCc3ccccc3-n3cccn3)nc(N3CCC(O)(Cn4cc(N)cn4)CC3)nc21] |
| I-32 | 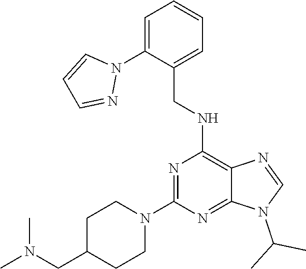 [SMILES: CC(C)n1cnc2c(NCc3ccccc3-n3cccn3)nc(N3CCC(CN(C)C)CC3)nc21] |
| I-33 | 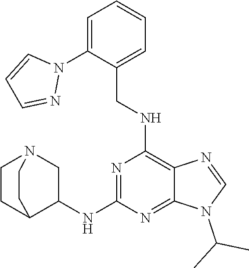 [SMILES: CC(C)n1cnc2c(NCc3ccccc3-n3cccn3)nc(NC3CN4CCC3CC4)nc21] |
| I-34 | 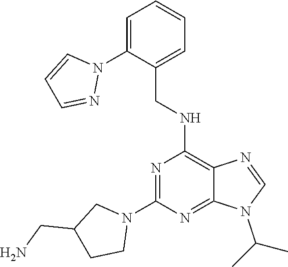 [SMILES: CC(C)n1cnc2c(NCc3ccccc3-n3cccn3)nc(N3CCC(CN)C3)nc21] |
| I-35 | 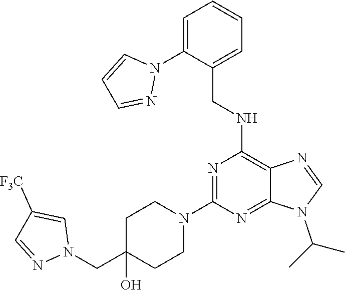 [SMILES: CC(C)n1cnc2c(NCc3ccccc3-n3cccn3)nc(N3CCC(O)(Cn4cc(C(F)(F)F)cn4)CC3)nc21] |
| I-36 | 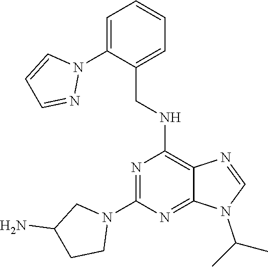 [SMILES: CC(C)n1cnc2c(NCc3ccccc3-n3cccn3)nc(N3CCC(N)C3)nc21] |
| I-37 | 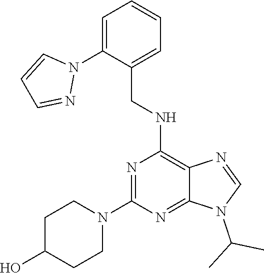 [SMILES: CC(C)n1cnc2c(NCc3ccccc3-n3cccn3)nc(N3CCC(O)CC3)nc21] |
| I-38 | 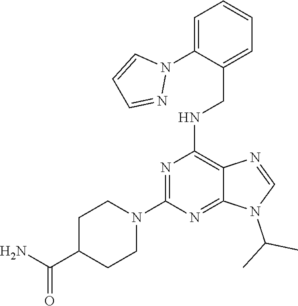 [SMILES: CC(C)n1cnc2c(NCc3ccccc3-n3cccn3)nc(N3CCC(C(N)=O)CC3)nc21] |
| I-39 | 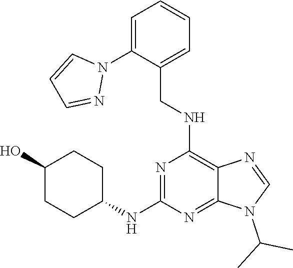 [SMILES: CC(C)n1cnc2c(NCc3ccccc3-n3cccn3)nc(N[C@H]3CC[C@H](O)CC3)nc21] |
| I-40 | 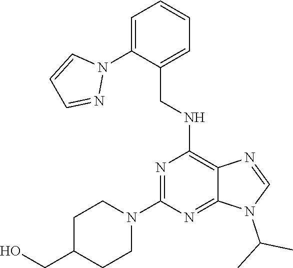 [SMILES: CC(C)n1cnc2c(NCc3ccccc3-n3cccn3)nc(N3CCC(CO)CC3)nc21] |
| I-41 | 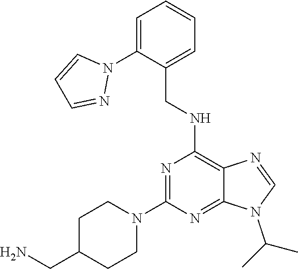 [SMILES: CC(C)n1cnc2c(NCc3ccccc3-n3cccn3)nc(N3CCC(CN)CC3)nc21] |

In another aspect, the present application provides a compound having the structure of formula (II),

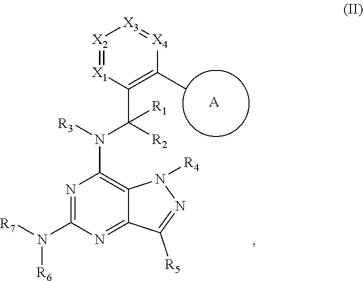

or a pharmaceutically acceptable salt, or prodrug thereof, or a solvate or hydrate of any of the forgoing, wherein, each X₁, X₂, X₃, and X₄ may be independently selected from optionally substituted —CH═, and —N═, each R₁, R₂, R₃, R₄, R₅, R₆, and R₇, may be independently absent or may be independently selected from optional substituents, or R₆ and R₇ combined with the atoms to which they are attached may form an optionally substituted ring, wherein, ring A may be optionally substituted ring.

For example, as a compound having the structure of formula (II), said X₁ may be optionally substituted —CH═.

For example, as a compound having the structure of formula (II), said X₂ may be optionally substituted —CH═.

For example, as a compound having the structure of formula (II), said X₃ may be optionally substituted —CH═.

For example, as a compound having the structure of formula (II), said X₄ may be optionally substituted —CH═.

For example, as a compound having the structure of formula (II), said R₁ may be selected from the group consisting of hydrogen, protium, deuterium, tritium, and halogen. For example, said R₁ may be H(hydrogen), F(fluorine) or Cl (chlorine). For example, said R₁ may be H(hydrogen).

For example, as a compound having the structure of formula (II), said R₂ may be H.

For example, as a compound having the structure of formula (II), said R₃ may be H.

For example, as a compound having the structure of formula (II), said R₄ may be H.

For example, as a compound having the structure of formula (II), said R₅ may be selected from the group consisting of hydrogen, protium, deuterium, tritium, and optionally substituted (C₁-C₆)alkyl. For example, R₅ may be optionally substituted propyl. For example, R₅ may be optionally substituted isopropyl.

For example, as a compound having the structure of formula (II), said R₆ may be selected from the group consisting of hydrogen, protium, deuterium, tritium, and optionally substituted 3 to 12-ring-membered heterocycle. For example, said R₆ may be optionally substituted 6-ring-membered heterocycle. For example, said R₆ may be optionally substituted piperidinyl. For example, said R₇ may be H.

For example, as a compound having the structure of formula (II), said R₆ and said R₇ combined with the atoms to which they are attached may form an optionally substituted ring B. For example, said ring B is optionally substituted 3 to 12-ring-membered heterocycle. For example, said ring B is optionally substituted 6-ring-membered heterocycle. For example, said ring B is optionally substituted piperidinyl.

For example, as a compound having the structure of formula (II), said ring B may be substituted with one or more R_{B-1}, each said R_{B-1} may be independently optional substituent. For example, said R_{B-1} may be selected from the group consisting of hydrogen, protium, deuterium, tritium, and optionally substituted amino.

For example, as a compound having the structure of formula (II), said ring A may be optionally substituted 5 to 12-ring-membered heteroaryl. For example, said ring A may be optionally substituted 5-ring-membered heteroaryl. For example, said ring A may be optionally substituted 5-ring-membered heteroaryl containing two N. For example, said ring A may be optionally substituted pyrazolyl.

In some cases, the present application provides a compound or a pharmaceutically acceptable salt, prodrug, or metabolite thereof, or a solvate or hydrate of any of the foregoing, wherein, said compound may be selected from the group consisting of

| Compound | Structure |
|---|---|
| II-1 | 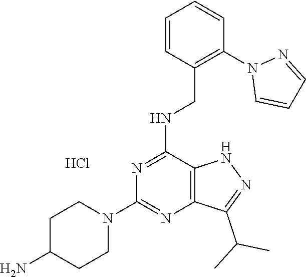 |
| II-2 | 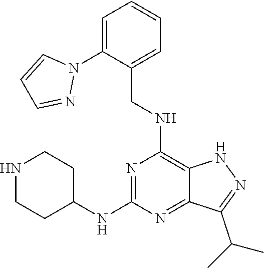 [SMILES: CC(C)c1n[nH]c2c(NCc3ccccc3-n3cccn3)nc(NC3CCNCC3)nc12] |

In one aspect, the present application provides a method for inhibiting cyclin-dependent kinase (CDK) activity, said method comprising administering to a subject in need thereof an effective amount of the present application, or a pharmaceutically acceptable salt, prodrug, or metabolite thereof, or a solvate or hydrate of any of the foregoing. For example, said cyclin-dependent kinase (CDK) may be CDK 7. For example, said method may be selected from the group consisting of an in vitro method, an ex vivo method, and an in vivo method.

In another embodiment, the present application provides use the compound of the present application, or a pharmaceutically acceptable salt, prodrug, or metabolite thereof, or a solvate or hydrate of any of the foregoing of the present application in the preparation of a drug and/or a kit for use in inhibiting cyclin-dependent kinase (CDK) activity. For example, said cyclin-dependent kinase (CDK) may be CDK 7. For example, said method of using said drug and/or said kit may be selected from the group consisting of an in vitro method, an ex vivo method, and an in vivo method.

In another embodiment, the present application provides the compound of the present application, or a pharmaceutically acceptable salt, prodrug, or metabolite thereof, or a solvate or hydrate of any of the foregoing of the present application for use in inhibiting cyclin-dependent kinase (CDK) activity. For example, said cyclin-dependent kinase (CDK) may be CDK 7. For example, said method or said use may be selected from the group consisting of an in vitro method, an ex vivo method, and an in vivo method.

In one aspect, the present application provides a composition comprising a compound of the present application, or a pharmaceutically acceptable salt, prodrug, or metabolite thereof, or a solvate or hydrate of any of the foregoing, and optionally a pharmaceutically acceptable carrier.

The compounds of the application may be administered orally. Oral administration may involve swallowing, so that the compound enters the gastrointestinal tract, or buccal or sublingual administration may be employed by which the compound enters the blood stream directly from the mouth.

In some cases, the compounds of the present application may also be administered directly into the blood stream, into muscle, or into an internal organ. Suitable means for parenteral administration may include intravenous, intraarterial, intraperitoneal, intrathecal, intraventricular, intraurethral, intrasternal, intracranial, intramuscular and subcutaneous. Suitable devices for parenteral administration may include needle (including microneedle) injectors, needle-free injectors and infusion techniques.

The compounds of the present application may also be administered topically to the skin or mucosa, that is, dermally or transdermally. In some cases, the compounds of the present application may also be administered intranasally or by inhalation. In some cases, the compounds of the present application may be administered rectally or vaginally. In another embodiment, the compounds of the present application may also be administered directly to the eye or ear.

The dosage regimen for the compounds and/or compositions containing the compounds is based on a variety of factors, including the type, age, weight, sex and condition of the patient; the severity of the condition; the route of administration; and the activity of the particular compound employed. Thus, the dosage regimen may vary widely. Dosage levels of body weight per day may be useful.

Suitable subjects according to the present invention include mammalian subjects. Mammals according to the present invention may include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the present application provides use of one or more compounds of the present application for the preparation of a medicament.

For example, the compounds of the present application may be administered as compound per se. Alternatively, pharmaceutically acceptable salts may be suitable for medical applications because of their greater aqueous solubility relative to the parent compound.

In another embodiment, the present application provides compositions. Such compositions may comprise a compound of the present application presented with a pharmaceutically acceptable carrier. The carrier may be a solid product, a liquid, or both, and may be formulated with the compound as a unit-dose composition, for example, a tablet, which may contain the active compounds. A compound of the present application may be coupled with suitable polymers as targetable drug carriers. Other pharmacologically active substances may also be present.

The compounds of the present invention may be administered by any suitable route, maybe in the form of a pharmaceutical composition adapted to such a route. The active compounds and compositions, for example, may be administered orally, rectally, parenterally, or topically.

The compounds of the present application may be used, alone or in combination with other therapeutic agents. The compound(s) of the present application and other therapeutic agent(s) may be administered simultaneously (either in the same dosage form or in separate dosage forms) or sequentially.

The administration of two or more compounds “in combination” may mean that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” may mean that the compounds are administered in combination.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Preparation of Compounds

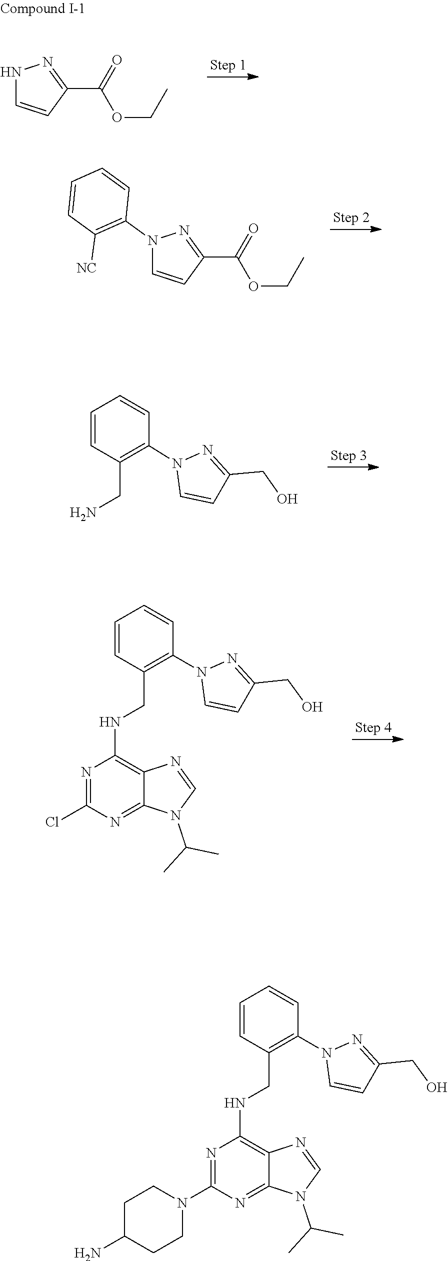

### Step 1

### ethyl 1-(2-cyanophenyl)-1H-pyrazole-3-carboxylate

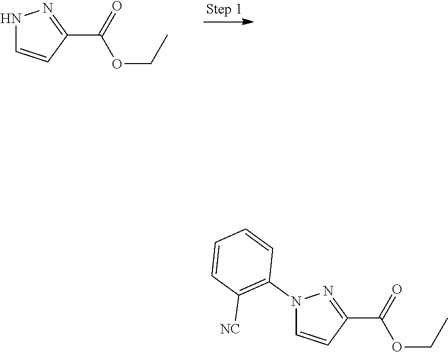

To a solution of ethyl 1H-pyrazole-3-carboxylate (6.94 g, 49.54 mmol) in DMF (50 mL) was added 2-fluorobenzonitrile (5 g, 41.28 mmol) and Cs₂CO₃ (26.90 g, 82.57 mmol), then stirred at 20° C. for 16 hrs. The reaction was poured into water (500 mL), extracted with EtOAc (500 mL*2), the organic layer was washed with water (500 mL*2), dried over Na₂SO₄, filtered and concentrated to give a residue. The residue was purified by column chromatography to give C₁₃H₁₁N₃O₂ (7.43 g, 29.26 mmol, 70.87% yield, 95% purity) as a yellow solid.

LCMS [M+H]⁺. 242.33

### Step 2

### (1-(2-(aminomethyl)phenyl)-1H-pyrazol-3-yl)methanol

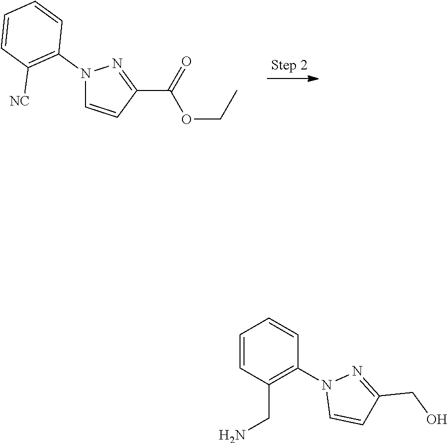 [SMILES: CCOC(=O)c1ccn(-c2ccccc2C#N)n1 ; NCc1ccccc1-n1ccc(CO)n1]

To a solution of ethyl 1-(2-cyanophenyl)-1H-pyrazole-3-carboxylate (200 mg, 829.03 μmol) in THF (10 mL) was added LiAlH₄ (34.61 mg, 911.94 μmol). After being stirred for 1 h, the reaction was diluted with THF and quenched with water (100 μL), NaOH (15% in water, 100 μL). The reaction mixture was stirred at room temperature for 1 h, then dried over Na₂SO₄. After filtration and evaporation of the solvent, the crude mixture was purified by column chromatography on silica gel. The product (1-(2-(aminomethyl)phenyl)-1H-pyrazol-3-yl)methanol (70.45 mg, 329.30 μmol, 39.72% yield, 95% purity) was obtained as a thick oil.

### Step 3

### Synthesis of (1-(2-(((2-chloro-9-isopropyl-9H-purin-6-yl)amino)methyl)phenyl)-1H-pyrazol-3-yl)methanol

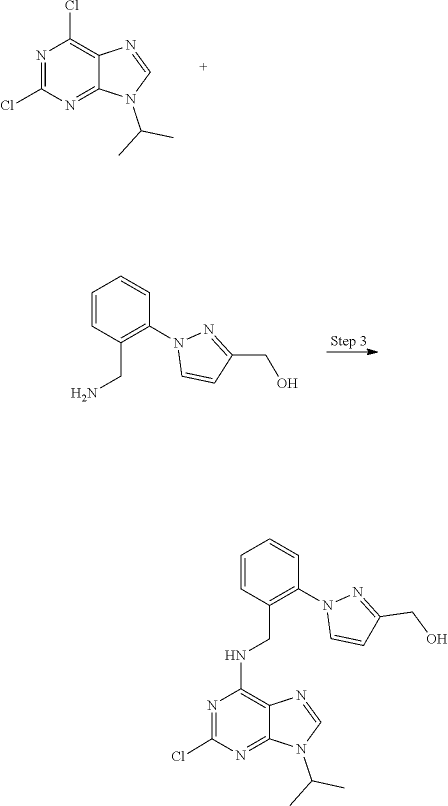 [SMILES: CC(C)n1cnc2c(Cl)nc(Cl)nc21 ; NCc1ccccc1-n1ccc(CO)n1 ; CC(C)n1cnc2c(NCc3ccccc3-n3ccc(CO)n3)nc(Cl)nc21]

TEA (262.74 mg, 2.60 mmol, 362.15 μL) and the (1-(2-(aminomethyl)phenyl)-1H-pyrazol-3-yl)methanol (184.70 mg, 908.77 μmol) were added to a solution of the 2,6-dichloro-9-isopropyl-9H-purine (200 mg, 865.50 μmol) in EtOH (10 mL) and the reaction mixture was heated at 110° C. overnight. Then, the solvent was removed under vacuum and the crude product was purified by column chromatographic on silica gel to give (1-(2-(((2-chloro-9-isopropyl-9H-purin-6-yl)amino)methyl)phenyl)-1H-pyrazol-3-yl)methanol (321.25 mg, 767.07 μmol, 88.63% yield, 95% purity) as a white solid. LCMS [M+H]⁺: 398.45

### Step 4

### Synthesis of (1-(2-(((2-(4-aminopiperidin-1-yl)-9-isopropyl-9H-purin-6-yl)amino)methyl)phenyl)-1H-pyrazol-3-yl)methanol

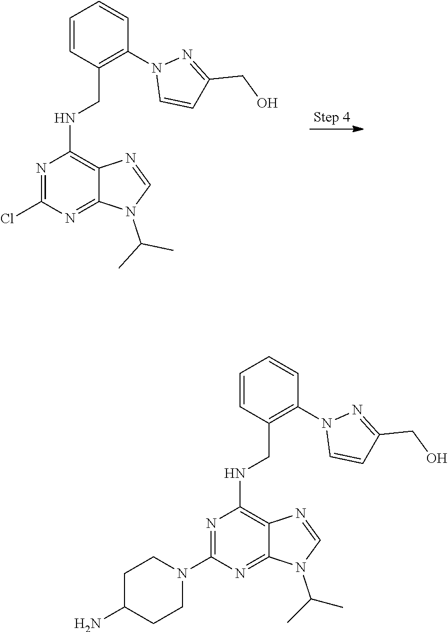 [SMILES: CC(C)n1cnc2c(NCc3ccccc3-n3ccc(CO)n3)nc(Cl)nc21 ; CC(C)n1cnc2c(NCc3ccccc3-n3ccc(CO)n3)nc(N3CCC(N)CC3)nc21]

To a solution of (1-(2-(((2-chloro-9-isopropyl-9H-purin-6-yl)amino)methyl)phenyl)-1H-pyrazol-3-yl)methanol (100 mg, 251.34 μmol) and piperidin-4-amine (30.21 mg, 301.61 μmol) in DMSO (2 mL) was added TEA (76.30 mg, 754.03 μmol, 105.17 μL). The mixture was heated at 160° C. for 16 hrs. LCMS showed one main peak with desired mass. The reaction mixture was filtered and concentrated under reduced pressure to give a residue. The residue was purified by column chromatography to give (1-(2-(((2-(4-aminopiperidin-1-yl)-9-isopropyl-9H-purin-6-yl)amino)methyl)phenyl)-1H-pyrazol-3-yl)methanol (69 mg, 142.02 μmol, 56.50% yield, 95% purity) as a white solid.

¹H NMR (400 MHz, Methanol-d4) δ 7.87 (d, J=3.4 Hz, 2H), 7.69-7.59 (m, 1H), 7.41 (q, J=3.6 Hz, 3H), 6.56 (d, J=2.4 Hz, 1H), 4.77-4.60 (m, 5H), 3.63 (q, J=7.0 Hz, 1H), 3.33 (p, J=1.7 Hz, 2H), 2.98-2.81 (m, 2H), 2.08-1.91 (m, 2H), 1.57 (d, J=6.7 Hz, 6H), 1.52-1.41 (m, 2H). LCMS [M+H]⁺. 462.24

### Compound I-2

### Step 1

### ethyl 1-(2-cyanophenyl)-1H-pyrazole-3-carboxylate

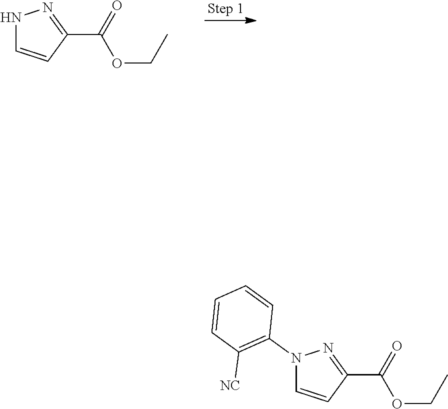

To a solution of ethyl 1H-pyrazole-3-carboxylate (6.94 g, 49.54 mmol) in DMF (50 mL) was added 2-fluorobenzonitrile (5 g, 41.28 mmol) and Cs₂CO₃ (26.90 g, 82.57 mmol), then stirred at 20° C. for 16 hrs. The reaction was poured into water (500 mL), extracted with EtOAc (500 mL*2), the organic layer was washed with water (500 mL*2), dried over Na₂SO₄, filtered and concentrated to give a residue. The residue was purified by column chromatography to give ethyl 1-(2-cyanophenyl)-1H-pyrazole-3-carboxylate(7.43 g, 29.26 mmol, 70.87% yield, 95% purity) as a yellow solid.

LCMS [M+H]⁺: 242.33

### Step 2

### 1-(2-cyanophenyl)-1H-pyrazole-3-carboxylic acid

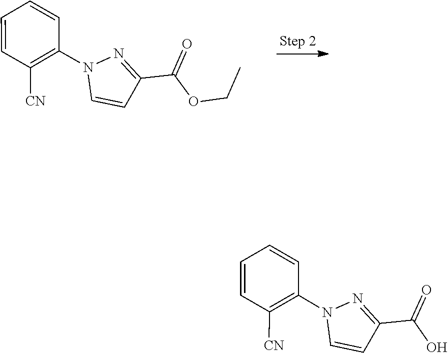 [SMILES: CCOC(=O)c1ccn(-c2ccccc2C#N)n1 ; N#Cc1ccccc1-n1ccc(C(=O)O)n1]

To a solution of ethyl 1-(2-cyanophenyl)-1H-pyrazole-3-carboxylate (3 g, 12.44 mmol) in MeOH (20 mL), H₂O (10 mL), THE (20 mL) was added LiOH (1.49 g, 62.18 mmol) then stirred at 20° C. for 16 hrs. The reaction was concentrated to give a residue. The pH of this residue was adjusted to 2 with diluted hydrochloric acid, then filtered and dried to give 1-(2-cyanophenyl)-1H-pyrazole-3-carboxylic acid (2.56 g, 11.41 mmol, 91.73% yield, 95% purity) as a white solid.

LCMS [M+H]⁺: 213.56

### Step 3

### Synthesis of 2-(3-(4-(2-hydroxyethyl)piperazine-1-carbonyl)-1H-pyrazol-1-yl)benzonitrile

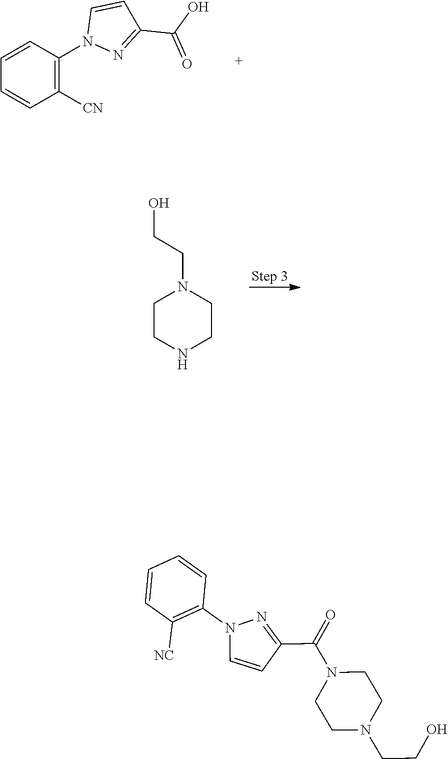 [SMILES: N#Cc1ccccc1-n1ccc(C(=O)O)n1 ; OCCN1CCNCC1 ; N#Cc1ccccc1-n1ccc(C(=O)N2CCN(CCO)CC2)n1]

To a solution of 1-(2-cyanophenyl)-1H-pyrazole-3-carboxylic acid (500 mg, 2.35 mmol) in DMF (15 mL) was added 2-(piperazin-1-yl)ethan-1-ol (458.00 mg, 3.52 mmol), DIPEA (909.32 mg, 7.04 mmol, 1.23 mL), HATU (1.33 g, 3.52 mmol), then stirred at 20° C. for 16 hrs. The reaction was poured into water (100 mL), extracted with EtOAc (100 mL*2), the organic layer was washed with water (100 mL*2), dried over Na₂SO₄, filtered and concentrated to give a residue. The residue was purified by column chromatography to give 2-(3-(4-(2-hydroxyethyl)piperazine-1-carbonyl)-1H-pyrazol-1-yl)benzonitrile (587.45 mg, 1.72 mmol, 73.1% yield, 95% purity) as a yellow solid.

LCMS [M+H]⁺: 325.46

### Step 4

### Synthesis of (1-(2-(aminomethyl)phenyl)-1H-pyrazol-3-yl)(4-(2-hydroxyethyl)piperazin-1-yl)methanone

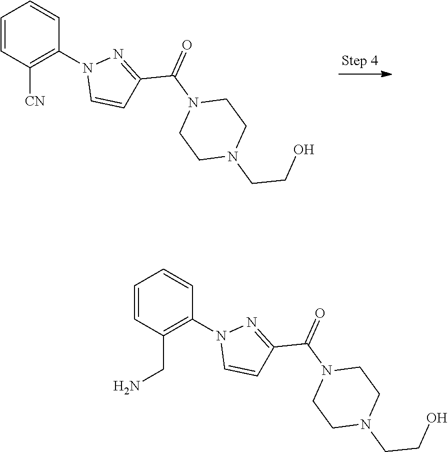 [SMILES: N#Cc1ccccc1-n1ccc(C(=O)N2CCN(CCO)CC2)n1 ; NCc1ccccc1-n1ccc(C(=O)N2CCN(CCO)CC2)n1]

To a solution of 2-(3-(4-(2-hydroxyethyl)piperazine-1-carbonyl)-1H-pyrazol-1-yl)benzonitrile (500 mg, 1.54 mmol) in MeOH (10 mL) was added Raney-Ni (0.1 g, 1.54 mmol) and ammonia water (1 mL), then stirred at 15° C. for 5 hrs. The reaction was filtered and concentrated to give a residue. The residue was purified by column chromatography to give (1-(2-(aminomethyl)phenyl)-1H-pyrazol-3-yl)(4-(2-hydroxyethyl)piperazin-1-yl)methanone (467.73 mg, 1.44 mmol, 93.5% yield) as a colourless oil.

LCMS [M+H]⁺: 326.43

### Step 5

### Synthesis of (1-(2-(((2-chloro-9-isopropyl-9H-purin-6-yl)amino)methyl)phenyl)-1H-pyrazol-3-yl)(4-(2-hydroxyethyl)piperazin-1-yl)methanone

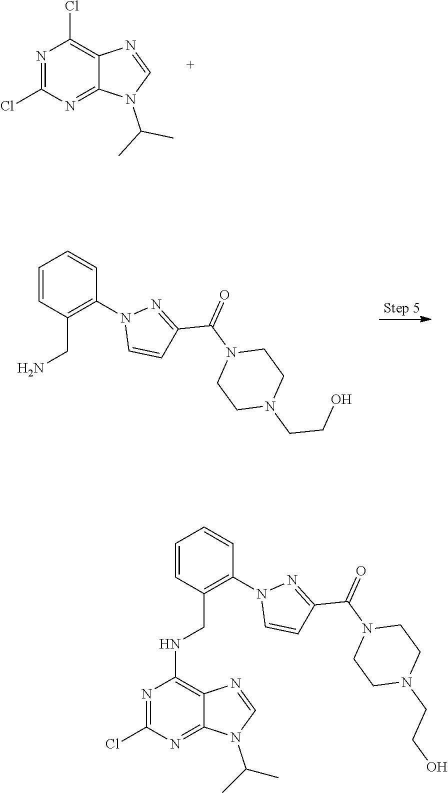 [SMILES: CC(C)n1cnc2c(Cl)nc(Cl)nc21 ; NCc1ccccc1-n1ccc(C(=O)N2CCN(CCO)CC2)n1 ; CC(C)n1cnc2c(NCc3ccccc3-n3ccc(C(=O)N4CCN(CCO)CC4)n3)nc(Cl)nc21]

TEA (131.37 mg, 1.30 mmol, 181.07 μL) and (1-(2-(aminomethyl)phenyl)-1H-pyrazol-3-yl)(4-(2-hydroxyethyl)piperazin-1-yl)methanone (149.67 mg, 454.38 μmol) were added to a solution of the 2,6-dichloro-9-isopropyl-9H-purine (100 mg, 432.75 μmol) in EtOH (15 mL) and the reaction mixture was heated at 80° C. for 8 hrs. Then, the solvent was removed under vacuum and the crude product was purified by column chromatographic on silica gel to give (1-(2-(((2-chloro-9-isopropyl-9H-purin-6-yl)amino)methyl)phenyl)-1H-pyrazol-3-yl)(4-(2-hydroxyethyl)piperazin-1-yl)methanone (201.34 mg, 365.01 μmol, 84.3% yield, 95% purity) a white solid.

LCMS [M+H]⁺: 525.45

### Step 6

### Synthesis of (1-(2-(((2-(4-aminopiperidin-1-yl)-9-isopropyl-9H-purin-6-yl)amino)methyl)phenyl)-1H-pyrazol-3-yl)(4-(2-hydroxyethyl)piperazin-1-yl)methanone

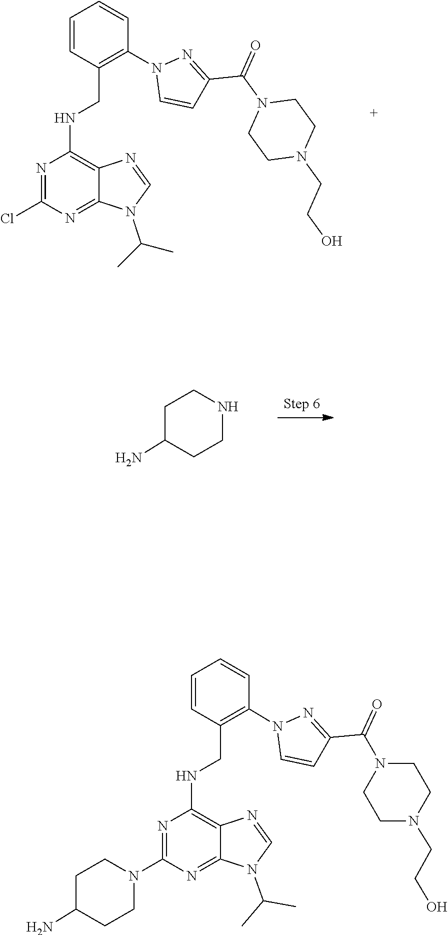 [SMILES: CC(C)n1cnc2c(NCc3ccccc3-n3ccc(C(=O)N4CCN(CCO)CC4)n3)nc(Cl)nc21 ; NC1CCNCC1 ; CC(C)n1cnc2c(NCc3ccccc3-n3ccc(C(=O)N4CCN(CCO)CC4)n3)nc(N3CCC(N)CC3)nc21]

To a solution of (1-(2-(((2-chloro-9-isopropyl-9H-purin-6-yl)amino)methyl)phenyl)-1H-pyrazol-3-yl)(4-(2-hydroxyethyl)piperazin-1-yl)methanone (100 mg, 190.83 μmol) and piperidin-4-amine (22.94 mg, 229.00 μmol) in DMSO (2 mL) was added TEA (57.93 mg, 572.50 μmol, 79.85 L) at 160° C. for 8 hrs. The reaction was concentrated to give a residue. The residue was purified by column chromatography to give (1-(2-(((2-(4-aminopiperidin-1-yl)-9-isopropyl-9H-purin-6-yl)amino)methyl)phenyl)-1H-pyrazol-3-yl)(4-(2-hydroxyethyl)piperazin-1-yl)methanone (76.21 mg, 123.19 μmol, 64.55% yield, 95% purity) as a white solid.

¹H NMR (400 MHz, Methanol-d4) δ 8.01 (d, J=2.5 Hz, 1H), 7.79 (s, 1H), 7.69-7.57 (m, 1H), 7.53-7.38 (m, 3H), 6.87 (d, J=2.4 Hz, 1H), 4.77-4.56 (m, 5H), 4.01 (d, J=5.2 Hz, 2H), 3.80 (t, J=5.2 Hz, 2H), 3.72-3.53 (m, 2H), 2.97-2.76 (m, 2H), 2.60 (t, J=5.2 Hz, 2H), 2.49 (dt, J=13.8, 5.5 Hz, 3H), 1.96 (s, 2H), 1.82 (dd, J=13.1, 3.8 Hz, 2H), 1.56 (d, J=6.8 Hz, 6H), 1.33-1.15 (m, 4H). LCMS [M+H]⁺: 588.26

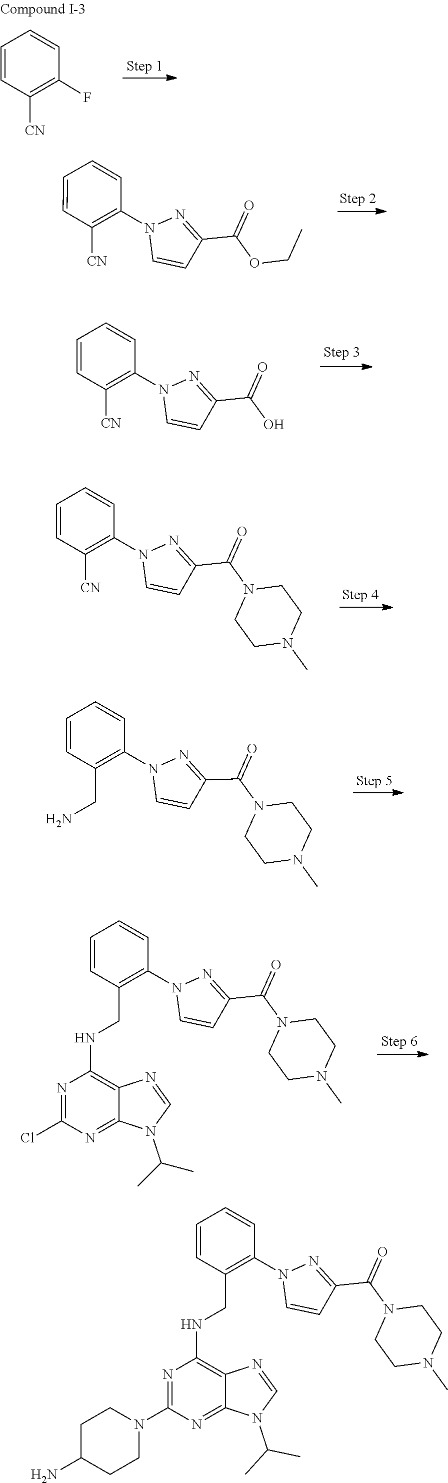

### Step 1

### ethyl 1-(2-cyanophenyl)-1H-pyrazole-3-carboxylate

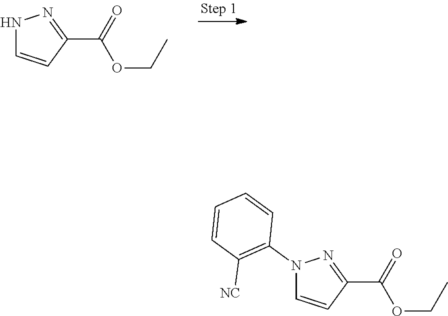

To a solution of ethyl 1H-pyrazole-3-carboxylate (6.94 g, 49.54 mmol) in DMF (50 mL) was added 2-fluorobenzonitrile (5 g, 41.28 mmol) and Cs₂CO₃ (26.90 g, 82.57 mmol), then stirred at 20° C. for 16 hrs. The reaction was poured into water (500 mL), extracted with EtOAc (500 mL*2), the organic layer was washed with water (500 mL*2), dried over Na₂SO₄, filtered and concentrated to give a residue. The residue was purified by column chromatography to give ethyl 1-(2-cyanophenyl)-1H-pyrazole-3-carboxylate(7.43 g, 29.26 mmol, 70.87% yield, 95% purity) as a yellow solid.

LCMS [M+H]⁺: 242.33

### Step 2

### 1-(2-cyanophenyl)-1H-pyrazole-3-carboxylic acid

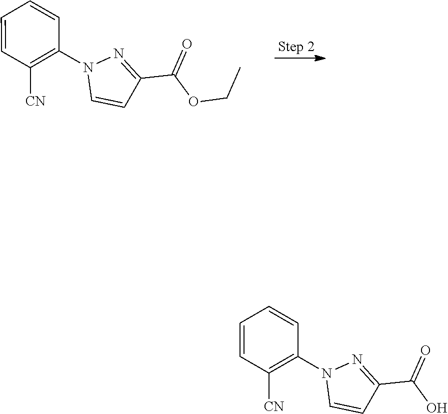 [SMILES: CCOC(=O)c1ccn(-c2ccccc2C#N)n1 ; N#Cc1ccccc1-n1ccc(C(=O)O)n1]

To a solution of ethyl 1-(2-cyanophenyl)-1H-pyrazole-3-carboxylate (3 g, 12.44 mmol) in MeOH (20 mL), H₂O (10 mL), THE (20 mL) was added LiOH (1.49 g, 62.18 mmol) then stirred at 20° C. for 16 hrs. The reaction was concentrated to give a residue. The pH of this residue was adjusted to 2 with diluted hydrochloric acid, filtered and dried to give 1-(2-cyanophenyl)-1H-pyrazole-3-carboxylic acid (2.56 g, 11.41 mmol, 91.73% yield, 95% purity) as a white solid.

LCMS [M+H]⁺: 213.56

### Step 3

### Synthesis of 2-(3-(4-methylpiperazine-1-carbonyl)-1H-pyrazol-1-yl)benzonitrile

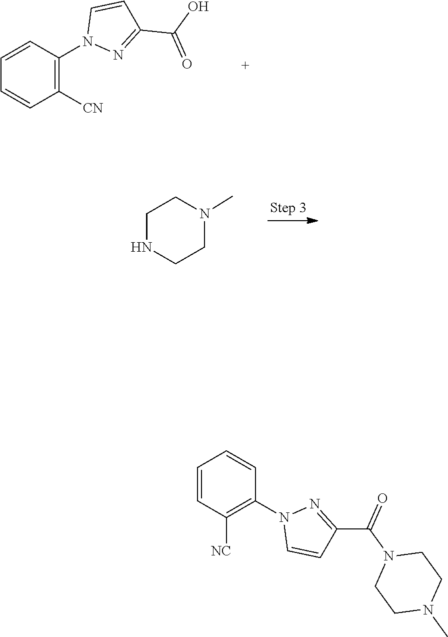

To a solution of 1-(2-cyanophenyl)-1H-pyrazole-3-carboxylic acid (500 mg, 2.35 mmol) in DMF (10 mL) was added 1-methylpiperazine (352.37 mg, 3.52 mmol), DIPEA (909.32 mg, 7.04 mmol, 1.23 mL) and HATU (1.33 g, 3.52 mmol), then stirred at 20° C. for 16 hrs. The reaction was poured into water (100 mL), extracted with EtOAc (100 mL*2), the organic layer was washed with water (100 mL*2), dried over Na₂SO₄, filtered and concentrated to give a residue. The residue was purified by column chromatography to give 2-(3-(4-methylpiperazine-1-carbonyl)-1H-pyrazol-1-yl)benzonitrile (678.43 mg, 2.30 mmol, 97.95% yield) as a colourless oil.

LCMS [M+H]⁺: 296.34

### Step 4

### Synthesis of (1-(2-(aminomethyl)phenyl)-1H-pyrazol-3-yl)(4-methylpiperazin-1-yl)methanone

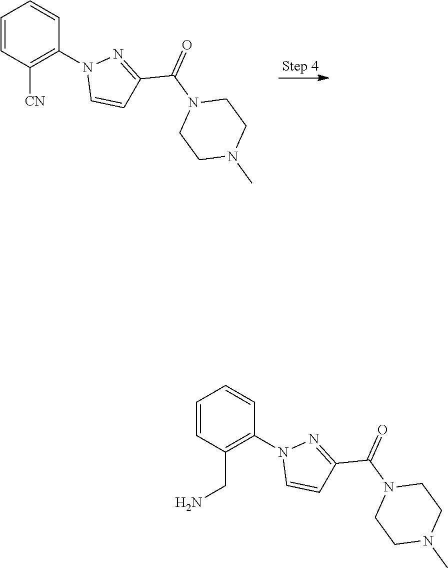 [SMILES: CN1CCN(C(=O)c2ccn(-c3ccccc3C#N)n2)CC1 ; CN1CCN(C(=O)c2ccn(-c3ccccc3CN)n2)CC1]

To a solution of 2-(3-(4-methylpiperazine-1-carbonyl)-1H-pyrazol-1-yl)benzonitrile (300 mg, 1.02 mmol) in MeOH (10 mL) was added Raney-Ni (0.1 g, 1.02 mmol) and ammonia water (2 mg, 1.02 mmol), then stirred at 20° C. for 16 hrs. The reaction was filtered and concentrated to give a residue. The residue was purified by column chromatography to give (1-(2-(aminomethyl)phenyl)-1H-pyrazol-3-yl)(4-methylpiperazin-1-yl)methanone (291.22 mg, 924.14 μmol, 90.98% yield, 95% purity) as a colourless oil.

LCMS [M+H]⁺: 292.34

### Step 5

### Synthesis of (1-(2-(((2-chloro-9-isopropyl-9H-purin-6-yl)amino)methyl)phenyl)-1H-pyrazol-3-yl)(4-methylpiperazin-1-yl)methanone

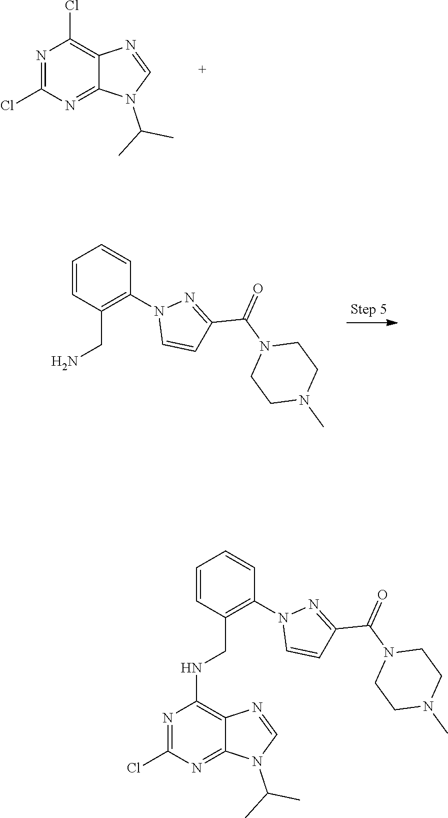 [SMILES: CC(C)n1cnc2c(Cl)nc(Cl)nc21 ; CN1CCN(C(=O)c2ccn(-c3ccccc3CN)n2)CC1 ; CC(C)n1cnc2c(NCc3ccccc3-n3ccc(C(=O)N4CCN(C)CC4)n3)nc(Cl)nc21]

TEA (131.37 mg, 1.30 mmol, 181.07 μL) and (1-(2-(aminomethyl)phenyl)-1H-pyrazol-3-yl)(4-methylpiperazin-1-yl)methanone (129.55 mg, 432.75 μmol) were added to a solution of 2,6-dichloro-9-isopropyl-9H-purine (100 mg, 432.75 μmol) in EtOH (15 mL) and the reaction mixture was heated at 80° C. overnight. The reaction was concentrated to give a residue. The residue was purified by column chromatography to give (1-(2-(((2-chloro-9-isopropyl-9H-purin-6-yl)amino)methyl)phenyl)-1H-pyrazol-3-yl)(4-methylpiperazin-1-yl)methanone (200.24 mg, 385.08 mol, 88.99% yield, 95% purity) as a white solid.

LCMS [M+H]⁺. 494.56

### Step 6

### Synthesis of (1-(2-(((2-(4-aminopiperidin-1-yl)-9-isopropyl-9H-purin-6-yl)amino)methyl)phenyl)-1H-pyrazol-3-yl)(4-methylpiperazin-1-yl)methanone

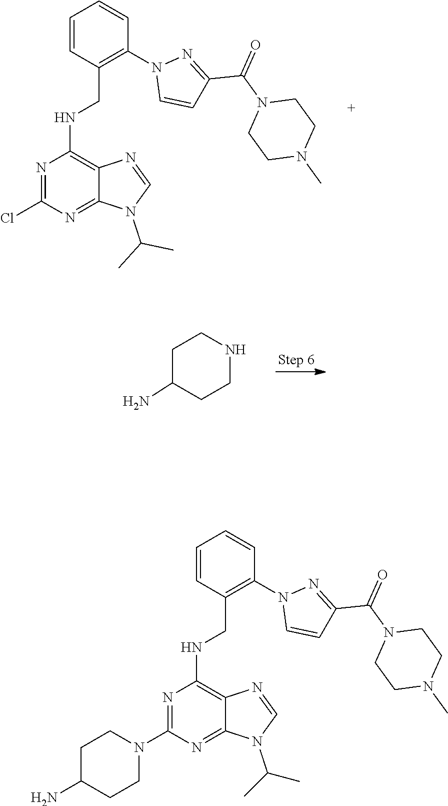 [SMILES: CC(C)n1cnc2c(NCc3ccccc3-n3ccc(C(=O)N4CCN(C)CC4)n3)nc(Cl)nc21 ; NC1CCNCC1 ; CC(C)n1cnc2c(NCc3ccccc3-n3ccc(C(=O)N4CCN(C)CC4)n3)nc(N3CCC(N)CC3)nc21]

To a solution of (1-(2-(((2-chloro-9-isopropyl-9H-purin-6-yl)amino)methyl)phenyl)-1H-pyrazol-3-yl)(4-methylpiperazin-1-yl)methanone (100 mg, 202.43 μmol) and piperidin-4-amine (24.33 mg, 242.92 μmol) in DMSO (2 mL) was added TEA (61.45 mg, 607.30 μmol, 84.70 μL) at 160° C. for 8 hrs. The reaction was concentrated to give a residue. The residue was purified by column chromatography to give (1-(2-(((2-(4-aminopiperidin-1-yl)-9-isopropyl-9H-purin-6-yl)amino)methyl)phenyl)-1H-pyrazol-3-yl)(4-methylpiperazin-1-yl)methanone (87 mg, 148.20 μmol, 73.21% yield, 95% purity) as a white solid.

¹H NMR (400 MHz, Methanol-d4) δ 8.04 (d, J=2.5 Hz, 1H), 7.84 (s, 1H), 7.70-7.60 (m, 1H), 7.55-7.39 (m, 3H), 6.91 (d, J=2.4 Hz, 1H), 4.80 (s, 1H), 4.73-4.65 (m, 3H), 4.20 (s, 2H), 3.90 (s, 2H), 3.63 (q, J=7.0 Hz, 1H), 2.95-2.81 (m, 4H), 2.53 (s, 3H), 1.96 (m, 4H), 1.57 (d, J=6.8 Hz, 6H), 1.46 (qd, J=12.2, 4.3 Hz, 2H), 1.31 (d, J=4.0 Hz, 1H), 1.20 (t, J=7.0 Hz, 1H). LCMS [M+H]⁺: 558.12

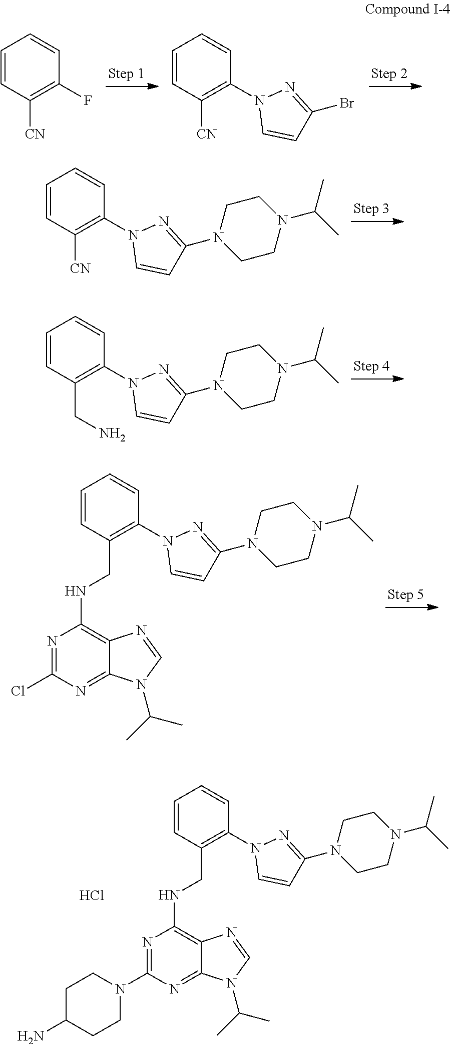

### Step 1

### 2-(3-bromo-1H-pyrazol-1-yl)benzonitrile

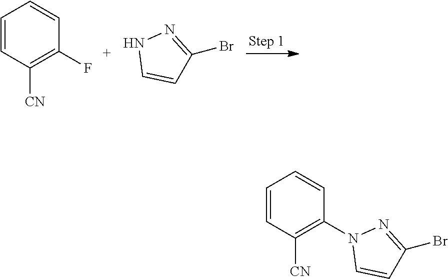 [SMILES: N#Cc1ccccc1F ; Brc1cc[nH]n1 ; N#Cc1ccccc1-n1ccc(Br)n1]

To a mixture of compound 2-fluorobenzonitrile (1 g, 8.26 mmol) and 3-bromo-1H-pyrazole (1.46 g, 9.91 mmol) in DMF (10 mL) was added Cs₂CO₃ (5.38 g, 17.15 mmol). The mixture was stirred at 15° C. for 15 hrs to form a white suspension. TLC showed the reaction was completed. The mixture was partitioned between EtOAc(150 mL) and H₂O (150 mL). The aqueous phase was extracted with EtOAc (150 mL×2). The combined organic extracts were washed with brine (100 mL×3), dried over anhydrous Na₂SO₄, filtered and concentrated under reduced pressure to give the crude product, which was purified by combi flash to give compound 2-(3-bromo-1H-pyrazol-1-yl)benzonitrile (1.87 g, 7.54 mmol, 91.29% yield) as a yellow solid. LCMS [M+H]⁺: 249.45

### Step 2

### 2-(3-(4-isopropylpiperazin-1-yl)-1H-pyrazol-1-yl)benzonitrile

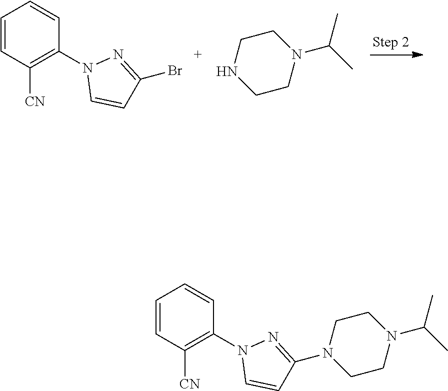

To a mixture of compound 2-(3-bromo-1H-pyrazol-1-yl)benzonitrile (200 mg, 806.20 μmol), 1-isopropylpiperazine (124.04 mg, 967.44 μmol) in toluene (10 mL) was added BINAP (150.60 mg, 241.86 μmol). The mixture was stirred at 110° C. for 16 hrs. TLC showed the reaction was completed. The mixture was partitioned between EtOAc (100 mL) and water (100 mL), the aqueous phase was extracted with EtOAc (80 mL×2), the combined extracted phase was washed with water (80 mL), dried over Na₂SO₄, filtered, concentrated under reduced pressure to give a yellow oil, which was purified by Combi flash to give 2-(3-(4-isopropylpiperazin-1-yl)-1H-pyrazol-1-yl)benzonitrile (156.33 mg, 502.79 μmol, 62.37% yield, 95% purity).

LCMS [M+H]⁺: 296.21

### Step 3

### Synthesis of (2-(3-(4-isopropylpiperazin-1-yl)-1H-pyrazol-1-yl)phenyl)methanamine

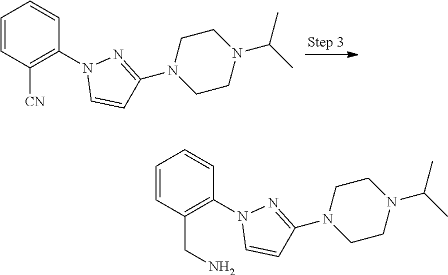 [SMILES: CC(C)N1CCN(c2ccn(-c3ccccc3C#N)n2)CC1 ; CC(C)N1CCN(c2ccn(-c3ccccc3CN)n2)CC1]

To a solution of Raney-Ni (100 mg) in MeOH (5 mL) was added 2-(3-(4-isopropylpiperazin-1-yl)-1H-pyrazol-1-yl)benzonitrile (64 mg, 216.67 μmol) and ammonia water (37.96 mg, 1.08 mmol), then stirred at 20° C. for 10 hrs under H₂ (15 Psi) atmosphere. The reaction mixture was filtered to give (2-(3-(4-isopropylpiperazin-1-yl)-1H-pyrazol-1-yl)phenyl)methanamine (54.33 mg, 181.46 μmol, 83.75% yield) as a yellow oil. LCMS [M+H]⁺: 300.23

### Step 4

### Synthesis of 2-chloro-9-isopropyl-N-(2-(3-(4-isopropylpiperazin-1-yl)-1H-pyrazol-1-yl)benzyl)-9H-purin-6-amine

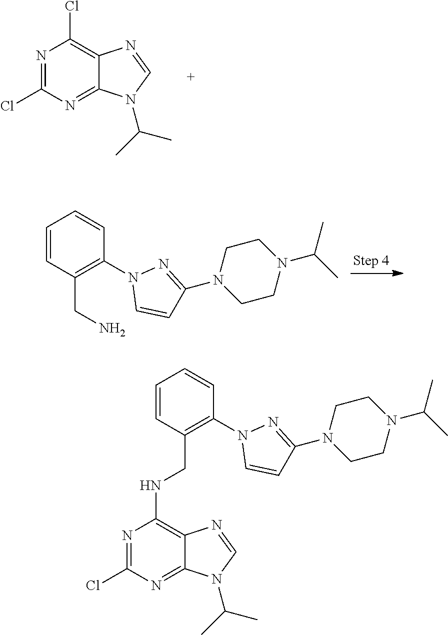 [SMILES: CC(C)n1cnc2c(Cl)nc(Cl)nc21 ; CC(C)N1CCN(c2ccn(-c3ccccc3CN)n2)CC1 ; CC(C)N1CCN(c2ccn(-c3ccccc3CNc3nc(Cl)nc4c3ncn4C(C)C)n2)CC1]

To a solution of 2,6-dichloro-9-isopropyl-9H-purine (73.50 mg, 318.08 μmol) and (2-(3-(4-isopropylpiperazin-1-yl)-1H-pyrazol-1-yl)phenyl)methanamine (100 mg, 333.99 μmol) in EtOH (10 mL) was added TEA (96.56 mg, 954.25 μmol, 133.09 μL), then stirred at 70° C. for 10 hrs under N₂ atmosphere. The reaction mixture was poured into petroleum ether (10 mL), and then filtered to give 2-chloro-9-isopropyl-N-(2-(3-(4-isopropylpiperazin-1-yl)-1H-pyrazol-1-yl)benzyl)-9H-purin-6-amine (145.63 mg, 280.04 μmol, 88.04% yield, 95% purity) as a white solid. LCMS [M+H]⁺: 495.36

### Step 5

### Synthesis of 2-(4-aminopiperidin-1-yl)-9-isopropyl-N-(2-(3-(4-isopropylpiperazin-1-yl)-1H-pyrazol-1-yl)benzyl)-9H-purin-6-amine hydrochloride

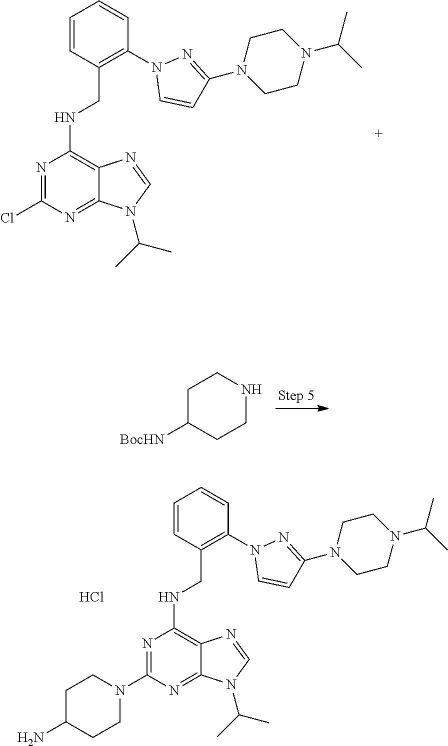 [SMILES: CC(C)N1CCN(c2ccn(-c3ccccc3CNc3nc(Cl)nc4c3ncn4C(C)C)n2)CC1 ; CC(C)(C)OC(=O)NC1CCNCC1 ; CC(C)N1CCN(c2ccn(-c3ccccc3CNc3nc(N4CCC(N)CC4)nc4c3ncn4C(C)C)n2)CC1 ; Cl]

To a solution of 2-chloro-9-isopropyl-N-(2-(3-(4-isopropylpiperazin-1-yl)-1H-pyrazol-1-yl)benzyl)-9H-purin-6-amine (43 mg, 87.04 μmol) and tert-butyl piperidin-4-ylcarbamate (20.92 mg, 104.45 μmol) in DMSO (1.98 mL) was added TEA (26.42 mg, 261.12 μmol, 36.42 μL). The mixture was heated at 160° C. for 10 hrs. The reaction was poured into water (30 mL), extracted with EtOAc (10 mL*2), the organic layer was washed with water (10 mL*2), dried over Na₂SO₄, filtered and concentrated to give a residue. The residue was purified by column chromatography to give a white solid. The solid was dissolved in methanol, then HCl/MeOH (20 mg, 261.12 μmol) was added and stirred at rt for 6 hrs. Then the mixture was concentrated to give 2-(4-aminopiperidin-1-yl)-9-isopropyl-N-(2-(3-(4-isopropylpiperazin-1-yl)-1H-pyrazol-1-yl)benzyl)-9H-purin-6-amine hydrochloride (35.23 mg, 60.01 μmol, 68.94% yield, 95% purity).

LCMS [M+H]⁺: 558.36

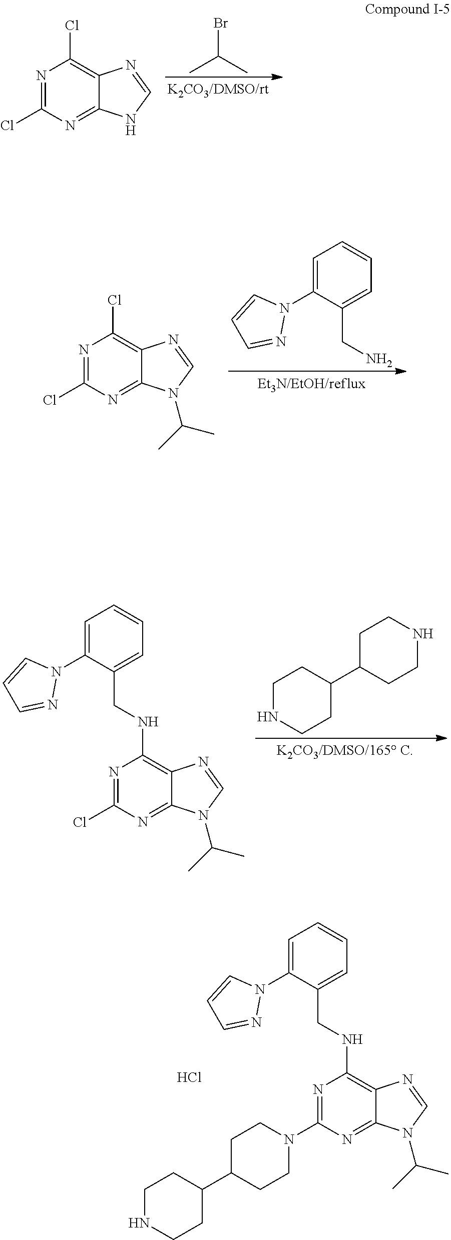

2,6-Dichloro-9-isopropyl-9H-purine: 2,6-dichloro-9H-purine (10.0 g, 52.91 mmol), isopropyl bromide (32.5 g, 264.55 mmol), potassium carbonate (21.9 g, 158.73 mmol) and dimethyl sulfoxide (50 mL) were added to a 250 mL two-necked flask, and the mixture was stirred at room temperature for 96 hrs. The reaction solution was added dropwise to water, ethyl acetate was added for extraction three times. The organic phases were combined, washed twice with saturated brine, dried over sodium sulfate, filtered and concentrated in vacuo to afford 9.8 g of light-yellow solid. The crude product was passed through a silica gel column (ethyl acetate/n-heptane=0-60%) to afford 4.941 g of white solid.

N-(2-(1H-pyrazol-1-yl)benzyl)-2-chloro-9-isopropyl-9H-purin-6-amine: 2,6-dichloro-9-isopropyl-9H-purine (2.082 g, 9.011 mmol), (2-(1H-pyrazol-1-yl)phenyl)methanamine (1.639 g, 9.462 mmol), triethylamine(1.368 g, 13.517 mmol) and ethanol(42 mL) were added to a 100 mL single-necked flask, and the mixture was heated and refluxed for 5 hrs. The reaction was concentrated in vacuum and purified by silica gel column (Methanol/dichloromethane=0-10%) to afford 3304 mg of off-white solid.

### N-(2-(1H-pyrazol-1-yl)benzyl)-2-([4,4′-bipiperidin]-1-yl)-9-isopropyl-9H-purin-6-amine hydrochloride

¹H NMR (400 MHz, DMSO-d6) δ 9.19 (s, 1H), 9.10 (d, J=10.5 Hz, 1H), 8.89 (s, 2H), 8.13 (s, 1H), 7.78 (s, 1H), 7.71-7.56 (m, 1H), 7.42 (d, J=3.3 Hz, 3H), 6.54 (s, 1H), 4.76-4.70 (m, 1H), 4.65 (d, J=5.6 Hz, 2H), 4.48 (d, J=12.8 Hz, 2H), 3.24 (d, J=12.0 Hz, 2H), 2.72 (dt, J=45.5, 12.1 Hz, 4H), 1.76 (d, J=12.6 Hz, 2H), 1.64 (d, J=12.5 Hz, 2H), 1.51 (d, J=6.5 Hz, 6H), 1.43-1.27 (m, 4H), 0.93 (q, J=11.8 Hz, 2H).

MS: cacl. for C28H38ClN9 499.32. Found: [M+H]⁺:500.42

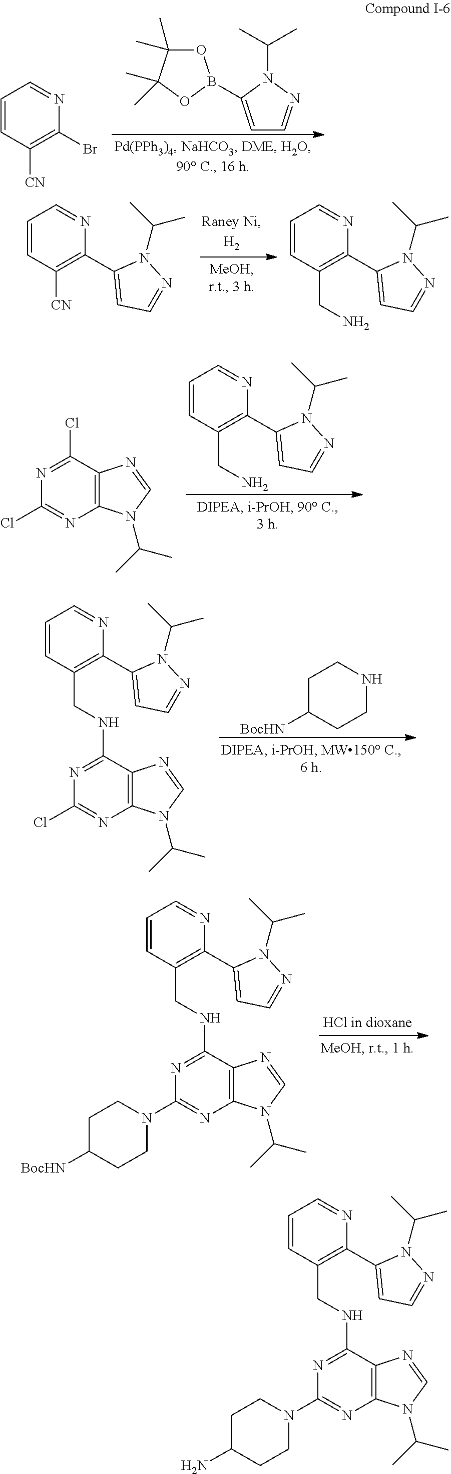

### Synthesis of 2-(1-isopropyl-1H-pyrazol-5-yl)nicotinonitrile

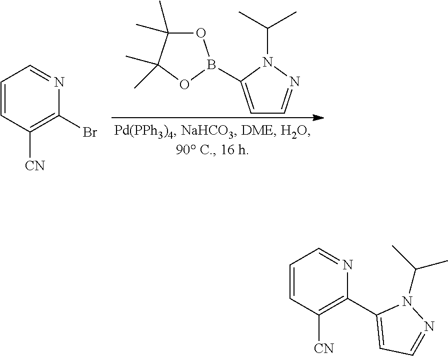 [SMILES: N#Cc1cccnc1Br ; CC(C)n1nccc1B1OC(C)(C)C(C)(C)O1 ; CC(C)n1nccc1-c1ncccc1C#N]

To a mixture of 2-bromonicotinonitrile (500 mg, 2.7 mmol), 1-isopropyl-5-(4,4,5,5-tetramethyl-1,3,2-dioxaborolan-2-yl)-1H-pyrazole (968 mg, 4.1 mmol) and NaHCO₃ (680 mg, 8.1 mmol) in DME (15 mL) and water (1.5 mL) was added Pd(PPh₃)₄ (312 mg, 0.27 mmol). The mixture was stirred at 90° C. for 16 hrs under N₂ atmosphere. The reaction mixture was cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=30/1) to give 2-(1-isopropyl-1H-pyrazol-5-yl)nicotinonitrile (350 mg, yield: 61%) as a colourless oil. ESI-MS [M+H]⁺: 213.2.

### Synthesis of (2-(1-isopropyl-1H-pyrazol-5-yl)pyridin-3-yl)methanamine

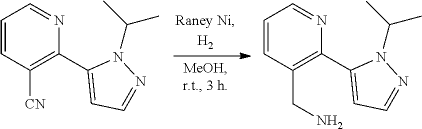 [SMILES: CC(C)n1nccc1-c1ncccc1C#N ; CC(C)n1nccc1-c1ncccc1CN]

To a mixture of 2-(1-isopropyl-1H-pyrazol-5-yl)nicotinonitrile (250 mg, 1.2 mmol) in NH₃ (7M solution in MeOH, 5 mL) was added Raney Ni (50 mg). The reaction mixture was stirred at room temperature for 3 hrs under H₂ atmosphere. The reaction mixture was filtered through Celite® and the filter cake was washed with DCM/MeOH (10/1, 20 mL). The filtrate was concentrated to give the crude, which was purified by Prep-TLC (eluent: DCM/MeOH=10/1) to give (2-(1-isopropyl-1H-pyrazol-5-yl)pyridin-3-yl)methanamine (200 mg, yield: 77%) as a yellow oil. ESI-MS [M+H]⁺: 217.2.

### Synthesis of 2-chloro-9-isopropyl-N-((2-(1-isopropyl-1H-pyrazol-5-yl)pyridin-3-yl)methyl)-9H-purin-6-amine

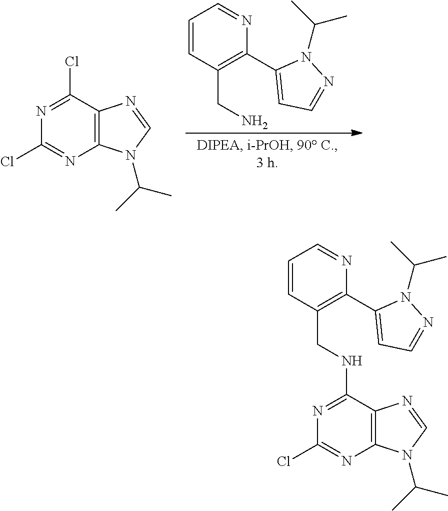 [SMILES: CC(C)n1cnc2c(Cl)nc(Cl)nc21 ; CC(C)n1nccc1-c1ncccc1CN ; CC(C)n1nccc1-c1ncccc1CNc1nc(Cl)nc2c1ncn2C(C)C]

A mixture of 2,6-dichloro-9-isopropyl-9H-purine (110 mg, 0.48 mmol), (2-(1-isopropyl-1H-pyrazol-5-yl)pyridin-3-yl)methanamine (127 mg, 0.59 mmol) and DIPEA (310 mg, 2.4 mmol) in i-PrOH (5 mL) was stirred at 90° C. for 3 hrs. The reaction mixture was concentrated to give the crude, which was purified by Prep-TLC (eluent: DCM/MeOH=20/1) to give 2-chloro-9-isopropyl-N-((2-(1-isopropyl-1H-pyrazol-5-yl)pyridin-3-yl)methyl)-9H-purin-6-amine (160 mg, yield: 81%) as a white solid. ESI-MS [M+H]⁺: 411.2.

### Synthesis of tert-butyl (1-(9-isopropyl-6-(((2-(1-isopropyl-1H-pyrazol-5-yl)pyridin-3-yl)methyl)amino)-9H-purin-2-yl)piperidin-4-yl)carbamate

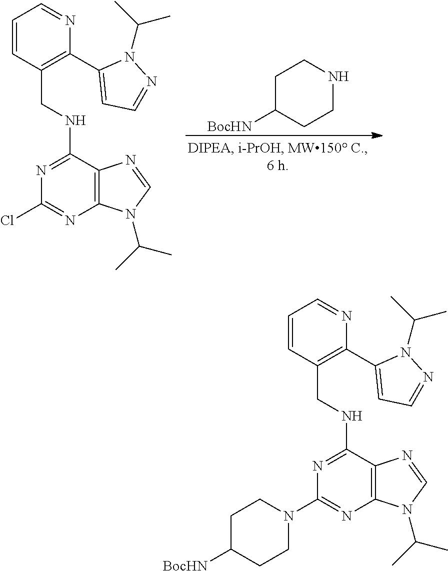 [SMILES: CC(C)n1nccc1-c1ncccc1CNc1nc(Cl)nc2c1ncn2C(C)C ; CC(C)(C)OC(=O)NC1CCNCC1 ; CC(C)n1nccc1-c1ncccc1CNc1nc(N2CCC(NC(=O)OC(C)(C)C)CC2)nc2c1ncn2C(C)C]

A mixture of 2-chloro-9-isopropyl-N-((2-(1-isopropyl-1H-pyrazol-5-yl)pyridin-3-yl)methyl)-9H-purin-6-amine (100 mg, 0.24 mmol), tert-butyl piperidin-4-ylcarbamate (60 mg, 0.30 mmol) and DIPEA (155 mg, 1.2 mmol) in i-PrOH (5 mL) was irradiated in microwave at 150° C. for 6 hrs. The reaction mixture was concentrated to give the crude, which was purified by Prep-TLC (eluent: DCM/MeOH=20/1) to give tert-butyl (1-(9-isopropyl-6-(((2-(1-isopropyl-1H-pyrazol-5-yl)pyridin-3-yl)methyl)amino)-9H-purin-2-yl)piperidin-4-yl)carbamate (80 mg, yield: 58%) as a white solid. ESI-MS [M+H]⁺: 575.2.

### Synthesis of 2-(4-aminopiperidin-1-yl)-9-isopropyl-N-((2-(1-isopropyl-1H-pyrazol-5-yl)pyridin-3-yl)methyl)-9H-purin-6-amine

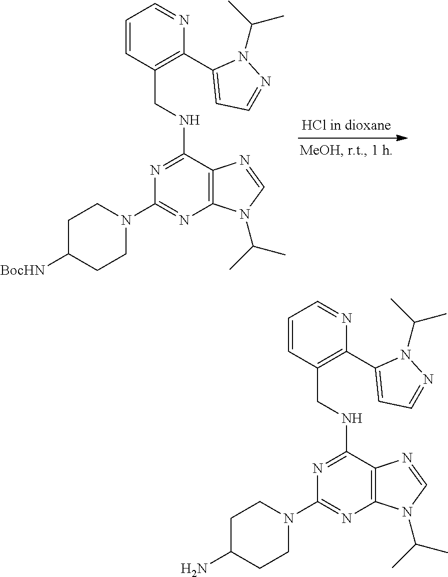 [SMILES: CC(C)n1nccc1-c1ncccc1CNc1nc(N2CCC(NC(=O)OC(C)(C)C)CC2)nc2c1ncn2C(C)C ; CC(C)n1nccc1-c1ncccc1CNc1nc(N2CCC(N)CC2)nc2c1ncn2C(C)C]

To a mixture of tert-butyl (1-(9-isopropyl-6-(((2-(1-isopropyl-1H-pyrazol-5-yl)pyridin-3-yl)methyl)amino)-9H-purin-2-yl)piperidin-4-yl)carbamate (80 mg, 0.14 mmol) in MeOH (2 mL) was added HCl (4M solution in 1,4-dioxane, 2 mL). The reaction mixture was stirred at room temperature for 1 h. The reaction mixture was quenched with NH₃ (7M solution in MeOH, 2 mL) and concentrated in vacuo to give the crude, which was purified by Prep-TLC (eluent: DCM/MeOH=10/1) to give 2-(4-aminopiperidin-1-yl)-9-isopropyl-N-((2-(1-isopropyl-1H-pyrazol-5-yl)pyridin-3-yl)methyl)-9H-purin-6-amine (47 mg, yield: 71%) as a white solid.

ESI-MS [M+H]⁺: 475.3.

¹HNMR (400 MHz, Methanol-d4) δ 8.58-8.54 (m, 1H), 7.99-7.95 (m, 1H), 7.79 (s, 1H), 7.62 (d, J=1.8 Hz, 1H), 7.48-7.45 (m, 1H), 6.50 (d, J=1.9 Hz, 1H), 4.68-4.65 (m, 3H), 4.59 (d, J=13.3 Hz, 2H), 4.41-4.34 (m, 1H), 2.86-2.76 (m, 3H), 1.76 (d, J=9.8 Hz, 2H), 1.54 (d, J=6.8 Hz, 6H), 1.40 (d, J=6.6 Hz, 6H), 124-1.13 (m, 2H).

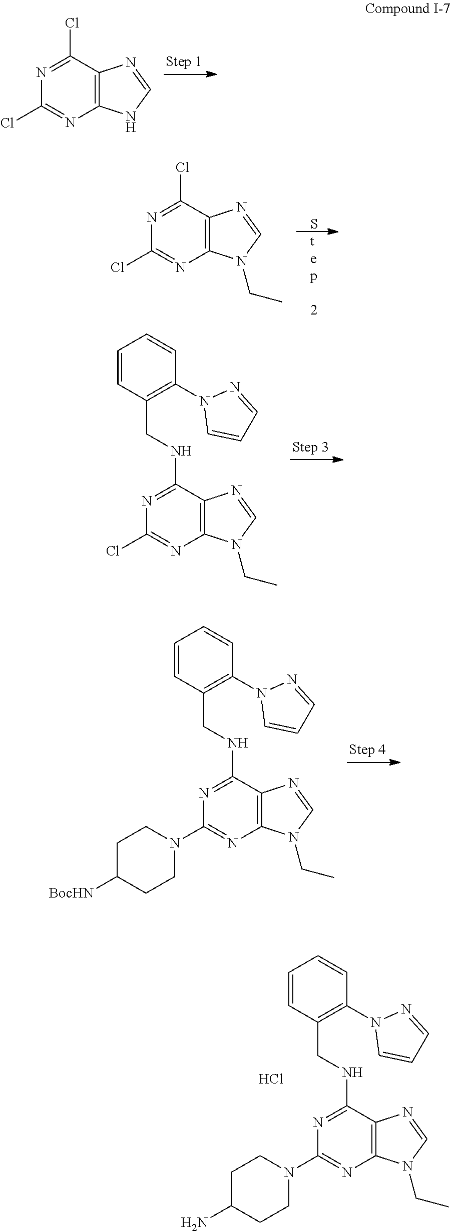 [SMILES: Clc1nc(Cl)c2nc[nH]c2n1 ; CCn1cnc2c(Cl)nc(Cl)nc21 ; CCn1cnc2c(NCc3ccccc3-n3cccn3)nc(Cl)nc21 ; CCn1cnc2c(NCc3ccccc3-n3cccn3)nc(N3CCC(NC(=O)OC(C)(C)C)CC3)nc21 ; CCn1cnc2c(NCc3ccccc3-n3cccn3)nc(N3CCC(N)CC3)nc21 ; Cl]

### Step 1

### 2,6-dichloro-9-ethyl-9H-purine

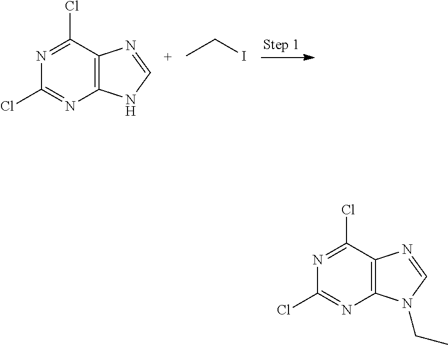

To a solution of 2,6-dichloro-9H-purine (1 g, 5.29 mmol) in DMF (10 mL) was added K₂CO₃ (731.24 mg, 5.29 mmol) and iodoethane (1.65 g, 10.58 mmol) at 0° C., then stirred for 16 hrs. The reaction was poured into water (30 mL), extracted with EtOAc (10 mL*2), the organic layer was washed with water (10 mL*2), dried over Na₂SO₄, filtered and concentrated to give a residue. The residue was purified by column chromatography to give 2,6-dichloro-9-ethyl-9H-purine (800 mg, 3.61 mmol, 68.27% yield, 98% purity) as a white solid.

LCMS [M+H]⁺: 218.32

### Step 2

### N-(2-(1H-pyrazol 1-yl)benzyl)-2-chloro-9-ethyl-9H-purin-6-amine

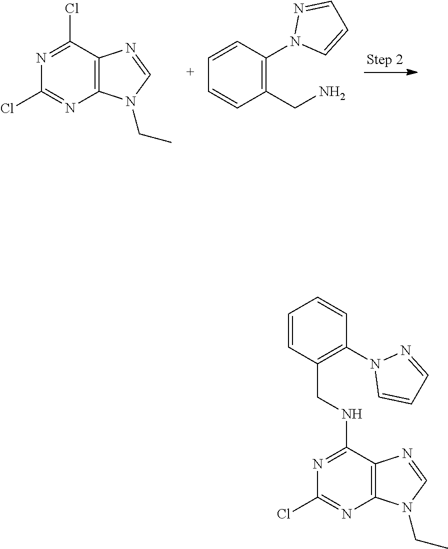 [SMILES: CCn1cnc2c(Cl)nc(Cl)nc21 ; NCc1ccccc1-n1cccn1 ; CCn1cnc2c(NCc3ccccc3-n3cccn3)nc(Cl)nc21]

To a solution of 2,6-dichloro-9-ethyl-9H-purine (150 mg, 691.07 μmol) in EtOH (10 mL) was added (2-(1H-pyrazol-1-yl)phenyl)methanamine (125.69 mg, 725.62 μmol) and TEA (209.79 mg, 2.07 mmol, 289.16 μL), then stirred at 80° C. for 6 hrs. The reaction was concentrated to give a residue. The residue was purified by column chromatography to give N-(2-(1H-pyrazol-1-yl)benzyl)-2-chloro-9-ethyl-9H-purin-6-amine (230.23 mg, 637.71 μmol, 92.3% yield, 98% purity) as a white solid.

LCMS [M+H]⁺: 354.22

### Step 3

### Synthesis of tert-butyl (1-(6-((2-(1H-pyrazol-1-yl)benzyl)amino)-9-ethyl-9H-purin-2-yl)piperidin-4-yl)carbamate

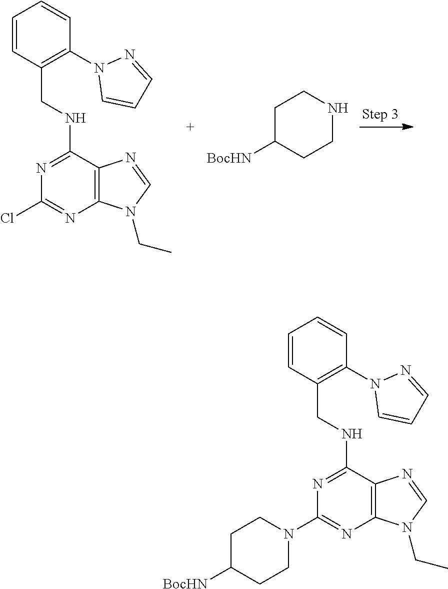 [SMILES: CCn1cnc2c(NCc3ccccc3-n3cccn3)nc(Cl)nc21 ; CC(C)(C)OC(=O)NC1CCNCC1 ; CCn1cnc2c(NCc3ccccc3-n3cccn3)nc(N3CCC(NC(=O)OC(C)(C)C)CC3)nc21]

To a solution of N-(2-(1H-pyrazol-1-yl)benzyl)-2-chloro-9-ethyl-9H-purin-6-amine (96.19 mg, 271.86 μmol) in DMSO (2.5 mL) was added tert-butyl piperidin-4-ylcarbamate (108.90 mg, 543.72 mol) and TEA (82.53 mg, 815.58 μmol, 113.75 μL), then stirred at 160° C. for 10 hrs. The reaction was poured into water (30 mL), extracted with EtOAc (10 mL*2), the organic layer was washed with water (10 mL*2), dried over Na₂SO₄, filtered and concentrated to give a residue. The residue was purified by column chromatography to give tert-butyl (1-(6-((2-(1H-pyrazol-1-yl)benzyl)amino)-9-ethyl-9H-purin-2-yl)piperidin-4-yl)carbamate (132 mg, 244.81 μmol, 90.05% yield, 96% purity) as a white solid.

LCMS [M+H]⁺: 518.42

### Step 4

### Synthesis of N-(2-(1H-pyrazol-1-yl)benzyl)-2-(4-aminopiperidin-1-yl)-9-ethyl-9H-purin-6-amine hydrochloride

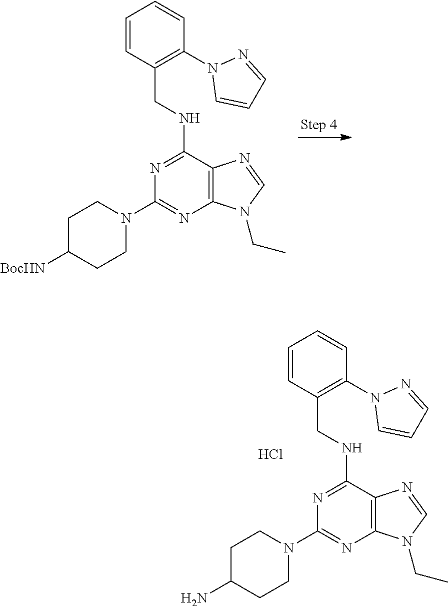 [SMILES: CCn1cnc2c(NCc3ccccc3-n3cccn3)nc(N3CCC(NC(=O)OC(C)(C)C)CC3)nc21 ; CCn1cnc2c(NCc3ccccc3-n3cccn3)nc(N3CCC(N)CC3)nc21 ; Cl]

To a solution of tert-butyl (1-(6-((2-(1H-pyrazol-1-yl)benzyl)amino)-9-ethyl-9H-purin-2-yl)piperidin-4-yl)carbamate (97.36 mg, 188.09 μmol) in MeOH (5 mL) was added HCl/MeOH (564.28 μmol), then stirred at 20° C. for 6 hrs. The reaction was concentrated to give N-(2-(1H-pyrazol-1-yl)benzyl)-2-(4-aminopiperidin-1-yl)-9-ethyl-9H-purin-6-amine hydrochloride (70 mg, 159.28 mol, 84.68% yield, 95% purity) as yellow solid.

¹H NMR (400 MHz, Methanol-d4) δ 7.92 (d, J=2.3 Hz, 1H), 7.86-7.74 (m, 2H), 7.71-7.58 (m, 1H), 7.42 (dqd, J=8.3, 3.6, 2.1 Hz, 3H), 6.56 (q, J=2.2, 1.7 Hz, 1H), 4.68 (d, J=13.8 Hz, 4H), 2.96-2.69 (m, 2H), 1.90-1.72 (m, 2H), 1.58 (m, 3H), 1.56 (s, 5H). LCMS [M+H]⁺: 418.23

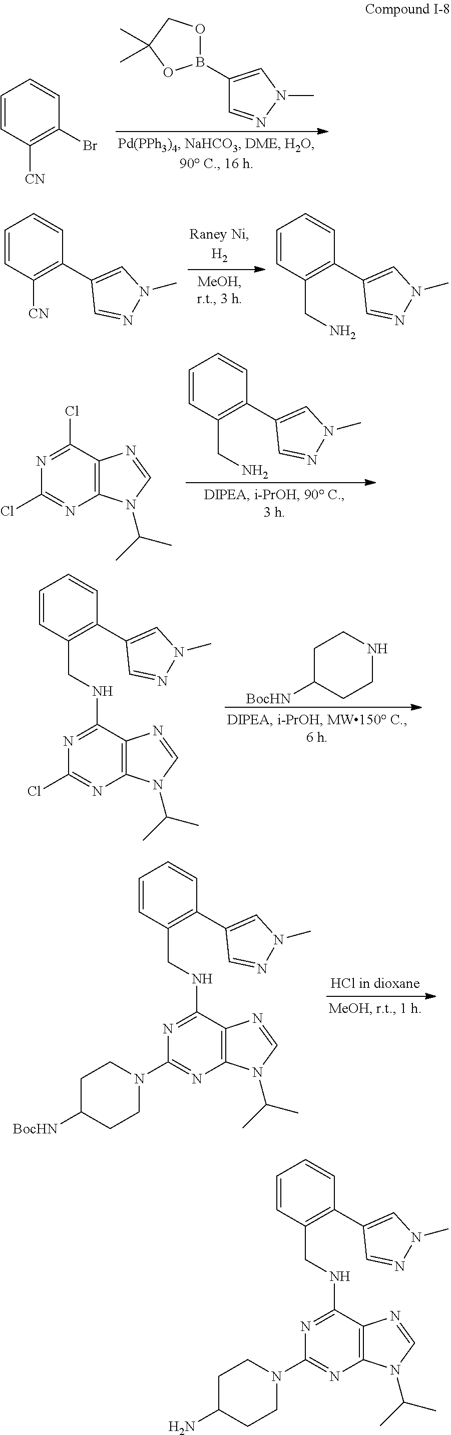

### Synthesis of 2-(1-methyl-1H-pyrazol-4-yl)benzonitrile

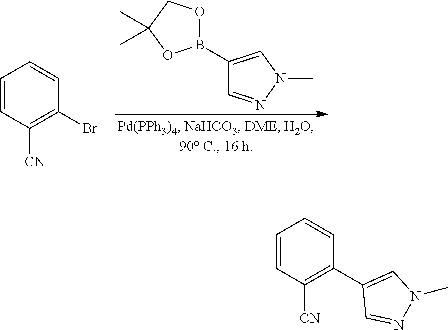

To a mixture of 2-bromobenzonitrile (1.0 g, 5.5 mmol), 1-methyl-4-(4,4,5,5-tetramethyl-1,3,2-dioxaborolan-2-yl)-1H-pyrazole (1.7 g, 8.2 mmol) and NaHCO₃ (1.4 g, 16.5 mmol) in DME (25 mL) and water (2.5 mL) was added Pd(PPh₃)₄ (635 mg, 0.55 mmol). The mixture was stirred at 90° C. for 16 hrs under N₂ atmosphere. The reaction mixture was cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=30/1) to give 2-(1-methyl-1H-pyrazol-4-yl)benzonitrile (920 mg, yield: 92%) as a colourless oil. ESI-MS [M+H]⁺: 184.2.

### Synthesis of (2-(1-methyl-1H-pyrazol-4-yl)phenyl)methanamine

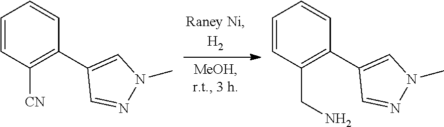

To a mixture of 2-(1-methyl-1H-pyrazol-4-yl)benzonitrile (320 mg, 1.75 mmol) in NH₃ (7M solution in MeOH, 5 mL) was added Raney Ni (50 mg). The reaction mixture was stirred at room temperature for 3 hrs under H₂ atmosphere. The reaction mixture was filtered through Celite® and the filter cake was washed with DCM/MeOH (10/1, 20 mL). The filtrate was concentrated to give the crude, which was purified by Prep-TLC (eluent: DCM/MeOH=10/1) to give (2-(1-methyl-1H-pyrazol-4-yl)phenyl)methanamine (260 mg, yield: 79%) as a yellow oil. ESI-MS [M+H]⁺: 188.2.

### Synthesis of 2-chloro-9-isopropyl-N-(2-(1-methyl-1H-pyrazol-4-yl)benzyl)-9H-purin-6-amine

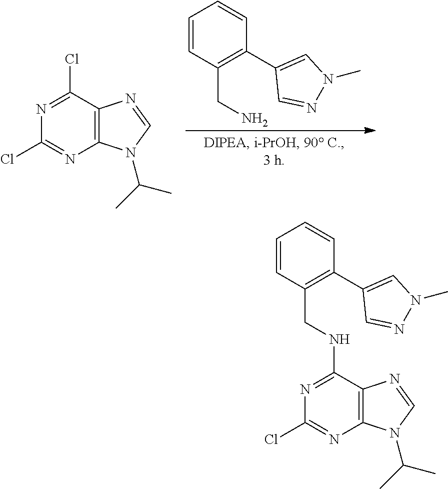 [SMILES: CC(C)n1cnc2c(Cl)nc(Cl)nc21 ; Cn1cc(-c2ccccc2CN)cn1 ; CC(C)n1cnc2c(NCc3ccccc3-c3cnn(C)c3)nc(Cl)nc21]

A mixture of 2,6-dichloro-9-isopropyl-9H-purine (100 mg, 0.43 mmol), (2-(1-methyl-1H-pyrazol-4-yl)phenyl)methanamine (101 mg, 0.54 mmol) and DIPEA (284 mg, 2.2 mmol) in i-PrOH (5 mL) was stirred at 90° C. for 3 hrs. The reaction mixture was concentrated to give the crude, which was purified by Prep-TLC (eluent: DCM/MeOH=20/1) to give 2-chloro-9-isopropyl-N-(2-(1-methyl-1H-pyrazol-4-yl)benzyl)-9H-purin-6-amine (150 mg, yield: 91%) as a white solid. ESI-MS [M+H]⁺: 382.2.

### Synthesis of tert-butyl (1-(9-isopropyl-6-((2-(1-methyl-1H-pyrazol-4-yl)benzyl)amino)-9H-purin-2-yl)piperidin-4-yl)carbamate

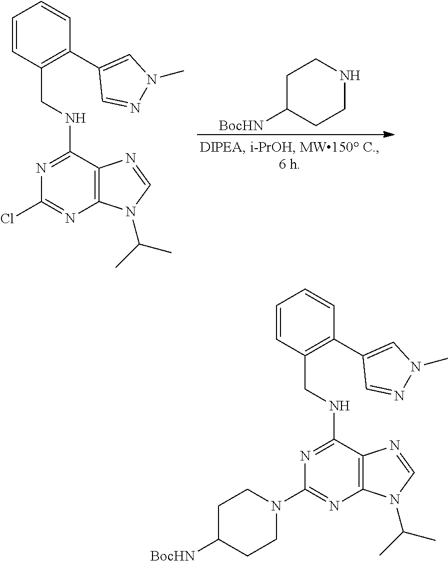 [SMILES: CC(C)n1cnc2c(NCc3ccccc3-c3cnn(C)c3)nc(Cl)nc21 ; CC(C)(C)OC(=O)NC1CCNCC1 ; CC(C)n1cnc2c(NCc3ccccc3-c3cnn(C)c3)nc(N3CCC(NC(=O)OC(C)(C)C)CC3)nc21]

A mixture of 2-chloro-9-isopropyl-N-(2-(1-methyl-1H-pyrazol-4-yl)benzyl)-9H-purin-6-amine (150 mg, 0.39 mmol), tert-butyl piperidin-4-ylcarbamate (98 mg, 0.49 mmol) and DIPEA (252 mg, 1.95 mmol) in i-PrOH (8 mL) was irradiated in microwave at 150° C. for 6 hrs. The reaction mixture was concentrated to give the crude, which was purified by Prep-TLC (eluent: DCM/MeOH=20/1) to give tert-butyl (1-(9-isopropyl-6-((2-(1-methyl-1H-pyrazol-4-yl)benzyl)amino)-9H-purin-2-yl)piperidin-4-yl)carbamate (108 mg, yield: 51%) as a white solid. ESI-MS [M+H]⁺: 546.2.

### Synthesis of 2-(4-aminopiperidin-1-yl)-9-isopropyl-N-(2-(1-methyl-1H-pyrazol-4-yl)benzyl)-9H-purin-6-amine

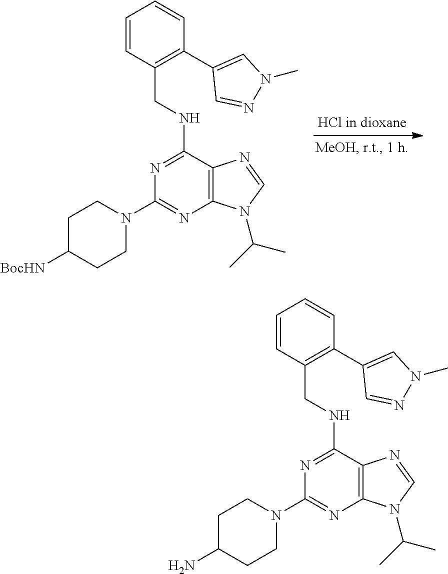 [SMILES: CC(C)n1cnc2c(NCc3ccccc3-c3cnn(C)c3)nc(N3CCC(NC(=O)OC(C)(C)C)CC3)nc21 ; CC(C)n1cnc2c(NCc3ccccc3-c3cnn(C)c3)nc(N3CCC(N)CC3)nc21]

To a mixture of tert-butyl (1-(9-isopropyl-6-((2-(1-methyl-1H-pyrazol-4-yl)benzyl)amino)-9H-purin-2-yl)piperidin-4-yl)carbamate (108 mg, 0.20 mmol) in MeOH (2 mL) was added HCl (4M solution in 1,4-dioxane, 2 mL). The reaction mixture was stirred at room temperature for 1 h. The reaction mixture was quenched with NH₃ (7M solution in MeOH, 2 mL) and concentrated in vacuo to give the crude, which was purified by Prep-TLC (eluent: DCM/MeOH=10/1) to give 2-(4-aminopiperidin-1-yl)-9-isopropyl-N-(2-(1-methyl-1H-pyrazol-4-yl)benzyl)-9H-purin-6-amine (40 mg, yield: 45%) as a white solid.

ESI-MS [M+H]⁺: 446.2.

¹H NMR (400 MHz, Methanol-d4) δ 7.77 (s, 1H), 7.74 (s, 1H), 7.60 (d, J=0.6 Hz, 1H), 7.49-7.46 (m, 1H), 7.37-7.34 (m, 1H), 7.26-7.23 (m, 2H), 4.77 (s, 2H), 4.70-4.61 (m, 3H), 3.90 (s, 3H), 2.88-2.77 (m, 3H), 1.81-1.77 (m, 2H), 1.55 (d, J=6.8 Hz, 6H), 1.29-1.18 (m, 2H).

### Compound I-9

### Synthesis of (S)—N-(2-(1H-pyrazol-1-yl)benzyl)-2-(3-aminopyrrolidin-1-yl)-9-isopropyl-9H-purin-6-amine

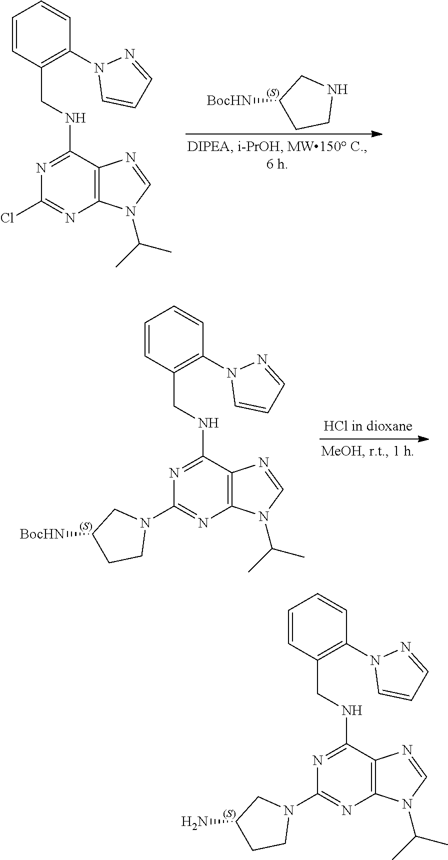

Synthesis of tert-butyl (S)-(1-(6-((2-(1H-pyrazol-1-yl)benzyl)amino)-9-isopropyl-9H-purin-2-yl)pyrrolidin-3-yl)carbamate

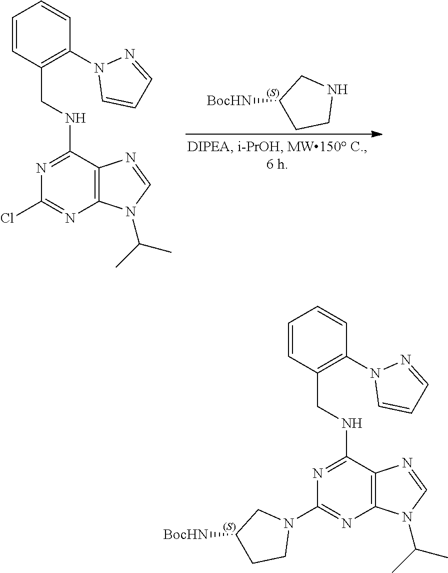

A mixture of N-(2-(1H-pyrazol-1-yl)benzyl)-2-chloro-9-isopropyl-9H-purin-6-amine (100 mg, 0.27 mmol), tert-butyl (S)-pyrrolidin-3-ylcarbamate (74 mg, 0.40 mmol) and DIPEA (174 mg, 1.35 mmol) in i-PrOH (8 mL) was irradiated in microwave at 150° C. for 6 hrs. The reaction mixture was concentrated to give the crude, which was purified by Prep-TLC (eluent: DCM/MeOH=20/1) to give tert-butyl (S)-(1-(6-((2-(1H-pyrazol-1-yl)benzyl)amino)-9-isopropyl-9H-purin-2-yl)pyrrolidin-3-yl)carbamate (70 mg, yield: 50%) as a white solid. ESI-MS [M+H]⁺: 518.3.

### Synthesis of (S)—N-(2-(1H-pyrazol-1-yl)benzyl)-2-(3-aminopyrrolidin-1-yl)-9-isopropyl-9H-purin-6-amine

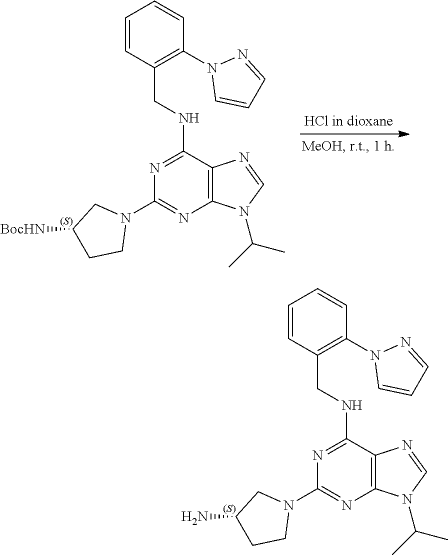

To a mixture of tert-butyl (S)-(1-(6-((2-(1H-pyrazol-1-yl)benzyl)amino)-9-isopropyl-9H-purin-2-yl)pyrrolidin-3-yl)carbamate (70 mg, 0.14 mmol) in MeOH (2 mL) was added HCl (4M solution in 1,4-dioxane, 2 mL). The reaction mixture was stirred at room temperature for 1 h. The reaction mixture was quenched with NH₃ (7M solution in MeOH, 2 mL) and concentrated in vacuo to give the crude, which was purified by Prep-TLC (eluent: DCM/MeOH=10/1) to give (S)—N-(2-(1H-pyrazol-1-yl)benzyl)-2-(3-aminopyrrolidin-1-yl)-9-isopropyl-9H-purin-6-amine (50 mg, yield: 86%) as a white solid. ESI-MS [M+H]⁺: 418.2.

ESI-MS [M+H]⁺: 418.2.

¹H NMR (400 MHz, Methanol-d4) δ 7.92 (d, J=2.4 Hz, 1H), 7.79 (d, J=1.5 Hz, 1H), 7.75 (s, 1H), 7.68-7.64 (m, 1H), 7.44-7.36 (m, 3H), 6.54 (t, J=2.1 Hz, 1H), 4.74-4.62 (m, 3H), 3.77-3.64 (m, 2H), 3.58-3.49 (m, 2H), 3.29-3.24 (m, 1H), 2.21-2.12 (m, 1H), 1.85-1.76 (m, 1H), 1.54 (d, J=6.8 Hz, 6H).

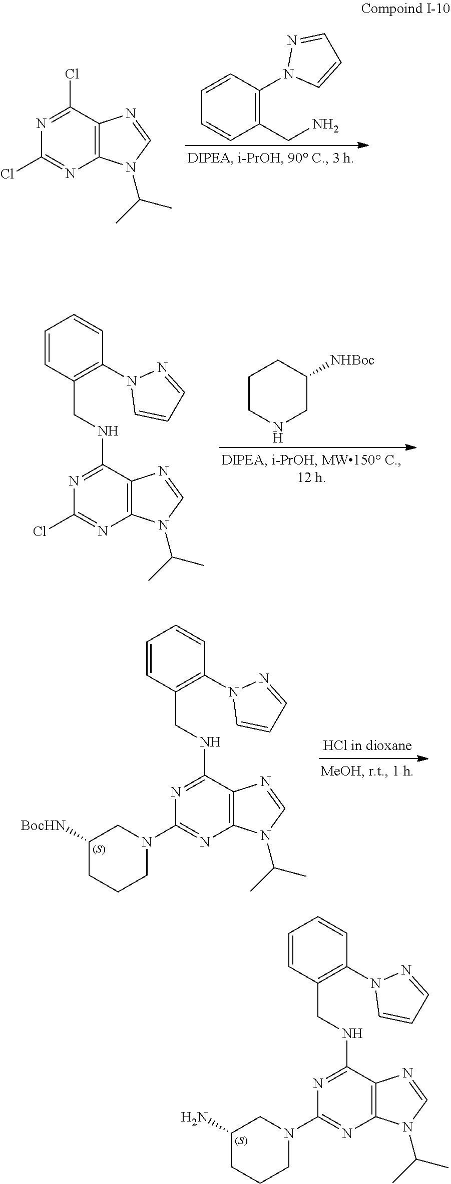

### Synthesis of N-(2-(1H-pyrazol-1-yl)benzyl)-2-chloro-9-isopropyl-9H-purin-6-amine

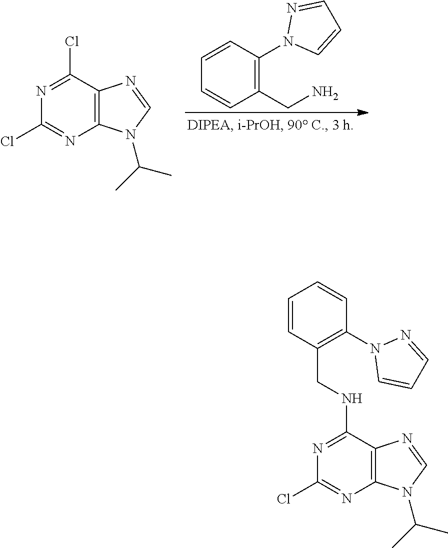 [SMILES: CC(C)n1cnc2c(Cl)nc(Cl)nc21 ; NCc1ccccc1-n1cccn1 ; CC(C)n1cnc2c(NCc3ccccc3-n3cccn3)nc(Cl)nc21]

A mixture of 2,6-dichloro-9-isopropyl-9H-purine (100 mg, 0.43 mmol), (2-(1H-pyrazol-1-yl)phenyl)methanamine (97 mg, 0.56 mmol) and DIPEA (166 mg, 1.29 mmol) in i-PrOH (5 mL) was stirred at 90° C. for 3 hrs. The reaction mixture was concentrated to give the crude, which was purified by Prep-TLC (eluent: DCM/MeOH=20/1) to give N-(2-(1H-pyrazol-1-yl)benzyl)-2-chloro-9-isopropyl-9H-purin-6-amine (100 mg, yield: 63%) as a white solid. ESI-MS [M+H]⁺: 368.2.

### Synthesis of tert-butyl (S)-(1-(6-((2-(1H-pyrazol-1-yl)benzyl)amino)-9-isopropyl-9H-purin-2-yl)piperidin-3-yl)carbamate

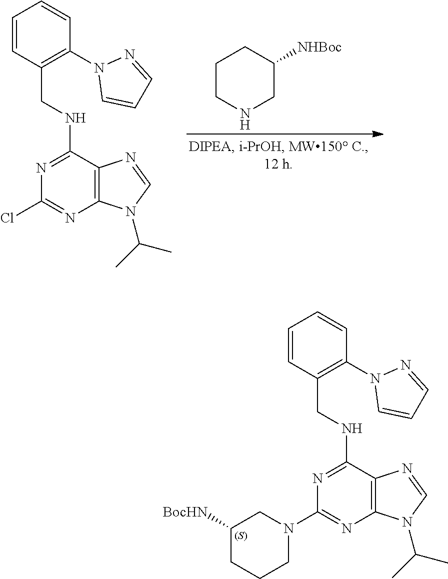

A mixture of N-(2-(1H-pyrazol-1-yl)benzyl)-2-chloro-9-isopropyl-9H-purin-6-amine (100 mg, 0.27 mmol), tert-butyl (S)-piperidin-3-ylcarbamate (80 mg, 0.40 mmol) and DIPEA (104 mg, 0.81 mmol) in i-PrOH (5 mL) was irradiated in microwave at 150° C. for 12 hrs. The reaction mixture was concentrated to give the crude, which was purified by Prep-TLC (eluent: DCM/MeOH=20/1) to give tert-butyl (S)-(1-(6-((2-(1H-pyrazol-1-yl)benzyl)amino)-9-isopropyl-9H-purin-2-yl)piperidin-3-yl)carbamate (70 mg, yield: 49%) as a white solid. ESI-MS [M+H]⁺: 532.2.

### Synthesis of (S)—N-(2-(1H-pyrazol-1-yl)benzyl)-2-(3-aminopiperidin-1-yl)-9-isopropyl-9H-purin-6-amine

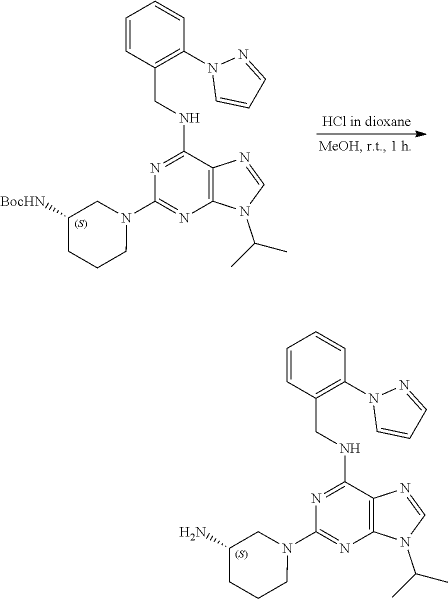

To a mixture of tert-butyl (S)-(1-(6-((2-(1H-pyrazol-1-yl)benzyl)amino)-9-isopropyl-9H-purin-2-yl)piperidin-3-yl)carbamate (70 mg, 0.13 mmol) in MeOH (2 mL) was added HCl (4M solution in 1,4-dioxane, 2 mL). The reaction mixture was stirred at room temperature for 1 h. The reaction mixture was quenched with NH₃ (7M solution in MeOH, 2 mL) and concentrated in vacuo to give the crude, which was purified by Prep-TLC (eluent: DCM/MeOH=10/1) to give (S)—N-(2-(1H-pyrazol-1-yl)benzyl)-2-(3-aminopiperidin-1-yl)-9-isopropyl-9H-purin-6-amine (40 mg, yield: 71%) as a white solid.

ESI-MS [M+H]⁺: 432.2.

¹H NMR (400 MHz, Methanol-d4) δ 7.92 (d, J=2.3 Hz, 1H), 7.78 (d, J=1.6 Hz, 2H), 7.63-7.56 (m, 1H), 7.43-7.34 (m, 3H), 6.54 (t, J=2.0 Hz, 1H), 4.70-4.59 (m, 3H), 4.50 (d, J=12.3 Hz, 1H), 4.32 (d, J=13.2 Hz, 1H), 3.00 (t, J=10.7 Hz, 1H), 2.88-2.72 (m, 2H), 1.98-1.95 (m, 1H), 1.75-1.64 (m, 1H), 1.57-1.39 (m, 8H)

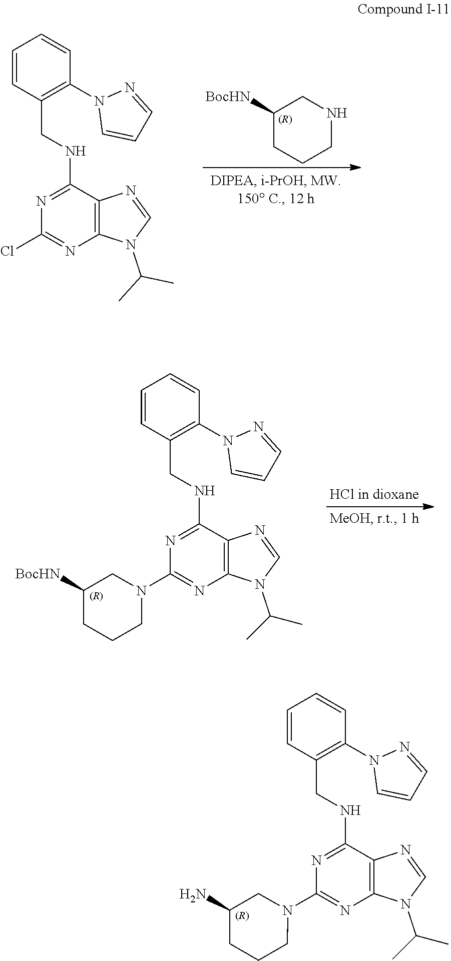

### Synthesis of tert-butyl (R)-(1-(6-((2-(1H-pyrazol-1-yl)benzyl)amino)-9-isopropyl-9H-purin-2-yl)piperidin-3-yl)carbamate

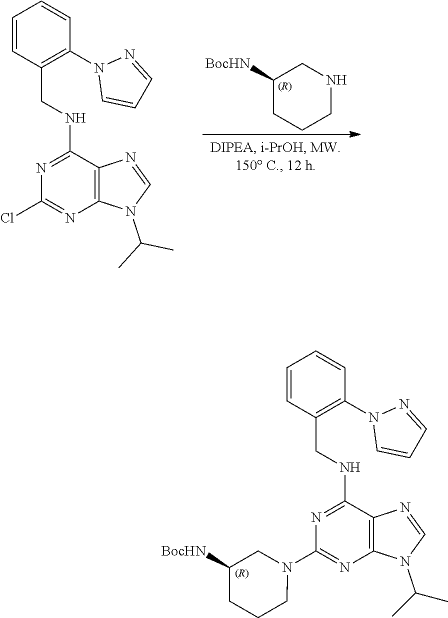

A mixture of N-(2-(1H-pyrazol-1-yl)benzyl)-2-chloro-9-isopropyl-9H-purin-6-amine (100 mg, 0.27 mmol), tert-butyl (R)-piperidin-3-ylcarbamate (82 mg, 0.41 mmol) and DIPEA (174 mg, 1.35 mmol) in i-PrOH (8 mL) was irradiated in microwave at 150° C. for 12 hrs. The reaction mixture was concentrated to give the crude, which was purified by Prep-TLC (eluent: DCM/MeOH=20/1) to give tert-butyl (R)-(1-(6-((2-(1H-pyrazol-1-yl)benzyl)amino)-9-isopropyl-9H-purin-2-yl)piperidin-3-yl)carbamate (60 mg, yield: 42%) as a white solid. ESI-MS [M+H]⁺: 532.3.

### Synthesis of (R)—N-(2-(1H-pyrazol-1-yl)benzyl)-2-(3-aminopiperidin-1-yl)-9-isopropyl-9H-purin-6-amine

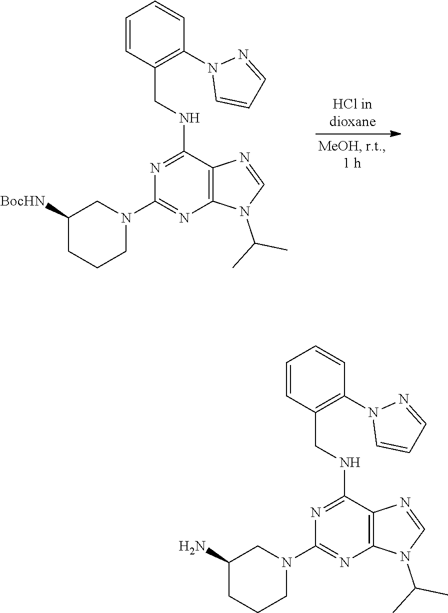 [SMILES: CC(C)n1cnc2c(NCc3ccccc3-n3cccn3)nc(N3CCC[C@@H](NC(=O)OC(C)(C)C)C3)nc21 ; CC(C)n1cnc2c(NCc3ccccc3-n3cccn3)nc(N3CCC[C@@H](N)C3)nc21]

To a mixture of tert-butyl (R)-(1-(6-((2-(1H-pyrazol-1-yl)benzyl)amino)-9-isopropyl-9H-purin-2-yl)piperidin-3-yl)carbamate (60 mg, 0.11 mmol) in MeOH (2 mL) was added HCl (4M solution in 1,4-dioxane, 2 mL). The reaction mixture was stirred at room temperature for 1 h. The reaction mixture was quenched with NH₃ (7M solution in MeOH, 2 mL) and concentrated in vacuo to give the crude, which was purified by Prep-TLC (eluent: DCM/NH₃-MeOH=20/1) to give (R)—N-(2-(1H-pyrazol-1-yl)benzyl)-2-(3-aminopiperidin-1-yl)-9-isopropyl-9H-purin-6-amine (35 mg, yield: 74%) as a white solid.

ESI-MS [M+H]⁺: 432.3.

¹H NMR (400 MHz, Methanol-d4) δ 7.92 (d, J=2.3 Hz, 1H), 7.82-7.74 (m, 2H), 7.65-7.57 (m, 1H), 7.45-7.33 (m, 3H), 6.54 (t, J=2.0 Hz, 1H), 4.71-4.62 (m, 3H), 4.53 (d, J=10.5 Hz, 1H), 4.39 (d, J=12.9 Hz, 1H), 3.02-2.85 (m, 1H), 2.73-2.65 (m, 2H), 1.95 (d, J=10.5 Hz, 1H), 1.77-1.64 (m, 1H), 1.55-1.53 (m, 6H), 1.41-1.21 (m, 2H).

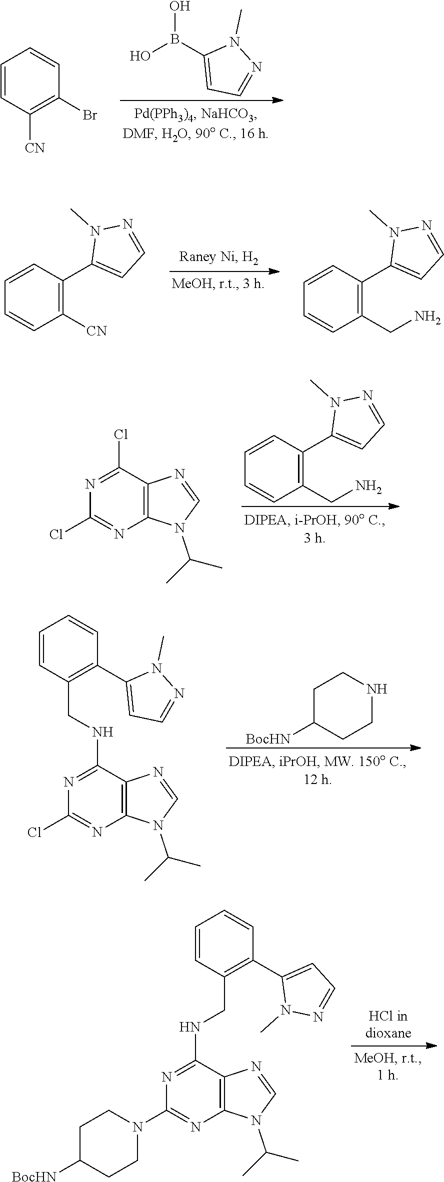

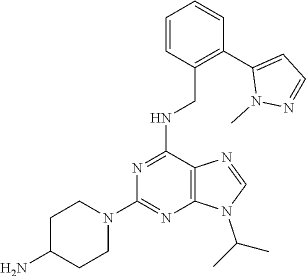 [SMILES: CC(C)n1cnc2c(NCc3ccccc3-c3ccnn3C)nc(N3CCC(N)CC3)nc21]

### Synthesis of 2-(1-methyl-1H-pyrazol-5-yl)benzonitrile

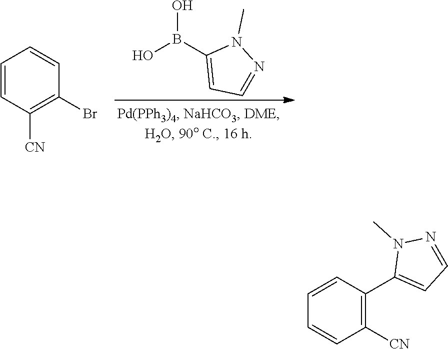

To a mixture of 2-bromobenzonitrile (2 g, 11.0 mmol), (1-methyl-1H-pyrazol-5-yl)boronic acid (2.1 g, 16.5 mmol) and NaHCO₃ (2.8 g, 33.0 mmol) in DME (50 mL) and water (5 mL) was added Pd(PPh₃)₄ (1.3 g, 1.1 mmol). The mixture was stirred at 90° C. for 16 hrs under N₂ atmosphere. The reaction mixture was cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=30/1) to give 2-(1-methyl-1H-pyrazol-5-yl)benzonitrile (800 mg, yield: 40%) as a colourless oil. ESI-MS [M+H]⁺: 184.2.

### Synthesis of (2-(1-methyl-1H-pyrazol-5-yl)phenyl)methanamine

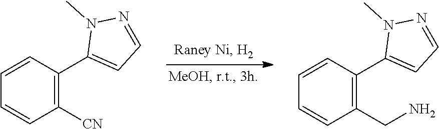

To a mixture of 2-(1-methyl-1H-pyrazol-5-yl)benzonitrile (200 mg, 1.1 mmol) in NH₃ (7M solution in MeOH, 5 mL) was added Raney Ni (50 mg). The reaction mixture was stirred at room temperature for 3 hrs under H₂ atmosphere. The reaction mixture was filtered through Celite® and the filter cake was washed with DCM/MeOH (10/1, 20 mL). The filtrate was concentrated to give the crude, which was purified by Prep-TLC (eluent: DCM/MeOH=10/1) to give (2-(1-methyl-1H-pyrazol-5-yl)phenyl)methanamine (120 mg, yield: 58%) as a yellow oil. ESI-MS [M+H]⁺: 188.2.

### Synthesis of 2-chloro-9-isopropyl-N-(2-(1-methyl-1H-pyrazol-5-yl)benzyl)-9H-purin-6-amine

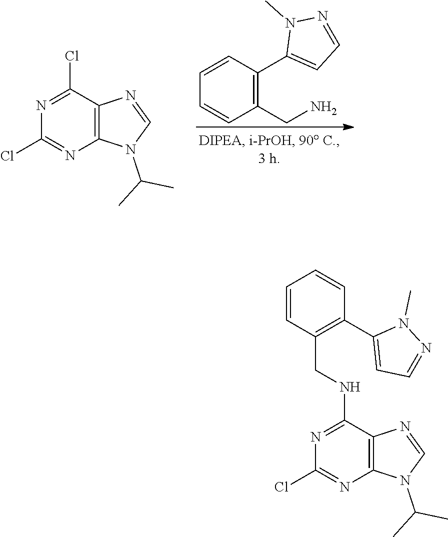 [SMILES: CC(C)n1cnc2c(Cl)nc(Cl)nc21 ; Cn1nccc1-c1ccccc1CN ; CC(C)n1cnc2c(NCc3ccccc3-c3ccnn3C)nc(Cl)nc21]

A mixture of 2,6-dichloro-9-isopropyl-9H-purine (100 mg, 0.43 mmol), (2-(1-methyl-1H-pyrazol-5-yl)phenyl)methanamine (105 mg, 0.56 mmol) and DIPEA (166 mg, 1.29 mmol) in i-PrOH (5 mL) was stirred at 90° C. for 3 hrs. The reaction mixture was concentrated to give the crude, which was purified by Prep-TLC (eluent: DCM/MeOH=20/1) to give 2-chloro-9-isopropyl-N-(2-(1-methyl-1H-pyrazol-5-yl)benzyl)-9H-purin-6-amine (100 mg, yield: 61%) as a white solid. ESI-MS [M+H]⁺: 382.2.

### Synthesis of tert-butyl (1-(9-isopropyl-6-((2-(1-methyl-1H-pyrazol-5-yl)benzyl)amino)-9H-purin-2-yl)piperidin-4-yl)carbamate

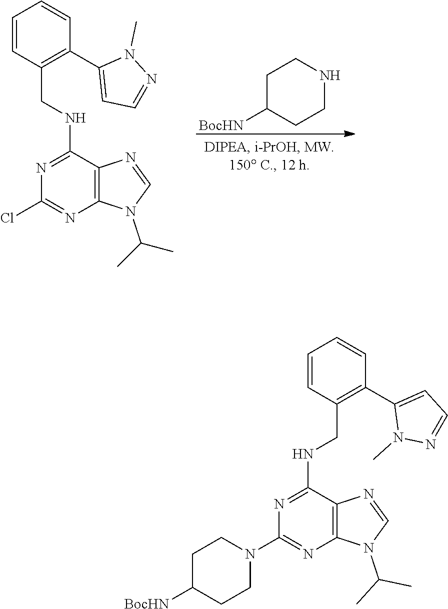 [SMILES: CC(C)n1cnc2c(NCc3ccccc3-c3ccnn3C)nc(Cl)nc21 ; CC(C)(C)OC(=O)NC1CCNCC1 ; CC(C)n1cnc2c(NCc3ccccc3-c3ccnn3C)nc(N3CCC(NC(=O)OC(C)(C)C)CC3)nc21]

A mixture of 2-chloro-9-isopropyl-N-(2-(1-methyl-1H-pyrazol-5-yl)benzyl)-9H-purin-6-amine (100 mg, 0.26 mmol), tert-butyl piperidin-4-ylcarbamate (78 mg, 0.39 mmol) and DIPEA (168 mg, 1.3 mmol) in i-PrOH (5 mL) was irradiated in microwave at 150° C. for 12 hrs. The reaction mixture was concentrated to give the crude, which was purified by Prep-TLC (eluent: DCM/MeOH=20/1) to give tert-butyl(1-(9-isopropyl-6-((2-(1-methyl-1H-pyrazol-5-yl)benzyl)amino)-9H-purin-2-yl)piperidin-4-yl)carbamate (100 mg, yield: 71%) as a white solid. ESI-MS [M+H]⁺: 546.2.

### Synthesis of 2-(4-aminopiperidin-1-yl)-9-isopropyl-N-(2-(1-methyl-1H-pyrazol-5-yl)benzyl)-9H-purin-6-amine

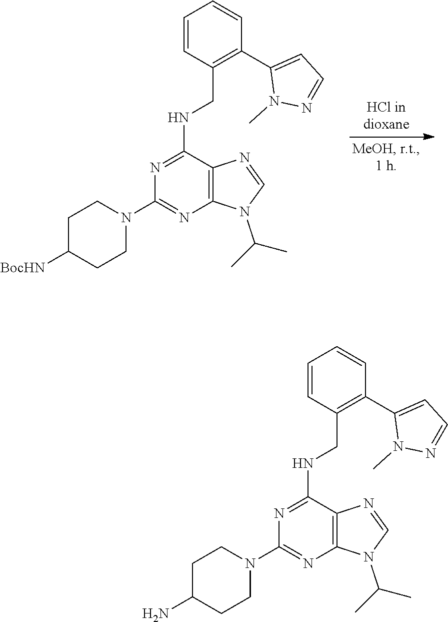 [SMILES: CC(C)n1cnc2c(NCc3ccccc3-c3ccnn3C)nc(N3CCC(NC(=O)OC(C)(C)C)CC3)nc21 ; CC(C)n1cnc2c(NCc3ccccc3-c3ccnn3C)nc(N3CCC(N)CC3)nc21]

To a mixture of tert-butyl (1-(9-isopropyl-6-((2-(1-methyl-1H-pyrazol-5-yl)benzyl)amino)-9H-purin-2-yl)piperidin-4-yl)carbamate (100 mg, 0.18 mmol) in MeOH (2 mL) was added HCl (4M solution in 1,4-dioxane, 2 mL). The reaction mixture was stirred at room temperature for 1 h. The reaction mixture was quenched with NH₃ (7M solution in MeOH, 2 mL) and concentrated in vacuo to give the crude, which was purified by Prep-TLC (eluent: DCM/MeOH=10/1) to give 2-(4-aminopiperidin-1-yl)-9-isopropyl-N-(2-(1-methyl-1H-pyrazol-5-yl)benzyl)-9H-purin-6-amine (35 mg, yield: 44%) as a white solid.

ESI-MS [M+H]⁺: 446.3.

1H NMR (400 MHz, Methanol-d4) δ 7.77 (s, 1H), 7.57 (d, J=7.6 Hz, 1H), 7.50 (d, J=1.9 Hz, 1H), 7.46-7.42 (m, 1H), 7.38-7.34 (m, 1H), 7.28-7.24 (m, 1H), 6.32 (d, J=1.9 Hz, 1H), 4.71-4.55 (m, 5H), 3.61 (s, 3H), 2.90-2.75 (m, 3H), 1.80 (d, J=10.1 Hz, 2H), 1.54 (d, J=6.8 Hz, 6H), 1.26-1.22 (m, 2H).

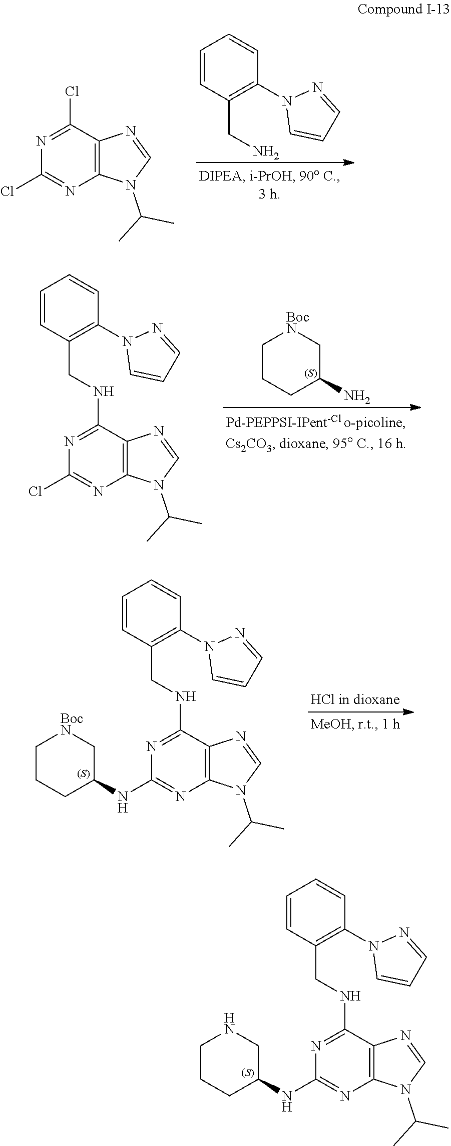

### Synthesis of N-(2-(1H-pyrazol-1-yl)benzyl)-2-chloro-9-isopropyl-9H-purin-6-amine

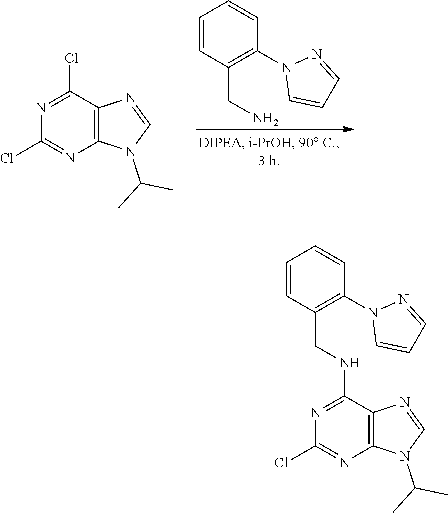 [SMILES: CC(C)n1cnc2c(Cl)nc(Cl)nc21 ; NCc1ccccc1-n1cccn1 ; CC(C)n1cnc2c(NCc3ccccc3-n3cccn3)nc(Cl)nc21]

A mixture of 2,6-dichloro-9-isopropyl-9H-purine (400 mg, 1.74 mmol), (2-(1H-pyrazol-1-yl)phenyl)methanamine (376 mg, 2.17 mmol) and DIPEA (673 mg, 5.22 mmol) in i-PrOH (20 mL) was stirred at 90° C. for 3 hrs. The reaction mixture was concentrated to give the crude, which was purified by Prep-TLC (eluent: DCM/MeOH=20/1) to give N-(2-(1H-pyrazol-1-yl)benzyl)-2-chloro-9-isopropyl-9H-purin-6-amine (520 mg, yield: 81%) as a white solid. ESI-MS [M+H]⁺: 368.1.

### Synthesis of tert-butyl (S)-3-((6-((2-(1H-pyrazol-1-yl)benzyl)amino)-9-isopropyl-9H-purin-2-yl)amino)piperidine-1-carboxylate

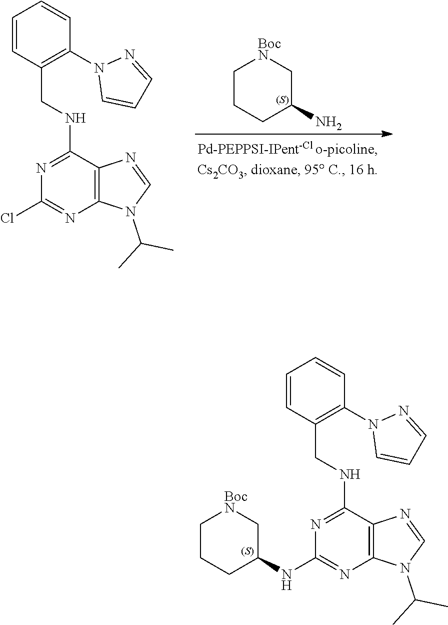

A mixture of N-(2-(1H-pyrazol-1-yl)benzyl)-2-chloro-9-isopropyl-9H-purin-6-amine (100 mg, 0.27 mmol), tert-butyl (S)-3-aminopiperidine-1-carboxylate (82 mg, 0.41 mmol), Pd-PEPPSI-IPent-Cl o-picoline (25 mg, 0.03 mmol) and Cs₂CO₃ (264 mg, 0.81 mmol) in dioxane (10 mL) was stirred at 95° C. for 16 hrs under N₂ atmosphere. The reaction mixture was filtered through Celite® and the filter cake was washed with DCM/MeOH (10/1, 20 mL). The filtrate was concentrated to give the crude, which was purified by Prep-TLC (eluent: DCM/MeOH=20/1) to give tert-butyl (S)-3-((6-((2-(1H-pyrazol-1-yl)benzyl)amino)-9-isopropyl-9H-purin-2-yl)amino)piperidine-1-carboxylate (70 mg, yield: 49%) as a white solid. ESI-MS [M+H]⁺: 532.3.

### Synthesis of (S)-N6-(2-(1H-pyrazol-1-yl)benzyl)-9-isopropyl-N2-(piperidin-3-yl)-9H-purine-2,6-diamine

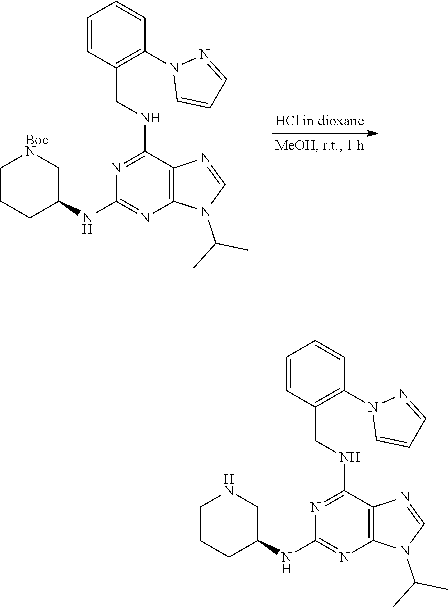 [SMILES: CC(C)n1cnc2c(NCc3ccccc3-n3cccn3)nc(N[C@H]3CCCN(C(=O)OC(C)(C)C)C3)nc21 ; CC(C)n1cnc2c(NCc3ccccc3-n3cccn3)nc(N[C@H]3CCCNC3)nc21]

To a mixture of tert-butyl (S)-3-((6-((2-(1H-pyrazol-1-yl)benzyl)amino)-9-isopropyl-9H-purin-2-yl)amino)piperidine-1-carboxylate (70 mg, 0.13 mmol) in MeOH (2 mL) was added HCl (4M solution in 1,4-dioxane, 2 mL). The reaction mixture was stirred at room temperature for 1 h. The reaction mixture was quenched with NH₃ (7M solution in MeOH, 2 mL) and concentrated in vacuo to give the crude, which was purified by Prep-TLC (eluent: DCM/NH₃·MeOH=10/1) to give (S)—N6-(2-(1H-pyrazol-1-yl)benzyl)-9-isopropyl-N2-(piperidin-3-yl)-9H-purine-2,6-diamine (40 mg, yield: 71%) as a white solid.

ESI-MS [M+H]⁺: 432.3.

¹H NMR (400 MHz, Methanol-d4) δ 7.96 (d, J=2.2 Hz, 1H), 7.81-7.77 (m, 2H), 7.60-7.58 (m, 1H), 7.44-7.40 (m, 3H), 6.59-6.50 (m, 1H), 4.70-4.54 (m, 3H), 3.93-3.88 (m, 1H), 3.13-3.07 (m, 2H), 2.69-2.63 (m, 1H), 2.47-2.41 (m, 1H), 2.02-1.98 (m, 1H), 1.88-1.83 (m, 1H), 1.68-1.64 (m, 1H), 1.53 (d, J=6.8 Hz, 6H), 1.47-1.34 (m, 1H).

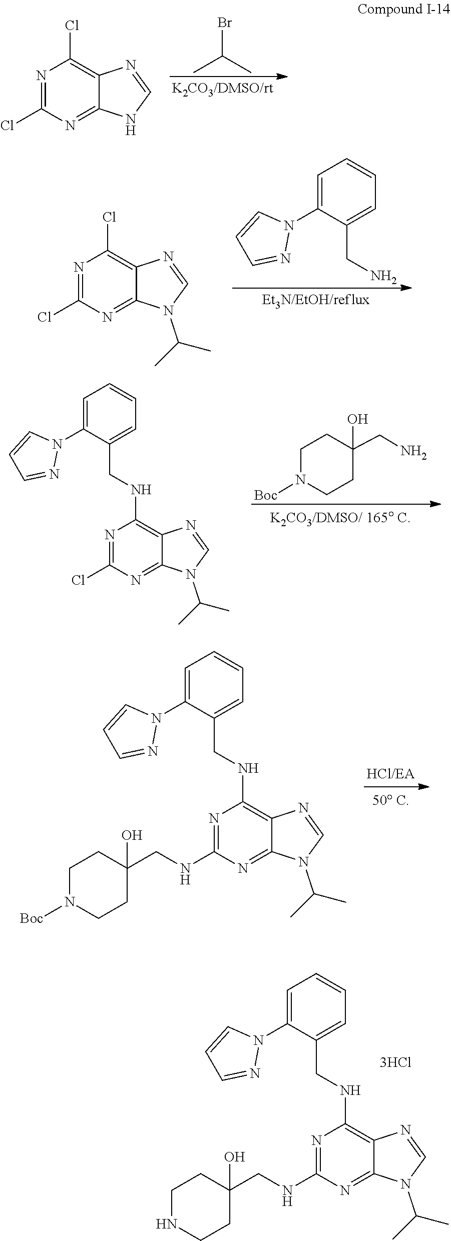

2,6-dichloro-9-isopropyl-9H-purine: 2,6-dichloro-9H-purine (10.0 g, 52.91 mmol), isopropyl bromide(32.5 g, 264.55 mmol), potassium carbonate(21.9 g, 158.73 mmol) and dimethyl sulfoxide(50 mL) were added to a 250 mL two-necked flask, and the mixture was stirred at room temperature for 96 hrs. The reaction solution was added dropwise to water, ethyl acetate was added for extraction three times. The organic phases were combined, washed twice with saturated brine, dried over sodium sulfate, filtered and concentrated in vacuo to afford 9.8 g of light-yellow solid. The crude product was passed through a silica gel column (ethyl acetate/n-heptane=0-60%) to afford 4.941 g of white solid.

N-(2-(1H-pyrazol-1-yl)benzyl)-2-chloro-9-isopropyl-9H-purin-6-amine: 2,6-dichloro-9-isopropyl-9H-purine (2.082 g, 9.011 mmol), (2-(1H-pyrazol-1-yl)phenyl)methanamine (1.639 g, 9.462 mmol), triethylamine (1.368 g, 13.517 mmol) and ethanol (42 mL) were added to a 100 mL single-necked flask, and the mixture was heated and refluxed for 5 h. The reaction was concentrated in vacuum and purified by silica gel column (Methanol/dichloromethane=0-10%) to afford 3304 mg of off-white solid.

tert-butyl 4-(((6-((2-(1H-pyrazol-1-yl)benzyl)amino)-9-isopropyl-9H-purin-2-yl)amino)methyl)-4-hydroxypiperidine-1-carboxylate: To a solution of N-(2-(1H-pyrazol-1-yl)benzyl)-2-chloro-9-isopropyl-9H-purin-6-amine (200 mg, 543.72 μmol) in DMSO (2 mL) was added tert-butyl 4-(aminomethyl)-4-hydroxypiperidine-1-carboxylate (187.83 mg, 815.58 μmol) and potassium carbonate (150.30 mg, 1.09 mmol), the mixture was stirred at 165° C. for 10 hrs. The reaction was poured into water (12 mL), extracted with EA (12 mL*2). The organic layer was washed with brine (12 mL*2), dried over Na₂SO₄, filtered and concentrated to give a residue. The residue was purified by column(SiO₂, MeOH:DCM=0% to 15%) to give tert-butyl 4-(((6-((2-(1H-pyrazol-1-yl)benzyl)amino)-9-isopropyl-9H-purin-2-yl)amino)methyl)-4-hydroxypiperidine-1-carboxylate (248 mg, 416.19 μmol, 76.54% yield, 94.26% purity) as an off-white solid.

4-(((6-((2-(1H-pyrazol-1-yl)benzyl)amino)-9-isopropyl-9H-purin-2-yl)amino)methyl)piperidin-4-ol trihydrochloride: To a solution of N-(2-(1H-pyrazol-1-yl)benzyl)-2-chloro-9-isopropyl-9H-purin-6-amine (248 mg, 441.53 μmol) in EA (5 mL) was added HCl (3 M, 5 mL), the mixture was stirred at room temperature for 2 hrs. The reaction solution was concentrated in vacuo to afford 229 mg off-white solid. The solid was dissolved in water, and then freeze-dried with a freeze dryer to give 4-(((6-((2-(1H-pyrazol-1-yl)benzyl)amino)-9-isopropyl-9H-purin-2-yl)amino)methyl)piperidin-4-ol (220 mg, 385.33 μmol, 87.27% yield, 3HCl) as an off-white solid.

¹H NMR (400 MHz, Methanol-d4) δ 8.41 (s, 1H), 8.06 (s, 1H), 7.86 (s, 1H), 7.68 (s, 1H), 7.51 (dp, J=9.2, 4.6 Hz, 3H), 6.59 (d, J=1.8 Hz, 1H), 4.78 (s, 3H), 3.58 (s, 2H), 3.28 (d, J=9.6 Hz, 4H), 1.88 (s, 4H), 1.62 (d, J=6.8 Hz, 6H).

MS: cacl. for C24H34Cl3N9O 461.27. Found: [M+H]⁺:462.42

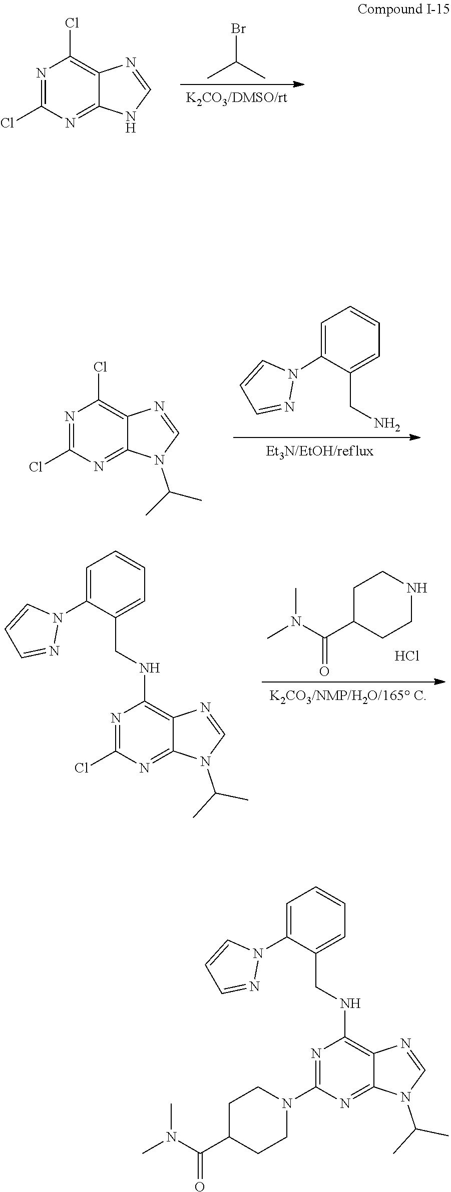

2,6-dichloro-9-isopropyl-9H-purine: 2,6-dichloro-9H-purine (10.0 g, 52.91 mmol), isopropyl bromide (32.5 g, 264.55 mmol), potassium carbonate (21.9 g, 158.73 mmol) and dimethyl sulfoxide (50 mL) were added to a 250 mL two-necked flask, and the mixture was stirred at room temperature for 96 hrs. The reaction solution was added dropwise to water, ethyl acetate was added for extraction three times. The organic phases were combined, washed twice with saturated brine, dried over sodium sulfate, filtered and concentrated in vacuo to afford 9.8 g of light-yellow solid. The crude product was passed through a silica gel column (ethyl acetate/n-heptane=0-60%) to afford 4.941 g of white solid.

N-(2-(1H-pyrazol-1-yl)benzyl)-2-chloro-9-isopropyl-9H-purin-6-amine: 2,6-dichloro-9-isopropyl-9H-purine (2.082 g, 9.011 mmol), (2-(1H-pyrazol-1-yl)phenyl)methanamine (1.639 g, 9.462 mmol), triethylamine(1.368 g, 13.517 mmol) and ethanol(42 mL) were added to a 100 mL single-necked flask, and the mixture was heated and refluxed for 5 hrs. The reaction was concentrated in vacuum and purified by silica gel column (Methanol/dichloromethane=0-10%) to afford 3304 mg of off-white solid.

1-(6-((2-(1H-pyrazol-1-yl)benzyl)amino)-9-isopropyl-9H-purin-2-yl)-N,N-dimethylpiperidine-4-carboxamide: N-(2-(1H-pyrazol-1-yl)benzyl)-2-chloro-9-isopropyl-9H-purin-6-amine (100 mg, 0.272 mmol), N,N-dimethylpiperidine-4-carboxamide hydrochloride (131 mg, 0.680 mmol), potassium carbonate (150 mg, 1.087 mmol) and 99% NMP (2 mL) were added to a 4 mL reaction flask, heated to 165° C. and stirred for 16 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo to afford 138 mg oil. The crude was purified by silica gel column (Methanol/dichloromethane=0-10%) to afford 88 mg oil.

¹H NMR (400 MHz, DMSO-d6) δ 8.11 (d, J=2.4 Hz, 1H), 7.85 (s, 1H), 7.80 (d, J=1.8 Hz, 2H), 7.53 (d, J=5.7 Hz, 1H), 7.45-7.30 (m, 3H), 6.56 (s, 1H), 4.74-4.33 (m, 5H), 3.05 (s, 3H), 2.81 (s, 6H), 1.57 (d, J=12.8 Hz, 2H), 1.46 (d, J=6.7 Hz, 6H), 1.35 (q, J=14.4, 12.9 Hz, 2H).

MS: cacl. for C26H33N9O 487.28. Found: [M+H]⁺:488.42

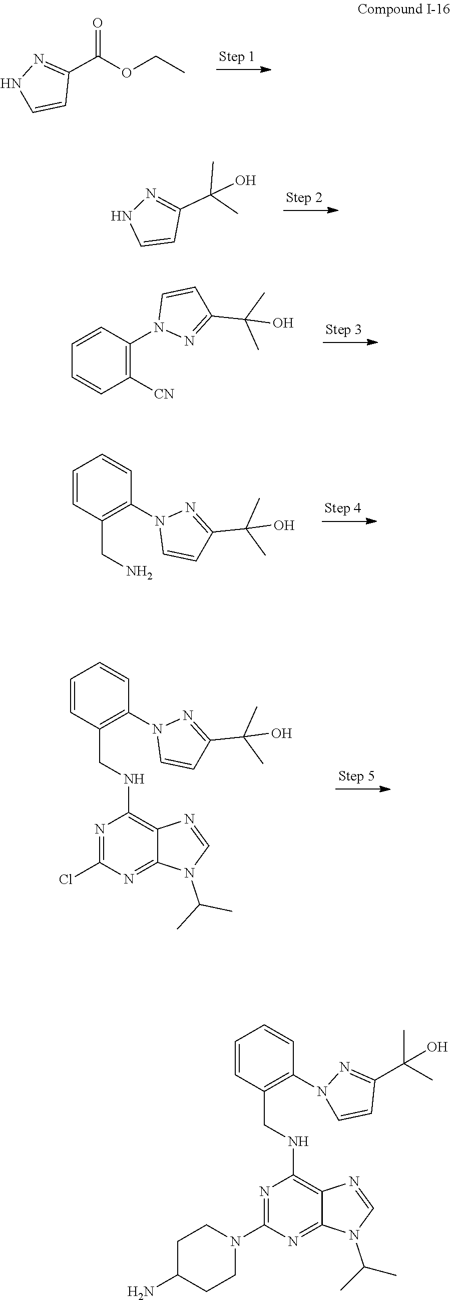

### Step 1

### 2-(1H-pyrazol-3-yl)propan-2-ol

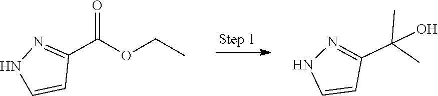

To a solution of ethyl 1H-pyrazole-3-carboxylate (5 g, 35.68 mmol) in THE (50 mL) was added MeMgBr (124.88 mmol, 5 mL) dropwise at 0-5° C. under N₂ atmosphere. The resulting mixture was stirred at 10° C. for 15 hrs to form a white suspension. TLC showed the reaction was completed. The mixture was poured into saturated aqueous NH₄Cl (50 mL). The mixture was extracted with EtOAc (50 mL×3). The combined organic extract was washed with brine (80 mL×2), dried over anhydrous Na₂SO₄, filtered and concentrated under reduced pressure to give compound 2-(1H-pyrazol-3-yl)propan-2-ol (4.33 g, 33.64 mmol, 94.28% yield, 98% purity) as a white solid.

LCMS [M+H]⁺: 127.23

### Step 2

### 2-(3-(2-hydroxypropan-2-yl)-1H-pyrazol-1-yl)benzonitrile

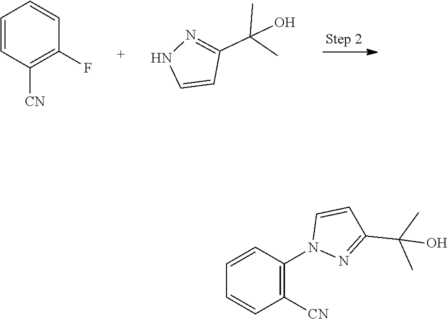

To a mixture of compound 2-(1H-pyrazol-3-yl)propan-2-ol (1.3 g, 10.30 mmol) and 2-fluorobenzonitrile (1.50 g, 12.37 mmol) in DMF (15 mL) was added Cs₂CO₃ (6.7 g, 20.61 mmol). The mixture was stirred at 15° C. for 15 hrs to form a white suspension. TLC showed the reaction was completed. The mixture was partitioned between EtOAc (150 mL) and H₂O (150 mL). The aqueous phase was extracted with EtOAc (150 mL×2). The combined organic extracts were washed with brine (100 mL×3), dried over anhydrous Na₂SO₄, filtered and concentrated under reduced pressure to give the crude product, which was purified by combi flash to give compound 2-(3-(2-hydroxypropan-2-yl)-1H-pyrazol-1-yl)benzonitrile (1.3 g, 5.43 mmol, 52.74% yield, 95% purity) as a yellow solid.

LCMS [M+H]⁺: 228.43

### Step 3

### 2-(1-(2-(aminomethyl)phenyl)-1H-pyrazol-3-yl)propan-2-ol

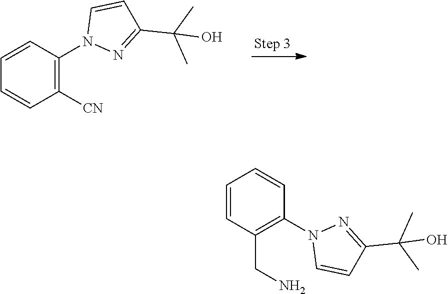 [SMILES: CC(C)(O)c1ccn(-c2ccccc2C#N)n1 ; CC(C)(O)c1ccn(-c2ccccc2CN)n1]

To a solution of 2-(3-(2-hydroxypropan-2-yl)-1H-pyrazol-1-yl)benzonitrile (200 mg, 880.04 mol) in THE (10 mL) was added LiAlH₄ (36.74 mg, 968.05 μmol). After being stirred for 1 h, the reaction was diluted with THF and quenched with water (100 μL), NaOH (15% in water, 100 μL). The reaction mixture was stirred at room temperature for 1 h, then dried over Na₂SO₄. After filtration and evaporation of the solvent, the crude mixture was purified by column chromatography on silica gel to afford the product as a thick oil (168 mg, 726.35 μmol, 82.54% yield).

LCMS [M+H]⁺: 231.29

### Step 4

### 2-(1-(2-(((2-chloro-9-isopropyl-9H-purin-6-yl)amino)methyl)phenyl)-1H-pyrazol-3-yl)propan-2-ol

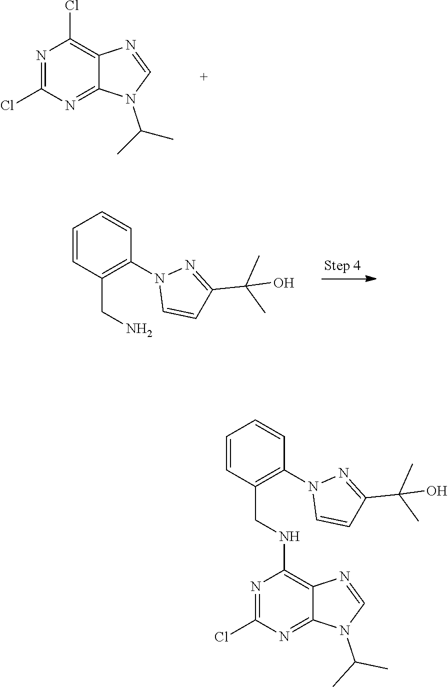 [SMILES: CC(C)n1cnc2c(Cl)nc(Cl)nc21 ; CC(C)(O)c1ccn(-c2ccccc2CN)n1 ; CC(C)n1cnc2c(NCc3ccccc3-n3ccc(C(C)(C)O)n3)nc(Cl)nc21]

To a mixture of 2,6-dichloro-9-isopropyl-9H-purine (610 mg, 2.64 mmol), 2-(1-(2-(aminomethyl)phenyl)-1H-pyrazol-3-yl)propan-2-ol (641.09 mg, 2.77 mmol) in EtOH (20 mL) was added TEA (801.35 mg, 7.92 mmol, 1.10 mL). The mixture was stirred at 78° C. for 6 hrs to give a green suspension. TLC showed the reaction was completed. The mixture was partitioned between EtOAc (100 mL) and water (100 mL), the aqueous phase was extracted with EtOAc (80 mL×2). The combined organic phase was washed with water (80 mL), dried over Na₂SO₄, filtered and concentrated under reduced pressure to give a green oil, which was purified by Combi flash to give 2-(1-(2-(((2-chloro-9-isopropyl-9H-purin-6-yl)amino)methyl)phenyl)-1H-pyrazol-3-yl)propan-2-ol (908 mg, 2.05 mmol, 77.53% yield, 96% purity) as a white solid.

LCMS [M+H]⁺. 426.41

### Step 5

### Synthesis of 2-(1-(2-(((2-(4-aminopiperidin-1-yl)-9-isopropyl-9H-purin-6-yl)amino)methyl)phenyl)-1H-pyrazol-3-yl)propan-2-ol

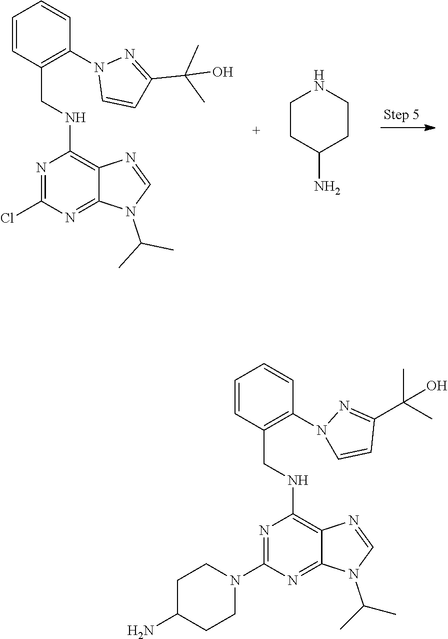 [SMILES: CC(C)n1cnc2c(NCc3ccccc3-n3ccc(C(C)(C)O)n3)nc(Cl)nc21 ; NC1CCNCC1 ; CC(C)n1cnc2c(NCc3ccccc3-n3ccc(C(C)(C)O)n3)nc(N3CCC(N)CC3)nc21]

To a solution of 2-(1-(2-(((2-chloro-9-isopropyl-9H-purin-6-yl)amino)methyl)phenyl)-1H-pyrazol-3-yl)propan-2-ol (200 mg, 469.58 μmol) in DMSO (2.5 mL) were added piperidin-4-amine (56.44 mg, 563.49 μmol) and TEA (142.55 mg, 1.41 mmol, 196.49 μL), then stirred at 160° C. for 10 hrs. The reaction was poured into water (30 mL), extracted with EtOAc (10 mL*2). The organic layer was washed with water (10 mL*2), dried over Na₂SO₄, filtered and concentrated to give a residue. The residue was purified by column chromatography to give 2-(1-(2-(((2-(4-aminopiperidin-1-yl)-9-isopropyl-9H-purin-6-yl)amino)methyl)phenyl)-1H-pyrazol-3-yl)propan-2-ol (168.3 mg, 343.74 mol, 73.2% yield) as a white solid.

¹H NMR (400 MHz, Methanol-d4) δ 7.80 (d, J=2.4 Hz, 1H), 7.77 (s, 1H), 7.63 (tt, J=4.4, 2.4 Hz, 1H), 7.39 (d, J=2.7 Hz, 3H), 6.53 (d, J=2.4 Hz, 1H), 4.78-4.53 (m, 5H), 2.84 (ddt, J=16.9, 11.0, 3.6 Hz, 3H), 1.88-1.76 (m, 2H), 1.62 (s, 6H), 1.55 (d, J=6.8 Hz, 6H), 1.34-1.18 (m, 2H). LCMS [M+H]⁺: 490.32.

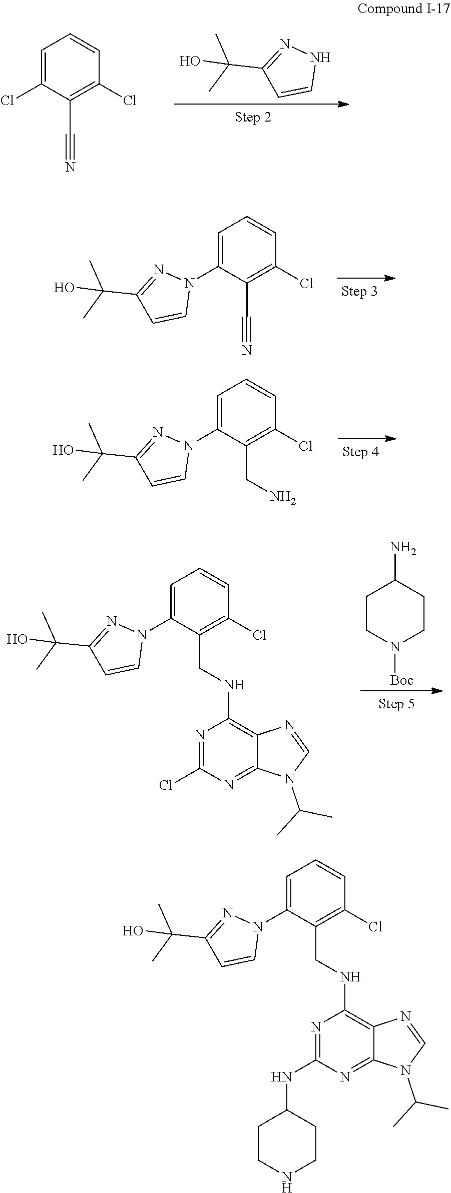

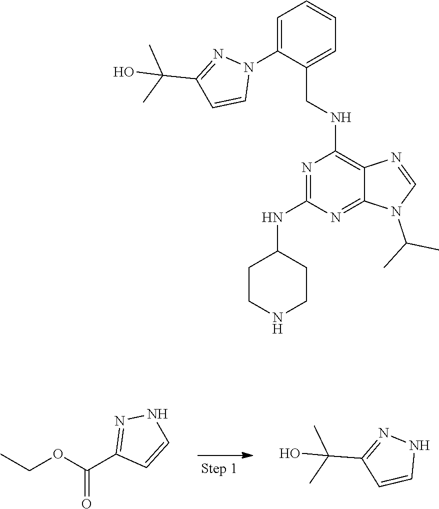

### Step 1

### 2-(1H-pyrazol-3-yl)propan-2-ol

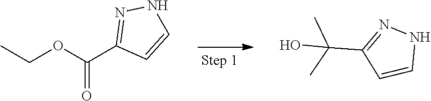

To a solution of ethyl 1H-pyrazole-3-carboxylate (5 g, 35.68 mmol, 1 eq) in THF (50 mL) was added MeMgBr (3 M, 47.60 mL, 4.00 eq) under N₂ atmosphere, then stirred at 20° C. for 10 hrs. The reaction mixture was poured into sat.NH₄Cl (100 mL), extracted with EtOAc (50 mL*3), the organic layer was dried over Na₂SO₄, filtered and concentrated to give 2-(1H-pyrazol-3-yl)propan-2-ol (2.6 g, 20.61 mmol, 57.76% yield) as a white solid. ¹HNMR (400 MHz, CDCl₃): δ 7.38 (d, J=1.9 Hz, 1H), 6.06 (d, J=1.4 Hz, 1H), 1.52 (s, 6H).

### Step 2

### 2-chloro-6-(3-(2-hydroxypropan-2-yl)-1H-pyrazol-1-yl)benzonitrile

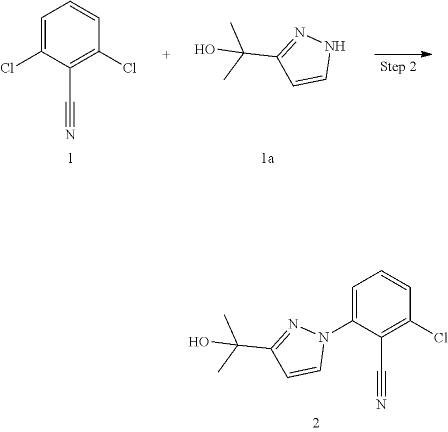

To a solution of 2-(1H-pyrazol-3-yl)propan-2-ol (1 g, 7.93 mmol, 1 eq) and 2,6-dichlorobenzonitrile (1.36 g, 7.93 mmol, 1 eq) in DMF (10 mL) was added Cs₂CO₃ (5.17 g, 15.85 mmol, 2 eq) and stirred at 20° C. for 10 hrs. The reaction mixture was poured into water (30 mL), extracted with EtOAc (30 mL*2), the organic layer was dried over Na₂SO₄, filtered and concentrated to give a residue. The residue was purified by column chromatography (SiO₂, Petroleum ether:Ethyl acetate=20:1 to 1:1, TLC: Petroleum ether:Ethyl acetate=1:1, P1 R_{f}=0.41) to give 2-chloro-6-[3-(1-hydroxy-1-methyl-ethyl)pyrazol-1-yl]benzonitrile (1.2 g, 4.59 mmol, 57.85% yield) as a white solid. LCMS [M-OH]*: 244.1

### Step 3

### 2-(1-(2-(aminomethyl)-3-chlorophenyl)-1H-pyrazol-3-yl)propan-2-ol

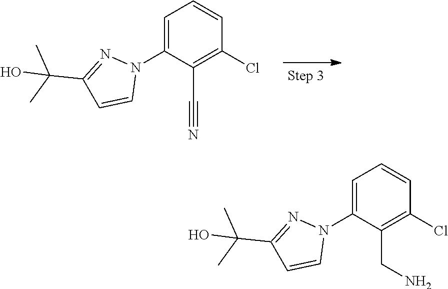 [SMILES: CC(C)(O)c1ccn(-c2cccc(Cl)c2C#N)n1 ; CC(C)(O)c1ccn(-c2cccc(Cl)c2CN)n1]

To a solution of Raney-Ni (0.1 g) in MeOH (5 mL) was added 2-chloro-6-[3-(1-hydroxy-1-methyl-ethyl)pyrazol-1-yl]benzonitrile (1 g, 3.82 mmol, 1 eq) and NH₃·H₂O (910.00 mg, 7.79 mmol, 1 mL, 30% purity, 2.04 eq) stirred at 20° C. for 10 hrs under H₂ atmosphere(15 Psi). The reaction mixture was filtered to give 2-[1-[2-(aminomethyl)-3-chloro-phenyl]pyrazol-3-yl]propan-2-ol (0.9 g, crude) as a yellow solid. LCMS [M+H]⁺: 266.1

### Step 4

### 2-(1-(3-chloro-2-(((2-chloro-9-isopropyl-9H-purin-6-yl)amino)methyl)phenyl)-1H-pyrazol-3-yl)propan-2-ol

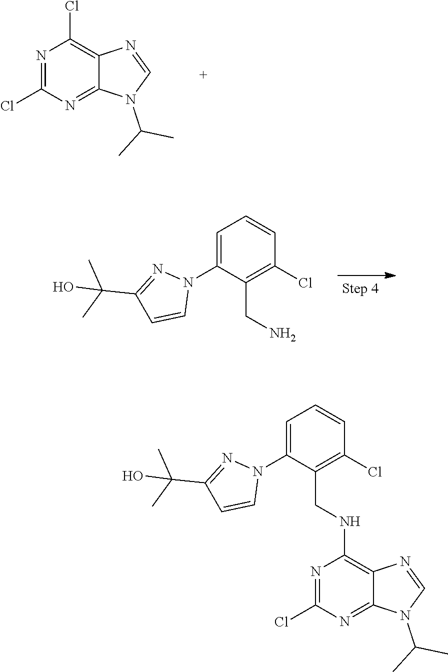 [SMILES: CC(C)n1cnc2c(Cl)nc(Cl)nc21 ; CC(C)(O)c1ccn(-c2cccc(Cl)c2CN)n1 ; CC(C)n1cnc2c(NCc3c(Cl)cccc3-n3ccc(C(C)(C)O)n3)nc(Cl)nc21]

To a solution of 2-[1-[2-(aminomethyl)-3-chloro-phenyl]pyrazol-3-yl]propan-2-ol (758.98 mg, 2.86 mmol, 1.1 eq) and 2,6-dichloro-9-isopropyl-purine (0.6 g, 2.60 mmol, 1 eq) in n-BuOH (10 mL) was added DIEA (1.01 g, 7.79 mmol, 1.36 mL, 3 eq) and stirred at 110° C. for 2 hrs. The reaction mixture was concentrated to give a residue. The residue was purified by column chromatography (SiO₂, Petroleum ether:Ethyl acetate=20:1 to 1:1, TLC: Petroleum ether:Ethyl acetate=1:1, P1 R_{f}=0.3) to give 2-[1-[3-chloro-2-[[(2-chloro-9-isopropyl-purin-6-yl)amino]methyl]phenyl]pyrazol-3-yl]propan-2-ol (0.8 g, 1.74 mmol, 66.93% yield) as a yellow solid. LCMS [M+Na]*: 482.1

### Step 5

### 2-(1-(2-(((9-isopropyl-2-(piperidin-4-ylamino)-9H-purin-6-yl)amino)methyl)phenyl)-1H-pyrazol-3-yl)propan-2-ol

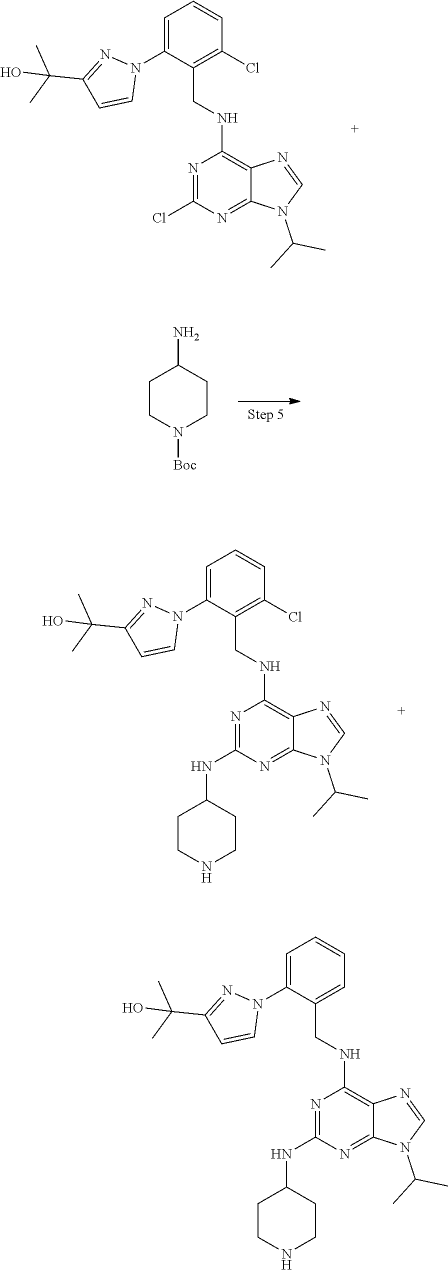 [SMILES: CC(C)n1cnc2c(NCc3c(Cl)cccc3-n3ccc(C(C)(C)O)n3)nc(Cl)nc21 ; CC(C)(C)OC(=O)N1CCC(N)CC1 ; CC(C)n1cnc2c(NCc3c(Cl)cccc3-n3ccc(C(C)(C)O)n3)nc(NC3CCNCC3)nc21 ; CC(C)n1cnc2c(NCc3ccccc3-n3ccc(C(C)(C)O)n3)nc(NC3CCNCC3)nc21]

To a solution of 2-[1-[3-chloro-2-[[(2-chloro-9-isopropyl-purin-6yl)amino]methyl]phenyl]pyrazol-3-yl]propan-2-ol (0.2 g, 434.44 umol, 1 eq) and tert-butyl 4-aminopiperidine-1-carboxylate (0.2 g, 998.62 umol, 2.30 eq) in n-BuOH (2 mL) was added DIEA (371.00 mg, 2.87 mmol, 0.5 mL, 6.61 eq) at 160° C. for 8 hrs under microwave. The reaction was concentrated to give a residue. The residue was purified by prep-HPLC(column: Phenomenex Synergi Polar-RP 100*25 mm*4 um; mobile phase: [water(0.225% FA)-ACN];B %: 5%-35%, 8 min) to give 2-[1-[3-chloro-2-[[[9-isopropyl-2-(4-piperidylamino)purin-6-yl]amino]methyl]phenyl]pyrazol-3-yl]propan-2-ol (0.02 g, 34.73 umol, 7.99% yield, 91% purity) as a yellow solid. ¹HNMR (400 MHz, Methanol-d4): 7.78 (d, J=2.4 Hz, 1H), 7.74 (s, 1H), 7.56 (dd, J=1.2, 7.9 Hz, 1H), 7.47-7.41 (m, 1H), 7.40-7.35 (m, 1H), 6.49 (d, J=2.4 Hz, 1H), 4.84-4.74 (m, 5H), 4.71-4.62 (m, 1H), 2.93-2.82 (m, 2H), 1.85 (dd, J=2.2, 12.4 Hz, 2H), 1.55 (d, J=6.9 Hz, 6H), 1.35-1.23 (m, 2H). LCMS [M+H]⁺: 524.3. give 2-[1-[2-[[[9-isopropyl-2-(4-piperidylamino)purin-6-yl]amino]methyl]phenyl]pyrazol-3-yl]propan-2-ol (0.02 g, 38.81 umol, 8.93% yield, 95% purity) as a yellow solid. ¹HNMR (400 MHz, Methanol-d4): 7.78 (d, J=2.3 Hz, 1H), 7.76 (s, 1H), 7.64-7.57 (m, 1H), 7.38 (d, J=2.8 Hz, 3H), 6.52 (d, J=2.4 Hz, 1H), 4.76-4.60 (m, 5H), 2.95-2.75 (m, 3H), 1.83 (br d, J=10.0 Hz, 2H), 1.54 (d, J=6.8 Hz, 6H), 1.26 (dq, J=4.0, 11.9 Hz, 2H). LCMS [M+H]⁺: 490.4.

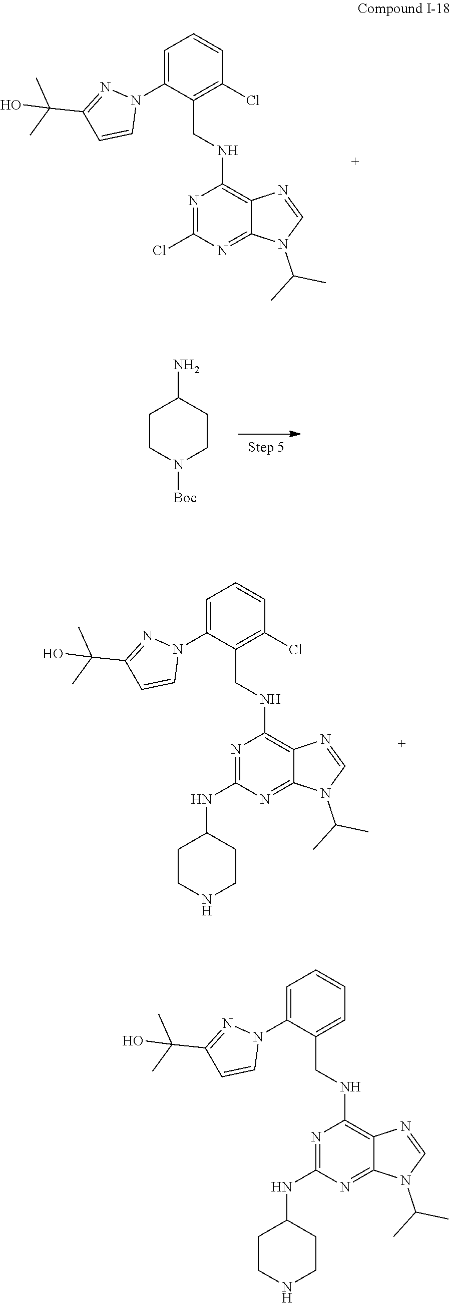 [SMILES: CC(C)n1cnc2c(NCc3c(Cl)cccc3-n3ccc(C(C)(C)O)n3)nc(Cl)nc21 ; CC(C)(C)OC(=O)N1CCC(N)CC1 ; CC(C)n1cnc2c(NCc3c(Cl)cccc3-n3ccc(C(C)(C)O)n3)nc(NC3CCNCC3)nc21 ; CC(C)n1cnc2c(NCc3ccccc3-n3ccc(C(C)(C)O)n3)nc(NC3CCNCC3)nc21]

To a solution of 2-[1-[3-chloro-2-[[(2-chloro-9-isopropyl-purin-6yl)amino]methyl]phenyl]pyrazol-3-yl]propan-2-ol (0.2 g, 434.44 umol, 1 eq) and tert-butyl 4-aminopiperidine-1-carboxylate (0.2 g, 998.62 umol, 2.30 eq) in n-BuOH (2 mL) was added DIEA (371.00 mg, 2.87 mmol, 0.5 mL, 6.61 eq) at 160° C. for 8 hrs under microwave. The reaction was concentrated to give a residue. The residue was purified by prep-HPLC(column: Phenomenex Synergi Polar-RP 100*25 mm*4 um; mobile phase: [water(0.225% FA)-ACN];B %: 5%-35%, 8 min) to give 2-[-[3-chloro-2-[[[9-isopropyl-2-(4-piperidylamino)purin-6-yl]amino]methyl]phenyl]pyrazol-3-yl]propan-2-ol (0.02 g, 34.73 umol, 7.99% yield, 91% purity) as a yellow solid. ¹HNMR (400 MHz, Methanol-d4): 7.78 (d, J=2.4 Hz, 1H), 7.74 (s, 1H), 7.56 (dd, J=1.2, 7.9 Hz, 1H), 7.47-7.41 (m, 1H), 7.40-7.35 (m, 1H), 6.49 (d, J=2.4 Hz, 1H), 4.84-4.74 (m, 5H), 4.71-4.62 (m, 1H), 2.93-2.82 (m, 2H), 1.85 (dd, J=2.2, 12.4 Hz, 2H), 1.55 (d, J=6.9 Hz, 6H), 1.35-1.23 (m, 2H). LCMS [M+H]⁺: 524.3. give 2-[1-[2-[[[9-isopropyl-2-(4-piperidylamino)purin-6-yl]amino]methyl]phenyl]pyrazol-3-yl]propan-2-ol (0.02 g, 38.81 umol, 8.93% yield, 95% purity) as a yellow solid. ¹HNMR (400 MHz, Methanol-d4): 7.78 (d, J=2.3 Hz, 1H), 7.76 (s, 1H), 7.64-7.57 (m, 1H), 7.38 (d, J=2.8 Hz, 3H), 6.52 (d, J=2.4 Hz, 1H), 4.76-4.60 (m, 5H), 2.95-2.75 (m, 3H), 1.83 (br d, J=10.0 Hz, 2H), 1.54 (d, J=6.8 Hz, 6H), 1.26 (dq, J=4.0, 11.9 Hz, 2H). LCMS [M+H]⁺: 490.4.

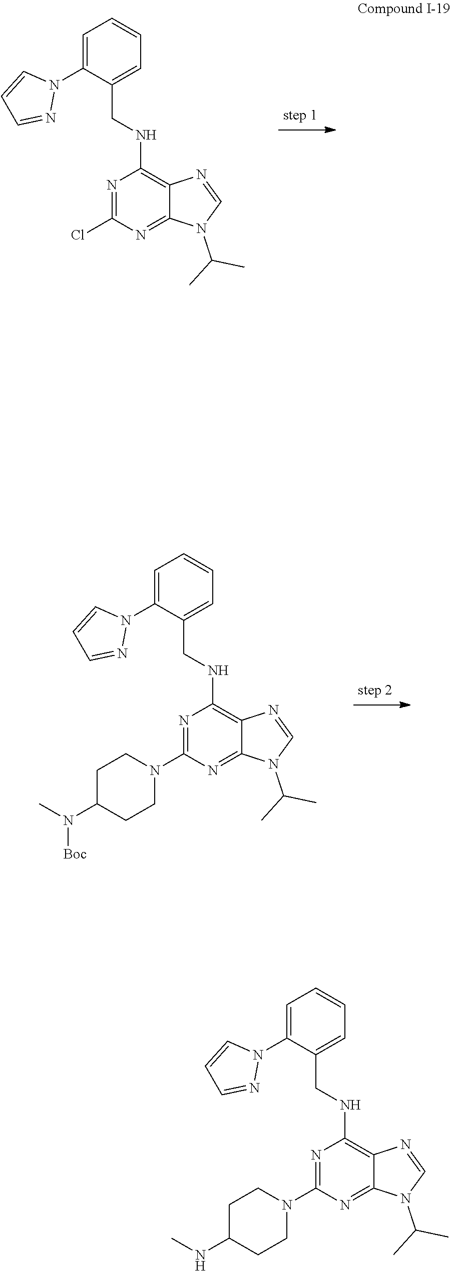

### Step 1

### tert-butyl (1-(6-((2-(1H-pyrazol-1-yl)benzyl)amino)-9-isopropyl-9H-purin-2-yl)piperidin-4-yl)(methyl)carbamate (compound 2)

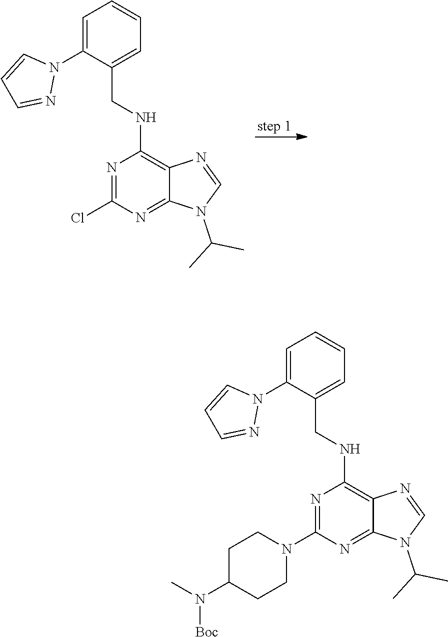 [SMILES: CC(C)n1cnc2c(NCc3ccccc3-n3cccn3)nc(Cl)nc21 ; CC(C)n1cnc2c(NCc3ccccc3-n3cccn3)nc(N3CCC(N(C)C(=O)OC(C)(C)C)CC3)nc21]

To a solution of 2-chloro-9-isopropyl-N-[(2-pyrazol-1-ylphenyl)methyl]purin-6-amine (200 mg, 543.72 umol, 1 eq) and tert-butyl N-methyl-N-(4-piperidyl)carbamate (198.09 mg, 924.33 umol, 1.7 eq) in dioxane (10 mL) was added Cs₂CO₃ (708.62 mg, 2.17 mmol, 4 eq), RuPhos Pd G3 (90.95 mg, 108.74 umol, 0.2 eq), the mixture was stirred at 100° C. for 12 hrs. LCMS showed one main peak with desired mass. The reaction mixture was filtered and concentrated under reduced pressure to give a residue. Without purification Compound tert-butyl N-[1-[9-isopropyl-6-[(2-pyrazol-1-ylphenyl)methylamino]purin-2-yl]-4-piperidyl]-N-methyl-carbamate (100 mg, 183.26 umol, 33.700% yield) was obtained as a black brown oil. LCMS [M+H]. 546.3

### Step 2

### N-(2-(1H-pyrazol-1-yl)benzyl)-9-isopropyl-2-(4-(methylamino)piperidin-1-yl)-9H-purin-6-amine (compound I-29)

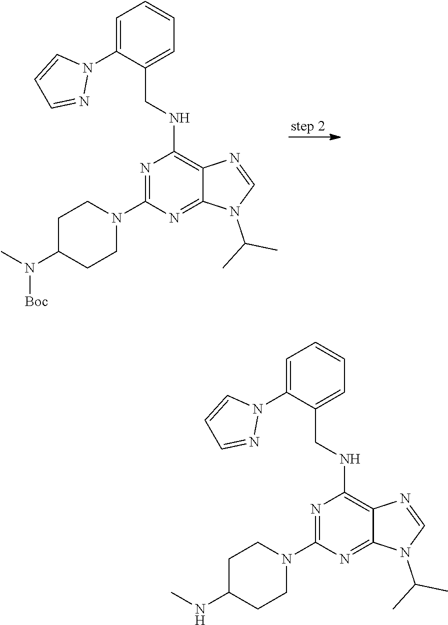

To a solution of tert-butyl N-[1-[9-isopropyl-6-[(2-pyrazol-1-ylphenyl)methylamino]purin-2-yl]-4-piperidyl]-N-methyl-carbamate (100 mg, 183.26 umol, 1 eq) in DCM (5 mL) was added TFA (208.96 mg, 1.83 mmol, 135.69 uL, 10 eq) and stirred at 20° C. for 5 hrs. LCMS showed reactant 1 was consumed completely and one main peak with desired mass was detected. The reaction mixture was filtered and concentrated under reduced pressure to give a residue. The residue was purified by prep-HPLC (TFA condition; column: Welch Xtimate C18 150*25 mm*5 um; mobile phase: [water(0.1% TFA)-ACN];B %:10%-40%,10 min). Compound 9-isopropyl-2-[4-(methylamino)-1-piperidyl]-N-[(2-pyrazol-1-ylphenyl)methyl]purin-6-amine (50 mg, 111.66 umol, 60.93% yield, 99.5% purity) was obtained as a yellow solid. ¹HNMR (400 MHz, Methanol-d4): δ ppm 8.54-8.67 (m, 1H) 7.93 (d, J=2.25 Hz, 1H) 7.78 (s, 1H) 7.61-7.65 (m, 1H) 7.40-7.46 (m, 3H) 6.55 (t, J=1.69 Hz, 1H) 4.78-4.84 (m, 2H) 4.72 (br s, 2H) 3.31 (br s, 2H) 2.86-2.99 (m, 2H) 2.73 (s, 3H) 2.10 (br d, J=10.63 Hz, 2H) 1.61 (d, J=6.63 Hz, 6H) 1.44 (qd, J=12.13, 3.63 Hz, 2H). LCMS [M+H]⁺: 446.2

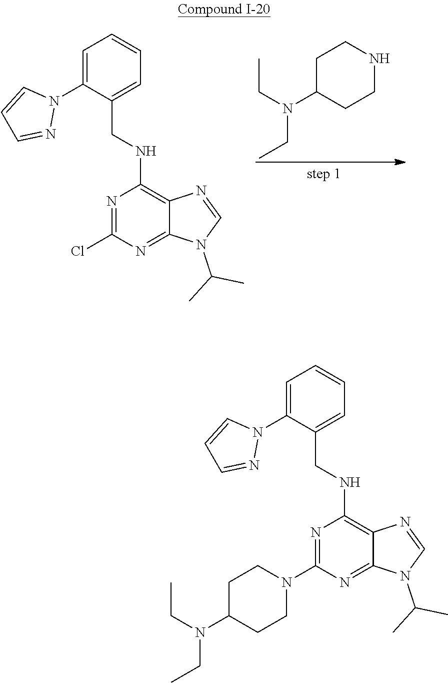 [SMILES: CC(C)n1cnc2c(NCc3ccccc3-n3cccn3)nc(Cl)nc21 ; CCN(CC)C1CCNCC1 ; CCN(CC)C1CCN(c2nc(NCc3ccccc3-n3cccn3)c3ncn(C(C)C)c3n2)CC1]

### Step 1

### 2-[4-(diethylamino)-1-piperidyl]-9-isopropyl-N-[(2-pyrazol-1-ylphenyl)methyl]purin-6-amine (compound I-28)

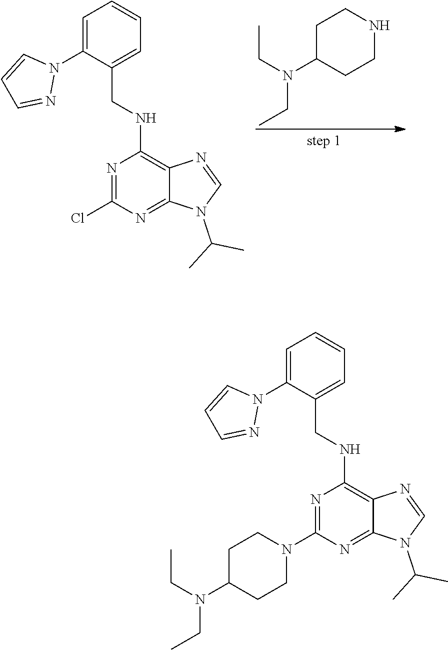 [SMILES: CC(C)n1cnc2c(NCc3ccccc3-n3cccn3)nc(Cl)nc21 ; CCN(CC)C1CCNCC1 ; CCN(CC)C1CCN(c2nc(NCc3ccccc3-n3cccn3)c3ncn(C(C)C)c3n2)CC1]

To a solution of 2-chloro-9-isopropyl-N-[(2-pyrazol-1-ylphenyl)methyl]purin-6-amine (0.2 g, 543.72 umol, 1 eq) and N,N-diethylpiperidin-4-amine (84.97 mg, 543.72 umol, 1 eq) in dioxane (2 mL) was added RuPhos Pd G₃ (45.48 mg, 54.37 umol, 0.1 eq) and Cs₂CO₃ (531.47 mg, 1.63 mmol, 3 eq), then stirred at 100° C. for 10 hrs under N₂ atmosphere. The reaction mixture was poured into water (2 mL), extracted with EtOAc (2 mL). The organic layer dried over Na₂SO₄, filtered and concentrated to give a residue. The residue was purified by prep-HPLC (column: Phenomenex Luna C18 150*25 mm*10 um; mobile phase: [water(0.225% FA)-ACN];B %: 10%-40%,10 min) to give 2-[4-(diethylamino)-1-piperidyl]-9-isopropyl-N-[(2-pyrazol-1-ylphenyl)methyl]purin-6-amine (30 mg, 61.52 umol, 11.31% yield) as a yellow solid.

¹H NMR (400 MHz, DMSO-d₆) δ 8.28 (s, 1H), 8.12 (d, J=2.3 Hz, 1H), 7.85-7.80 (m, 2H), 7.55-7.50 (m, 1H), 7.43-7.33 (m, 3H), 6.56 (t, J=2.1 Hz, 1H), 4.55 (td, J=6.5, 13.3 Hz, 5H), 2.88-2.78 (m, 1H), 2.69-2.54 (m, 6H), 1.69 (br d, J=10.8 Hz, 2H), 1.46 (d, J=6.8 Hz, 6H), 1.23 (br d, J=9.5 Hz, 2H), 1.01 (t, J=7.1 Hz, 6H). LCMS [M+H]⁺: 488.2

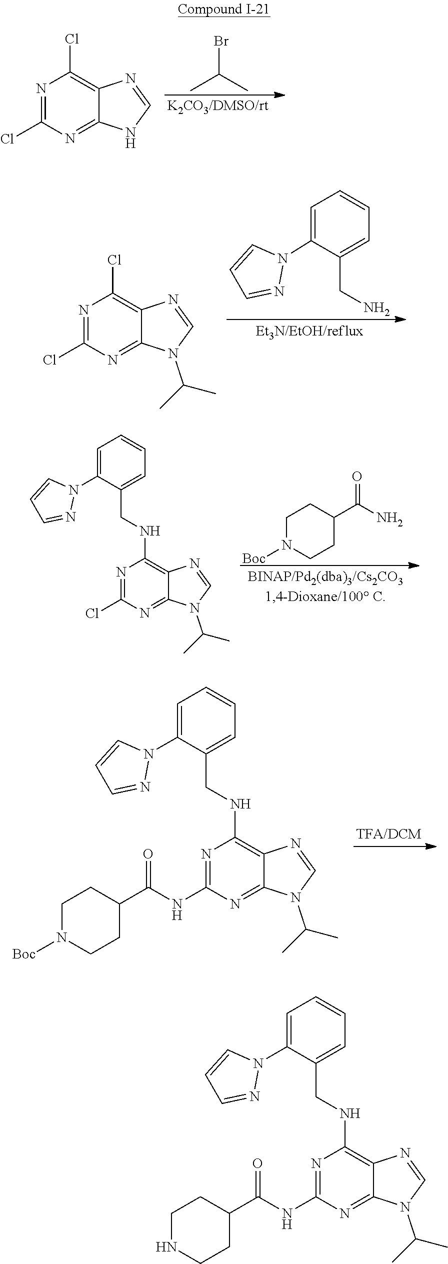

tert-butyl 4-((6-((2-(1H-pyrazol-1-yl)benzyl)amino)-9-isopropyl-9H-purin-2-yl)carbamoyl)piperidine-1-carboxylate: N-(2-(1H-pyrazol-1-yl)benzyl)-2-chloro-9-isopropyl-9H-purin-6-amine (100 mg, 0.272 mmol), tert-butyl pyrrolidin-3-ylcarbamate (109 mg, 0.544 mmol), potassium carbonate (75 mg, 0.544 mmol) and 99% NMP (2 mL) were added to a 4 mL reaction flask, heated to 165° C. and stirred for 2.5 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo to afford 138 mg oil. The crude was purified by silica gel column (Methanol/dichloromethane=0-10%) to afford 88 mg oil.

N-(6-((2-(1H-pyrazol-1-yl)benzyl)amino)-9-isopropyl-9H-purin-2-yl)piperidine-4-carboxamide: To a solution of tert-butyl 4-((6-((2-(1H-pyrazol-1-yl)benzyl)amino)-9-isopropyl-9H-purin-2-yl)carbamoyl)piperidine-1-carboxylate in DCM(3 mL) was added TFA(6 mL), the mixture was stirred at room temperature for 2 hrs. The reaction solution was concentrated in vacuo to afford 467 mg oil. The oil was dissolved in water and ethyl acetate was added to extract the impurities. Then potassium carbonate was added to the aqueous phase to adjust the pH>10, and extracted with EA twice. The combined organic phases were dried with sodium sulfate, filtered and concentrated in vacuo to afford 86 mg of a white solid.

¹H NMR (400 MHz, DMSO-d6) δ 9.81 (s, 1H), 8.18 (d, J=2.4 Hz, 1H), 8.14 (s, 1H), 8.02 (s, 1H), 7.83 (d, J=1.9 Hz, 1H), 7.62-7.54 (m, 1H), 7.39 (qq, J=7.2, 3.5, 2.8 Hz, 3H), 6.56 (d, J=2.2 Hz, 1H), 4.66 (h, J=6.7, 5.7 Hz, 4H), 3.13 (d, J=12.1 Hz, 2H), 3.00 (d, J=27.4 Hz, 1H), 2.63 (t, J=12.2 Hz, 2H), 1.88-1.76 (m, 2H), 1.63 (qd, J=12.8, 4.0 Hz, 2H), 1.51 (d, J=6.8 Hz, 6H).

MS: cacl. for C24H29N9O 459.25. Found: [M+H]⁺: 460.35

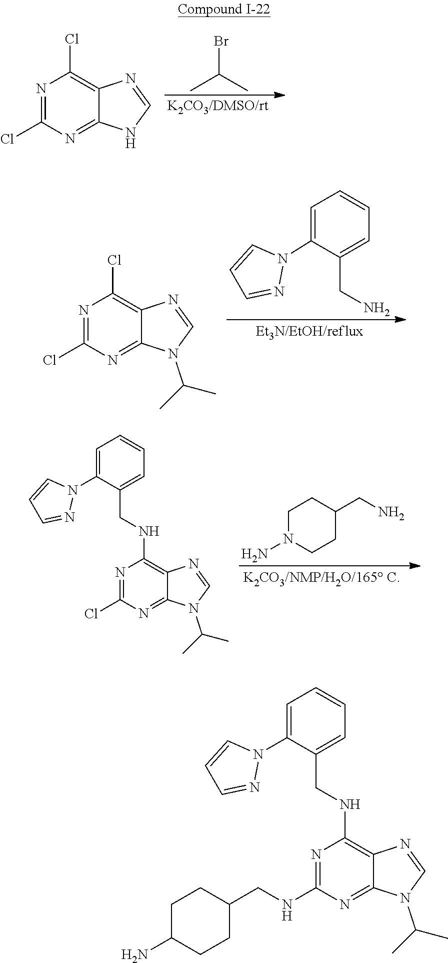

N6-(2-(1H-pyrazol-1-yl)benzyl)-N2-((4-aminocyclohexyl)methyl)-9-isopropyl-9H-purine-2,6-diamine: N-(2-(1H-pyrazol-1-yl)benzyl)-2-chloro-9-isopropyl-9H-purin-6-amine (100 mg, 0.272 mmol), 4-(aminomethyl)cyclohexan-1-amine (70 mg, 0.544 mmol), potassium carbonate (75 mg, 0.544 mmol) and 990% NMP (2 mL) were added to a 4 mL reaction flask, heated to 165° C. and stirred for 14 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo to afford 82 mg oil. The crude was purified by silica gel column (10% ammonia methanol solution:DCM=0% to 20%) to afford 41 mg off-white solid.

¹H NMR (400 MHz, Methanol-d4) δ 7.94 (q, J=2.1 Hz, 1H), 7.85-7.76 (m, 2H), 7.67-7.59 (m, 1H), 7.49-7.37 (m, 3H), 6.55 (t, J=2.2 Hz, 1H), 4.73-4.59 (m, 3H), 3.22 (dd, J=6.7, 1.7 Hz, 1H), 2.94 (s, 1H), 1.85 (dt, J=32.4, 15.5 Hz, 2H), 1.70-1.45 (m, 12H), 1.39-1.29 (m, 2H).

MS: cacl. for C25H33N9 459.29. Found: [M+H]⁺:460.33

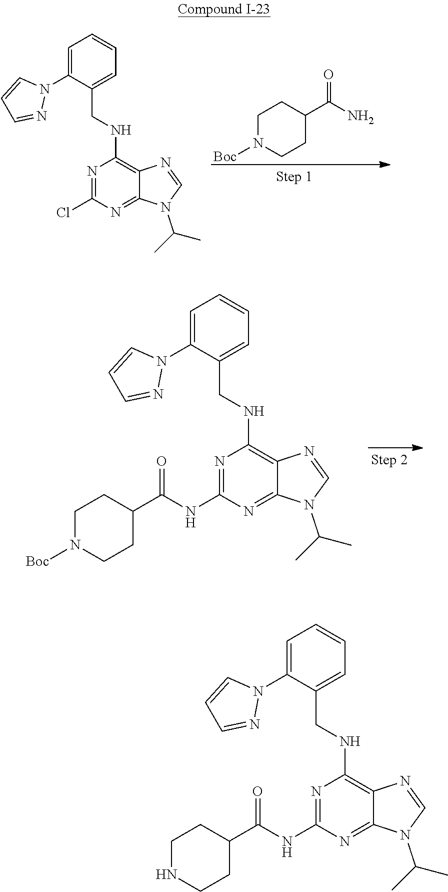 [SMILES: CC(C)n1cnc2c(NCc3ccccc3-n3cccn3)nc(Cl)nc21 ; CC(C)(C)OC(=O)N1CCC(C(N)=O)CC1 ; CC(C)n1cnc2c(NCc3ccccc3-n3cccn3)nc(NC(=O)C3CCN(C(=O)OC(C)(C)C)CC3)nc21 ; CC(C)n1cnc2c(NCc3ccccc3-n3cccn3)nc(NC(=O)C3CCNCC3)nc21]

### Step 1

### tert-butyl 4-[[9-isopropyl-6-[(2-pyrazol-1-ylphenyl)methylamino]purin-2-yl]carbamoyl]piperidine-1-carboxylate (compound I-18-A)

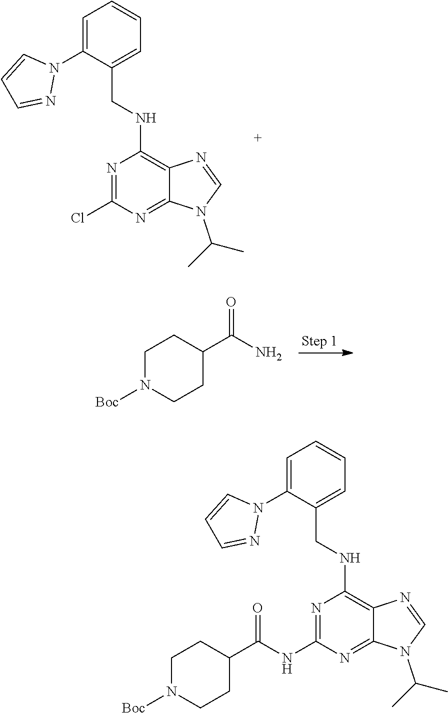 [SMILES: CC(C)n1cnc2c(NCc3ccccc3-n3cccn3)nc(Cl)nc21 ; CC(C)(C)OC(=O)N1CCC(C(N)=O)CC1 ; CC(C)n1cnc2c(NCc3ccccc3-n3cccn3)nc(NC(=O)C3CCN(C(=O)OC(C)(C)C)CC3)nc21]

To a solution of compound int.3 (62.1 mg, 271 umol, 1 eq) and compound B (100 mg, 271 umol, 1 eq) in dioxane (1 mL) was added Cs₂CO₃ (124 mg, 380 umol, 1.4 eq), Pd(dba)₂ (15.6 mg, 27.2 umol, 0.1 eq) and Xantphos (23.6 mg, 40.8 umol, 0.15 eq), then stirred at 100° C. for 10 hrs under N₂ atmosphere. The reaction mixture was poured into water (2 mL), extracted with EtOAc (2*2 mL), the organic layer was washed with water (2 mL*2), dried over Na₂SO₄, filtered and concentrated to give tert-butyl 4-[[9-isopropyl-6-[(2-pyrazol-1-ylphenyl)methylamino]purin-2-yl]carbamoyl]piperidine-1-carboxylate (0.314 g, crude) as an orange solid. LCMS [M+H]⁺: 560.3

### Step 2

### N-[9-isopropyl-6-[(2-pyrazol-1-ylphenyl)methylamino]purin-2-yl]piperidine-4-carboxamide (compound I-18)

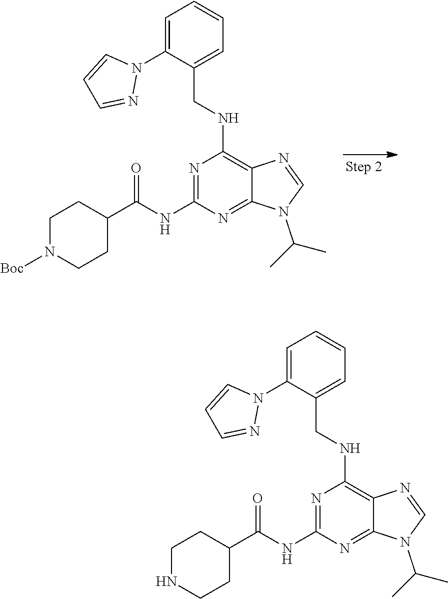 [SMILES: CC(C)n1cnc2c(NCc3ccccc3-n3cccn3)nc(NC(=O)C3CCN(C(=O)OC(C)(C)C)CC3)nc21 ; CC(C)n1cnc2c(NCc3ccccc3-n3cccn3)nc(NC(=O)C3CCNCC3)nc21]

To a solution of tert-butyl 4-[[9-isopropyl-6-[(2-pyrazol-1-ylphenyl)methylamino]purin-2-yl]carbamoyl]piperidine-1-carboxylate (0.15 g, 268.02 umol, 1 eq) in DCM (1 mL) was added TFA (770.00 mg, 6.75 mmol, 0.5 mL, 25.20 eq), then stirred at 20° C. for 1 hr. The reaction mixture was concentrated to give a residue. The residue was purified by prep-HPLC (column: Phenomenex Synergi C18 100*21.2 mm*4 um; mobile phase: [water(0.225% FA)-ACN];B %: 7%-37%,11.5 min) to give N-[9-isopropyl-6-[(2-pyrazol-1-ylphenyl)methylamino]purin-2-yl]piperidine-4-carboxamide (10 mg, 21.52 umol, 8.03% yield, 98.9% purity) as a white solid. ¹HNMR (400 MHz, DMSO-d₆) δ 9.81 (br s, 1H), 8.39 (s, 1H), 8.24-8.09 (m, 2H), 8.05-7.94 (m, 1H), 7.83 (s, 1H), 7.61-7.53 (m, 1H), 7.44-7.36 (m, 3H), 6.57 (s, 1H), 4.76-4.57 (m, 3H), 3.14 (br d, J=12.5 Hz, 2H), 3.09-3.00 (m, 1H), 2.69-2.62 (m, 2H), 1.83 (br d, J=11.8 Hz, 2H), 1.71-1.60 (m, 2H), 1.51 (d, J=6.8 Hz, 6H). LCMS [M+H]⁺: 460.1

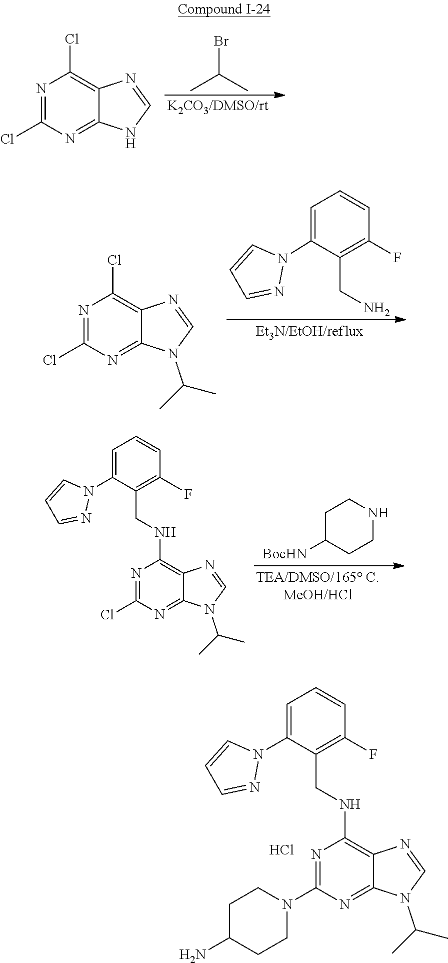

### Step 1. 2,6-dichloro-9-isopropyl-9H-purine

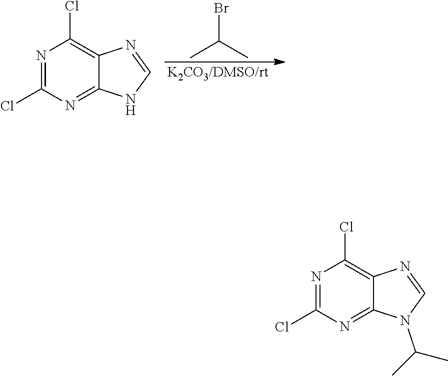

2,6-dichloro-9H-purine (10.0 g, 52.91 mmol), isopropyl bromide(32.5 g, 264.55 mmol), potassium carbonate(21.9 g, 158.73 mmol) and dimethyl sulfoxide(50 mL) were added to a 250 mL two-necked flask, and the mixture was stirred at room temperature for 96 hrs. The reaction solution was added dropwise to water, ethyl acetate was added for extraction three times. The organic phases were combined, washed twice with saturated brine, dried over sodium sulfate, filtered and concentrated in vacuo to afford 9.8 g of light yellow solid. The crude product was passed through a silica gel column (ethyl acetate/n-heptane=0-60%) to afford 4.941 g of white solid.

### Step 2. 2-chloro-N-(2-fluoro-6-(1H-pyrazol-1-yl)benzyl)-9-isopropyl-9H-purin-6-amine

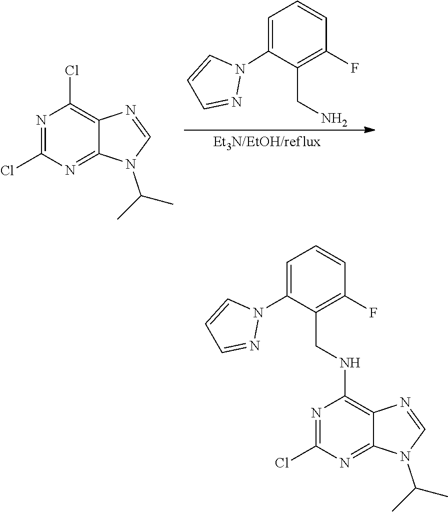 [SMILES: CC(C)n1cnc2c(Cl)nc(Cl)nc21 ; NCc1c(F)cccc1-n1cccn1 ; CC(C)n1cnc2c(NCc3c(F)cccc3-n3cccn3)nc(Cl)nc21]

To a solution of 2,6-dichloro-9-isopropyl-9H-purine (200 mg, 865.50 μmol) in EtOH (10 mL) were added TEA (262.74 mg, 2.60 mmol, 362.15 μL) and (2-fluoro-6-(1H-pyrazol-1-yl)phenyl)methanamine (330.97 mg, 1.73 mmol), then stirred at 80° C. for 10 hrs. The reaction was concentrated to give a residue. The residue was purified by column chromatography to give 2-chloro-N-(2-fluoro-6-(1H-pyrazol-1-yl)benzyl)-9-isopropyl-9H-purin-6-amine (300.23 mg, 754.81 μmol, 87.21% yield, 97% purity) as a white solid. LCMS [M+H]⁺: 386.47

### Step 3. 2-(4-aminopiperidin-1-yl)-N-(2-fluoro-6-(1H-pyrazol-1-yl)benzyl)-9-isopropyl-9H-purin-6-amine hydrochloride

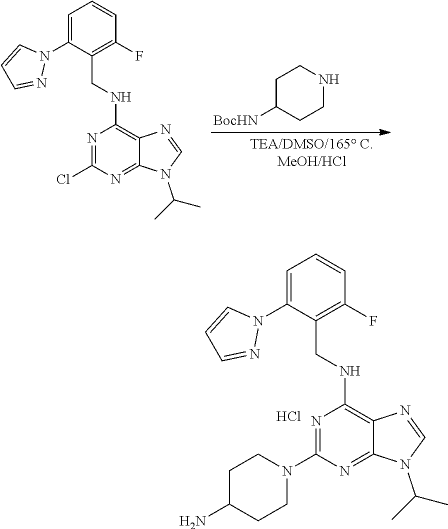 [SMILES: CC(C)n1cnc2c(NCc3c(F)cccc3-n3cccn3)nc(Cl)nc21 ; CC(C)(C)OC(=O)NC1CCNCC1 ; CC(C)n1cnc2c(NCc3c(F)cccc3-n3cccn3)nc(N3CCC(N)CC3)nc21 ; Cl]

To a solution of 2-chloro-N-(2-fluoro-6-(1H-pyrazol-1-yl)benzyl)-9-isopropyl-9H-purin-6-amine (100 mg, 259.18 μmol) in DMSO (2.5 mL) was added tert-butyl piperidin-4-ylcarbamate (51.91 mg, 259.18 μmol) and TEA (78.68 mg, 777.55 μmol, 108.45 μL), then stirred at 160° C. for 10 hrs. The reaction was poured into water (30 mL), extracted with EtOAc (10 mL*2), the organic layer was washed with water (10 mL*2), dried over Na₂SO₄, filtered and concentrated to give a residue. The residue was purified by column chromatography to give a white solid. Then MeOH/HCl was added and stirred at rt for 5 hrs. The mixture was concentrated to give 2-(4-aminopiperidin-1-yl)-N-(2-fluoro-6-(1H-pyrazol-1-yl)benzyl)-9-isopropyl-9H-purin-6-amine hydrochloride (87.43 mg, 184.77 μmol, 71.29% yield, 95% purity) as a yellow solid.

¹HNMR (400 MHz, Methanol-d4) δ 7.92 (d, J=2.3 Hz, 1H), 7.84-7.74 (m, 2H), 7.70-7.60 (m, 1H), 7.42 (dqd, J=8.3, 3.6, 2.1 Hz, 2H), 6.56 (q, J=2.2, 1.7 Hz, 1H), 4.68 (d, J=13.8 Hz, 3H), 2.96-2.73 (m, 3H), 1.89-1.71 (m, 3H), 1.56 (dd, J=6.8, 1.1 Hz, 6H), 1.36-1.11 (m, 3H). LCMS [M+H]⁺: 450.36

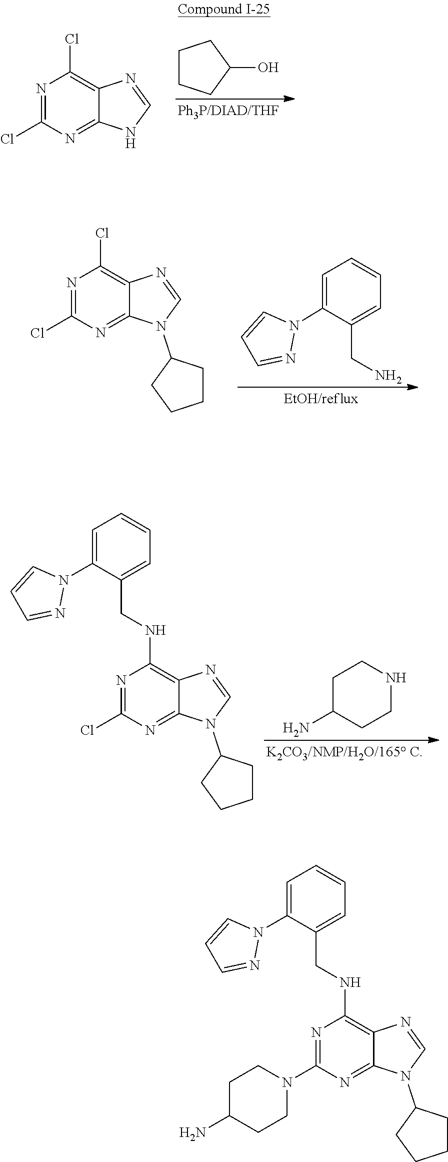

### Step 1. 2,6-dichloro-9-cyclopentyl-9H-purine

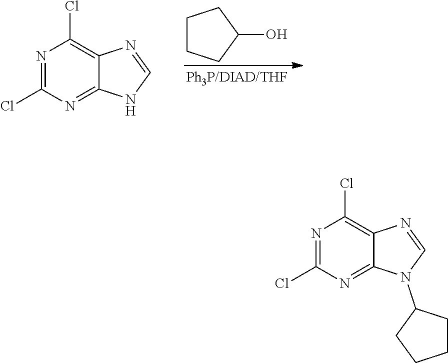

2,6-Dichloropurine(1000 mg, 5.29 mmol), triphenylphosphine oxide(1804 mg, 6.88 mmol), cyclopentanol(1367 mg, 15.87 mmol) and anhydrous tetrahydrofuran(20 mL) were added into a 100 mL three-necked flask, protected with nitrogen and cooled with ice ethanol. DIAD was added dropwise to the mixture. The temperature was naturally raised to room temperature and stirred for 16 hrs. The reaction solution was concentrated in vacuo to afford 5.7 g of oil, which was dissolved in ethyl acetate, washed twice with water, dried over sodium sulfate, filtered and concentrated in vacuo to afford 5.0 g of oil. The crude product was purified by silica gel column to afford 1.540 g of light-yellow solid. m/z:257.2[M+H]+

### Step 2. N-(2-(1H-pyrazol-1-yl)benzyl)-2-chloro-9-cyclopentyl-9H-purin-6-amine

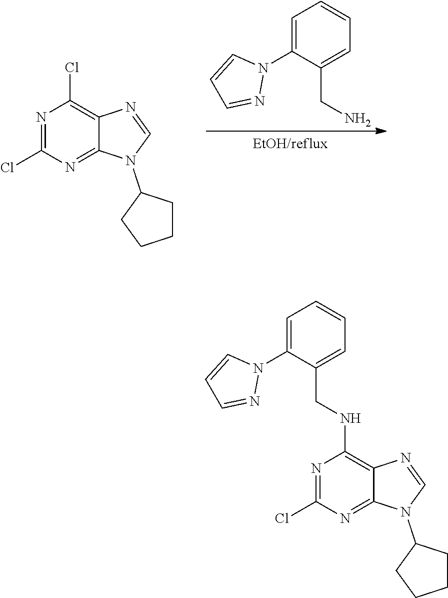 [SMILES: Clc1nc(Cl)c2ncn(C3CCCC3)c2n1 ; NCc1ccccc1-n1cccn1 ; Clc1nc(NCc2ccccc2-n2cccn2)c2ncn(C3CCCC3)c2n1]

2,6-Dichloro-9-cyclopentyl-9H-purine (670 mg, 2.606 mmol), (2-(1H-pyrazol-1-yl)phenyl)methanamine (948 mg, 5.472 mmol), and ethanol(14 mL) were added to a 100 mL single-necked flask, and the mixture was heated and refluxed for 2 hrs. The reaction was concentrated in vacuum and purified by silica gel column (Methanol/dichloromethane=0 to 10%) to afford 890 mg of off-white solid.

### Step 3. N-(2-(1H-pyrazol-1-yl)benzyl)-2-(4-aminopiperidin-1-yl)-9-cyclopentyl-9H-purin-6-amine

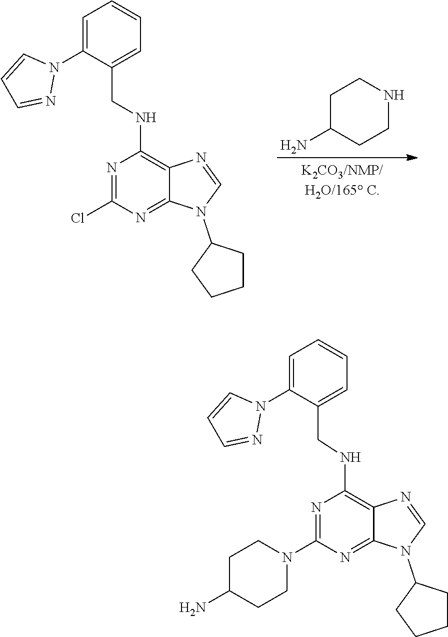 [SMILES: Clc1nc(NCc2ccccc2-n2cccn2)c2ncn(C3CCCC3)c2n1 ; NC1CCNCC1 ; NC1CCN(c2nc(NCc3ccccc3-n3cccn3)c3ncn(C4CCCC4)c3n2)CC1]

N-(2-(1H-pyrazol-1-yl)benzyl)-2-chloro-9-cyclopentyl-9H-purin-6-amine (100 mg, 0.254 mmol), piperidin-4-amine (51 mg, 0.508 mmol), potassium carbonate (70 mg, 0.508 mmol) and 99% NMP (2 mL) were added to a 4 mL reaction flask, heated to 165° C. and stirred for 9 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 30%) to afford 116 mg off-white solid. ¹HNMR:(DMSO-d6, 400 MHz) δ 8.10 (1H, s), 7.80-7.78 (3H, d), 7.54 (1H, s), 7.39-7.36 (3H, m), 6.55 (1H, s), 4.70-4.58 (3H, m), 4.37-4.34 (2H, d), 2.90-2.71 (4H, m), 2.07-1.91 (6H, m), 1.66-1.63 (5H, d), 1.08-1.02 (2H, t). MS: cacl. for C25H31N9 457.27. Found: 458.38[M+H]+

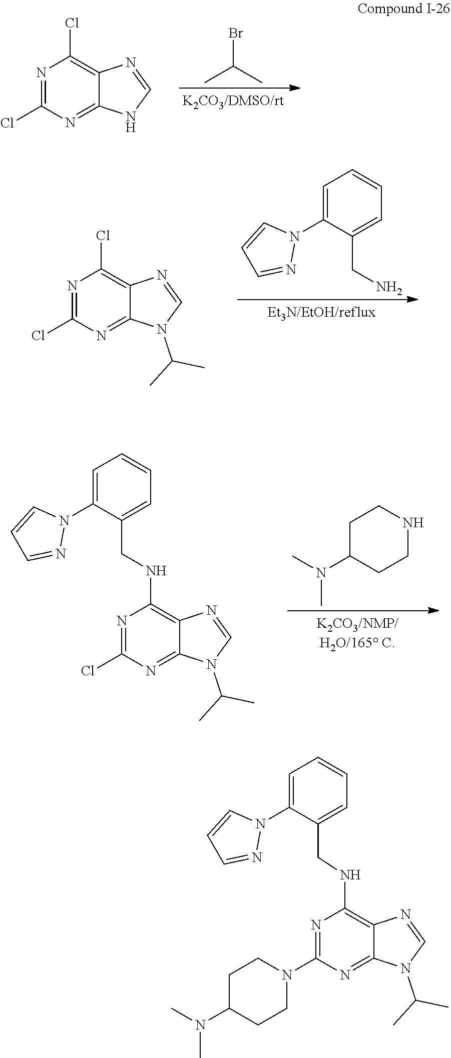

### Step 1. 2,6-dichloro-9-isopropyl-9H-purine

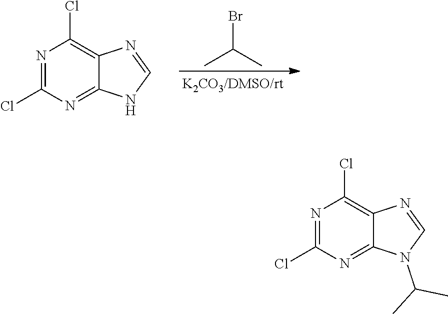

2,6-dichloro-9H-purine (10.0 g, 52.91 mmol), isopropyl bromide(32.5 g, 264.55 mmol), potassium carbonate(21.9 g, 158.73 mmol) and dimethyl sulfoxide(50 mL) were added to a 250 mL two-necked flask, and the mixture was stirred at room temperature for 96 hrs. The reaction solution was added dropwise to water, ethyl acetate was added for extraction three times. The organic phases were combined, washed twice with saturated brine, dried over sodium sulfate, filtered and concentrated in vacuo to afford 9.8 g of light yellow solid. The crude product was passed through a silica gel column (ethyl acetate/n-heptane=0-60%) to afford 4.941 g of white solid.

### Step 2. N-(2-(1H-pyrazol-1-yl)benzyl)-2-chloro-9-isopropyl-9H-purin-6-amine

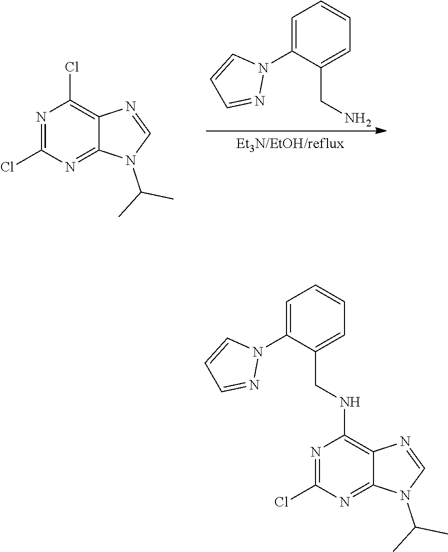 [SMILES: CC(C)n1cnc2c(Cl)nc(Cl)nc21 ; NCc1ccccc1-n1cccn1 ; CC(C)n1cnc2c(NCc3ccccc3-n3cccn3)nc(Cl)nc21]

2,6-dichloro-9-isopropyl-9H-purine (2.082 g, 9.011 mmol), (2-(1H-pyrazol-1-yl)phenyl)methanamine (1.639 g, 9.462 mmol), triethylamine(1.368 g, 13.517 mmol) and ethanol(42 mL) were added to a 100 ml single-necked flask, and the mixture was heated and refluxed for 5 hrs. The reaction was concentrated in vacuum and purified by silica gel column (Methanol/dichloromethane=0-10%) to afford 3304 mg of off-white solid.

### Step 3. N-(2-(1H-pyrazol-1-yl)benzyl)-2-(4-(dimethylamino)piperidin-1-yl)-9-isopropyl-9H-purin-6-amine

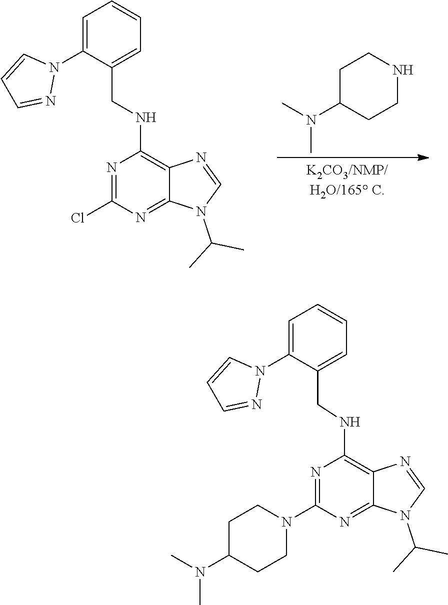

N-(2-(1H-pyrazol-1-yl)benzyl)-2-chloro-9-isopropyl-9H-purin-6-amine (100 mg, 0.272 mmol), N,N-dimethylpiperidin-4-amine (70 mg, 0.544 mmol), potassium carbonate (75 mg, 0.544 mmol) and 99% NMP (2 mL) were added to a 4 mL reaction flask, heated to 165° C. and stirred for 5 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 20%) to afford 84 mg off-white solid. ¹HNMR:(DMSO-d6, 400 MHz) δ 8.12-8.12 (1H, d), 7.83-7.81 (2H, d), 7.78 (1H, s), −7.54-7.51 (1H, t), 7.42-7.40 (1H, m), 7.38-7.35 (2H, m), 6.57-6.56 (1H, t), 4.59-4.46 (5H, m), 2.68-2.62 (2H, t), 2.27-2.22 (1H, t), 2.15 (6H, s), 1.70-1.67 (2H, d), 1.47-1.45 (6H, d), 1.15-1.12 (2H, d). MS: cacl. for C25H33N9 459.29. Found: 460.40[M+H]⁺.

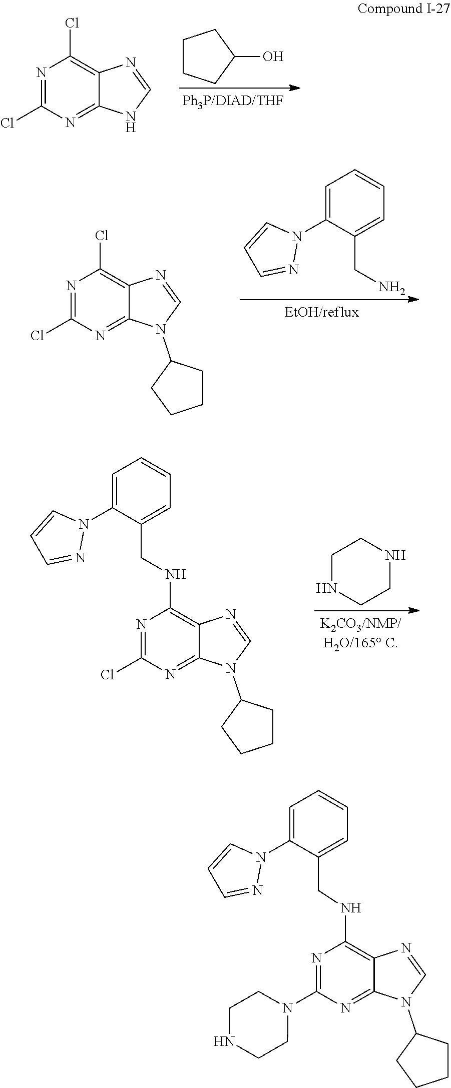

### Step 1. 2,6-dichloro-9-cyclopentyl-9H-purine

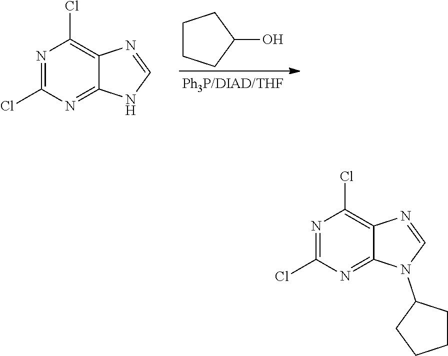

2,6-Dichloropurine(1000 mg, 5.29 mmol), triphenylphosphine oxide(1804 mg, 6.88 mmol), cyclopentanol(1367 mg, 15.87 mmol) and anhydrous tetrahydrofuran(20-mL) were added into a 100 mL three-necked flask, protected with nitrogen and cooled with ice ethanol. DIAD was added dropwise to the mixture. The temperature was naturally raised to room temperature and stirred for 16 hrs. The reaction solution was concentrated in vacuo to afford 5.7 g of oil, which was dissolved in ethyl acetate, washed twice with water, dried over sodium sulfate, filtered and concentrated in vacuo to afford 5.0 g of oil. The crude product was purified by silica gel column to afford 1.540 g of light-yellow solid. m/z:257.2[M+H]+

### Step 2. N-(2-(1H-pyrazol-1-yl)benzyl)-2-chloro-9-cyclopentyl-9H-purin-6-amine

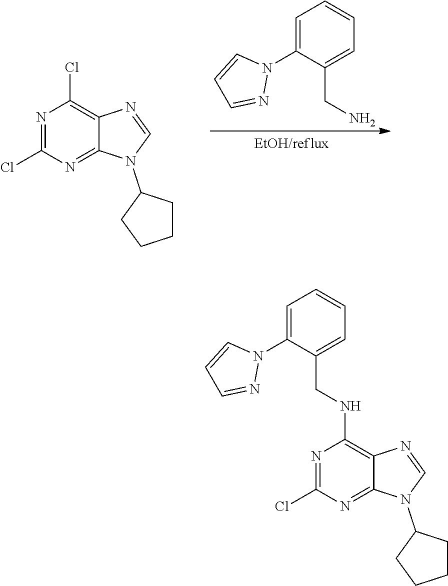 [SMILES: Clc1nc(Cl)c2ncn(C3CCCC3)c2n1 ; NCc1ccccc1-n1cccn1 ; Clc1nc(NCc2ccccc2-n2cccn2)c2ncn(C3CCCC3)c2n1]

2,6-Dichloro-9-cyclopentyl-9H-purine (670 mg, 2.606 mmol), (2-(1H-pyrazol-1-yl)phenyl)methanamine (948 mg, 5.472 mmol), and ethanol(14 mL) were added to a 100 mL single-necked flask, and the mixture was heated and refluxed for 2 hrs. The reaction was concentrated in vacuum and purified by silica gel column (Methanol/dichloromethane=0 to 10%) to afford 890 mg of off-white solid.

### Step 3. N-(2-(1H-pyrazol-1-yl)benzyl)-9-cyclopentyl-2-(piperazin-1-yl)-9H-purin-6-amine

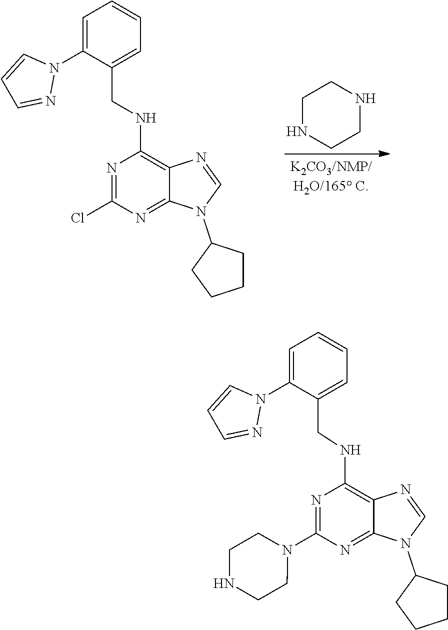 [SMILES: Clc1nc(NCc2ccccc2-n2cccn2)c2ncn(C3CCCC3)c2n1 ; C1CNCCN1 ; c1ccc(-n2cccn2)c(CNc2nc(N3CCNCC3)nc3c2ncn3C2CCCC2)c1]

N-(2-(1H-pyrazol-1-yl)benzyl)-2-chloro-9-cyclopentyl-9H-purin-6-amine (100 mg, 0.254 mmol), piperazine (44 mg, 0.508 mmol), potassium carbonate (70 mg, 0.508 mmol) and 99% NMP (2 mL) were added to a 4 mL reaction flask, heated to 165° C. and stirred for 9 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 20%) to obtain 83 mg off-white solid. ¹HNMR:(DMSO-d6, 400 MHz) δ 8.11 (1H, s), 7.80(3H), 7.55-7.53(1H, m), 7.41-7.35 (1H, m), 6.55 (1H, s), 4.70-4.58 (3H, m), 3.46 (4H, s), 2.67-2.64 (4H, m), 2.11-2.03 (2H, s), 1.97-1.80 (4H, m), 1.66-1.62 (2H, s). MS: cacl. for C24H29N9 443.25. Found:444.59[M+H]⁺.

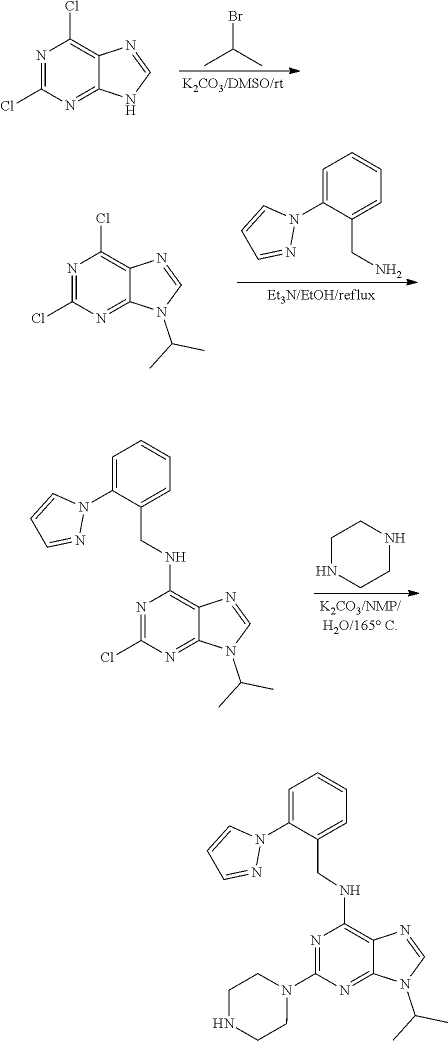

### Step 3. N-(2-(1H-pyrazol-1-yl)benzyl)-9-isopropyl-2-(piperazin-1-yl)-9H-purin-6-amine

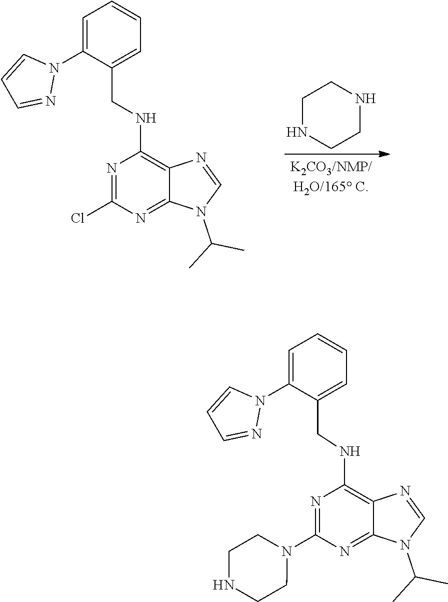 [SMILES: CC(C)n1cnc2c(NCc3ccccc3-n3cccn3)nc(Cl)nc21 ; C1CNCCN1 ; CC(C)n1cnc2c(NCc3ccccc3-n3cccn3)nc(N3CCNCC3)nc21]

N-(2-(1H-pyrazol-1-yl)benzyl)-2-chloro-9-isopropyl-9H-purin-6-amine (150 mg, 0.408 mmol), piperazine (105 mg, 1223 mmol), potassium carbonate (169 mg, 1.223 mmol) and 99% NMP (2 mL) were added to a 4 mL reaction flask, heated to 165° C. and stirred for 12 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 20%) to afford 127 mg off-white solid. ¹HNMR:(DMSO-d6, 400 MHz) δ 8.12 (1H, s), 7.86-7.80 (3H, d), 7.54 (1H, s), 7.39-7.35 (3H, t), 6.55 (1H, s), 4.59-4.54 (3H, m), 3.93 (5H, m), 2.76 (4H, m), 1.46-1.44 (6H, d) MS: cacl. for C22H27N9 417.24. Found:418.31[M+H]⁺.

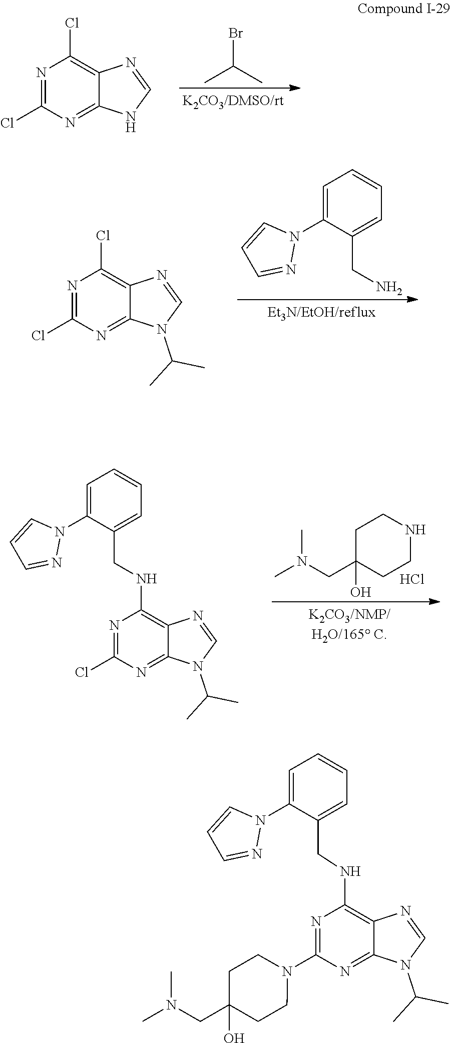

### Step 3. 1-(6-((2-(1H-pyrazol-1-yl)benzyl)amino)-9-isopropyl-9H-purin-2-yl)-4-((dimethylamino)methyl)piperidin-4-ol

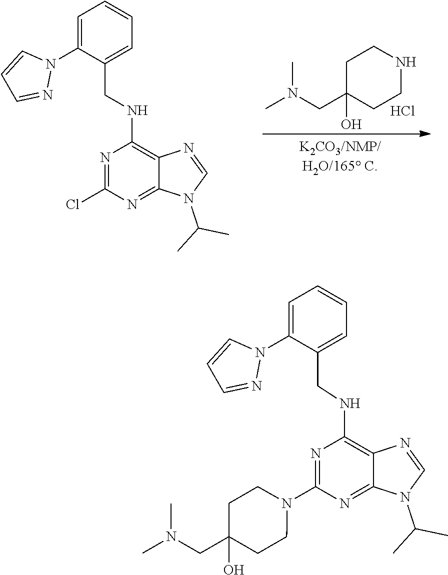 [SMILES: CC(C)n1cnc2c(NCc3ccccc3-n3cccn3)nc(Cl)nc21 ; CN(C)CC1(O)CCNCC1 ; Cl ; CC(C)n1cnc2c(NCc3ccccc3-n3cccn3)nc(N3CCC(O)(CN(C)C)CC3)nc21]

N-(2-(1H-pyrazol-1-yl)benzyl)-2-chloro-9-isopropyl-9H-purin-6-amine (100 mg, 0.272 mmol), 4-((dimethylamino)methyl)piperidin-4-ol hydrochloride (106 mg, 0.544 mmol), potassium carbonate (150 mg, 1.087 mmol) and 99% NMP (2 mL) were added to a 4 mL reaction flask, heated to 165° C. and stirred for 8 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (10% ammonia methanol solution 1/dichloromethane=0 to 15%) to afford 139 mg off-white solid. ¹HNMR:(DMSO-d6, 400 MHz): δ 8.09-8.08 (1H, d), 7.81-7.8(2H, s), 7.73 (1H, s), 7.54-7.52 (1H, t), 7.41-7.39 (1H, t), 7.36-7.34 (2H, m), 6.55 (1H, s), 4.58-4.51 (3H, m), 4.05-4.02 (3H, d), 3.24-3.17 (2H, m), 2.22 (6H, s), 2.17 (2H, s), 1.46-1.44 (6H, d), 1.37 (4H, s). MS: cacl. for C26H35N9O 489.30. Found:490.39[M+H]⁺.

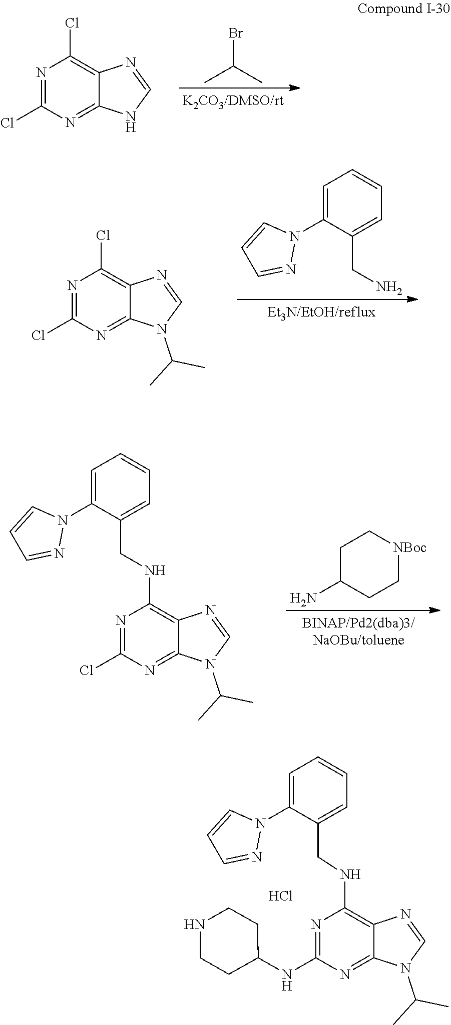

### N6-(2-(1H-pyrazol-1-yl)benzyl)-9-isopropyl-N2-(piperidin-4-yl)-9H-purine-2,6-diamine hydrochloride

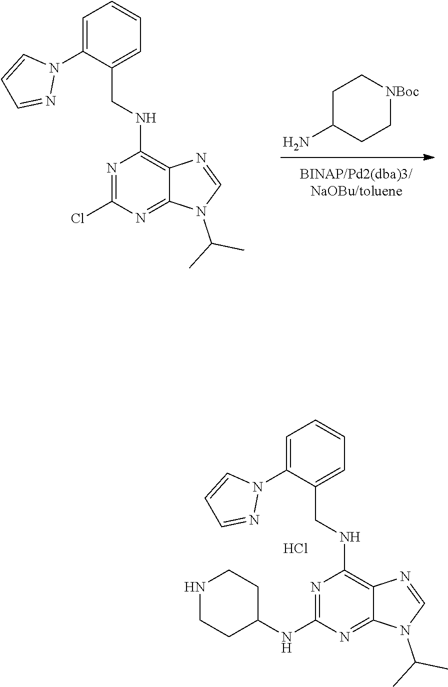 [SMILES: CC(C)n1cnc2c(NCc3ccccc3-n3cccn3)nc(Cl)nc21 ; CC(C)(C)OC(=O)N1CCC(N)CC1 ; CC(C)n1cnc2c(NCc3ccccc3-n3cccn3)nc(NC3CCNCC3)nc21 ; Cl]

To a solution of N-(2-(1H-pyrazol-1-yl)benzyl)-2-chloro-9-isopropyl-9H-purin-6-amine (100 mg, 271.86 μmol) in toluene (20 mL) was added NaOtBu (52.25 mg, 543.72 μmol), Pd₂(dba)₃ (24.89 mg, 27.19 μmol) and BINAP (50.78 mg, 81.56 μmol) under N₂ atmosphere, then stirred at 110° C. for 8 hrs. The reaction was poured into water (300 mL), extracted with EtOAc (100 mL*2), the organic layer was washed with water (100 mL*2), dried over Na₂SO₄, filtered and concentrated to give a residue. The residue was purified by column chromatography to give a yellow solid. MeOH/HCl was added and stirred at rt for 5 hrs, then concentrated to get N6-(2-(1H-pyrazol-1-yl)benzyl)-9-isopropyl-N2-(piperidin-4-yl)-9H-purine-2,6-diamine hydrochloride (67.34 mg, 120.33 μmol, 44.26% yield, 95% purity) as a yellow solid.

¹HNMR (400 MHz, DMSO-d6) δ 8.11 (d, J=2.4 Hz, 1H), 7.86-7.79 (m, 2H), 7.54 (dd, J=5.6, 3.6 Hz, 1H), 7.46-7.32 (m, 3H), 6.56 (t, J=2.2 Hz, 1H), 4.55 (p, J=6.7, 6.3 Hz, 3H), 4.34 (d, J=12.9 Hz, 2H), 2.83-2.64 (m, 3H), 1.74-1.59 (m, 2H), 1.57 (s, 1H), 1.46 (d, J=6.8 Hz, 6H), 1.03 (d, J=11.8 Hz, 2H). LCMS [M+H]⁺: 532.42

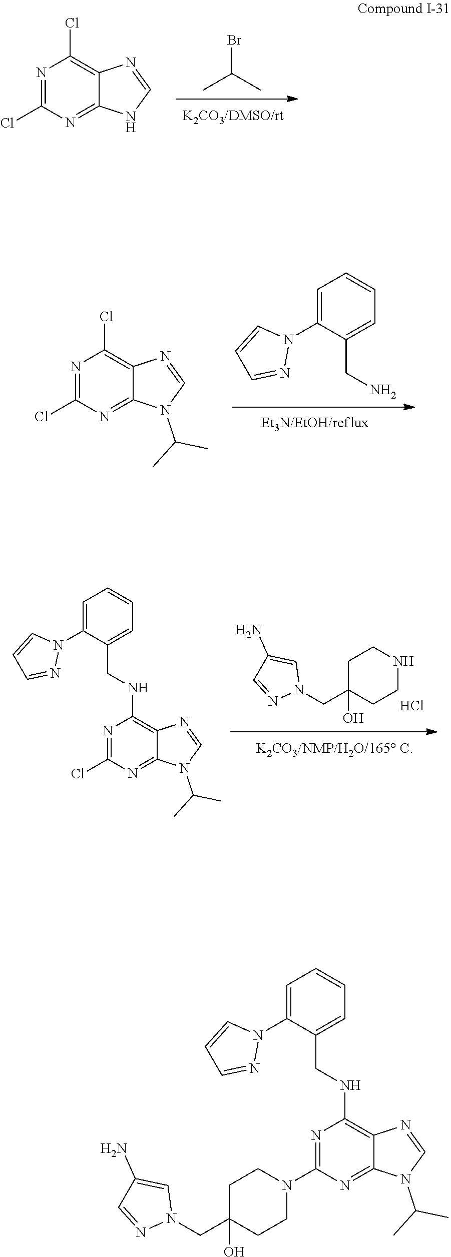

### 1-(6-((2-(1H-pyrazol-1-yl)benzyl)amino)-9-isopropyl-9H-purin-2-yl)-4-((4-amino-1H-pyrazol-1-yl)methyl)piperidin-4-ol

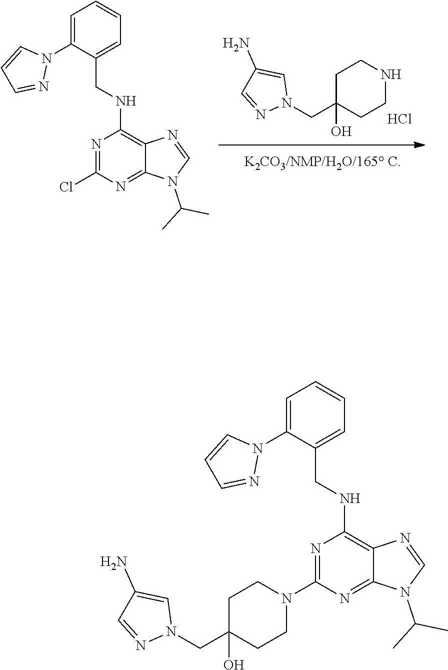 [SMILES: CC(C)n1cnc2c(NCc3ccccc3-n3cccn3)nc(Cl)nc21 ; Nc1cnn(CC2(O)CCNCC2)c1 ; Cl ; CC(C)n1cnc2c(NCc3ccccc3-n3cccn3)nc(N3CCC(O)(Cn4cc(N)cn4)CC3)nc21]

N-(2-(1H-pyrazol-1-yl)benzyl)-2-chloro-9-isopropyl-9H-purin-6-amine (100 mg, 0.272 mmol), 4-((4-amino-1H-pyrazol-1-yl)methyl)piperidin-4-ol hydrochloride (183 mg, 0680 mmol), potassium carbonate (225 mg, 1.631 mmol) and 99% NMP (2 mL) were added to a 4 mL reaction flask, heated to 165° C. and stirred for 14 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (10% ammonia methanol solution 1/dichloromethane=0 to 20%) to afford 16 mg off-white solid. ¹H NMR (400 MHz, DMSO-d6) δ 8.76 (d, J=16.1 Hz, 1H), 8.17 (dd, J=5.1, 2.5 Hz, 1H), 7.91 (m, 4H), 7.40 (d, J=1.8 Hz, 15H), 6.57 (dt, J=7.7, 2.3 Hz, 1H), 4.99 (s, 1H), 4.68-4.58 (m, 3H), 3.97 (d, J=28.5 Hz, 2H), 2.96 (d, J=15.2 Hz, 3H), 2.00 (p, J=7.0 Hz, 1H), 1.67-1.34 (m, 10H), 1.24 (s, 1H). MS: cacl. for C27H33N11O 527.29. Found:528.37[M+H]⁺.

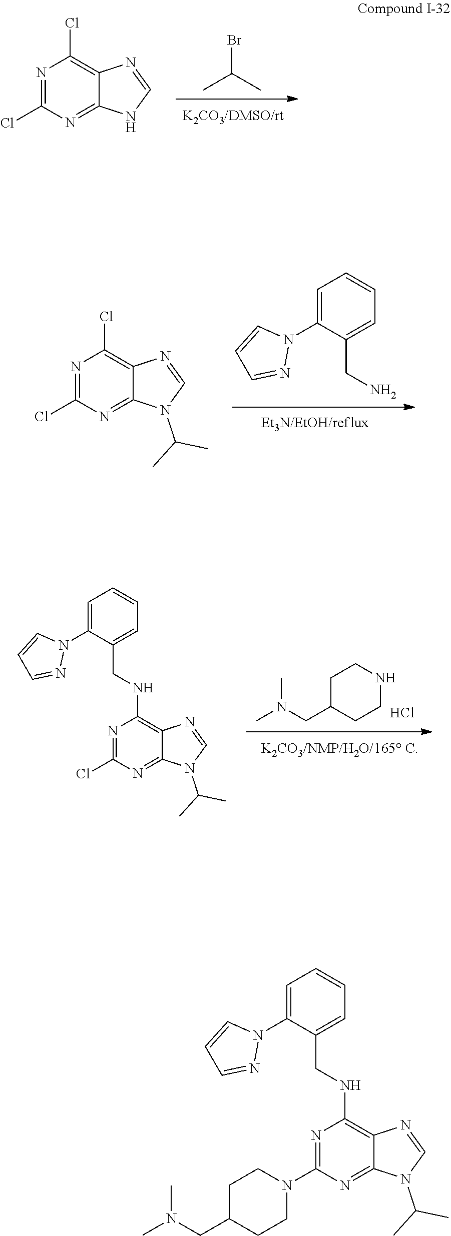

### N-(2-(1H-pyrazol-1-yl)benzyl)-2-(4-((dimethylamino)methyl)piperidin-1-yl)-9-isopropyl-9H-purin-6-amine

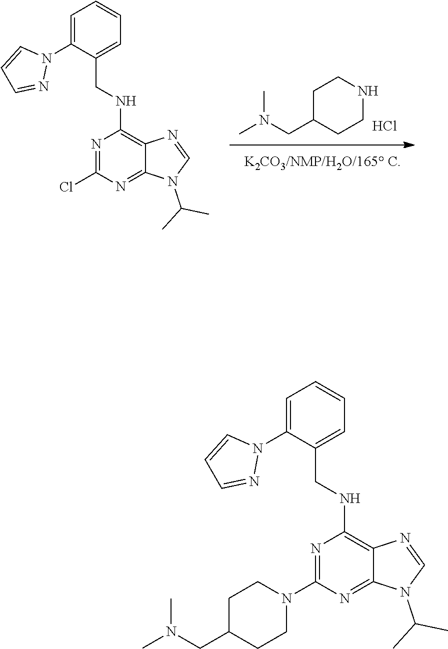

N-(2-(1H-pyrazol-1-yl)benzyl)-2-chloro-9-isopropyl-9H-purin-6-amine (100 mg, 0.272 mmol), N,N-dimethyl-1-(piperidin-4-yl)methanamine hydrochloride (146 mg, 0.816 mmol), potassium carbonate (188 mg, 1.359 mmol) and 99% NMP (2 mL) were added to a 4 mL reaction flask, heated to 165° C. and stirred for 8 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 30%) to afford 72 mg light yellow solid. ¹HNMR:(DMSO-d6, 400 MHz) δ 8.10-8.10 (1H, d), 7.82-7.80 (2H, d), 7.75 (1H, s), 7.55-7.52 (1H, m), 7.41-7.38 (1H, m), 7.37-7.33 (2H, m), 6.56-6.55 (1H, t), 4.59-4.55 (3H, m), 4.53-4.46 (2H, m), 2.70-2.61 (2H, m), 2.10 (6H, s), 2.02-2.00 (2H, d), 1.66-1.60 (3H, d), 1.46-1.44 (6H, d), 0.94-0.85 (2H, m) MS: cacl. for C26H35N9 473.30. Found:474.41[M+H]⁺.

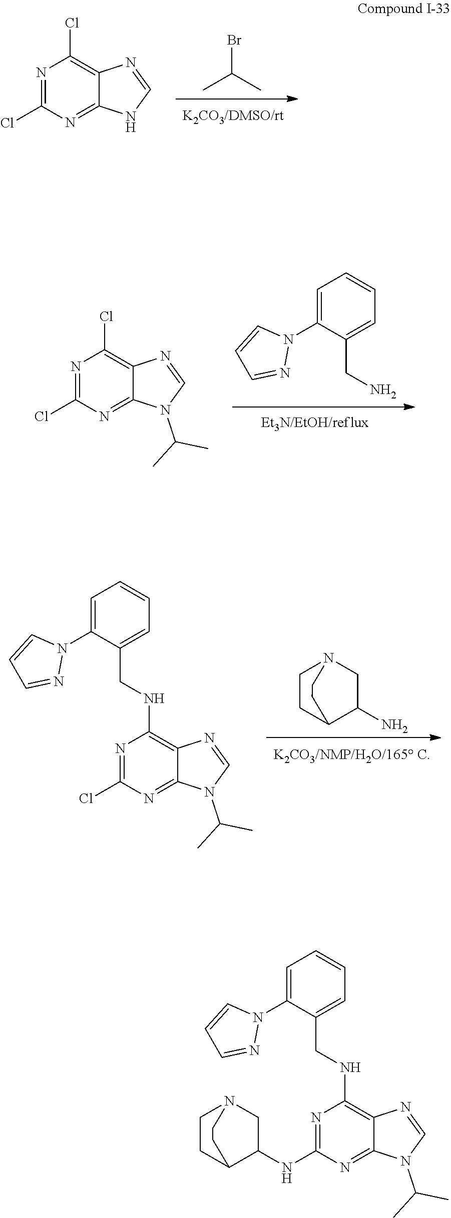

### N6-(2-(1H-pyrazol-1-yl)benzyl)-9-isopropyl-N2-(quinuclidin-3-yl)-9H-purine-2,6-diamine

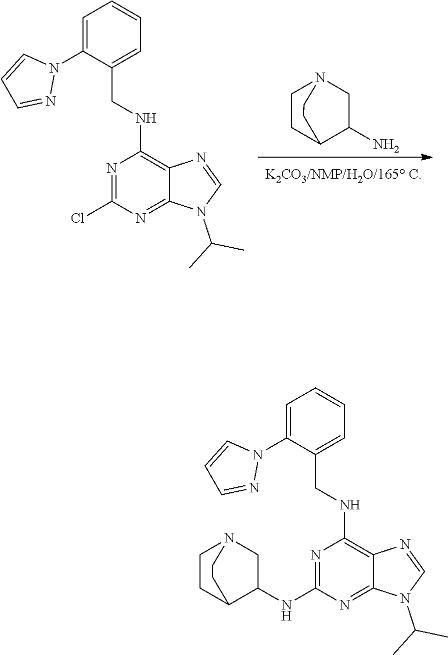 [SMILES: CC(C)n1cnc2c(NCc3ccccc3-n3cccn3)nc(Cl)nc21 ; NC1CN2CCC1CC2 ; CC(C)n1cnc2c(NCc3ccccc3-n3cccn3)nc(NC3CN4CCC3CC4)nc21]

N-(2-(1H-pyrazol-1-yl)benzyl)-2-chloro-9-isopropyl-9H-purin-6-amine (100 mg, 0.272 mmol), quinuclidin-3-amine (108 mg, 0.543 mmol), potassium carbonate (225 mg, 1.628 mmol) and 99% NMP (2 mL) were added to a 4 mL reaction flask, heated to 165° C. and stirred for 22 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 10%) to afford 62 mg off-white solid. ¹HNMR:(DMSO-d6, 400 MHz) δ 8.40 (1H, s), 8.17-8.07 (1H, m), 7.88-7.71 (3H, m), 7.55-7.48 (1H, m), 7.43-7.31 (3H, m), 6.58-6.49 (1H, m), 4.60-4.53 (4H, m), 3.51-3.55 (1H, m), 3.21-3.07 (2H, m), 2.34-2.30 (1H, m), 2.05-1.96 (1H, m), 1.76-1.65 (2H, m), 1.47-1.38 (7H, m), 1.25-1.24 (3H, t) MS: cacl. for C25H31N9 457.27. Found:458. 31[M+H]⁺.

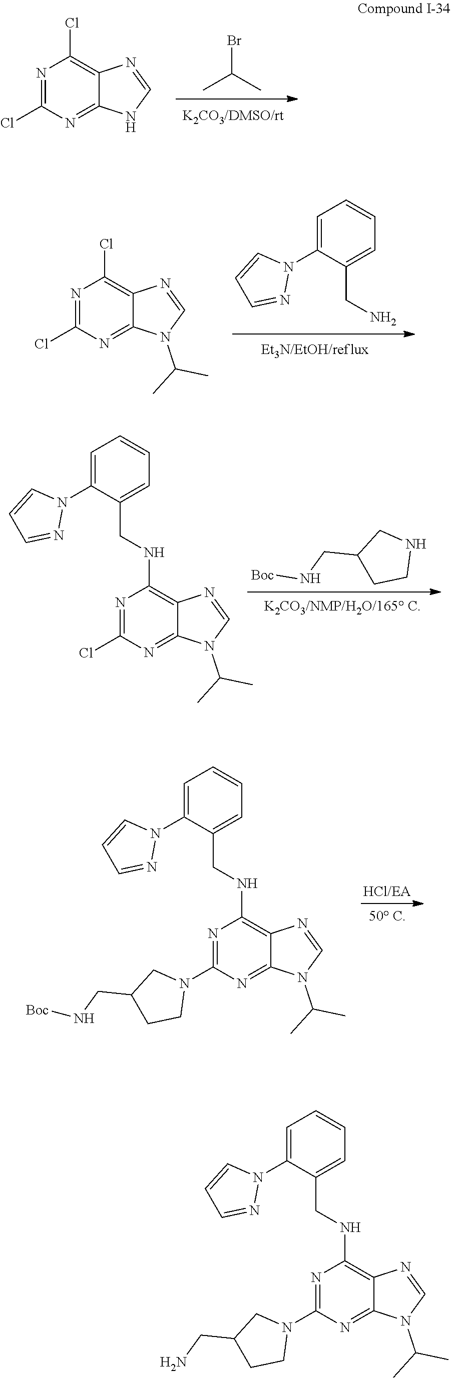

### Tert-butyl ((1-(6-((2-(1H-pyrazol-1-yl)benzyl)amino)-9-isopropyl-9H-purin-2-yl)pyrrolidin-3-yl)methyl)carbamate

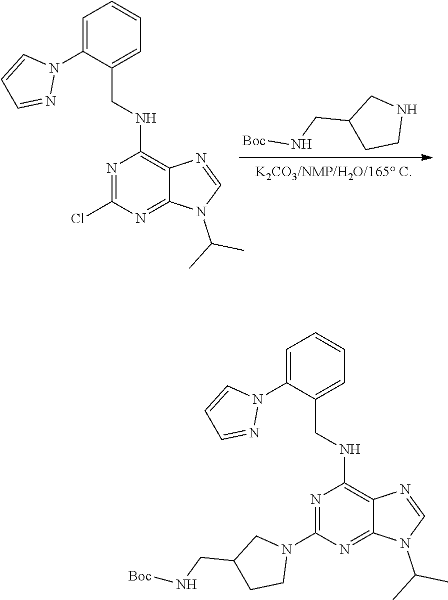 [SMILES: CC(C)n1cnc2c(NCc3ccccc3-n3cccn3)nc(Cl)nc21 ; CC(C)(C)OC(=O)NCC1CCNC1 ; CC(C)n1cnc2c(NCc3ccccc3-n3cccn3)nc(N3CCC(CNC(=O)OC(C)(C)C)C3)nc21]

N-(2-(1H-pyrazol-1-yl)benzyl)-2-chloro-9-isopropyl-9H-purin-6-amine (100 mg, 0.272 mmol), tert-butyl pyrrolidin-3-ylcarbamate (109 mg, 0.544 mmol), potassium carbonate (75 mg, 0.544 mmol) and 99% NMP (2 mL) were added to a 4 mL reaction flask, heated to 165° C. and stirred for 2.5 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo to afford 138 mg oil. The crude was purified by silica gel column (Methanol/dichloromethane=0-10%) to afford 88 mg oil.

### Step 4. N-(2-(1H-pyrazol-1-yl)benzyl)-2-(3-(aminomethyl)pyrrolidin-1-yl)-9-isopropyl-9H-purin-6-amine

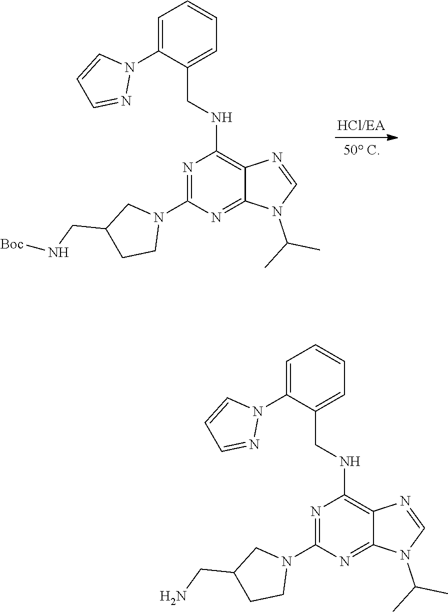 [SMILES: CC(C)n1cnc2c(NCc3ccccc3-n3cccn3)nc(N3CCC(CNC(=O)OC(C)(C)C)C3)nc21 ; CC(C)n1cnc2c(NCc3ccccc3-n3cccn3)nc(N3CCC(CN)C3)nc21]

Ethyl acetate(5 mL) was added to a 25 mL single-necked flask to dissolve tert-butyl ((1-(6-((2-(1H-pyrazol-1-yl)benzyl)amino)-9-isopropyl-9H-purin-2-yl)pyrrolidin-3-yl)methyl)carbamate, 3M hydrochloric acid ethyl acetate solution(5 mL) was added dropwise, heated to 50° C. and stirred for 4 hrs. The reaction solution was concentrated in vacuo to afford 262 mg off-white solid. The crude product was dissolved in water, and ethyl acetate was added to extract the impurities. Potassium carbonate was added to the aqueous phase to adjust the pH>10, and ethyl acetate was added for extraction twice. The combined organic phases was dried with sodium sulfate, filtered and concentrated in vacuo to afford 37 mg of white solid. ¹HNMR: (DMSO-d6, 400 MHz) δ 8.15-8.13 (1H, m), 7.81-7.79 (2H, t), 7.64 (1H, s), 7.57-7.54 (1H, m), 7.42-7.35 (3H, m), 6.56-6.55 (1H, m), 4.60-4.52 (3H, m), 3.58-3.54 (3H, m), 3.02-2.99 (1H, m), 2.65-2.63 (2H, d), 2.26-2.18 (1H, m), 2.03-1.93 (1H, m), 1.63-1.54 (1H, m), 1.47-1.46 (6H, d), 1.25-1.24 (2H, m). MS: cacl. for C23H29N9 431.25. Found: 432.37[M+H]⁺.

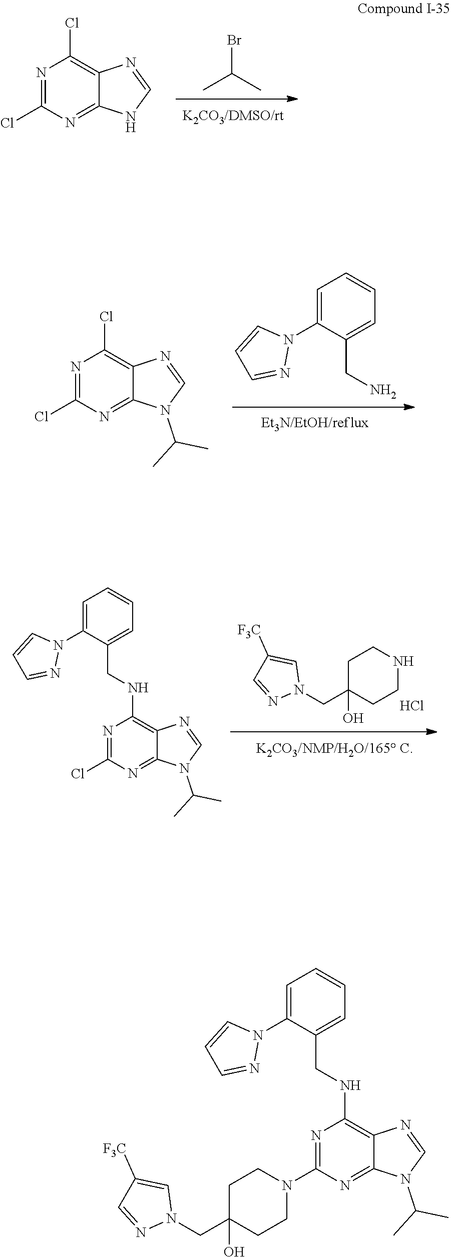

### 1-(6-((2-(1H-pyrazol-1-yl)benzyl)amino)-9-isopropyl-9H-purin-2-yl)-4-((4-(trifluoromethyl)-1H-pyrazol-1-yl)methyl)piperidin-4-ol

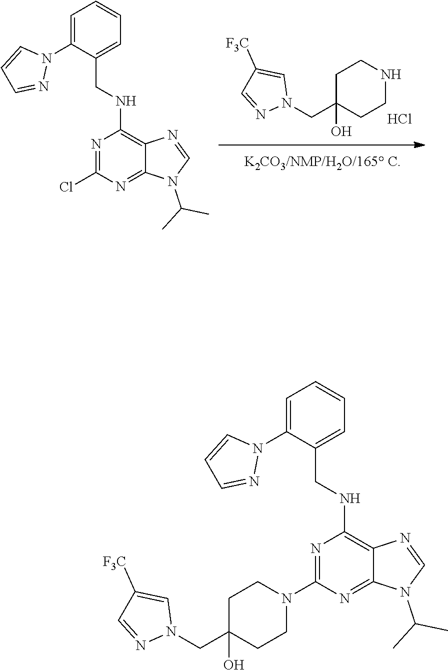 [SMILES: CC(C)n1cnc2c(NCc3ccccc3-n3cccn3)nc(Cl)nc21 ; OC1(Cn2cc(C(F)(F)F)cn2)CCNCC1 ; Cl ; CC(C)n1cnc2c(NCc3ccccc3-n3cccn3)nc(N3CCC(O)(Cn4cc(C(F)(F)F)cn4)CC3)nc21]

N-(2-(1H-pyrazol-1-yl)benzyl)-2-chloro-9-isopropyl-9H-purin-6-amine (100 mg, 0.272 mmol), 4-((4-(trifluoromethyl)-1H-pyrazol-1-yl)methyl)piperidin-4-ol hydrochloride (155 mg, 0.544 mmol), potassium carbonate (150 mg, 1.087 mmol) and 99% NMP (2 mL) were added to a 4 mL reaction flask, heated to 165° C. and stirred for 22 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 10%) to afford 146 mg off-white solid. ¹HNMR: (DMSO-d6, 400 MHz): δ 8.21 (1H, s), 8.09 (1H, s), 7.90-7.75 (4H, m), 7.55-7.52 (1H, t), 7.40-7.35 (3H, m), 6.53 (1H, s), 4.78 (1H, s), 4.58-4.53 (3H, m), 4.12-4.08 (2H, s), 2.72-2.69 (1H, t), 2.50 (1H, s), 2.19-2.16 (1H, t), 1.93-1.87 (1H, m), 1.46-1.44 (6H, t), −1.37-1.23 (4H, m) MS: cacl. for C28H31F3N10O 580.26. Found:581.40[M+H]⁺.

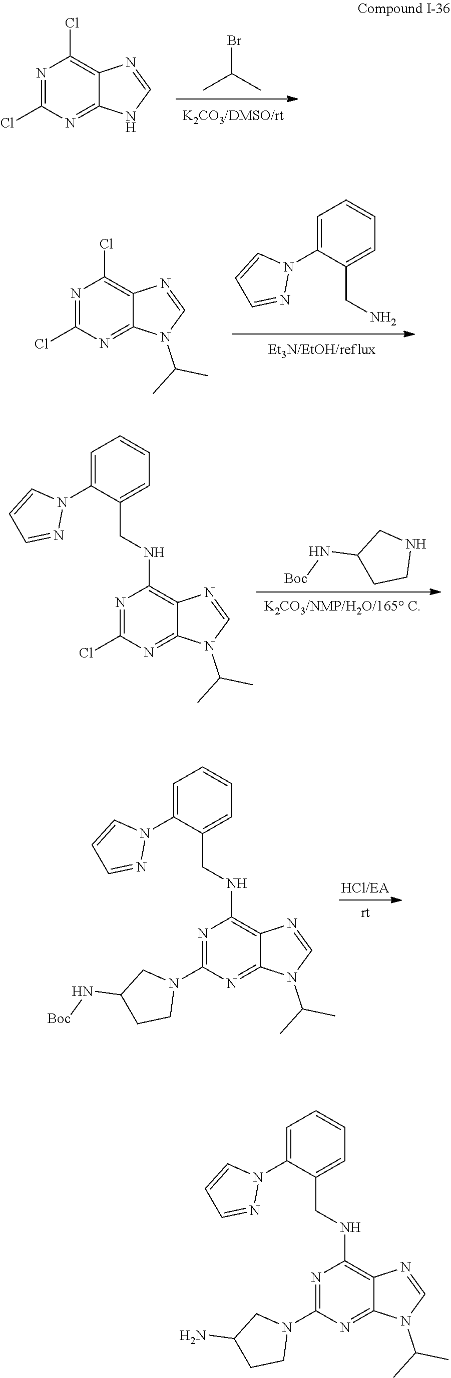

### Tert-butyl (1-(6-((2-(1H-pyrazol-1-yl)benzyl)amino)-9-isopropyl-9H-purin-2-yl)pyrrolidin-3-yl)carbamate

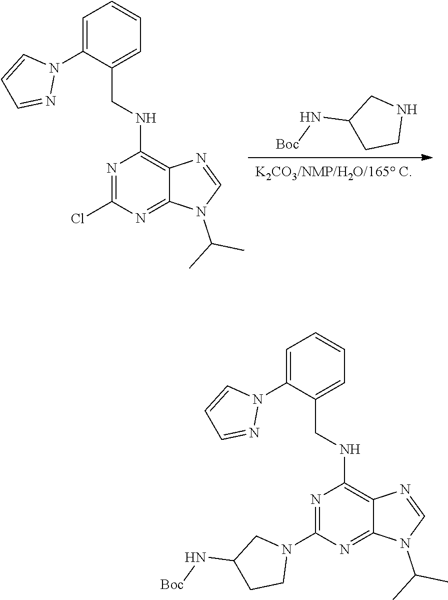 [SMILES: CC(C)n1cnc2c(NCc3ccccc3-n3cccn3)nc(Cl)nc21 ; CC(C)(C)OC(=O)NC1CCNC1 ; CC(C)n1cnc2c(NCc3ccccc3-n3cccn3)nc(N3CCC(NC(=O)OC(C)(C)C)C3)nc21]

N-(2-(1H-pyrazol-1-yl)benzyl)-2-chloro-9-isopropyl-9H-purin-6-amine (100 mg, 0.272 mmol), tert-butyl pyrrolidin-3-ylcarbamate (101 mg, 0.544 mmol), potassium carbonate (75 mg, 0.544 mmol) and 99% NMP (2 mL) were added to a 4 mL reaction flask, heated to 165° C. and stirred for 4.5 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo to afford 141 mg off-white solid.

### N-(2-(1H-pyrazol-1-yl)benzyl)-2-(3-aminopyrrolidin-1-yl)-9-isopropyl-9H-purin-6-amine

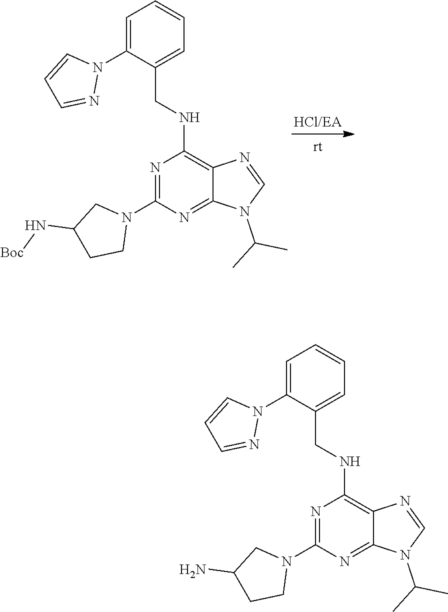 [SMILES: CC(C)n1cnc2c(NCc3ccccc3-n3cccn3)nc(N3CCC(NC(=O)OC(C)(C)C)C3)nc21 ; CC(C)n1cnc2c(NCc3ccccc3-n3cccn3)nc(N3CCC(N)C3)nc21]

Ethyl acetate(5 mL) was added to a 25 mL single-necked flask to dissolve tert-butyl (1-(6-((2-(1H-pyrazol-1-yl)benzyl)amino)-9-isopropyl-9H-purin-2-yl)pyrrolidin-3-yl)carbamate, then 3M hydrochloric acid ethyl acetate solution(10 mL) was added dropwise, heated to 50° C. and stirred for 3 hrs. The reaction solution was concentrated in vacuo to afford 151 mg light yellow solid. The crude product was dissolved in water, ethyl acetate was added to extract the impurities. Potassium carbonate was added to the aqueous phase to adjust the pH>9, and ethyl acetate was added for extraction twice. The combined organic phases were dried with sodium sulfate, filtered and concentrated in vacuo. The crude product was passed through a silica gel column to afford 78 mg of a white solid. ¹HNMR:(DMSO-d6, 400 MHz) δ 8.14-8.13 (1H, d), 7.82-7.81 (1H, d), 7.78 (1H, s), 7.59-7.54 (2H, m), 7.42-7.35 (3H, m), 6.57-6.56 (1H, t), 4.60-4.52 (3H, m), 3.54-3.44 (3H, m), 3.05-3.00 (1H, m), 2.00-1.87 (3H, m), 1.62-1.56 (1H, m), 1.48-1.46 (6H, d), 1.25-1.24 (1H, d). MS: cacl. for C22H27N9 417.24. Found: 418.48[M+H]⁺.

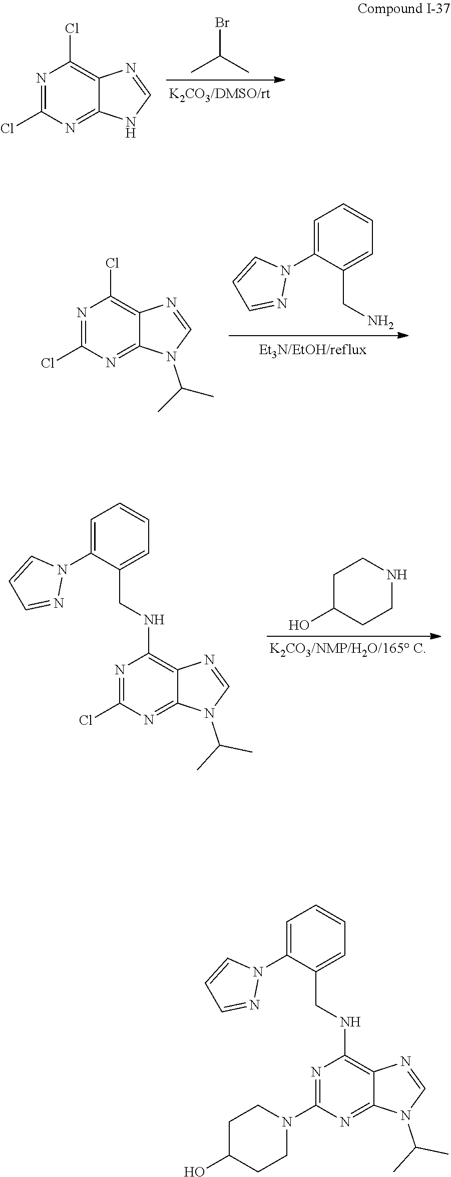

### 1-(6-((2-(1H-pyrazol-1-yl)benzyl)amino)-9-isopropyl-9H-purin-2-yl)piperidin-4-ol

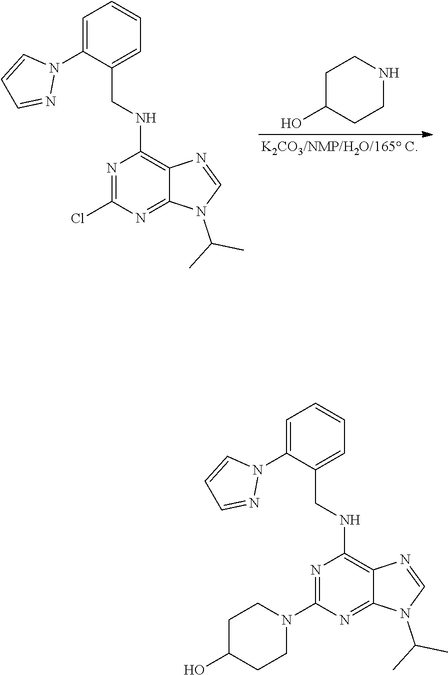 [SMILES: CC(C)n1cnc2c(NCc3ccccc3-n3cccn3)nc(Cl)nc21 ; OC1CCNCC1 ; CC(C)n1cnc2c(NCc3ccccc3-n3cccn3)nc(N3CCC(O)CC3)nc21]

N-(2-(1H-pyrazol-1-yl)benzyl)-2-chloro-9-isopropyl-9H-purin-6-amine (100 mg, 0.272 mmol), piperidin-4-ol (55 mg, 0.544 mmol), potassium carbonate (75 mg, 0.544 mmol) and 99% NMP (2 mL) were added to a 4 mL reaction flask, heated to 165° C. and stirred for 5 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (Methanol/dichloromethane=0 to 10%) to afford 122 mg off-white solid. ¹HNMR:(DMSO-d6, 400 MHz) δ 8.12-8.11 (1H, d), 7.83 (1H, s), 7.81-7.80 (1H, d), 7.77 (1H, s), 7.56-7.51 (1H, m), 7.42-7.34 (3H, m), 6.56-6.55 (1H, m), 4.60-4.50 (4H, m), 4.17-4.14 (2H, t), 3.64-3.59 (1H, m), 3.01-2.95 (2H, t), 1.70-1.65 (2H, m), 1.47-1.45 (6H, d), 1.21-1.16 (2H, m). MS: cacl. for C23H28N8O 432.24. Found: 433.36[M+H]⁺.

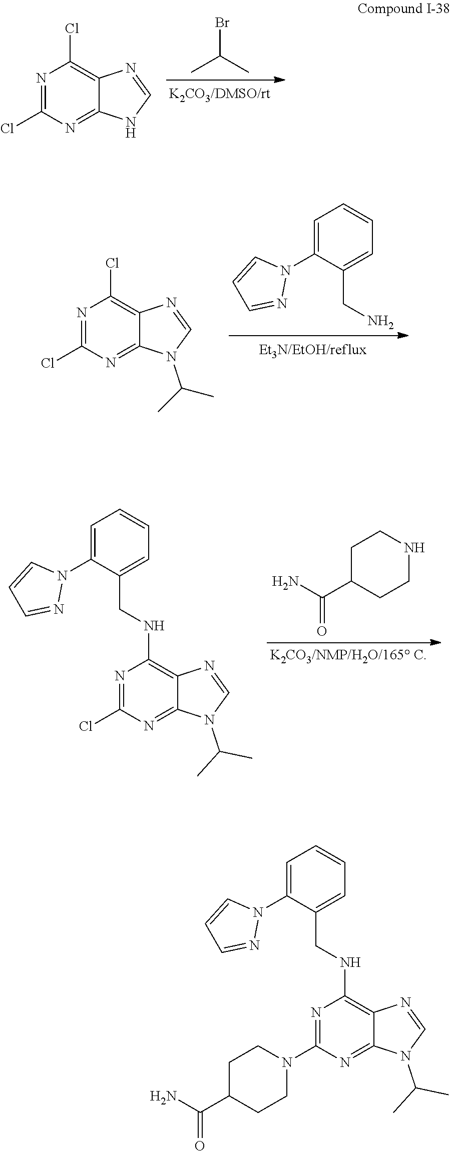

### 1-(6-((2-(1H-pyrazol-1-yl)benzyl)amino)-9-isopropyl-9H-purin-2-yl)piperidine-4-carboxamide

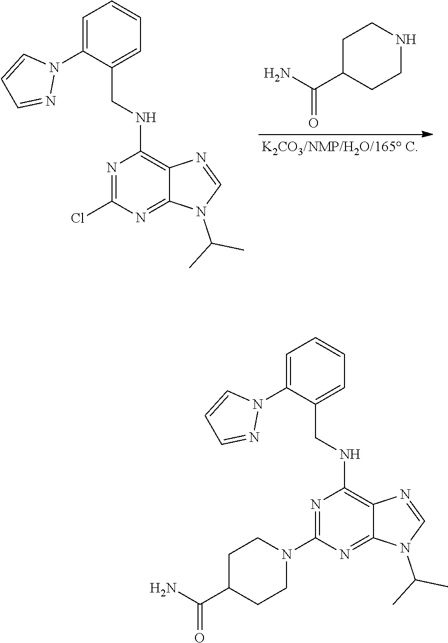 [SMILES: CC(C)n1cnc2c(NCc3ccccc3-n3cccn3)nc(Cl)nc21 ; NC(=O)C1CCNCC1 ; CC(C)n1cnc2c(NCc3ccccc3-n3cccn3)nc(N3CCC(C(N)=O)CC3)nc21]

N-(2-(1H-pyrazol-1-yl)benzyl)-2-chloro-9-isopropyl-9H-purin-6-amine (100 mg, 0.272 mmol), piperidine-4-carboxamide (70 mg, 0.544 mmol), potassium carbonate (75 mg, 0.544 mmol) and 99% NMP (2 mL) were added to a 4 mL reaction flask, heated to 165° C. and stirred for 10 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (methanol/dichloromethane=0 to 10%) to afford 108 mg off-white solid. ¹HNMR:(DMSO-d6, 400 MHz) δ 8.11-8.11 (1H, d), 7.84-7.80 (3H, t), 7.56-7.53 (1H, m), 7.42-7.34 (3H, m), 7.25 (1H, s), 6.75 (1H, s), 6.56-6.55 (1H, t), 4.61-4.54 (3H, m), 4.53-4.47 (2H, m), 2.70-2.63 (2H, t), 2.31-2.24 (1H, m), 1.67-1.64 (2H, d), 1.47-1.45 (6H, d), 1.41-1.31 (2H, m). MS: cacl. for C24H29N9O 459.25. Found:460.35[M+H]⁺.

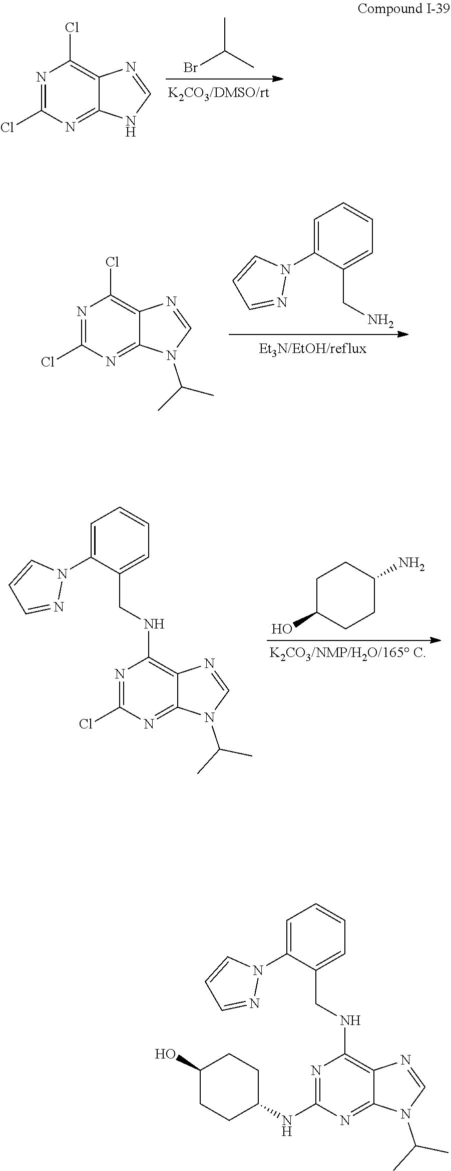

### (1R,4R)-4-((6-((2-(1H-pyrazol-1-yl)benzyl)amino)-9-isopropyl-9H-purin-2-yl)amino)cyclohexan-1-ol

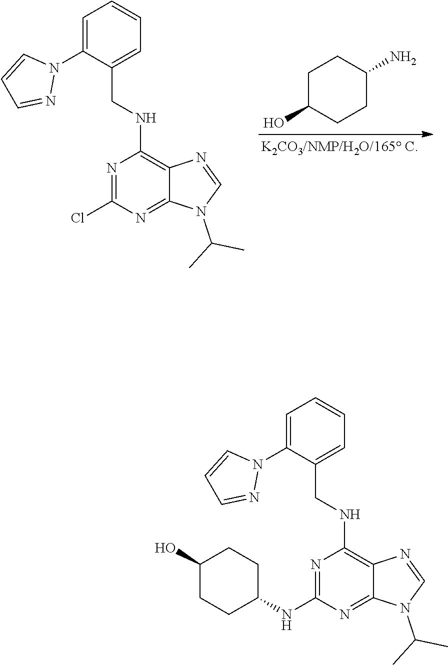 [SMILES: CC(C)n1cnc2c(NCc3ccccc3-n3cccn3)nc(Cl)nc21 ; N[C@H]1CC[C@H](O)CC1 ; CC(C)n1cnc2c(NCc3ccccc3-n3cccn3)nc(N[C@H]3CC[C@H](O)CC3)nc21]

N-(2-(1H-pyrazol-1-yl)benzyl)-2-chloro-9-isopropyl-9H-purin-6-amine (100 mg, 0.273 mmol), (1R,4R)-4-aminocyclohexan-1-ol (63 mg, 0.545 mmol), potassium carbonate (75 mg, 0.545 mmol) and 99% NMP (2 mL) were added to a 4 mL reaction flask, heated to 165° C. and stirred for 46 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (methanol/dichloromethane=0 to 10%) to afford 103 mg off-white solid. ¹HNMR:(DMSO-d6, 400 MHz) δ 8.13-8.12 (1H, t), 7.82-7.81 (2H, d), 7.50-7.48 (2H, t), 7.41-7.35 (3H, m), 6.57-6.56 (1H, t), 5.99-5.97 (1H, d), 4.61-4.61 (2H, d), 4.55-4.49 (1H, m), 4.47-4.45 (1H, dd), 3.53 (1H, s), 1.78-1.77 (4H, m), 1.47-1.46 (6H, d), 1.24-1.22 (1H, m), 1.19-1.15 (4H, t). MS: cacl. for C24H30N8O 446.25. Found:447.47 [M+H]⁺.

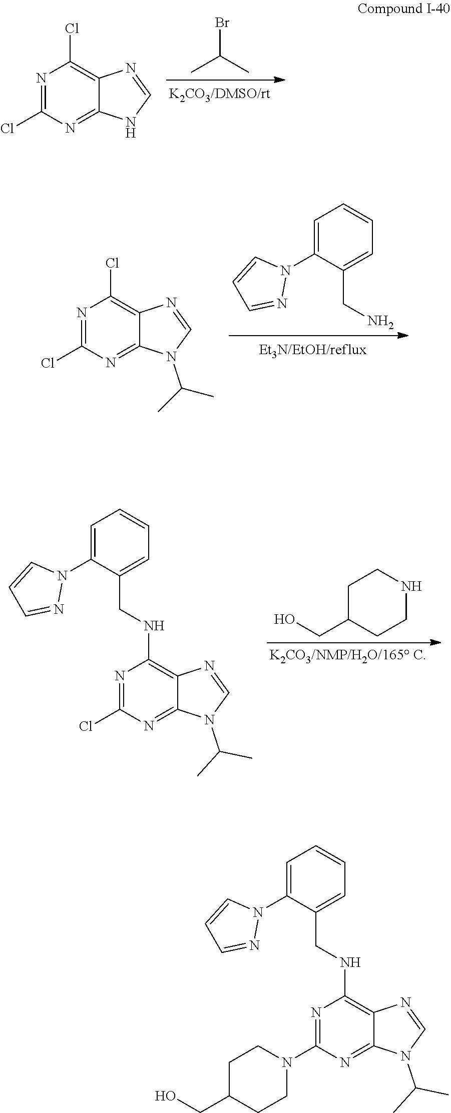

### (1-(6-((2-(1H-pyrazol-1-yl)benzyl)amino)-9-isopropyl-9H-purin-2-yl)piperidin-4-yl)methanol

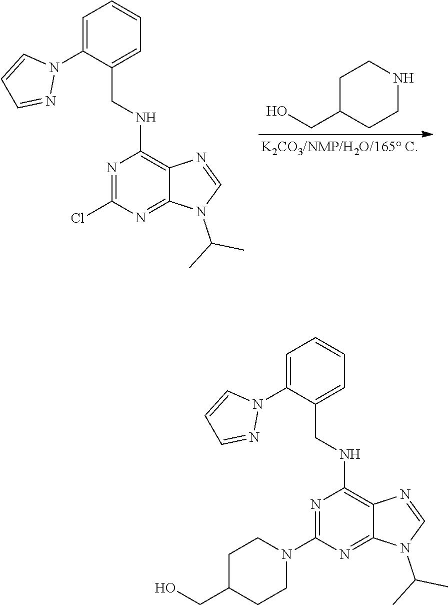 [SMILES: CC(C)n1cnc2c(NCc3ccccc3-n3cccn3)nc(Cl)nc21 ; OCC1CCNCC1 ; CC(C)n1cnc2c(NCc3ccccc3-n3cccn3)nc(N3CCC(CO)CC3)nc21]

N-(2-(1H-pyrazol-1-yl)benzyl)-2-chloro-9-isopropyl-9H-purin-6-amine (100 mg, 0.273-mmol), piperidin-4-ylmethanol (63 mg, 0.545 mmol), acetic acid (75 mg, 0.545 mmol) and 99% NMP (2 mL) were added to a 4 mL reaction flask, heated to 165° C. and stirred for 5 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (methanol/dichloromethane=0 to 10%) to afford 127 mg off-white solid. ¹HNMR:(DMSO-d6, 400 MHz) δ 8.11-8.11 (1H, d), 7.82 (1H, s), 7.81-7.80 (1H, d), 7.75 (1H, s), 7.55-7.52 (1H, m), 7.42-7.34 (3H, m), 6.57-6.55 (1H, t), 4.59-4.48 (5H, m), 4.43-4.41 (1H, t), 3.25-3.22 (2H, t), 2.70-2.59 (2H, m), 1.63-1.53 (3H, m), 1.47-1.45 (6H, d), 0.99-0.94 (2H, m). MS: cacl. for C24H30N8O 446.25. Found:447.44[M+H]+

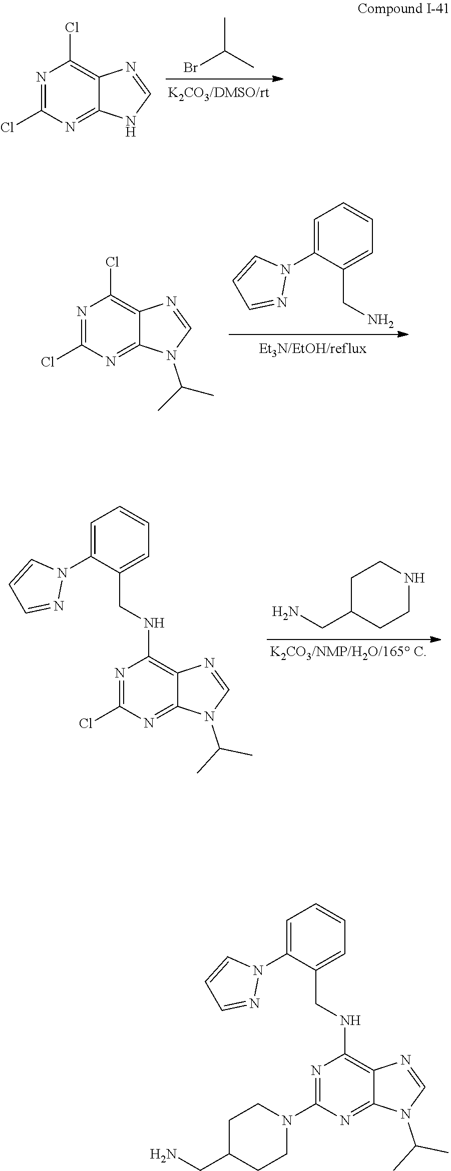

N-(2-(1H-pyrazol-1-yl)benzyl)-2-(4-(aminomethyl)piperidin-1-yl)-9-isopropyl-9H-purin-6-amine: N-(2-(1H-pyrazol-1-yl)benzyl)-2-chloro-9-isopropyl-9H-purin-6-amine (100 mg, 0.273 mmol), piperidin-4-ylmethanamine (62 mg, 0.545 mmol), potassium carbonate (75 mg, 0.545 mmol) and 99% NMP (2 mL) were added to a 4 mL reaction flask, heated to 165° C. and stirred for 10 hrs. The reaction solution was added dropwise to water, extracted twice with ethyl acetate, and the organic phases were combined. The organic phase was washed twice with saturated sodium chloride, dried over sodium sulfate, filtered, and concentrated in vacuo. The crude product was purified by silica gel column (methanol/dichloromethane=0 to 20%) to afford 69 mg off-white solid

¹HNMR:(DMSO-d6, 400 MHz) δ 8.11 (1H, s), 7.82-7.78 (3H, t), 7.55-7.52 (1H, t), 7.41-7.35 (3H, m), 6.56 (1H, s), 4.58-4.48 (5H, m), 2.64-2.58 (2H, t), 1.66-1.63 (2H, d), 1.46-1.45 (6H, d), 1.41-1.22 (3H, m), 0.95-0.86 (2H, m)

MS: cacl. for C24H31N9 445.27. Found: 446.49[M+H]+

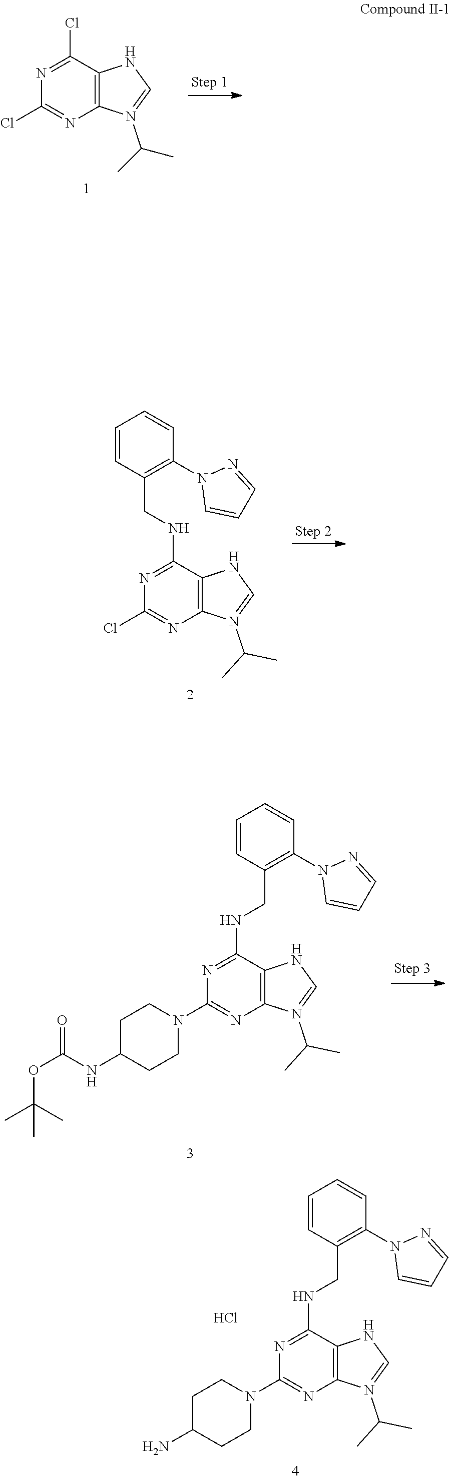

### Step 1

### N-(2-(1H-pyrazol-1-yl)benzyl)-5-chloro-3-isopropyl-1H-pyrazolo[4,3-d]pyrimidin-7-amine

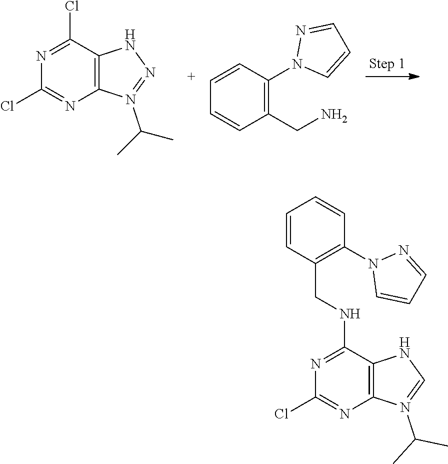

To a solution of 5,7-dichloro-3-isopropyl-1H-pyrazolo[4,3-d]pyrimidine (100 mg, 432.75 mol) in EtOH (10 mL) was added (2-(1H-pyrazol-1-yl)phenyl)methanamine (78.71 mg, 454.38 mol) and TEA (131.37 mg, 1.30 mmol, 181.07 L), then stirred at 80° C. for 6 hrs. The reaction was concentrated to give a residue. The residue was purified by column chromatography to give N-(2-(1H-pyrazol-1-yl)benzyl)-5-chloro-3-isopropyl-1H-pyrazolo[4,3-d]pyrimidin-7-amine (135 mg, 359.67 mol, 83.11% yield, 98% purity) as a white solid. LCMS [M+H]⁺. 368.32

### Step 2

### tert-butyl (1-(7-((2-(1H-pyrazol-1-yl)benzyl)amino)-3-isopropyl-1H-pyrazolo[4,3-d]pyrimidin-5-yl)piperidin-4-yl)carbamate

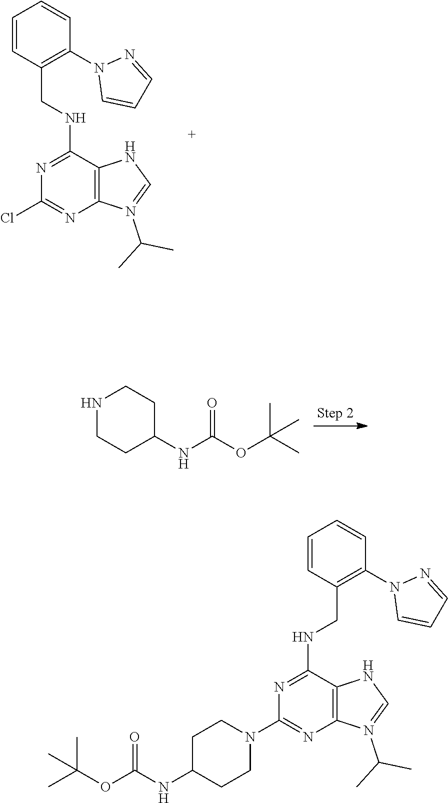

To a solution of N-(2-(1H-pyrazol-1-yl)benzyl)-5-chloro-3-isopropyl-1H-pyrazolo[4,3-d]pyrimidin-7-amine (100 mg, 271.86 μmol) in DMSO (2.5 mL) was added tert-butyl piperidin-4-ylcarbamate (108.90 mg, 543.72 μmol) and TEA (82.53 mg, 815.58 μmol, 113.75 μL), then stirred at 160° C. for 8 hrs. The reaction was poured into water (30 mL), extracted with EtOAc (10 mL*2), the organic layer was washed with water (10 mL*2), dried over Na₂SO₄, filtered and concentrated to give a residue. The residue was purified by column chromatography to give tert-butyl (1-(7-((2-(1H-pyrazol-1-yl)benzyl)amino)-3-isopropyl-1H-pyrazolo[4,3-d]pyrimidin-5-yl)piperidin-4-yl)carbamate (76 mg, 138.66 mol, 51.00% yield, 97% purity) as a yellow solid.

LCMS [M+H]⁺: 532.23

### Step 3

### N-(2-(1H-pyrazol-1-yl)benzyl)-5-(4-aminopiperidin-1-yl)-3-isopropyl-1H-pyrazolo[4,3-d]pyrimidin-7-amine hydrochloride

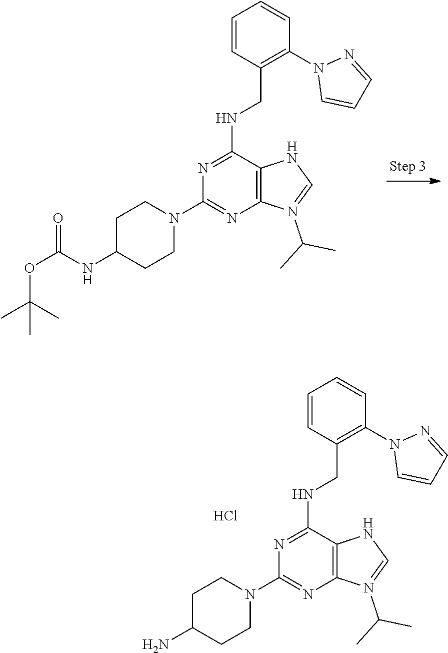

To a solution of tert-butyl (1-(7-((2-(1H-pyrazol-1-yl)benzyl)amino)-3-isopropyl-1H-pyrazolo[4,3-d]pyrimidin-5-yl)piperidin-4-yl)carbamate (76 mg, 142.95 μmol) in MeOH (5 mL) was added and HCl/MeOH (428.85 μmol), then stirred at 20° C. for 6 hrs. The reaction was concentrated to give N-(2-(1H-pyrazol-1-yl)benzyl)-5-(4-aminopiperidin-1-yl)-3-isopropyl-1H-pyrazolo[4,3-d]pyrimidin-7-amine hydrochloride (43 mg, 94.66 μmol, 66.22% yield, 95% purity) as a white solid.

¹HNMR (400 MHz, Methanol-d4) δ 8.10 (s, 1H), 7.95 (m, 1H), 7.81-7.62 (m, 1H), 7.51 (m, 3H), 6.66 (m, 1H), 4.85 (s, 2H), 4.65 (m, 1H), 3.78-3.45 (m, 2H), 3.35 (d, J=15.5 Hz, 3H), 2.21 (m, 2H), 1.73 (m, 2H), 1.40 (s, 6H). LCMS [M+H]⁺: 432.24

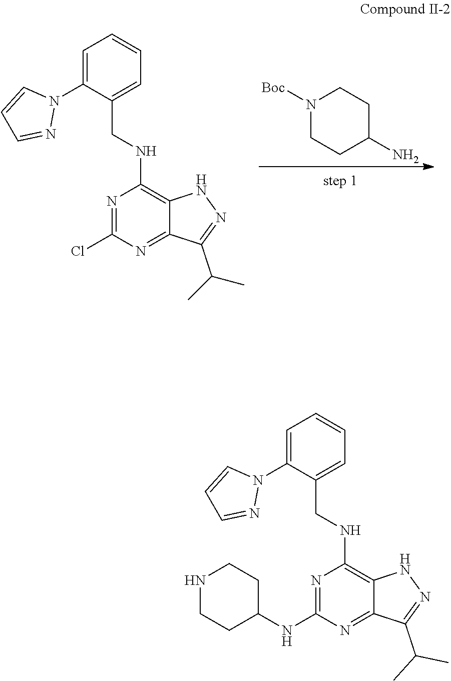 [SMILES: CC(C)c1n[nH]c2c(NCc3ccccc3-n3cccn3)nc(Cl)nc12 ; CC(C)(C)OC(=O)N1CCC(N)CC1 ; CC(C)c1n[nH]c2c(NCc3ccccc3-n3cccn3)nc(NC3CCNCC3)nc12]

### Step 1

### N7-(2-(1H-pyrazol-1-yl)benzyl)-3-isopropyl-N5-(piperidin-4-yl)-1H-pyrazolo[4,3-d]pyrimidine-5,7-diamine (compound II-4)

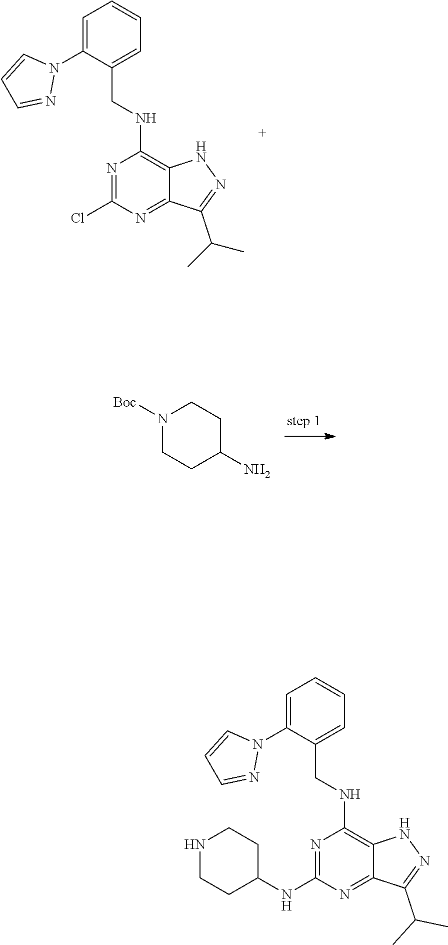 [SMILES: CC(C)c1n[nH]c2c(NCc3ccccc3-n3cccn3)nc(Cl)nc12 ; CC(C)(C)OC(=O)N1CCC(N)CC1 ; CC(C)c1n[nH]c2c(NCc3ccccc3-n3cccn3)nc(NC3CCNCC3)nc12]

To a solution of 5-chloro-3-isopropyl-N-[(2-pyrazol-1-ylphenyl)methyl]-1H-pyrazolo[4,3-d]pyrimidin-7-amine (50 mg, 135.93 umol, 1 eq) and tert-butyl 4-aminopiperidine-1-carboxylate (54.45 mg, 271.86 umol, 2 eq) in n-BuOH (1 mL) was added DIEA (52.70 mg, 407.79 umol, 71.03 uL, 3 eq), The mixture was heated under microwave condition at 160° C. for 6 hrs. LCMS showed one main peak with desired mass. The reaction mixture was filtered and concentrated under reduced pressure to give a residue. The residue was purified by prep-HPLC (neutral condition; column: Waters xbridge 150*25 mm 10 um; mobile phase: [water (10 mM NH4HCO3)-ACN]; B %: 12%-42%, 11 min). Compound tert-butyl 4-[[3-isopropyl-7-[(2-pyrazol-1-ylphenyl) methylamino]-1H-pyrazolo [4,3-d]pyrimidin-5-yl]amino]piperidine-1-carboxylate (30 mg, 54.40 umol, 40.02% yield, 96.4% purity) was obtained as a white solid. ¹H NMR (400 MHz, Methanol-d4): δ 7.89 (d, J=2.38 Hz, 1H) 7.77-7.79 (m, 1H) 7.63-7.66 (m, 1H) 7.39-7.45 (m, 3H) 6.53 (t, J=2.13 Hz, 1H) 4.68 (s, 2H) 4.63 (br d, J=13.38 Hz, 2H) 2.85-2.93 (m, 2H) 2.78-2.85 (m, 2H) 1.83 (br dd, J=11.82, 1.94 Hz, 2H) 1.38 (d, J=6.88 Hz, 6H) 1.25-1.33 (m, 2H). LCMS [M+H]⁺: 432.3

### Example 2 Biological Activity

### Materials

Human CDKs, cyclins and other partner proteins were expressed in insect cell, purified individually, reconstituted and then purified to homogeneity before being used for kinase activity assay. Fluorescence-labelled substrate peptides used in the assays are listed in Table 1.

TABLE 1
List of CDKs peptides used in kinase assay

| CDK/Cyclin | Substrate Peptides |
|---|---|
| CDK1/Cyclin | 5-FAM-YSPSTSPSYSTPSPSYSPTSPSKKKK |
| A2 | (SEQ ID NO: 1) |
| CDK2/Cyclin | 5-FAM-YSPSTSPSYSTPSPSYSPTSPSKKKK |
| A2 | (SEQ ID NO: 1) |
| CDK3/Cyclin | 5-FAM-YSPSTSPSYSTPSPSYSPTSPSKKKK |
| C | (SEQ ID NO: 1) |
| CDK4/Cyclin | 5-FAM-YSPSTSPSYSTPSPSYSPTSPSKKKK |
| D1 | (SEQ ID NO: 1) |
| CDK5/P35 | 5-FAM-YSPSTSPSYSTPSPSYSPTSPSKKKK |
| | (SEQ ID NO: 1) |
| CDK6/Cyclin | 5-FAM-YSPSTSPSYSTPSPSYSPTSPSKKKK |
| D1 | (SEQ ID NO: 1) |
| CDK7/Cyclin | 5-FAM-YSPSTSPSYSTPSPSYSPTSPSKKKK |
| H/MAT1 | (SEQ ID NO: 1) |
| CDK9/Cyclin | 5-FITC- X-GSRTPMY(SEQ ID NO: 2)- |
| T1 | NH2 |
| CDK12/Cyclin | 5-FITC- X-GSRTPMY(SEQ ID NO: 2)- |
| K | NH2 |

### Methods

Kinase activity of CDKs were determined by mobility shift assay (MSA), in which the formation of a phosphorylated fluorescence-labelled peptide in reaction mixture was monitored through capillary electrophoresis in a microfluidic environment. Kinase reaction was carried out in a 384-well plate with a total volume of 12 μl, containing 20 mM MES, pH 6.75, 0.01% Tween20, 0.05 mg/ml BSA, 6 mM MgCl₂, 2 μM substrate peptide, 2 mM ATP, 3 nM CDK/Cyclin, 2% DMSO and compound of 10-dose 3× serial dilution starting from 10 μM. The mixtures were incubated at 27° C. for 40 min before being quenched with 4 μl 80 mM EDTA, then read on a LabChip EZ Reader II (Caliper Life Science). IC₅₀ of compound for CDKs was calculated by Prism (GraphPad) using a four-parameter nonlinear curve fit.

The CDK inhibition results (IC₅₀) are summarized in Table 2. The CDK may be CDK7.

TABLE 2
List of CDK inhibition

| Compound | IC₅₀ |
|---|---|
| I-1 | A |
| I-2 | A |
| I-3 | A |
| I-4 | A |
| I-5 | B |
| I-6 | A |
| I-7 | A |
| I-8 | A |
| I-9 | A |
| I-10 | A |
| I-11 | A |
| I-12 | A |
| I-13 | B |
| I-14 | B |
| I-15 | D |
| I-16 | A |
| I-17 | A |
| I-18 | A |
| I-19 | A |
| I-20 | A |
| I-21 | D |
| I-23 | D |
| I-24 | A |
| I-25 | A |
| I-26 | A |
| I-27 | A |
| I-28 | A |
| I-29 | B |
| I-30 | B |
| I-31 | B |
| I-32 | B |
| I-33 | B |
| I-34 | B |
| I-35 | D |
| I-36 | B |
| I-37 | B |
| I-38 | B |
| I-39 | C |
| I-40 | C |
| I-41 | B |
| II-1 | A |
| II-2 | A |

For evaluating the CDK inhibitory activity of the present application, the following ranges for the IC₅₀ [nM] were applied:
A: IC₅₀≤100 nM;
B: 100 nM<IC₅₀≤500 nM;
C: 500 nM<IC₅₀≤1000 nM;
D: 1000 nM<IC₅₀.

### Example 3 Inhibition of Cell Proliferation

Exemplary compounds of the invention were tested at different concentrations (from 10 μM to 610 pM; 4-fold serial dilutions) for their ability to inhibit the proliferation of various cancer cell lines. Known kinase inhibitor staurosporine were used as positive controls. Cells were grown in the indicated media listed below. All cell lines were supplemented with FBS (Life Technologies) and 100 U-mL⁻¹ penicillin, 100 μg·mL⁻¹ streptomycin (Invitrogen) and cultured at 37° C. in a humidified chamber in the presence of 5% CO₂. Proliferation assays were conducted over a 120 hour time period. CyQuant assay kit (ThermoFisher) was used to assess the anti-proliferative effects of the compounds following manufacturer's directions.

The following cancer cell lines may be tested with the exemplary media conditions indicated:

### Breast Cancer Cell Lines

HCC70: RPMI1640+10% FBS+1% Glutamax

### Ovarian Cancer Cell Line

OVCAR3: RPMI 1640+0.01 mg/mi bovine insulin+20% FBS+1% Glutamax

### Prostate Cancer Cell Line

DU145: EMEM+10% FBS+1% Glutamax

### Colon Cancer Cell Line

HCT-116: McCoy'5A+10% FBS+1% Glutamax

Exemplary results of these assays are set forth in Tables 3, where “A” represents an IC₅₀ value of less than 0.2 μM; “B” represents an IC₅₀ value of between 0.2 μM and 5 μM, inclusive; and “C” represents an IC₅₀ value of greater than 5 μM. “NT” represents that the specified compound was not tested in the specified assay.

TABLE 3
IC₅₀ Value of Inhibition of cell proliferation

| Compound | HCC70 | OVCAR3 |
|---|---|---|
| I-10 | B | B |
| I-12 | A | A |
| I-16 | A | A |
| I-19 | A | A |
| I-2 | B | B |
| I-3 | B | B |
| I-4 | A | A |
| I-6 | A | A |
| I-7 | B | B |
| I-8 | B | A |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A compound having the structure of formula (I) 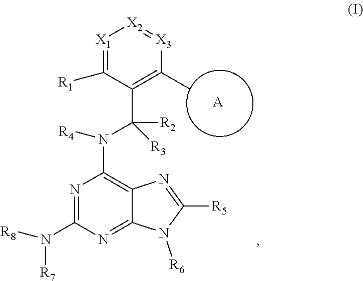
or a pharmaceutically acceptable salt, or prodrug thereof, or a solvate or hydrate of any of the forgoing,
wherein, each X₁, X₂, and X₃ is independently selected from optionally substituted —CH═, and —N═, each R₁, R₂, R₃, R₄, R₅, R₆, R₇, and R₈, is independently absent or is independently selected from optional substituents, or R₇ and R₈ combined with the atoms to which they are attached form an optionally substituted ring,
wherein, ring A is optionally substituted 5-ring-membered heteroaryl containing one or more N(nitrogen).

2. The compound of claim 1, said X₁ is optionally substituted —CH═, said X₂ is optionally substituted —CH, and said X₃ is optionally substituted —CH═ or —N═.

3-4. (canceled)

5. The compound of claim 1, said R₁ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl-;
said R₂ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl;
said R₃ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl;
said R₄ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl;
said R₅ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl;
said R₆ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl;
said R₇ is selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl;
and said R₈ is H;
or said R₇ and said R₈ combined with the atoms to which they are attached form an optionally substituted ring B, said ring B is selected from the group consisting of optionally substituted 3 to 12-ring-membered heterocycle, and optionally substituted 5 to 12-ring-membered heteroaryl.

6. The compound of claim 1, said R₁ is selected from the group consisting of hydrogen, protium, deuterium, tritium, and halogen, said R₂ is H, said R₃ is H, said R₄ is H, said R₅ is H, said R₆ is selected from the group consisting of hydrogen, protium, deuterium, tritium, optionally substituted (C₁-C₆)alkyl, and 3 to 12-ring-membered carbocycle, said R₇ is selected from the group consisting of hydrogen, protium, deuterium, tritium, optionally substituted (C₁-C₆)acyl, optionally substituted (C₁-C₆)alkyl, optionally substituted 3 to 12-ring-membered carbocycle, and optionally substituted 3 to 12-ring-membered heterocycle.

7. The compound of claim 1, said R₁ is H(hydrogen), F(fluorine) or Cl (chlorine).

8-17. (canceled)

18. The compound of claim 1, said R₆ is optionally substituted (C₁-C₆)alkyl or optionally substituted 3 to 12-ring-membered carbocycle.

19. The compound of claim 1, said R₆ is optionally substituted ethyl or optionally substituted propyl, or said R₆ is optionally substituted 5-ring-membered carbocycle.

20. The compound of claim 1, said R₆ is optionally substituted isopropyl or optionally substituted cyclopentanyl.

21-35. (canceled)

36. The compound of claim 1, said R₇ is substituted with one or more R₇₋₁, each said R₇₋₁ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

37-44. (canceled)

45. The compound of claim 36, said R₇₋₁ is substituted with one or more R₇₋₂, each said R₇₋₂ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

46-52. (canceled)

53. The compound of claim 5, said ring B is substituted with one or more R_{B-1}, each said R_{B-1} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

54-61. (canceled)

62. The compound of claim 53, said R_{B-1} is substituted with one or more R_{B-2}, each said R_{B-2} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

63-68. (canceled)

69. The compound of claim 62, said R_{B-2} is substituted with one or more R_{B-3}, each said R_{B-3} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

70-72. (canceled)

73. The compound of claim 69, said R_{B-3} is substituted with one or more R_{B-4}, each said R_{B-4} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

74-77. (canceled)

78. The compound of claim 1, said ring A is substituted with one or more R_{A-1}, each said R_{A-1} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

79-87. (canceled)

88. The compound of claim 78, said R_{A-1} is substituted with one or more R_{A-2}, each said R_{A-2} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

89-95. (canceled)

96. The compound of claim 88, said R_{A-2} is substituted with one or more R_{A-3}, each said R_{A-3} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

97-99. (canceled)

100. The compound of claim 96, said R_{A-3} is substituted with one or more R_{A-4}, each said R_{A-4} is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted 3 to 12-ring-membered carbocycle, optionally substituted 3 to 12-ring-membered heterocycle, optionally substituted 5 to 12-ring-membered aryl, and optionally substituted 5 to 12-ring-membered heteroaryl.

101. (canceled)

102. A compound or a pharmaceutically acceptable salt, prodrug, or metabolite thereof, or a solvate or hydrate of any of the foregoing, wherein, said compound is selected from the group consisting of

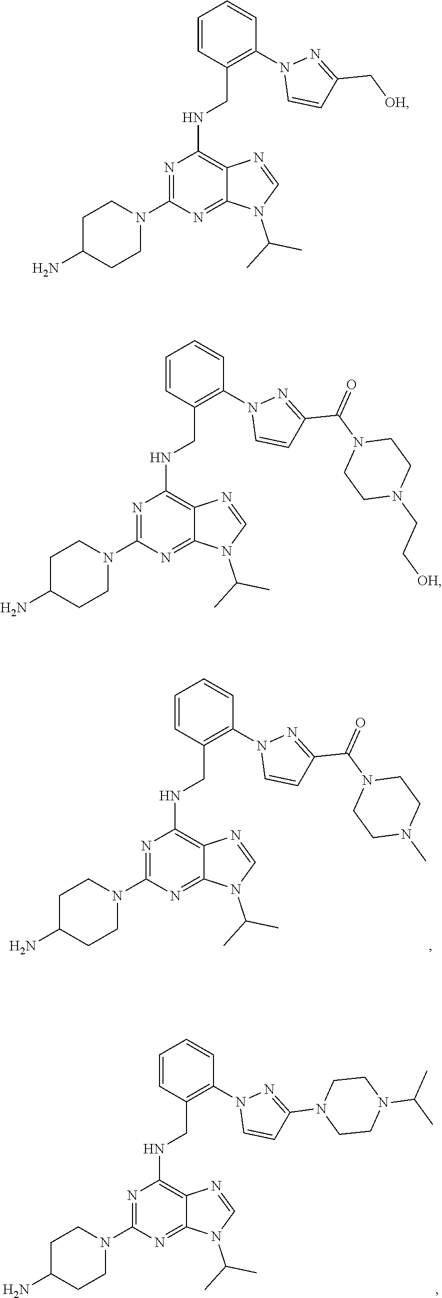 [SMILES: CC(C)n1cnc2c(NCc3ccccc3-n3ccc(CO)n3)nc(N3CCC(N)CC3)nc21 ; CC(C)n1cnc2c(NCc3ccccc3-n3ccc(C(=O)N4CCN(CCO)CC4)n3)nc(N3CCC(N)CC3)nc21 ; CC(C)n1cnc2c(NCc3ccccc3-n3ccc(C(=O)N4CCN(C)CC4)n3)nc(N3CCC(N)CC3)nc21 ; CC(C)N1CCN(c2ccn(-c3ccccc3CNc3nc(N4CCC(N)CC4)nc4c3ncn4C(C)C)n2)CC1]

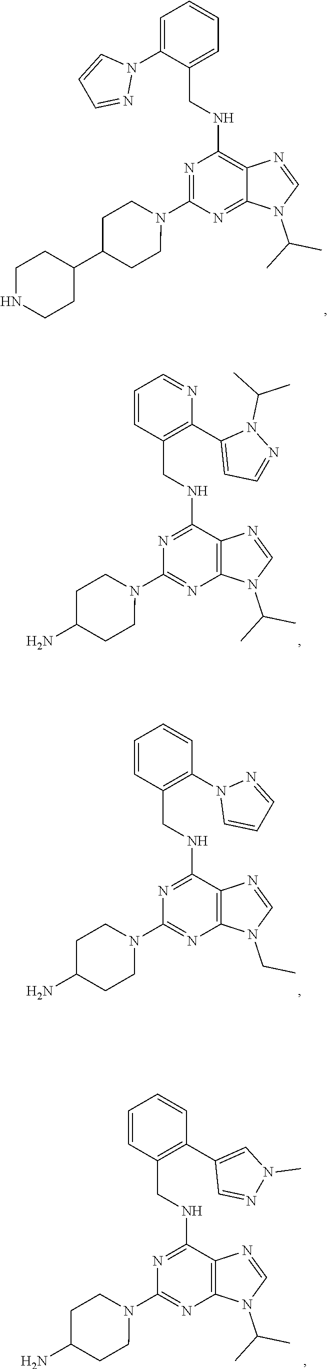 [SMILES: CC(C)n1cnc2c(NCc3ccccc3-n3cccn3)nc(N3CCC(C4CCNCC4)CC3)nc21 ; CC(C)n1nccc1-c1ncccc1CNc1nc(N2CCC(N)CC2)nc2c1ncn2C(C)C ; CCn1cnc2c(NCc3ccccc3-n3cccn3)nc(N3CCC(N)CC3)nc21 ; CC(C)n1cnc2c(NCc3ccccc3-c3cnn(C)c3)nc(N3CCC(N)CC3)nc21]

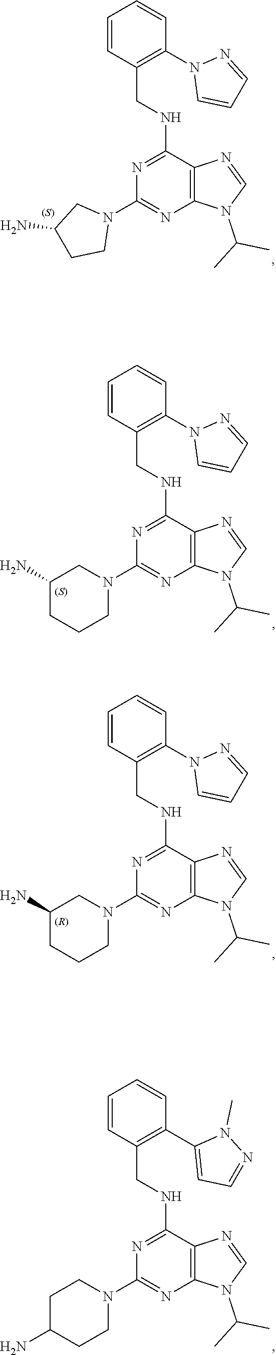

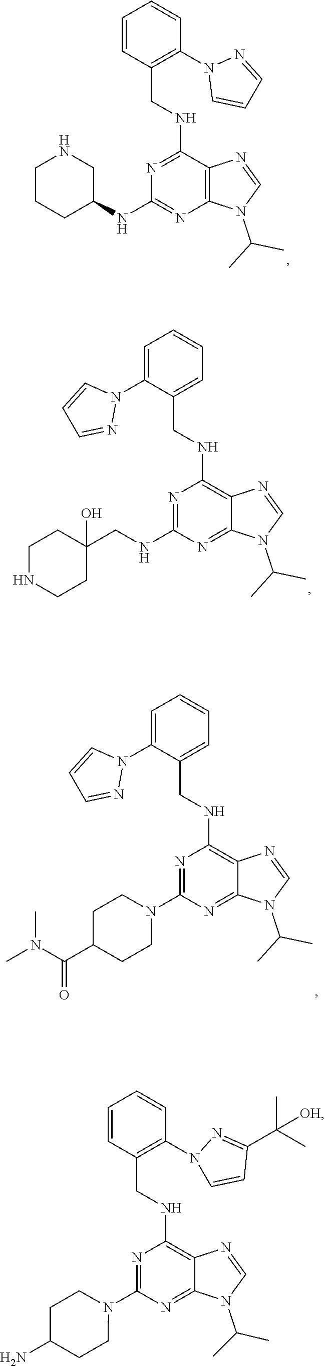

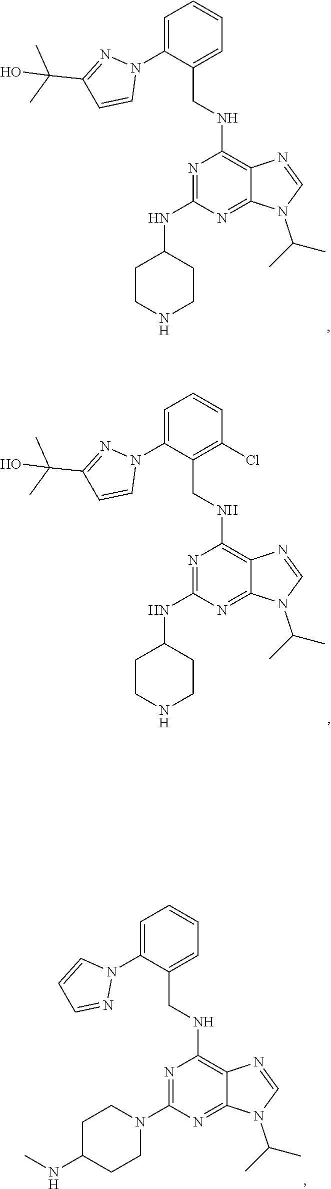

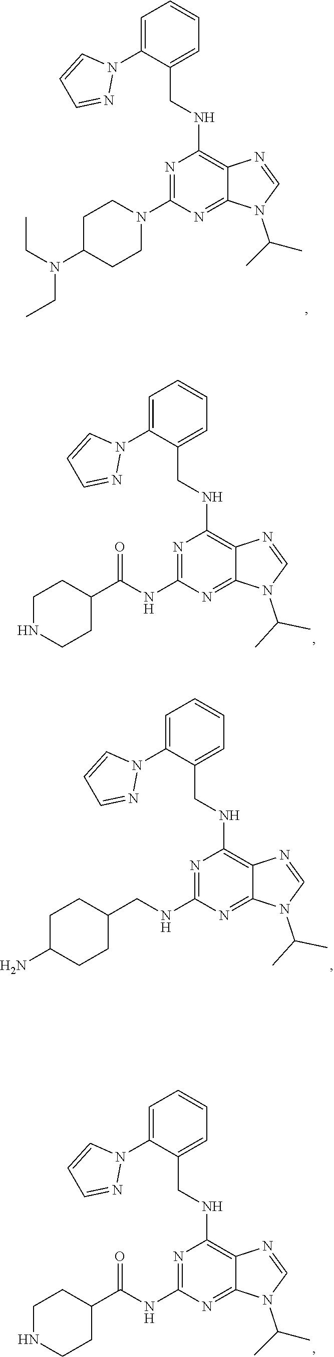 [SMILES: CCN(CC)C1CCN(c2nc(NCc3ccccc3-n3cccn3)c3ncn(C(C)C)c3n2)CC1 ; CC(C)n1cnc2c(NCc3ccccc3-n3cccn3)nc(NC(=O)C3CCNCC3)nc21 ; CC(C)n1cnc2c(NCc3ccccc3-n3cccn3)nc(NCC3CCC(N)CC3)nc21 ; CC(C)n1cnc2c(NCc3ccccc3-n3cccn3)nc(NC(=O)C3CCNCC3)nc21]

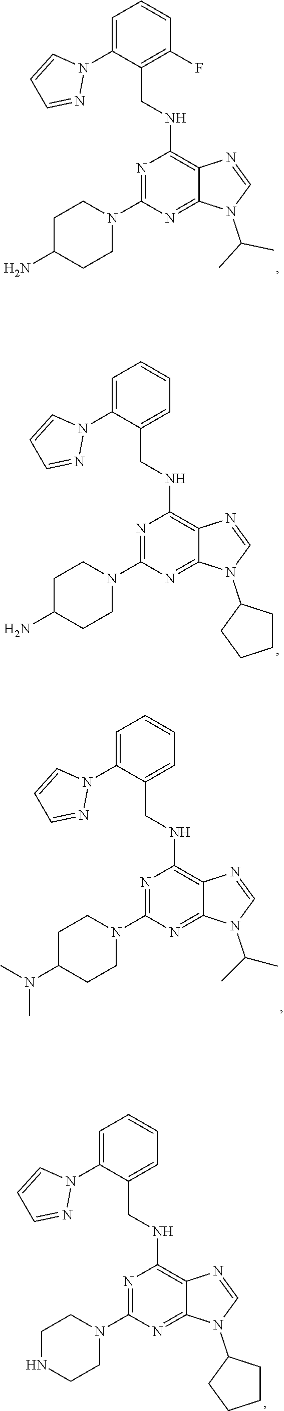

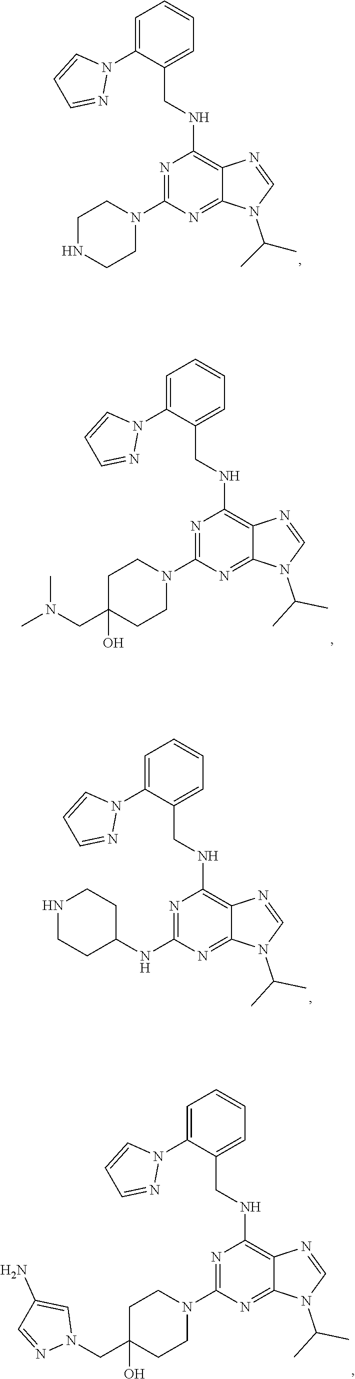

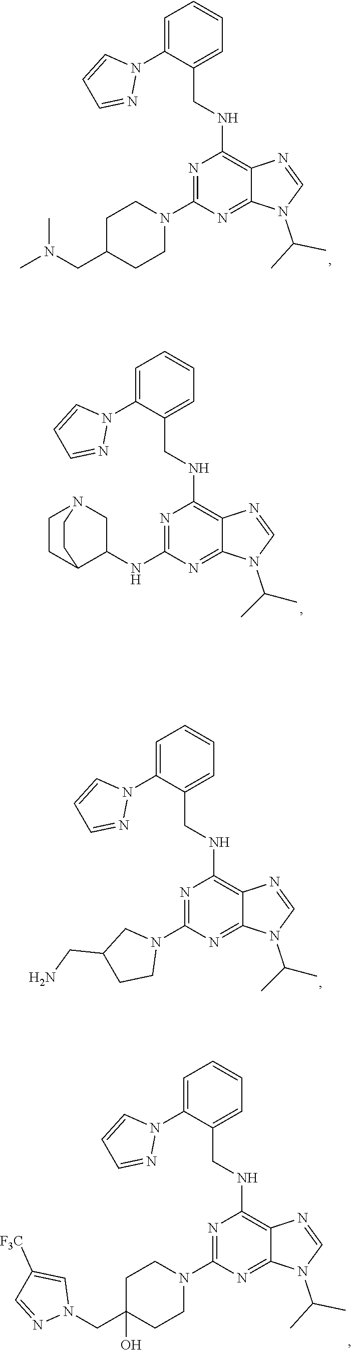 [SMILES: CC(C)n1cnc2c(NCc3ccccc3-n3cccn3)nc(N3CCC(CN(C)C)CC3)nc21 ; CC(C)n1cnc2c(NCc3ccccc3-n3cccn3)nc(NC3CN4CCC3CC4)nc21 ; CC(C)n1cnc2c(NCc3ccccc3-n3cccn3)nc(N3CCC(CN)C3)nc21 ; CC(C)n1cnc2c(NCc3ccccc3-n3cccn3)nc(N3CCC(O)(Cn4cc(C(F)(F)F)cn4)CC3)nc21]

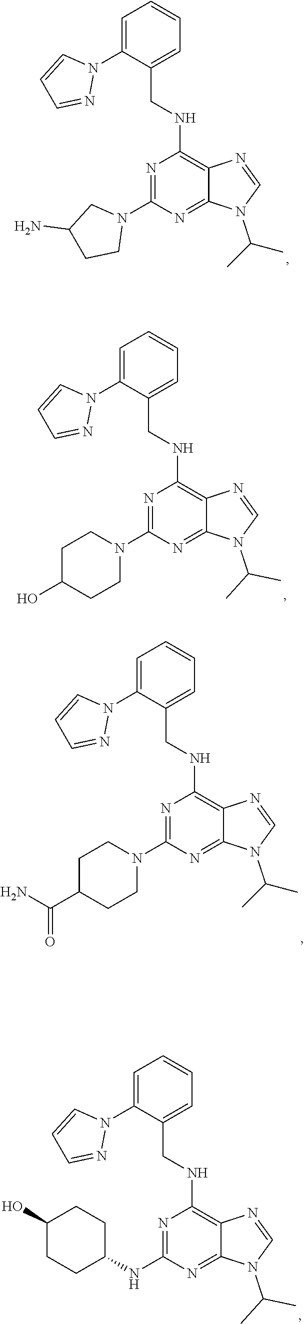 [SMILES: CC(C)n1cnc2c(NCc3ccccc3-n3cccn3)nc(N3CCC(N)C3)nc21 ; CC(C)n1cnc2c(NCc3ccccc3-n3cccn3)nc(N3CCC(O)CC3)nc21 ; CC(C)n1cnc2c(NCc3ccccc3-n3cccn3)nc(N3CCC(C(N)=O)CC3)nc21 ; CC(C)n1cnc2c(NCc3ccccc3-n3cccn3)nc(N[C@H]3CC[C@H](O)CC3)nc21]

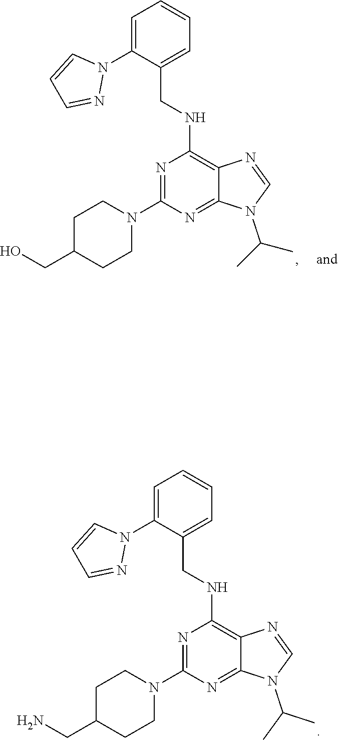 [SMILES: CC(C)n1cnc2c(NCc3ccccc3-n3cccn3)nc(N3CCC(CO)CC3)nc21 ; CC(C)n1cnc2c(NCc3ccccc3-n3cccn3)nc(N3CCC(CN)CC3)nc21]

103-257. (canceled)

258. A composition comprising a compound of claim 1, or a pharmaceutically acceptable salt, prodrug, or metabolite thereof, or a solvate or hydrate of any of the foregoing, and optionally a pharmaceutically acceptable carrier.

259. A method for inhibiting cyclin-dependent kinase (CDK) activity, said method comprising administering to a subject in need thereof an effective amount of the compound of claim 1, or a pharmaceutically acceptable salt, prodrug, or metabolite thereof, or a solvate or hydrate of any of the foregoing.

260-261. (canceled)


SEQUENCE LISTING (2 sequences)

SEQ ID NO: 1 - protein, 26 aa, synthetic construct
  REGION 1..26: note = CDKs peptide 1
YSPSTSPSYSTPSPSYSPTSPSKKKK

SEQ ID NO: 2 - protein, 7 aa, synthetic construct
  REGION 1..7: note = CDKs peptide 2
GSRTPMY